(19) United States
(12) Patent Application Publication
(10) Pub. No.: US 2024/0398799 A1
(43) Pub. Date: Dec. 5, 2024

(54) COMPOSITIONS AND METHODS FOR GENERATING SYNTHETIC LETHALITY IN TUMORS

(71) Applicant: Engine Biosciences Pte. Ltd., Singapore (SG)

(72) Inventors: Stephen David HARRISON, Sandy (GB); Michael David WINTHER, Singapore (SG); Yuanyuan XIAO, Los Altos, CA (US); Shawn YOST, Los Angeles, CA (US); Christine Taylor BREW, Montara, CA (US); Yaron TURPAZ, Singapore (SG)

(21) Appl. No.: 18/684,640

(22) PCT Filed: Aug. 17, 2022
(86) PCT No.: PCT/SG2022/050584

§ 371 Date: Feb. 16, 2024

Related U.S. Application Data
(60) Provisional application No. 63/237,031, filed on Aug. 25, 2021.
(60) Provisional application No. 63/237,040, filed on Aug. 25, 2021.
(60) Provisional application No. 63/237,052, filed on Aug. 25, 2021.
(60) Provisional application No. 63/237,063, filed on Aug. 25, 2021.
(60) Provisional application No. 63/237,066, filed on Aug. 25, 2021.
(60) Provisional application No. 63/237,060, filed on Aug. 25, 2021.
(60) Provisional application No. 63/237,060, filed on Aug. 25, 2021.

(30) Foreign Application Priority Data
Aug. 19, 2021 (SG) 10202109058V

(51) Int. Cl. A61K 31/506; A61K 31/277; A61K 31/4706; A61K 31/496; A61K 31/517; A61K 31/519; A61K 31/5377; A61K 31/7088; A61K 38/46; C07K 16/40; C12N 15/113; C12N 15/115; C12Q 1/6886

(52) U.S. Cl. CPC A61K 31/506; A61K 31/277; A61K 31/4706; A61K 31/496; A61K 31/517; A61K 31/519; A61K 31/5377; A61K 31/7088; A61K 38/465; C07K 16/40; C12N 15/1137; C12N 15/115; C12Q 1/6886; C12N 2310/14; C12N 2310/16; C12Q 2600/156; C12Q 2600/158

(57) ABSTRACT

The present disclosure provides methods for treating cancer comprising administering one or more therapeutic agents for manipulation of a target gene (e.g., protein kinase, membrane associated tyrosine/threonine 1 (PKMYT1)), wherein the cancer has a mutation in, an altered (e.g., increased or decreased) expression level and/or an altered activity of a biomarker. Provided herein are methods for identifying biomarkers of the disclosure that form a synthetic lethal pair with a target gene (e.g., PKMYT1).

### CROSS REFERENCE TO RELATED APPLICATIONS

This application claims the benefit of Singapore Provisional Application Serial No. 10202109058V filed Aug. 19, 2021, U.S. Provisional Application Ser. No. 63/237,052 filed Aug. 25, 2021, U.S. Provisional Application Ser. No. 63/237,031 filed Aug. 25, 2021, U.S. Provisional Application Ser. No. 63/237,060 filed Aug. 25, 2021, U.S. Provisional Application Ser. No. 63/237,063 filed Aug. 25, 2021, U.S. Provisional Application Ser. No. 63/237,066 filed Aug. 25, 2021, U.S. Provisional Application Ser. No. 63/237,040 filed Aug. 25, 2021, the entire contents of which are incorporated herein by reference.

### BACKGROUND

Despite advances, targeted cancer therapy has largely failed to produce durable complete responses and cures in large numbers of patients with cancer. Additionally, systemic treatments such as chemotherapies are often toxic and cause undesirable side effects for patients. The lack of specific biomarkers has also complicated development and application of targeted cancer treatments.

One approach for treating cancer cells includes identifying target genes and biomarkers which identify which cancer cells may be sensitive to alteration in the activity of those target genes. Recent advances in functional genomic screening have enabled identification of such target genes and biomarkers. However, for many human cancers, there remains limited suitable biomarkers that indicate the cancer will respond to a targeted cancer therapy.

### SUMMARY

In some aspects, the present disclosure provides a method for treating a subject having or suspected of having a cancer, comprising administering to the subject a therapeutically effective amount of a Protein Kinase Membrane Associated Tyrosine/Threonine 1 (PKMYT1)-targeting therapeutic agent that alters the expression and/or activity of PKMYT1 in the subject, wherein the cancer is associated with cancerous tissue comprising a cell or a plurality of cells comprising (i) a difference in expression or activity level of one or more genes compared to a healthy control, and/or (ii) a mutation or deletion in one or more genes as compared to a healthy control, wherein the one or more genes is in a list selected by computational inference of cancer cell susceptibility to decrease in activity of PKMYT1, thereby treating the cancer in the subject.

In some or any of the foregoing or related embodiments, the one or more genes is validated as a synthetic lethal pair with PKMYT1 using a method described herein. In some embodiments, the method comprises a gene knockout screen. In some embodiments, the method comprises a combinatorial genetics en masse (CombiGEM) screen described herein. In some embodiments, the CombiGEM screen comprises measuring growth of a cancer cell line comprising a Cas nuclease and a dual guide RNA (gRNA) combination that induces a double knockout of PKMYT1 and the one or more genes, wherein the dual gRNA combination comprises a gRNA (e.g., a single gRNA (sgRNA)) targeting PKMYT1 and one or more gRNAs (e.g., sgRNAs) targeting the one or more genes. In some embodiments, a double knockout of PKMYT1 and the one or more genes that results in decreased growth of cancer cells as compared to a single gene knockout of PKMYT1 or the one or more genes is used to identify a synthetic lethal pair with PKMYT1.

In some or any of the foregoing or related embodiments, the cancerous tissue comprises a cell or a plurality of cells comprising a difference in expression or activity level of one or more genes compared to a healthy control. In some embodiments, the cancerous tissue comprises a cell or a plurality of cells comprising a mutation and/or deletion in one or more genes as compared to a healthy control. In some aspect, the cancerous tissue comprises a cell or a plurality of cells comprising a difference in expression or activity level of one or more genes compared to a healthy control and a mutation and/or deletion in the one or more genes as compared to a healthy control. In some embodiments, the difference in expression or activity level is a decrease in expression or activity level of the one or more genes compared to a healthy control. In some embodiments, the mutation is a loss of function mutation. In some embodiments, the presence or absence of the mutation and/or deletion is identified by an assay of cells derived from a cancerous tissue sample obtained from the subject. In some embodiments, the assay is a next generation sequencing-based assay or oligomer hybridization.

In some or any of the foregoing or related embodiments, the one or more genes are selected using a predictive algorithm, a machine learning algorithm, or both. In some embodiments, the difference in expression or activity level of the one or more genes has a prevalence of about 5% or higher in at least one cancer. In some embodiments, the mutation or deletion in the one or more genes has a prevalence of about 5% or higher in at least one cancer. In some embodiments, the difference in expression or activity level of the one or more genes has a prevalence of about 3% or higher in at least one cancer. In some embodiments, the mutation or deletion in the one or more genes has a prevalence of about 3% or higher in at least one cancer. In some embodiments, the difference in expression or activity level of the one or more genes has a prevalence of about 1% or higher in at least one cancer. In some embodiments, the mutation or deletion in the one or more genes has a prevalence of about 1% or higher in at least one cancer. In some embodiments, the cancer is selected from a cancer type listed in the Cancer Genome Atlas (TCGA). In some embodiments, the cancer is selected from a leukemia, lymphoma, and myeloma. In some embodiments, the cancer is a solid tumor malignancy of the prostate, uterus, colon, rectum, liver, bladder, ovaries, lung, breast, skin, stomach, esophagus, cervix, pancreas, testes, eye, mucosal tissue, adrenal gland, brain, thyroid, or thymus.

In some or any of the foregoing or related embodiments, the one or more genes is selected from any one or any combination (e.g., 2, 3, 4, 5 or more) of biomarkers listed in Table 3. In some embodiments, the one or more gene is selected from any one or any combination (e.g., 2, 3, 4, 5 or more) of biomarkers listed in Table 8 and 9. In some embodiments, the one or more gene is selected from any one or any combination (e.g., 2, 3, 4, 5 or more) of biomarkers listed in Table 10.

In some aspects, the disclosure provides a method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 3.

In some aspects, the disclosure provides a method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 8 and 9.

In some aspects, the disclosure provides a method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 10.

In some aspects, the disclosure provides a method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 3, wherein the diseased tissue sample comprises (a) a decreased expression level and/or decreased activity in the one or more biomarkers relative to a reference tissue sample; and/or (b) a loss-of-function mutation in the one or more biomarkers relative to a reference tissue sample.

In some aspects, the disclosure provides a method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 8 and 9, wherein the diseased tissue sample comprises (a) a decreased expression level and/or decreased activity in the one or more biomarkers relative to a reference tissue sample; and/or (b) a loss-of-function mutation in the one or more biomarkers relative to a reference tissue sample.

In some aspects, the disclosure provides a method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 10, wherein the diseased tissue sample comprises (a) a decreased expression level and/or decreased activity in the one or more biomarkers relative to a reference tissue sample; and/or (b) a loss-of-function mutation in the one or more biomarkers relative to a reference tissue sample.

In some aspects, the disclosure provides a method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.

In some aspects, the disclosure provides a method of identifying a subject having a disease or disorder for treatment with one or more PKMYT1 therapeutic agents, the method comprising determining the expression level and/or activity of one or more biomarkers in a diseased tissue sample obtained from the subject, wherein the one or more biomarkers is selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.

In some aspects, the disclosure provides a method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some aspects, the disclosure provides a method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some aspects, the disclosure provides a method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some aspects, the disclosure provides a method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some aspects, the disclosure provides a method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 1.

In some aspects, the disclosure provides a method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.

In some aspects, the disclosure provides a method of identifying a subject having a disease or disorder for treatment with one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 3.

In some aspects, the disclosure provides a method of identifying a subject having a disease or disorder for treatment with one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 8 and 9.

In some aspects, the disclosure provides a method of identifying a subject having a disease or disorder for treatment with one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 10.

In some aspects, the disclosure provides a method of identifying a subject having a disease or disorder for treatment with one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 3, wherein the diseased tissue sample comprises a loss-of-function mutation in the one or more biomarkers relative to a reference tissue sample.

In some aspects, the disclosure provides a method of identifying a subject having a disease or disorder for treatment with one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 8 and 9, wherein the diseased tissue sample comprises a loss-of-function mutation in the one or more biomarkers relative to a reference tissue sample.

In some aspects, the disclosure provides a method of identifying a subject having a disease or disorder for treatment with one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 10, wherein the diseased tissue sample comprises a loss-of-function mutation in the one or more biomarkers relative to a reference tissue sample.

In some aspects, the disclosure provides a method of identifying a subject having a disease or disorder for treatment with one or more PKMYT1 therapeutic agents, the method comprising determining the presence of a mutation in one or more biomarkers in a diseased tissue sample obtained from the subject, wherein the one or more biomarkers is selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.

In some aspects, the disclosure provides a method of identifying a subject having a disease or disorder for treatment with one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some aspects, the disclosure provides a method of identifying a subject having a disease or disorder for treatment with one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some aspects, the disclosure provides a method of identifying a subject having a disease or disorder for treatment with one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some aspects, the disclosure provides a method of identifying a subject having a disease or disorder for treatment with one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some aspects, the disclosure provides a method of identifying a subject having a disease or disorder for treatment with one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 1.

In some aspects, the disclosure provides a method of identifying a subject having a disease or disorder for treatment with one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.

In some aspects, the disclosure provides a method of identifying a subject having a disease or disorder for treatment with one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 3.

In some aspects, the disclosure provides a method of identifying a subject having a disease or disorder for treatment with one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 8 and 9.

In some aspects, the disclosure provides a method of identifying a subject having a disease or disorder for treatment with one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 10.

In some aspects, the disclosure provides a method of identifying a subject having a disease or disorder for treatment with one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 3, wherein the diseased tissue sample comprises a decreased expression level of the one or more biomarkers relative to a reference tissue sample.

In some aspects, the disclosure provides a method of identifying a subject having a disease or disorder for treatment with one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 8 and 9, wherein the diseased tissue sample comprises a decreased expression level of the one or more biomarkers relative to a reference tissue sample.

In some aspects, the disclosure provides a method of identifying a subject having a disease or disorder for treatment with one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 10, wherein the diseased tissue sample comprises a decreased expression level of the one or more biomarkers relative to a reference tissue sample.

In some aspects, the disclosure provides a method of identifying a subject having a disease or disorder for treatment with one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.

In some aspects, the disclosure provides a method of identifying a subject having a disease or disorder for treatment with one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some aspects, the disclosure provides a method of identifying a subject having a disease or disorder for treatment with one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some aspects, the disclosure provides a method of identifying a subject having a disease or disorder for treatment with one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some aspects, the disclosure provides a method of identifying a subject having a disease or disorder for treatment with one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some aspects, the disclosure provides a method of identifying a subject having a disease or disorder for treatment with one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 1.

In some aspects, the disclosure provides a method of identifying a subject having a disease or disorder for treatment with one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.

In some aspects, the disclosure provides a method of identifying a subject having a disease or disorder for treatment with one or more PKMYT1 therapeutic agents, the method comprising determining the activity level of one or more biomarkers in a diseased tissue sample obtained from the subject, wherein the one or more biomarkers is selected from any one or any combination (e.g., 2, 3, 4, 5 or more) of biomarkers listed in Table 3.

In some aspects, the disclosure provides a method of identifying a subject having a disease or disorder for treatment with one or more PKMYT1 therapeutic agents, the method comprising determining the activity level of one or more biomarkers in a diseased tissue sample obtained from the subject, wherein the one or more biomarkers is selected from any one or any combination (e.g., 2, 3, 4, 5 or more) of biomarkers listed in Table 8 and 9.

In some aspects, the disclosure provides a method of identifying a subject having a disease or disorder for treatment with one or more PKMYT1 therapeutic agents, the method comprising determining the activity level of one or more biomarkers in a diseased tissue sample obtained from the subject, wherein the one or more biomarkers is selected from any one or any combination (e.g., 2, 3, 4, 5 or more) of biomarkers listed in Table 10.

In some aspects, the disclosure provides a method of identifying a subject having a disease or disorder for treatment with one or more PKMYT1 therapeutic agents, the method comprising determining the activity level of one or more biomarkers in a diseased tissue sample obtained from the subject, wherein the one or more biomarkers is selected from any one or any combination (e.g., 2, 3, 4, 5 or more) of biomarkers listed in Table 3, wherein the diseased tissue sample comprises a decreased activity level of the one or more biomarkers relative to a reference tissue sample.

In some aspects, the disclosure provides a method of identifying a subject having a disease or disorder for treatment with one or more PKMYT1 therapeutic agents, the method comprising determining the activity level of one or more biomarkers in a diseased tissue sample obtained from the subject, wherein the one or more biomarkers is selected from any one or any combination (e.g., 2, 3, 4, 5 or more) of biomarkers listed in Table 8 and 9, wherein the diseased tissue sample comprises a decreased activity level of the one or more biomarkers relative to a reference tissue sample.

In some aspects, the disclosure provides a method of identifying a subject having a disease or disorder for treatment with one or more PKMYT1 therapeutic agents, the method comprising determining the activity level of one or more biomarkers in a diseased tissue sample obtained from the subject, wherein the one or more biomarkers is selected from any one or any combination (e.g., 2, 3, 4, 5 or more) of biomarkers listed in Table 10, wherein the diseased tissue sample comprises a decreased activity level of the one or more biomarkers relative to a reference tissue sample.

In some aspects, the disclosure provides a method of identifying a subject having a disease or disorder for treatment with one or more PKMYT1 therapeutic agents, the method comprising determining the activity level of one or more biomarkers in a diseased tissue sample obtained from the subject, wherein the one or more biomarkers is selected from any one or any combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.

In some aspects, the disclosure provides a method of identifying a subject having a disease or disorder for treatment with one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some aspects, the disclosure provides a method of identifying a subject having a disease or disorder for treatment with one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some aspects, the disclosure provides a method of identifying a subject having a disease or disorder for treatment with one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some aspects, the disclosure provides a method of identifying a subject having a disease or disorder for treatment with one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some aspects, the disclosure provides a method of identifying a subject having a disease or disorder for treatment with one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of biomarkers listed in Table 1.

In some aspects, the disclosure provides a method of identifying a subject having a disease or disorder for treatment with one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.

In some aspects, the disclosure provides a method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 3.

In some aspects, the disclosure provides a method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 8 and 9.

In some aspects, the disclosure provides a method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 10.

In some aspects, the disclosure provides a method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 3, wherein the diseased tissue sample comprises (a) a decreased expression level and/or decreased activity in the one or more biomarkers relative to a reference tissue sample; and/or (b) a loss-of-function mutation in the one or more biomarkers relative to a reference tissue sample.

In some aspects, the disclosure provides a method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 8 and 9, wherein the diseased tissue sample comprises (a) a decreased expression level and/or decreased activity in the one or more biomarkers relative to a reference tissue sample; and/or (b) a loss-of-function mutation in the one or more biomarkers relative to a reference tissue sample.

In some aspects, the disclosure provides a method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 10, wherein the diseased tissue sample comprises (a) a decreased expression level and/or decreased activity in the one or more biomarkers relative to a reference tissue sample; and/or (b) a loss-of-function mutation in the one or more biomarkers relative to a reference tissue sample.

In some aspects, the disclosure provides a method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.

In some aspects, the disclosure provides a method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some aspects, the disclosure provides a method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some aspects, the disclosure provides a method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some aspects, the disclosure provides a method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some aspects, the disclosure provides a method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of biomarkers listed in Table 1.

In some aspects, the disclosure provides a method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.

In some aspects, the disclosure provides a method of determining responsiveness of a subject having a disease or disorder to one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 3.

In some aspects, the disclosure provides a method of determining responsiveness of a subject having a disease or disorder to one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 8 and 9.

In some aspects, the disclosure provides a method of determining responsiveness of a subject having a disease or disorder to one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 10.

In some aspects, the disclosure provides a method of determining responsiveness of a subject having a disease or disorder to one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 3, wherein the diseased tissue sample comprises a loss-of-function mutation in the one or more biomarkers relative to a reference tissue sample.

In some aspects, the disclosure provides a method of determining responsiveness of a subject having a disease or disorder to one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 8 and 9, wherein the diseased tissue sample comprises a loss-of-function mutation in the one or more biomarkers relative to a reference tissue sample.

In some aspects, the disclosure provides a method of determining responsiveness of a subject having a disease or disorder to one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 10, wherein the diseased tissue sample comprises a loss-of-function mutation in the one or more biomarkers relative to a reference tissue sample.

In some aspects, the disclosure provides a method of determining responsiveness of a subject having a disease or disorder to one or more PKMYT1 therapeutic agents, the method comprising determining the presence of a mutation in one or more biomarkers in a diseased tissue sample obtained from the subject, wherein the one or more biomarkers is selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.

In some aspects, the disclosure provides a method of determining responsiveness of a subject having a disease or disorder to one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some aspects, the disclosure provides a method of determining responsiveness of a subject having a disease or disorder to one or more PKMYT1 therapeutic agents, the method comprising determining the presence of a mutation in one or more biomarkers in a diseased tissue sample obtained from the subject, wherein the one or more biomarkers is selected from any one or any combination of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some aspects, the disclosure provides a method of determining responsiveness of a subject having a disease or disorder to one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some aspects, the disclosure provides a method of determining responsiveness of a subject having a disease or disorder to one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some aspects, the disclosure provides a method of determining responsiveness of a subject having a disease or disorder to one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of biomarkers listed in Table 1.

In some aspects, the disclosure provides a method of determining responsiveness of a subject having a disease or disorder to one or more PKMYT1 therapeutic agents, the method comprising determining the presence of a mutation in one or more biomarkers in a diseased tissue sample obtained from the subject, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.

In some aspects, the disclosure provides a method of determining responsiveness of a subject having a disease or disorder to one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 3.

In some aspects, the disclosure provides a method of determining responsiveness of a subject having a disease or disorder to one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 8 and 9.

In some aspects, the disclosure provides a method of determining responsiveness of a subject having a disease or disorder to one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 10.

In some aspects, the disclosure provides a method of determining responsiveness of a subject having a disease or disorder to one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 3, wherein the diseased tissue sample comprises a decreased expression level of the one or more biomarkers relative to a reference tissue sample.

In some aspects, the disclosure provides a method of determining responsiveness of a subject having a disease or disorder to one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 8 and 9, wherein the diseased tissue sample comprises a decreased expression level of the one or more biomarkers relative to a reference tissue sample.

In some aspects, the disclosure provides a method of determining responsiveness of a subject having a disease or disorder to one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 10, wherein the diseased tissue sample comprises a decreased expression level of the one or more biomarkers relative to a reference tissue sample.

In some aspects, the disclosure provides a method of determining responsiveness of a subject having a disease or disorder to one or more PKMYT1 therapeutic agents, the method comprising determining the expression level and/or activity of one or more biomarkers in a diseased tissue sample obtained from the subject, wherein the one or more biomarkers is selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.

In some aspects, the disclosure provides a method of determining responsiveness of a subject having a disease or disorder to one or more PKMYT1 therapeutic agents, the method comprising determining the expression level of one or more biomarkers in a diseased tissue sample obtained from the subject, wherein the one or more biomarkers is selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.

In some aspects, the disclosure provides a method of determining responsiveness of a subject having a disease or disorder to one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some aspects, the disclosure provides a method of determining responsiveness of a subject having a disease or disorder to one or more PKMYT1 therapeutic agents, the method comprising determining the expression level one or more biomarkers in a diseased tissue sample obtained from the subject, wherein the one or more biomarkers is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some aspects, the disclosure provides a method of determining responsiveness of a subject having a disease or disorder to one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some aspects, the disclosure provides a method of determining responsiveness of a subject having a disease or disorder to one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some aspects, the disclosure provides a method of determining responsiveness of a subject having a disease or disorder to one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of biomarkers listed in Table 1.

In some aspects, the disclosure provides a method of determining responsiveness of a subject having a disease or disorder to one or more PKMYT1 therapeutic agents, the method comprising determining the expression level of one or more biomarkers in a diseased tissue sample obtained from the subject, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.

In some aspects, the disclosure provides a method of determining responsiveness of a subject having a disease or disorder to one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of biomarkers listed in Table 3.

In some aspects, the disclosure provides a method of determining responsiveness of a subject having a disease or disorder to one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of biomarkers listed in Table 8 and 9.

In some aspects, the disclosure provides a method of determining responsiveness of a subject having a disease or disorder to one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of biomarkers listed in Table 10.

In some aspects, the disclosure provides a method of determining responsiveness of a subject having a disease or disorder to one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of biomarkers listed in Table 3, wherein the diseased tissue sample comprises a decreased activity of the one or more biomarkers relative to a reference tissue sample.

In some aspects, the disclosure provides a method of determining responsiveness of a subject having a disease or disorder to one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of biomarkers listed in Table 8 and 9, wherein the diseased tissue sample comprises a decreased activity of the one or more biomarkers relative to a reference tissue sample.

In some aspects, the disclosure provides a method of determining responsiveness of a subject having a disease or disorder to one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of biomarkers listed in Table 10, wherein the diseased tissue sample comprises a decreased activity of the one or more biomarkers relative to a reference tissue sample.

In some aspects, the disclosure provides a method of determining responsiveness of a subject having a disease or disorder to one or more PKMYT1 therapeutic agents, the method comprising determining the activity of one or more biomarkers in a diseased tissue sample obtained from the subject, wherein the one or more biomarkers is selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.

In some aspects, the disclosure provides a method of determining responsiveness of a subject having a disease or disorder to one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some aspects, the disclosure provides a method of determining responsiveness of a subject having a disease or disorder to one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some aspects, the disclosure provides a method of determining responsiveness of a subject having a disease or disorder to one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some aspects, the disclosure provides a method of determining responsiveness of a subject having a disease or disorder to one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some aspects, the disclosure provides a method of determining responsiveness of a subject having a disease or disorder to one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of biomarkers listed in Table 1.

In some aspects, the disclosure provides a method of determining responsiveness of a subject having a disease or disorder to one or more PKMYT1 therapeutic agents, the method comprising determining the activity of one or more biomarkers in a diseased tissue sample obtained from the subject, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.

In some embodiments, in any of the foregoing or related aspects, the diseased tissue sample comprises an altered expression level and/or activity of the one or more biomarkers relative to a reference tissue sample. In some aspects, the presence of an altered expression level and/or activity of the one or more biomarker is used to identify the subject for treatment with one or more PKMYT1 therapeutic agents. In some aspects, the presence of an altered expression level and/or activity of the one or more biomarker is used to determine the responsiveness of the subject to treatment with one or more PKMYT1 therapeutic agents. In some aspects, the presence of an altered expression level and/or activity of the one or more biomarker is used to determine the subject will respond or will likely respond to treatment with one or more PKMYT1 therapeutic agents.

In some embodiments, in any of the foregoing or related aspects, the expression level and/or activity of the one or more biomarkers is reduced relative to a reference tissue sample. In some aspects, the presence of a reduced expression level and/or activity of the one or more biomarker is used to identify the subject for treatment with one or more PKMYT1 therapeutic agents. In some aspects, the presence of a reduced expression level and/or activity of the one or more biomarker is used to determine the responsiveness of the subject to treatment with one or more PKMYT1 therapeutic agents. In some aspects, the presence of a reduced expression level and/or activity of the one or more biomarker is used to determine the subject will respond or will likely respond to treatment with one or more PKMYT1 therapeutic agents.

In some embodiments, in any of the foregoing or related aspects, the diseased tissue comprises a mutation in the one or more biomarkers. In some aspects, the mutation is a deletion. In some aspects, the mutation is a frameshift mutation. In some aspects, the biomarker has an open reading frame, wherein the frameshift mutation occurs at or near the 5′end of the open-reading frame of the biomarker. In some aspects, the mutation is detected by sequencing genomic DNA obtained from the diseased tissue sample. In some aspects, the sequencing comprises next generation sequencing. In some aspects, the presence of a mutation in the one or more biomarkers is used to identify the subject for treatment with one or more PKMYT1 therapeutic agents. In some aspects, the presence of a mutation in the one or more biomarkers is used to determine the responsiveness of the subject to treatment with one or more PKMYT1 therapeutic agents. In some aspects, the presence of a mutation in the one or more biomarkers is used to determine that the subject will respond or will likely respond to treatment with one or more PKMYT1 therapeutic agents.

In some embodiments, in any of the foregoing or related aspects, the diseased tissue sample comprises a mutation in the one or more biomarkers. In some aspects, the diseased tissue sample comprises a mutation in the one or more biomarkers relative to a reference tissue sample (e.g., healthy control tissue sample). In some embodiments, the mutation is a loss of function mutation. In some embodiments, the loss of function mutation results in the biomarker having reduced expression and/or activity. In some embodiments, the loss of function mutation abolishes expression and/or activity of the biomarker. In some embodiments, the loss of function mutation results in an inactivated or nonfunctional translational product. In some embodiments, the loss of function mutation is a deletion of the gene encoding the biomarker. In some embodiments, the mutation occurs in one or both gene alleles encoding the biomarker. In some embodiments, the mutation is a nonsynonymous mutation (e.g., a missense mutation, a nonstop mutation, a nonsense mutation). In some embodiments, the mutation (e.g., nonsynonymous mutation) is an insertion or deletion. In some embodiments, the insertion or deletion introduces a frameshift mutation (e.g., a frameshift mutation in the gene encoding the biomarker that results in an inactivated or nonfunctional translational product). In some embodiments, the mutation (e.g., nonsynonymous mutation) introduces a premature stop codon (e.g., introduces a premature stop codon in the gene encoding the biomarker that results in an inactivated or nonfunctional translational product). In some embodiments, the mutation is a full or partial deletion of the gene encoding the biomarker (e.g., a partial deletion that results in an inactivated or nonfunctional translational product). In some embodiments, the full or partial deletion occurs in one or both gene alleles encoding the biomarker. In some embodiments, the mutation is a full deletion of the gene encoding the biomarker. In some embodiments, the mutation is a frameshift mutation. In some embodiments, the biomarker has an open reading frame, wherein the frameshift mutation occurs at or proximal to the 5′end of the open-reading frame of the biomarker. In some aspects, the mutation is detected by sequencing genomic DNA obtained from the diseased tissue sample. In some aspects, the sequencing comprises next generation sequencing. In some aspects, the presence of a mutation in the one or more biomarkers is used to identify the subject for treatment with one or more PKMYT1 therapeutic agents. In some aspects, the presence of a mutation in the one or more biomarkers is used to determine the responsiveness of the subject to treatment with one or more PKMYT1 therapeutic agents. In some aspects, the presence of a mutation in the one or more biomarkers is used to determine that the subject will respond or will likely respond to treatment with one or more PKMYT1 therapeutic agents.

In some embodiments, in any of the foregoing or related aspects, the method further comprises administering one or more PKMYT1 therapeutic agents to the subject. In some aspects, a subject identified for treatment with one or more PKMYT1 therapeutic agents according to a method described herein is administered one or more PKMYT1 therapeutic agents. In some aspects, the subject is one determined to respond or to likely respond to treatment with one or more PKMYT1 therapeutic agents according to a method described herein. In some aspects, the administering results in a reduced expression level and/or activity of PKMYT1 in the subject. In some aspects, the administering results in a reduced expression level and/or activity of PKMYT1 in a diseased tissue of the subject. In some aspects, the reduced expression level and/or activity of PKMYT1 induces synthetic lethality. In some aspects, the reduced expression level and/or activity of PKMYT1 induces synthetic lethality in the diseased tissue. In some aspects, the synthetic lethality provides for treatment of the diseased tissue.

In some embodiments, in any of the foregoing or related aspects, the subject has tumor. In some aspects, the diseased tissue sample comprises a tumor sample, a circulating tumor DNA sample, a tumor biopsy sample, or a fixed tumor sample. In some aspects, the tumor comprises a plurality of tumor cells comprising the mutation.

In some aspects, the disclosure provides a method of treating a cancer or promoting tumor regression in a subject having a tumor comprising a mutation in, an altered expression level of, and/or an altered activity of one or more biomarkers, wherein the one or more biomarkers is selected from any one or any combination (e.g., 2, 3, 4, 5 or more) of biomarkers listed in Table 3, the method comprising: administering to the subject a therapeutically effective amount of one or more PKMYT1 therapeutic agents.

In some aspects, the disclosure provides a method of treating a cancer or promoting tumor regression in a subject having a tumor comprising a mutation in, an altered expression level of, and/or an altered activity of one or more biomarkers, wherein the one or more biomarkers is selected from any one or any combination (e.g., 2, 3, 4, 5 or more) of biomarkers listed in Table 8 and 9, the method comprising: administering to the subject a therapeutically effective amount of one or more PKMYT1 therapeutic agents.

In some aspects, the disclosure provides a method of treating a cancer or promoting tumor regression in a subject having a tumor comprising a mutation in, an altered expression level of, and/or an altered activity of one or more biomarkers, wherein the one or more biomarkers is selected from any one or any combination (e.g., 2, 3, 4, 5 or more) of biomarkers listed in Table 10, the method comprising: administering to the subject a therapeutically effective amount of one or more PKMYT1 therapeutic agents.

In some aspects, the disclosure provides a method of treating a cancer or promoting tumor regression in a subject having a tumor comprising a mutation in, an altered expression level of, and/or an altered activity of one or more biomarkers, wherein the one or more biomarkers is selected from any one or any combination (e.g., 2, 3, 4, 5 or more) of biomarkers listed in Table 3, wherein a tumor sample obtained from the subject comprises (a) a decreased expression level and/or decreased activity in the one or more biomarkers relative to a reference tissue sample; and/or (b) a loss-of-function mutation in the one or more biomarkers relative to a reference tissue sample, the method comprising: administering to the subject a therapeutically effective amount of one or more PKMYT1 therapeutic agents.

In some aspects, the disclosure provides a method of treating a cancer or promoting tumor regression in a subject having a tumor comprising a mutation in, an altered expression level of, and/or an altered activity of one or more biomarkers, wherein the one or more biomarkers is selected from any one or any combination (e.g., 2, 3, 4, 5 or more) of biomarkers listed in Table 8 and 9, wherein a tumor sample obtained from the subject comprises (a) a decreased expression level and/or decreased activity in the one or more biomarkers relative to a reference tissue sample; and/or (b) a loss-of-function mutation in the one or more biomarkers relative to a reference tissue sample, the method comprising: administering to the subject a therapeutically effective amount of one or more PKMYT1 therapeutic agents.

In some aspects, the disclosure provides a method of treating a cancer or promoting tumor regression in a subject having a tumor comprising a mutation in, an altered expression level of, and/or an altered activity of one or more biomarkers, wherein the one or more biomarkers is selected from any one or any combination (e.g., 2, 3, 4, 5 or more) of biomarkers listed in Table 10, wherein a tumor sample obtained from the subject comprises (a) a decreased expression level and/or decreased activity in the one or more biomarkers relative to a reference tissue sample; and/or (b) a loss-of-function mutation in the one or more biomarkers relative to a reference tissue sample, the method comprising: administering to the subject a therapeutically effective amount of one or more PKMYT1 therapeutic agents.

In some aspects, the disclosure provides a method of treating a cancer or promoting tumor regression in a subject having a tumor comprising a mutation in, an altered expression level of, and/or an altered activity of one or more biomarkers, wherein the one or more biomarkers is selected from any one or any combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, PPP3CC, and PPP2R1B, the method comprising: administering to the subject a therapeutically effective amount of one or more PKMYT1 therapeutic agents.

In some aspects, the disclosure provides a method of treating a cancer or promoting tumor regression in a subject having a tumor comprising a mutation in, an altered expression level of, and/or an altered activity of one or more biomarkers, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16, the method comprising: administering to the subject a therapeutically effective amount of one or more protein kinase, membrane associated tyrosine/threonine 1 (PKMYT1) therapeutic agents.

In some aspects, the disclosure provides a method of treating a cancer or promoting tumor regression in a subject having a tumor comprising a mutation in, an altered expression level of, and/or an altered activity of one or more biomarkers, wherein the one or more biomarkers is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF, the method comprising: administering to the subject a therapeutically effective amount of one or more protein kinase, membrane associated tyrosine/threonine 1 (PKMYT1) therapeutic agents.

In some aspects, the disclosure provides a method of treating a cancer or promoting tumor regression in a subject having a tumor comprising a mutation in, an altered expression level of, and/or an altered activity of one or more biomarkers, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB, the method comprising: administering to the subject a therapeutically effective amount of one or more protein kinase, membrane associated tyrosine/threonine 1 (PKMYT1) therapeutic agents.

In some aspects, the disclosure provides a method of treating a cancer or promoting tumor regression in a subject having a tumor comprising a mutation in, an altered expression level of, and/or an altered activity of one or more biomarkers, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1, the method comprising: administering to the subject a therapeutically effective amount of one or more protein kinase, membrane associated tyrosine/threonine 1 (PKMYT1) therapeutic agents.

In some aspects, the disclosure provides a method of treating a cancer or promoting tumor regression in a subject having a tumor comprising a mutation in, an altered expression level of, and/or an altered activity of one or more biomarkers, wherein the one or more biomarkers is selected from any one or any combination (e.g., 2, 3, 4, 5 or more) of a biomarker listed in Table 1, the method comprising: administering to the subject a therapeutically effective amount of one or more protein kinase, membrane associated tyrosine/threonine 1 (PKMYT1) therapeutic agents.

In some aspects, the disclosure provides a method of treating a cancer or promoting tumor regression in a subject having a tumor comprising a mutation in, an altered expression level of, and/or an altered activity of one or more biomarkers, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21, the method comprising: administering to the subject a therapeutically effective amount of one or more protein kinase, membrane associated tyrosine/threonine 1 (PKMYT1) therapeutic agents.

In some or any of the foregoing or related embodiments, the tumor comprises a loss of function mutation in the one or more biomarkers as measured in a tumor sample obtained from the subject relative to a reference tissue sample. In some embodiments, the tumor comprises a reduced expression level of one or more biomarkers as measured in a tumor sample obtained from the subject relative to a reference tissue sample. In some embodiments, the tumor comprises a reduced activity of one or more biomarkers as measured in a tumor sample obtained from the subject relative to a reference tissue sample.

In some aspects, the disclosure provides a method of treating a cancer or promoting tumor regression in a subject having a tumor comprising a mutation in one or more biomarkers, wherein the one or more biomarkers is selected from any one or any combination (e.g., 2, 3, 4, 5 or more) of biomarkers listed in Table 3, the method comprising: administering to the subject a therapeutically effective amount of one or more PKMYT1 therapeutic agents.

In some aspects, the disclosure provides a method of treating a cancer or promoting tumor regression in a subject having a tumor comprising a mutation in one or more biomarkers, wherein the one or more biomarkers is selected from any one or any combination (e.g., 2, 3, 4, 5 or more) of biomarkers listed in Table 8 and 9, the method comprising: administering to the subject a therapeutically effective amount of one or more PKMYT1 therapeutic agents.

In some aspects, the disclosure provides a method of treating a cancer or promoting tumor regression in a subject having a tumor comprising a mutation in one or more biomarkers, wherein the one or more biomarkers is selected from any one or any combination (e.g., 2, 3, 4, 5 or more) of biomarkers listed in Table 10, the method comprising: administering to the subject a therapeutically effective amount of one or more PKMYT1 therapeutic agents.

In some aspects, the disclosure provides a method of treating a cancer or promoting tumor regression in a subject having a tumor comprising a mutation in one or more biomarkers, wherein the one or more biomarkers is selected from any one or any combination (e.g., 2, 3, 4, 5 or more) of biomarkers listed in Table 3, wherein a tumor sample obtained from the subject comprises a loss-of-function mutation in the one or more biomarkers relative to a reference tissue sample, the method comprising: administering to the subject a therapeutically effective amount of one or more PKMYT1 therapeutic agents.

In some aspects, the disclosure provides a method of treating a cancer or promoting tumor regression in a subject having a tumor comprising a mutation in one or more biomarkers, wherein the one or more biomarkers is selected from any one or any combination (e.g., 2, 3, 4, 5 or more) of biomarkers listed in Table 8 and 9, wherein a tumor sample obtained from the subject comprises a loss-of-function mutation in the one or more biomarkers relative to a reference tissue sample, the method comprising: administering to the subject a therapeutically effective amount of one or more PKMYT1 therapeutic agents.

In some aspects, the disclosure provides a method of treating a cancer or promoting tumor regression in a subject having a tumor comprising a mutation in one or more biomarkers, wherein the one or more biomarkers is selected from any one or any combination (e.g., 2, 3, 4, 5 or more) of biomarkers listed in Table 10, wherein a tumor sample obtained from the subject comprises a loss-of-function mutation in the one or more biomarkers relative to a reference tissue sample, the method comprising: administering to the subject a therapeutically effective amount of one or more PKMYT1 therapeutic agents.

In some aspects, the disclosure provides a method of treating a cancer or promoting tumor regression in a subject having a tumor comprising a mutation in one or more biomarkers, wherein the one or more biomarkers is selected from any one or any combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, PPP3CC, and PPP2R1B, the method comprising: administering to the subject a therapeutically effective amount of one or more PKMYT1 therapeutic agents. In some aspects, the tumor comprises a loss of function mutation in the one or more biomarkers as measured in a tumor sample obtained from the subject relative to a reference tissue sample.

In some aspects, the disclosure provides a method of treating a cancer or promoting tumor regression in a subject having a tumor comprising a mutation in one or more biomarkers, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16, the method comprising: administering to the subject a therapeutically effective amount of one or more protein kinase, membrane associated tyrosine/threonine 1 (PKMYT1) therapeutic agents. In some aspects, the tumor comprises a loss of function mutation in the one or more biomarkers as measured in a tumor sample obtained from the subject relative to a reference tissue sample (e.g., healthy control tissue sample).

In some aspects, the disclosure provides a method of treating a cancer or promoting tumor regression in a subject having a tumor comprising a mutation in one or more biomarkers, wherein the one or more biomarkers is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF, the method comprising: administering to the subject a therapeutically effective amount of one or more protein kinase, membrane associated tyrosine/threonine 1 (PKMYT1) therapeutic agents.

In some aspects, the disclosure provides a method of treating a cancer or promoting tumor regression in a subject having a tumor comprising a mutation in one or more biomarkers, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB, the method comprising: administering to the subject a therapeutically effective amount of one or more protein kinase, membrane associated tyrosine/threonine 1 (PKMYT1) therapeutic agents.

In some aspects, the disclosure provides a method of treating a cancer or promoting tumor regression in a subject having a tumor comprising a mutation in one or more biomarkers, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1, the method comprising: administering to the subject a therapeutically effective amount of one or more protein kinase, membrane associated tyrosine/threonine 1 (PKMYT1) therapeutic agents.

In some aspects, the disclosure provides a method of treating a cancer or promoting tumor regression in a subject having a tumor comprising a mutation in one or more biomarkers, wherein the one or more biomarkers is selected from any one or any combination (e.g., 2, 3, 4, 5 or more) of a biomarker listed in Table 1, the method comprising: administering to the subject a therapeutically effective amount of one or more protein kinase, membrane associated tyrosine/threonine 1 (PKMYT1) therapeutic agents.

In some aspects, the disclosure provides a method of treating a cancer or promoting tumor regression in a subject having a tumor comprising a mutation in one or more biomarkers, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21, the method comprising: administering to the subject a therapeutically effective amount of one or more protein kinase, membrane associated tyrosine/threonine 1 (PKMYT1) therapeutic agents.

In some or any of the foregoing or related embodiments, the tumor comprises a loss of function mutation in the one or more biomarkers as measured in a tumor sample obtained from the subject relative to a reference tissue sample (e.g., healthy control tissue sample).

In some aspects, the disclosure provides a method of treating a cancer or promoting tumor regression in a subject having a tumor comprising an altered expression level of one or more biomarkers, wherein the one or more biomarkers is selected from any one or any combination (e.g., 2, 3, 4, 5 or more) of biomarkers listed in Table 3, the method comprising: administering to the subject a therapeutically effective amount of one or more PKMYT1 therapeutic agents.

In some aspects, the disclosure provides a method of treating a cancer or promoting tumor regression in a subject having a tumor comprising an altered expression level of one or more biomarkers, wherein the one or more biomarkers is selected from any one or any combination (e.g., 2, 3, 4, 5 or more) of biomarkers listed in Table 8 and 9, the method comprising: administering to the subject a therapeutically effective amount of one or more PKMYT1 therapeutic agents.

In some aspects, the disclosure provides a method of treating a cancer or promoting tumor regression in a subject having a tumor comprising an altered expression level of one or more biomarkers, wherein the one or more biomarkers is selected from any one or any combination (e.g., 2, 3, 4, 5 or more) of biomarkers listed in Table 10, the method comprising: administering to the subject a therapeutically effective amount of one or more PKMYT1 therapeutic agents.

In some aspects, the disclosure provides a method of treating a cancer or promoting tumor regression in a subject having a tumor comprising an altered expression level of one or more biomarkers, wherein the one or more biomarkers is selected from any one or any combination (e.g., 2, 3, 4, 5 or more) of biomarkers listed in Table 3, wherein a tumor sample obtained from the subject comprises a decreased expression level of the one or more biomarkers relative to a reference tissue sample, the method comprising: administering to the subject a therapeutically effective amount of one or more PKMYT1 therapeutic agents.

In some aspects, the disclosure provides a method of treating a cancer or promoting tumor regression in a subject having a tumor comprising an altered expression level of one or more biomarkers, wherein the one or more biomarkers is selected from any one or any combination (e.g., 2, 3, 4, 5 or more) of biomarkers listed in Table 8 and 9, wherein a tumor sample obtained from the subject comprises a decreased expression level of the one or more biomarkers relative to a reference tissue sample, the method comprising: administering to the subject a therapeutically effective amount of one or more PKMYT1 therapeutic agents.

In some aspects, the disclosure provides a method of treating a cancer or promoting tumor regression in a subject having a tumor comprising an altered expression level of one or more biomarkers, wherein the one or more biomarkers is selected from any one or any combination (e.g., 2, 3, 4, 5 or more) of biomarkers listed in Table 10, wherein a tumor sample obtained from the subject comprises a decreased expression level in the one or more biomarkers relative to a reference tissue sample, the method comprising: administering to the subject a therapeutically effective amount of one or more PKMYT1 therapeutic agents.

In some aspects, the disclosure provides a method of treating a cancer or promoting tumor regression in a subject having a tumor comprising an altered expression level and/or activity of one or more biomarkers, wherein the one or more biomarkers is selected from any one or a combination (e.g., 2, 3, 4, 5, or more) of ATM, MAP2K4, TP53, CDC25A, CACNA1H, CDKN1B, DUSP7, FOXO3, FZD3, JAK1, SMAD2, TGFBR2, MAP3K2, PPP2R1B, PPP3CC, and PPP2R1B, the method comprising: administering to the subject a therapeutically effective amount of one or more PKMYT1 therapeutic agents.

In some aspects, the disclosure provides a method of treating a cancer or promoting tumor regression in a subject having a tumor comprising an altered expression level of one or more biomarkers, wherein the one or more biomarkers is selected from any one or a combination (e.g., 2, 3, 4, 5, or more) of ATM, MAP2K4, TP53, CDC25A, CACNA1H, CDKN1B, DUSP7, FOXO3, FZD3, JAK1, SMAD2, TGFBR2, MAP3K2, PPP2R1B, PPP3CC, and PPP2R1B, the method comprising: administering to the subject a therapeutically effective amount of one or more PKMYT1 therapeutic agents.

In some aspects, the disclosure provides a method of treating a cancer or promoting tumor regression in a subject having a tumor comprising an altered expression level of one or more biomarkers, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16, the method comprising: administering to the subject a therapeutically effective amount of one or more PKMYT1 therapeutic agents. In some aspects, the tumor comprises a reduced expression level of one or more biomarkers as measured in a tumor sample obtained from the subject relative to a reference tissue sample (e.g., healthy control tissue sample).

In some aspects, the disclosure provides a method of treating a cancer or promoting tumor regression in a subject having a tumor comprising an altered expression level of one or more biomarkers, wherein the one or more biomarkers is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF, the method comprising: administering to the subject a therapeutically effective amount of one or more PKMYT1 therapeutic agents.

In some aspects, the disclosure provides a method of treating a cancer or promoting tumor regression in a subject having a tumor comprising an altered expression level of one or more biomarkers, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB, the method comprising: administering to the subject a therapeutically effective amount of one or more PKMYT1 therapeutic agents.

In some aspects, the disclosure provides a method of treating a cancer or promoting tumor regression in a subject having a tumor comprising an altered expression level of one or more biomarkers, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1, the method comprising: administering to the subject a therapeutically effective amount of one or more PKMYT1 therapeutic agents.

In some aspects, the disclosure provides a method of treating a cancer or promoting tumor regression in a subject having a tumor comprising an altered expression level of one or more biomarkers, wherein the one or more biomarkers is selected from any one or any combination (e.g., 2, 3, 4, 5 or more) of biomarkers listed in Table 1, the method comprising: administering to the subject a therapeutically effective amount of one or more PKMYT1 therapeutic agents.

In some aspects, the disclosure provides a method of treating a cancer or promoting tumor regression in a subject having a tumor comprising an altered expression level of one or more biomarkers, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21, the method comprising: administering to the subject a therapeutically effective amount of one or more PKMYT1 therapeutic agents.

In some or any of the foregoing or related embodiments, the tumor comprises a reduced expression level of one or more biomarkers as measured in a tumor sample obtained from the subject relative to a reference tissue sample (e.g., healthy control tissue sample).

In some aspects, the disclosure provides a method of treating a cancer or promoting tumor regression in a subject having a tumor comprising an altered activity of one or more biomarkers, wherein the one or more biomarkers is selected from any one or any combination (e.g., 2, 3, 4, 5 or more) of biomarkers listed in Table 3, the method comprising: administering to the subject a therapeutically effective amount of one or more PKMYT1 therapeutic agents.

In some aspects, the disclosure provides a method of treating a cancer or promoting tumor regression in a subject having a tumor comprising an altered activity of one or more biomarkers, wherein the one or more biomarkers is selected from any one or any combination (e.g., 2, 3, 4, 5 or more) of biomarkers listed in Table 8 and 9, the method comprising: administering to the subject a therapeutically effective amount of one or more PKMYT1 therapeutic agents.

In some aspects, the disclosure provides a method of treating a cancer or promoting tumor regression in a subject having a tumor comprising an altered activity of one or more biomarkers, wherein the one or more biomarkers is selected from any one or any combination (e.g., 2, 3, 4, 5 or more) of biomarkers listed in Table 10, the method comprising: administering to the subject a therapeutically effective amount of one or more PKMYT1 therapeutic agents.

In some aspects, the disclosure provides a method of treating a cancer or promoting tumor regression in a subject having a tumor comprising an altered activity of one or more biomarkers, wherein the one or more biomarkers is selected from any one or any combination (e.g., 2, 3, 4, 5 or more) of biomarkers listed in Table 3, wherein a tumor sample obtained from the subject comprises a decreased activity in the one or more biomarkers relative to a reference tissue sample, the method comprising: administering to the subject a therapeutically effective amount of one or more PKMYT1 therapeutic agents.

In some aspects, the disclosure provides a method of treating a cancer or promoting tumor regression in a subject having a tumor comprising an altered activity of one or more biomarkers, wherein the one or more biomarkers is selected from any one or any combination (e.g., 2, 3, 4, 5 or more) of biomarkers listed in Table 8 and 9, wherein a tumor sample obtained from the subject comprises a decreased activity in the one or more biomarkers relative to a reference tissue sample, the method comprising: administering to the subject a therapeutically effective amount of one or more PKMYT1 therapeutic agents.

In some aspects, the disclosure provides a method of treating a cancer or promoting tumor regression in a subject having a tumor comprising an altered activity of one or more biomarkers, wherein the one or more biomarkers is selected from any one or any combination (e.g., 2, 3, 4, 5 or more) of biomarkers listed in Table 10, wherein a tumor sample obtained from the subject comprises decreased activity in the one or more biomarkers relative to a reference tissue sample, the method comprising: administering to the subject a therapeutically effective amount of one or more PKMYT1 therapeutic agents.

In some aspects, the disclosure provides a method of treating a cancer or promoting tumor regression in a subject having a tumor comprising an altered activity of one or more biomarkers, wherein the one or more biomarkers is selected from any one or a combination (e.g., 2, 3, 4, 5, or more) of ATM, MAP2K4, TP53, CDC25A, CACNA1H, CDKN1B, DUSP7, FOXO3, FZD3, JAK1, SMAD2, TGFBR2, MAP3K2, PPP2R1B, PPP3CC, and PPP2R1B, the method comprising: administering to the subject a therapeutically effective amount of one or more PKMYT1 therapeutic agents.

In some aspects, the disclosure provides a method of treating a cancer or promoting tumor regression in a subject having a tumor comprising an altered activity of one or more biomarkers, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16, the method comprising: administering to the subject a therapeutically effective amount of one or more PKMYT1 therapeutic agents. In some aspects, the tumor comprises a reduced activity of one or more biomarkers as measured in a tumor sample obtained from the subject relative to a reference tissue sample (e.g., healthy control tissue sample).

In some aspects, the disclosure provides a method of treating a cancer or promoting tumor regression in a subject having a tumor comprising an altered activity of one or more biomarkers, wherein the one or more biomarkers is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF, the method comprising: administering to the subject a therapeutically effective amount of one or more PKMYT1 therapeutic agents.

In some aspects, the disclosure provides a method of treating a cancer or promoting tumor regression in a subject having a tumor comprising an altered activity of one or more biomarkers, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB, the method comprising: administering to the subject a therapeutically effective amount of one or more PKMYT1 therapeutic agents.

In some aspects, the disclosure provides a method of treating a cancer or promoting tumor regression in a subject having a tumor comprising an altered activity of one or more biomarkers, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1, the method comprising: administering to the subject a therapeutically effective amount of one or more PKMYT1 therapeutic agents.

In some aspects, the disclosure provides a method of treating a cancer or promoting tumor regression in a subject having a tumor comprising an altered activity of one or more biomarkers, wherein the one or more biomarkers is selected from any one or any combination (e.g., 2, 3, 4, 5 or more) of the biomarkers listed in Table 1, the method comprising: administering to the subject a therapeutically effective amount of one or more PKMYT1 therapeutic agents.

In some aspects, the disclosure provides a method of treating a cancer or promoting tumor regression in a subject having a tumor comprising an altered activity of one or more biomarkers, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21, the method comprising: administering to the subject a therapeutically effective amount of one or more PKMYT1 therapeutic agents.

In some or any of the foregoing or related embodiments, the tumor comprises a reduced activity of one or more biomarkers as measured in a tumor sample obtained from the subject relative to a reference tissue sample (e.g., healthy control tissue sample).

In some aspects, the disclosure provides a method of identifying a cancer subject to receive one or more PKMYT1 therapeutic agents, comprising (i) determining the presence of a mutation in, the expression level of, and/or the activity of one or more biomarkers in a tumor sample obtained from the subject, wherein the one or more biomarkers are selected from any one or any combination (e.g., 2, 3, 4, 5 or more) of biomarkers listed in Table 3; and (ii) administering one or more PKMYT1 therapeutic agents to the subject based on presence of a mutation in, a reduced expression level of, and/or a reduced activity of the one or more biomarkers relative to a healthy control.

In some aspects, the disclosure provides a method of identifying a cancer subject to receive one or more PKMYT1 therapeutic agents, comprising (i) determining the presence of a mutation in, the expression level of, and/or the activity of one or more biomarkers in a tumor sample obtained from the subject, wherein the one or more biomarkers are selected from any one or any combination (e.g., 2, 3, 4, 5 or more) of biomarkers listed in Table 8 and 9; and (ii) administering one or more PKMYT1 therapeutic agents to the subject based on presence of a mutation in, a reduced expression level of, and/or a reduced activity of the one or more biomarkers relative to a healthy control.

In some aspects, the disclosure provides a method of identifying a cancer subject to receive one or more PKMYT1 therapeutic agents, comprising (i) determining the presence of a mutation in, the expression level of, and/or the activity of one or more biomarkers in a tumor sample obtained from the subject, wherein the one or more biomarkers are selected from any one or any combination (e.g., 2, 3, 4, 5 or more) of biomarkers listed in Table 10; and (ii) administering one or more PKMYT1 therapeutic agents to the subject based on presence of a mutation in, a reduced expression level of, and/or a reduced activity of the one or more biomarkers relative to a healthy control.

In some aspects, the disclosure provides a method of identifying a cancer subject to receive one or more PKMYT1 therapeutic agents, comprising (i) determining the presence of a mutation in, the expression level of, and/or the activity of one or more biomarkers in a tumor sample obtained from the subject, wherein the one or more biomarkers are selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, PPP3CC, and PPP2R1B; and (ii) administering one or more PKMYT1 therapeutic agents to the subject based on the presence of a mutation in, a reduced expression level of, and/or a reduced activity of the one or more biomarkers relative to a reference tissue sample.

In some aspects, the disclosure provides a method of identifying a cancer subject to receive one or more PKMYT1 therapeutic agents, comprising (i) determining the presence of a mutation in, the expression level of, and/or the activity of one or more biomarkers in a tumor sample obtained from the subject, wherein the one or more biomarkers are selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16; and (ii) administering one or more PKMYT1 therapeutic agents to the subject based on the presence of a mutation in, a reduced expression level of, and/or a reduced activity of the one or more biomarkers relative to a healthy control.

In some aspects, the disclosure provides a method of identifying a cancer subject to receive one or more PKMYT1 therapeutic agents, comprising (i) determining the presence of a mutation in, the expression level of, and/or the activity of one or more biomarkers in a tumor sample obtained from the subject, wherein the one or more biomarkers are selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF; and (ii) administering one or more PKMYT1 therapeutic agents to the subject based on presence of a mutation in, a reduced expression level of, and/or a reduced activity of the one or more biomarkers relative to a healthy control.

In some aspects, the disclosure provides a method of identifying a cancer subject to receive one or more PKMYT1 therapeutic agents, comprising (i) determining the presence of a mutation in, the expression level of, and/or the activity of one or more biomarkers in a tumor sample obtained from the subject, wherein the one or more biomarkers are selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB; and (ii) administering one or more PKMYT1 therapeutic agents to the subject based on presence of a mutation in, a reduced expression level of, and/or a reduced activity of the one or more biomarkers relative to a healthy control.

In some aspects, the disclosure provides a method of identifying a cancer subject to receive one or more PKMYT1 therapeutic agents, comprising (i) determining the presence of a mutation in, the expression level of, and/or the activity of one or more biomarkers in a tumor sample obtained from the subject, wherein the one or more biomarkers are selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1; and (ii) administering one or more PKMYT1 therapeutic agents to the subject based on presence of a mutation in, a reduced expression level of, and/or a reduced activity of the one or more biomarkers relative to a healthy control.

In some aspects, the disclosure provides a method of identifying a cancer subject to receive one or more PKMYT1 therapeutic agents, comprising (i) determining the presence of a mutation in, the expression level of, and/or the activity of one or more biomarkers in a tumor sample obtained from the subject, wherein the one or more biomarkers are selected from any one or any combination (e.g., 2, 3, 4, 5 or more) of the biomarkers listed in Table 1, and (ii) administering one or more PKMYT1 therapeutic agents to the subject based on presence of a mutation in, a reduced expression level of, and/or a reduced activity of the one or more biomarkers relative to a healthy control.

In some aspects, the disclosure provides a method of identifying a cancer subject to receive one or more PKMYT1 therapeutic agents, comprising (i) determining the presence of a mutation in, the expression level of, and/or the activity of one or more biomarkers in a tumor sample obtained from the subject, wherein the one or more biomarkers are selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21; and (ii) administering one or more PKMYT1 therapeutic agents to the subject based on presence of a mutation in, a reduced expression level of, and/or a reduced activity of the one or more biomarkers relative to a healthy control.

In some aspects, the disclosure provides a method of identifying a cancer subject to receive one or more PKMYT1 therapeutic agents, comprising (i) determining the presence of a mutation in one or more biomarkers in a tumor sample obtained from the subject, wherein the one or more biomarkers are selected from any one or any combination (e.g., 2, 3, 4, 5 or more) of biomarkers listed in Table 3; and (ii) administering one or more PKMYT1 therapeutic agents to the subject based on presence of a mutation in the one or more biomarkers relative to a healthy control.

In some aspects, the disclosure provides a method of identifying a cancer subject to receive one or more PKMYT1 therapeutic agents, comprising (i) determining the presence of a mutation in one or more biomarkers in a tumor sample obtained from the subject, wherein the one or more biomarkers are selected from any one or any combination (e.g., 2, 3, 4, 5 or more) of biomarkers listed in Table 8 and 9; and (ii) administering one or more PKMYT1 therapeutic agents to the subject based on presence of a mutation in the one or more biomarkers relative to a healthy control.

In some aspects, the disclosure provides a method of identifying a cancer subject to receive one or more PKMYT1 therapeutic agents, comprising (i) determining the presence of a mutation in one or more biomarkers in a tumor sample obtained from the subject, wherein the one or more biomarkers are selected from any one or any combination (e.g., 2, 3, 4, 5 or more) of biomarkers listed in Table 10; and (ii) administering one or more PKMYT1 therapeutic agents to the subject based on presence of a mutation in the one or more biomarkers relative to a healthy control.

In some aspects, the disclosure provides a method of identifying a cancer subject to receive one or more PKMYT1 therapeutic agents, comprising (i) determining the presence of a mutation in one or more biomarkers in a tumor sample obtained from the subject, wherein the one or more biomarkers are selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, PPP3CC, and PPP2R1B; and (ii) administering one or more PKMYT1 therapeutic agents to the subject based on the presence of a mutation in the one or more biomarkers relative to a reference tissue sample. In some embodiments, the mutation is a loss of function mutation.

In some aspects, the disclosure provides a method of identifying a cancer subject to receive one or more PKMYT1 therapeutic agents, comprising (i) determining the presence of a mutation in one or more biomarkers in a tumor sample obtained from the subject, wherein the one or more biomarkers are selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16; and (ii) administering one or more PKMYT1 therapeutic agents to the subject based on the presence of a mutation in the one or more biomarkers. In some aspects, the tumor sample comprises a loss of function mutation in the one or more biomarkers. In some aspects, the tumor sample comprises a loss of function mutation in the one or more biomarkers relative to a reference tissue sample (e.g., healthy control sample).

In some aspects, the disclosure provides a method of identifying a cancer subject to receive one or more PKMYT1 therapeutic agents, comprising (i) determining the presence of a mutation in one or more biomarkers in a tumor sample obtained from the subject, wherein the one or more biomarkers are selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF; and (ii) administering one or more PKMYT1 therapeutic agents to the subject based on presence of a mutation in the one or more biomarkers.

In some aspects, the disclosure provides a method of identifying a cancer subject to receive one or more PKMYT1 therapeutic agents, comprising (i) determining the presence of a mutation in one or more biomarkers in a tumor sample obtained from the subject, wherein the one or more biomarkers are selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB; and (ii) administering one or more PKMYT1 therapeutic agents to the subject based on presence of a mutation in the one or more biomarkers.

In some aspects, the disclosure provides a method of identifying a cancer subject to receive one or more PKMYT1 therapeutic agents, comprising (i) determining the presence of a mutation in one or more biomarkers in a tumor sample obtained from the subject, wherein the one or more biomarkers are selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1; and (ii) administering one or more PKMYT1 therapeutic agents to the subject based on presence of a mutation in the one or more biomarkers.

In some aspects, the disclosure provides a method of identifying a cancer subject to receive one or more PKMYT1 therapeutic agents, comprising (i) determining the presence of a mutation in one or more biomarkers in a tumor sample obtained from the subject, wherein the one or more biomarkers are selected from any one or any combination (e.g., 2, 3, 4, 5 or more) of the biomarkers listed in Table 1; and (ii) administering one or more PKMYT1 therapeutic agents to the subject based on presence of a mutation in the one or more biomarkers.

In some aspects, the disclosure provides a method of identifying a cancer subject to receive one or more PKMYT1 therapeutic agents, comprising (i) determining the presence of a mutation in one or more biomarkers in a tumor sample obtained from the subject, wherein the one or more biomarkers are selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21; and (ii) administering one or more PKMYT1 therapeutic agents to the subject based on presence of a mutation in the one or more biomarkers.

In some or any of the foregoing or related embodiments, the tumor sample comprises a loss of function mutation in the one or more biomarkers. In some embodiments, the tumor sample comprises a loss of function mutation in the one or more biomarkers relative to a reference tissue sample (e.g., healthy control sample).

In some aspects, the disclosure provides a method of identifying a cancer subject to receive one or more PKMYT1 therapeutic agents, comprising (i) determining the expression level of one or more biomarkers in a tumor sample obtained from the subject, wherein the one or more biomarkers are selected from any one or any combination (e.g., 2, 3, 4, 5 or more) of biomarkers listed in Table 3; and (ii) administering one or more PKMYT1 therapeutic agents to the subject based on presence of a reduced expression level of the one or more biomarkers relative to a healthy control.

In some aspects, the disclosure provides a method of identifying a cancer subject to receive one or more PKMYT1 therapeutic agents, comprising (i) determining the expression level of one or more biomarkers in a tumor sample obtained from the subject, wherein the one or more biomarkers are selected from any one or any combination (e.g., 2, 3, 4, 5 or more) of biomarkers listed in Table 8 and 9; and (ii) administering one or more PKMYT1 therapeutic agents to the subject based a reduced expression level of the one or more biomarkers relative to a healthy control.

In some aspects, the disclosure provides a method of identifying a cancer subject to receive one or more PKMYT1 therapeutic agents, comprising (i) determining the expression level of one or more biomarkers in a tumor sample obtained from the subject, wherein the one or more biomarkers are selected from any one or any combination (e.g., 2, 3, 4, 5 or more) of biomarkers listed in Table 10; and (ii) administering one or more PKMYT1 therapeutic agents to the subject based on a reduced expression level of the one or more biomarkers relative to a healthy control.

In some aspects, the disclosure provides a method of identifying a cancer subject to receive one or more PKMYT1 therapeutic agents, comprising (i) determining the activity of one or more biomarkers in a tumor sample obtained from the subject, wherein the one or more biomarkers are selected from any one or any combination (e.g., 2, 3, 4, 5 or more) of biomarkers listed in Table 3; and (ii) administering one or more PKMYT1 therapeutic agents to the subject based on a reduced activity of the one or more biomarkers relative to a healthy control.

In some aspects, the disclosure provides a method of identifying a cancer subject to receive one or more PKMYT1 therapeutic agents, comprising (i) determining the activity of one or more biomarkers in a tumor sample obtained from the subject, wherein the one or more biomarkers are selected from any one or any combination (e.g., 2, 3, 4, 5 or more) of biomarkers listed in Table 8 and 9; and (ii) administering one or more PKMYT1 therapeutic agents to the subject based a reduced activity of the one or more biomarkers relative to a healthy control.

In some aspects, the disclosure provides a method of identifying a cancer subject to receive one or more PKMYT1 therapeutic agents, comprising (i) determining the activity of one or more biomarkers in a tumor sample obtained from the subject, wherein the one or more biomarkers are selected from any one or any combination (e.g., 2, 3, 4, 5 or more) of biomarkers listed in Table 10; and (ii) administering one or more PKMYT1 therapeutic agents to the subject based on presence of a reduced activity of the one or more biomarkers relative to a healthy control.

In some aspects, the disclosure provides a method of identifying a cancer subject to receive one or more PKMYT1 therapeutic agents, comprising (i) determining the expression level and/or activity of one or more biomarkers in a tumor sample obtained from the subject, wherein the one or more biomarkers are selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, PPP3CC, and PPP2R1B; and (ii) administering one or more PKMYT1 therapeutic agents to the subject based on an expression level and/or activity of the one or more biomarkers that is reduced relative to a reference tissue sample.

In some aspects, the disclosure provides a method of identifying a cancer subject to receive one or more PKMYT1 therapeutic agents, comprising (i) determining the expression level and/or activity of one or more biomarkers in a tumor sample obtained from the subject, wherein the one or more biomarkers are selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16; and (ii) administering one or more PKMYT1 therapeutic agents to the subject based on an expression level and/or activity of the one or more biomarkers that is reduced relative to a reference tissue sample. In some aspects, the tumor sample comprises a reduced expression level of one or more biomarkers relative to the reference tissue sample (e.g., healthy control). In some aspects, the tumor sample comprises a reduced activity of one or more biomarkers relative to the reference tissue sample (e.g., healthy control).

In some aspects, the disclosure provides a method of identifying a cancer subject to receive one or more PKMYT1 therapeutic agents, comprising (i) determining the expression level and/or activity of one or more biomarkers in a tumor sample obtained from the subject, wherein the one or more biomarkers are selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF; and (ii) administering one or more PKMYT1 therapeutic agents to the subject based on an expression level and/or activity of the one or more biomarkers that is reduced relative to a reference tissue sample.

In some aspects, the disclosure provides a method of identifying a cancer subject to receive one or more PKMYT1 therapeutic agents, comprising (i) determining the expression level and/or activity of one or more biomarkers in a tumor sample obtained from the subject, wherein the one or more biomarkers are selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB; and (ii) administering one or more PKMYT1 therapeutic agents to the subject based on an expression level and/or activity of the one or more biomarkers that is reduced relative to a reference tissue sample.

In some aspects, the disclosure provides a method of identifying a cancer subject to receive one or more PKMYT1 therapeutic agents, comprising (i) determining the expression level and/or activity of one or more biomarkers in a tumor sample obtained from the subject, wherein the one or more biomarkers are selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1; and (ii) administering one or more PKMYT1 therapeutic agents to the subject based on an expression level and/or activity of the one or more biomarkers that is reduced relative to a reference tissue sample.

In some aspects, the disclosure provides a method of identifying a cancer subject to receive one or more PKMYT1 therapeutic agents, comprising (i) determining the expression level and/or activity of one or more biomarkers in a tumor sample obtained from the subject, wherein the one or more biomarkers are selected from any one or any combination (e.g., 2, 3, 4, 5 or more) of the biomarkers listed in Table 1; and (ii) administering one or more PKMYT1 therapeutic agents to the subject based on an expression level and/or activity of the one or more biomarkers that is reduced relative to a reference tissue sample.

In some aspects, the disclosure provides a method of identifying a cancer subject to receive one or more PKMYT1 therapeutic agents, comprising (i) determining the expression level and/or activity of one or more biomarkers in a tumor sample obtained from the subject, wherein the one or more biomarkers are selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21; and (ii) administering one or more PKMYT1 therapeutic agents to the subject based on an expression level and/or activity of the one or more biomarkers that is reduced relative to a reference tissue sample.

In some or any of the foregoing or related embodiments, the tumor sample comprises a loss of function mutation in the one or more biomarkers. In some embodiments, the tumor sample comprises a loss of function mutation in the one or more biomarkers relative to a reference tissue sample (e.g., healthy control sample).

In some embodiments, in any of the foregoing or related aspects, the administering results in a reduced expression level and/or activity of PKMYT1 in a tumor of the subject. In some aspects, the reduced expression level and/or activity of PKMYT1 induces synthetic lethality in the tumor. In some aspects, the synthetic lethality promotes tumor regression.

In some embodiments, in any of the foregoing or related aspects, the tumor sample is a circulating tumor DNA sample, a tumor biopsy sample, or a fixed tumor sample. In some aspects, the expression level and/or activity of the one or more biomarkers is reduced in the tumor sample relative to a reference tissue sample. In some aspects, the tumor sample comprises a mutation in the one or more biomarkers. In some embodiments, the mutation is a loss of function mutation. In some embodiments, the loss of function mutation results in the biomarker having reduced expression and/or activity. In some embodiments, the loss of function mutation abolishes expression and/or activity of the biomarker. In some embodiments, the loss of function mutation results in an inactivated or nonfunctional translational product. In some embodiments, the loss of function mutation is a deletion of the gene encoding the biomarker. In some embodiments, the mutation occurs in one or both gene alleles encoding the biomarker. In some embodiments, the mutation is a nonsynonymous mutation (e.g., a missense mutation, a nonstop mutation, a nonsense mutation). In some embodiments, the mutation (e.g., nonsynonymous mutation) is an insertion or deletion. In some embodiments, the insertion or deletion introduces a frameshift mutation (e.g., a frameshift mutation in the gene encoding the biomarker that results in an inactivated or nonfunctional translational product). In some embodiments, the mutation (e.g., nonsynonymous mutation) introduces a premature stop codon (e.g., introduces a premature stop codon in the gene encoding the biomarker that results in an inactivated or nonfunctional translational product). In some embodiments, the mutation is a full or partial deletion of the gene encoding the biomarker (e.g., a partial deletion that results in an inactivated or nonfunctional translational product). In some embodiments, the full or partial deletion occurs in one or both gene alleles encoding the biomarker. In some embodiments, the mutation is a full deletion of the gene encoding the biomarker. In some aspects, the mutation is a frameshift mutation. In some embodiments, the biomarker has an open reading frame, wherein the frameshift mutation occurs at or proximal to the 5′end of the open-reading frame of the biomarker. In some aspects, the mutation is detected by sequencing genomic DNA obtained from the diseased tissue sample. In some aspects, the sequencing comprises next generation sequencing. In some aspects, the tumor sample comprises a plurality of tumor cells comprising the mutation.

In some aspects, the disclosure provides for the use of one or more PKMYT1 therapeutic agents for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some aspects, the disclosure provides for the use of one or more PKMYT1 therapeutic agents for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of biomarkers listed in Table 3.

In some aspects, the disclosure provides for the use of one or more PKMYT1 therapeutic agents for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of biomarkers listed in Table 8 and 9.

In some aspects, the disclosure provides for the use of one or more PKMYT1 therapeutic agents for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of biomarkers listed in Table 10.

In some aspects, the disclosure provides for the use of one or more PKMYT1 therapeutic agents for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of biomarkers listed in Table 3, wherein a tumor sample obtained from the subject comprises (a) a decreased expression level and/or decreased activity in the one or more biomarkers relative to a reference tissue sample; and/or (b) a loss-of-function mutation in the one or more biomarkers relative to a reference tissue sample.

In some aspects, the disclosure provides for the use of one or more PKMYT1 therapeutic agents for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of biomarkers listed in Table 8 and 9, wherein a tumor sample obtained from the subject comprises (a) a decreased expression level and/or decreased activity in the one or more biomarkers relative to a reference tissue sample; and/or (b) a loss-of-function mutation in the one or more biomarkers relative to a reference tissue sample.

In some aspects, the disclosure provides for the use of one or more PKMYT1 therapeutic agents for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of biomarkers listed in Table 10, wherein a tumor sample obtained from the subject comprises (a) a decreased expression level and/or decreased activity in the one or more biomarkers relative to a reference tissue sample; and/or (b) a loss-of-function mutation in the one or more biomarkers relative to a reference tissue sample.

In some aspects, the disclosure provides for the use of one or more PKMYT1 therapeutic agents for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, and PPP3CC.

In some aspects, the disclosure provides for the use of one or more PKMYT1 therapeutic agents for treating a cancer or promoting tumor regression in a subject, wherein the subject has been identified based on the presence of a mutation (e.g., loss of function mutation) in one or more biomarkers, wherein the one or more biomarkers is selected from any one or any combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, and PPP3CC.

In some aspects, the disclosure provides for the use of one or more PKMYT1 therapeutic agents for treating a cancer or promoting tumor regression in a subject, wherein the subject has been identified based on an altered expression level and/or activity of one or more biomarkers, wherein the one or more biomarkers is selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, and PPP3CC.

In some aspects, the disclosure provides for the use of one or more PKMYT1 therapeutic agents for treating a cancer or promoting tumor regression in a subject, wherein the subject has been identified based on an altered (e.g., decreased) expression level of and/or an altered (e.g., decreased) activity of one or more biomarkers, wherein the one or more biomarkers is selected from any one or any combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, and PPP3CC.

In some aspects, the disclosure provides for the use of one or more PKMYT1 therapeutic agents for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some aspects, the disclosure provides for the use of one or more PKMYT1 therapeutic agents for treating a cancer or promoting tumor regression in a subject, wherein the subject has been identified based the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some aspects, the disclosure provides for the use of one or more PKMYT1 therapeutic agents for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some aspects, the disclosure provides for the use of one or more PKMYT1 therapeutic agents for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of the biomarkers listed in Table 1.

In some aspects, the disclosure provides for the use of one or more PKMYT1 therapeutic agents for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.

In some aspects, the disclosure provides for the use of one or more PKMYT1 therapeutic agents in the manufacture of a medicament for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of biomarkers listed in Table 3.

In some aspects, the disclosure provides for the use of one or more PKMYT1 therapeutic agents in the manufacture of a medicament for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of biomarkers listed in Table 8 and 9.

In some aspects, the disclosure provides for the use of one or more PKMYT1 therapeutic agents in the manufacture of a medicament for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of biomarkers listed in Table 10.

In some aspects, the disclosure provides for the use of one or more PKMYT1 therapeutic agents in the manufacture of a medicament for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of biomarkers listed in Table 3, wherein a tumor sample obtained from the subject comprises (a) a decreased expression level and/or decreased activity in the one or more biomarkers relative to a reference tissue sample; and/or (b) a loss-of-function mutation in the one or more biomarkers relative to a reference tissue sample.

In some aspects, the disclosure provides for the use of one or more PKMYT1 therapeutic agents in the manufacture of a medicament for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of biomarkers listed in Table 8 and 9, wherein a tumor sample obtained from the subject comprises (a) a decreased expression level and/or decreased activity in the one or more biomarkers relative to a reference tissue sample; and/or (b) a loss-of-function mutation in the one or more biomarkers relative to a reference tissue sample.

In some aspects, the disclosure provides for the use of one or more PKMYT1 therapeutic agents in the manufacture of a medicament for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of biomarkers listed in Table 10, wherein a tumor sample obtained from the subject comprises (a) a decreased expression level and/or decreased activity in the one or more biomarkers relative to a reference tissue sample; and/or (b) a loss-of-function mutation in the one or more biomarkers relative to a reference tissue sample.

In some aspects, the disclosure provides for the use of one or more PKMYT1 therapeutic agents in the manufacture of a medicament for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, and PPP3CC.

In some aspects, the disclosure provides for the use of one or more PKMYT1 therapeutic agents in the manufacture of a medicament for treating a cancer or promoting tumor regression in a subject, wherein the subject has been identified based on the presence of a mutation (e.g., loss of function mutation) in one or more biomarkers, wherein the one or more biomarkers is selected from any one or any combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, and PPP3CC.

In some aspects, the disclosure provides for the use of one or more PKMYT1 therapeutic agents in the manufacture of a medicament for treating a cancer or promoting tumor regression in a subject, wherein the subject has been identified based on an altered expression level and/or activity of one or more biomarkers, wherein the one or more biomarkers is selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, and PPP3CC.

In some aspects, the disclosure provides for the use of one or more PKMYT1 therapeutic agents in the manufacture of a medicament for treating a cancer or promoting tumor regression in a subject, wherein the subject has been identified based on the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some aspects, the disclosure provides for the use of one or more PKMYT1 therapeutic agents in the manufacture of a medicament for treating a cancer or promoting tumor regression in a subject, wherein the subject has been identified based the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some aspects, the disclosure provides for the use of one or more PKMYT1 therapeutic agents in the manufacture of a medicament for treating a cancer or promoting tumor regression in a subject, wherein the subject has been identified based the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some aspects, the disclosure provides for the use of one or more PKMYT1 therapeutic agents in the manufacture of a medicament for treating a cancer or promoting tumor regression in a subject, wherein the subject has been identified based the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some aspects, the disclosure provides for the use of one or more PKMYT1 therapeutic agents in the manufacture of a medicament for treating a cancer or promoting tumor regression in a subject, wherein the subject has been identified based the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of the biomarkers listed in Table 1.

In some aspects, the disclosure provides for the use of one or more PKMYT1 therapeutic agents in the manufacture of a medicament for treating a cancer or promoting tumor regression in a subject, wherein the subject has been identified based the presence of a mutation (e.g., loss of function mutation) in, an altered (e.g., decreased) expression level of, and/or an altered (e.g., decreased) activity of one or more biomarkers, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.

In some aspects, the disclosure provides a kit comprising a PKMYT1 therapeutic agent, and a package insert comprising instructions for administering the PKMYT1 therapeutic agent to a subject having a cancer comprising a mutation in, an altered expression level of, and/or an altered activity of one or more biomarkers, wherein the one or more biomarkers is selected from any one or any combination (e.g., 2, 3, 4, 5 or more) of biomarkers listed in Table 3.

In some aspects, the disclosure provides a kit comprising a PKMYT1 therapeutic agent, and a package insert comprising instructions for administering the PKMYT1 therapeutic agent to a subject having a cancer comprising a mutation in, an altered expression level of, and/or an altered activity of one or more biomarkers, wherein the one or more biomarkers is selected from any one or any combination (e.g., 2, 3, 4, 5 or more) of biomarkers listed in Table 8 and 9.

In some aspects, the disclosure provides a kit comprising a PKMYT1 therapeutic agent, and a package insert comprising instructions for administering the PKMYT1 therapeutic agent to a subject having a cancer comprising a mutation in, an altered expression level of, and/or an altered activity of one or more biomarkers, wherein the one or more biomarkers is selected from any one or any combination (e.g., 2, 3, 4, 5 or more) of biomarkers listed in Table 10.

In some aspects, the disclosure provides a kit comprising a PKMYT1 therapeutic agent, and a package insert comprising instructions for administering the PKMYT1 therapeutic agent to a subject having a cancer comprising a mutation (e.g., loss of function mutation) in, an altered (e.g., decreased) expression level of, and/or altered (e.g., decreased) activity of one or more biomarkers, wherein the one or more biomarkers is selected from any one or any combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, and PPP3CC.

In some aspects, the disclosure provides a kit comprising a PKMYT1 therapeutic agent, and a package insert comprising instructions for administering the PKMYT1 therapeutic agent to a subject having a cancer comprising a mutation (e.g., loss of function mutation) in, an altered (e.g., decreased) expression level of, and/or altered (e.g., decreased) activity of one or more biomarkers, wherein the one or more biomarkers is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some aspects, the disclosure provides a kit comprising a PKMYT1 therapeutic agent, and a package insert comprising instructions for administering the PKMYT1 therapeutic agent to a subject having a cancer comprising a mutation (e.g., loss of function mutation) in, an altered (e.g., decreased) expression level of, and/or altered (e.g., decreased) activity of one or more biomarkers, wherein the one or more biomarkers is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some aspects, the disclosure provides a kit comprising a PKMYT1 therapeutic agent, and a package insert comprising instructions for administering the PKMYT1 therapeutic agent to a subject having a cancer a mutation (e.g., loss of function mutation) in, an altered (e.g., decreased) expression level of, and/or altered (e.g., decreased) activity of one or more biomarkers, wherein the one or more biomarkers is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some aspects, the disclosure provides a kit comprising a PKMYT1 therapeutic agent, and a package insert comprising instructions for administering the PKMYT1 therapeutic agent to a subject having a cancer comprising a mutation (e.g., loss of function mutation) in, an altered (e.g., decreased) expression level of, and/or altered (e.g., decreased) activity of one or more biomarkers, wherein the one or more biomarkers is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some aspects, the disclosure provides a kit comprising a PKMYT1 therapeutic agent, and a package insert comprising instructions for administering the PKMYT1 therapeutic agent to a subject having a cancer comprising a mutation (e.g., loss of function mutation) in, an altered (e.g., decreased) expression level of, and/or altered (e.g., decreased) activity of one or more biomarkers, wherein the one or more biomarkers is selected from any one or any combination (e.g., 2, 3, 4, 5 or more) of the biomarkers listed in Table 1.

In some aspects, the disclosure provides a kit comprising a PKMYT1 therapeutic agent, and a package insert comprising instructions for administering the PKMYT1 therapeutic agent to a subject having a cancer comprising a mutation (e.g., loss of function mutation) in, an altered (e.g., decreased) expression level of, and/or altered (e.g., decreased) activity of one or more biomarkers, wherein the one or more biomarkers is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.

In some aspects, the disclosure provides a kit comprising a PKMYT1 therapeutic agent, and a package insert comprising instructions for administering the PKMYT1 therapeutic agent to a subject having a cancer comprising a mutation in a gene encoding a biomarker, wherein the biomarker is selected from any one or any combination (e.g., 2, 3, 4, 5 or more) of biomarkers listed in Table 3.

In some aspects, the disclosure provides a kit comprising a PKMYT1 therapeutic agent, and a package insert comprising instructions for administering the PKMYT1 therapeutic agent to a subject having a cancer comprising a mutation in a gene encoding a biomarker, wherein the biomarker is selected from any one or any combination (e.g., 2, 3, 4, 5 or more) of biomarkers listed in Table 8 and 9.

In some aspects, the disclosure provides a kit comprising a PKMYT1 therapeutic agent, and a package insert comprising instructions for administering the PKMYT1 therapeutic agent to a subject having a cancer comprising a mutation in a gene encoding a biomarker, wherein the biomarker is selected from any one or any combination (e.g., 2, 3, 4, 5 or more) of biomarkers listed in Table 10.

In some aspects, the disclosure provides a kit comprising a PKMYT1 therapeutic agent, and a package insert comprising instructions for administering the PKMYT1 therapeutic agent to a subject having a cancer comprising a mutation in one or more biomarkers, wherein the one or more biomarkers is selected from any one or any combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, and PPP3CC.

In some aspects, the disclosure provides a kit comprising a PKMYT1 therapeutic agent, and a package insert comprising instructions for administering the PKMYT1 therapeutic agent to a subject having a cancer comprising an altered expression level and/or activity of one or more biomarkers, wherein the one or more biomarkers is selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, and PPP3CC.

In some aspects, the disclosure provides a kit comprising a PKMYT1 therapeutic agent, and a package insert comprising instructions for administering the PKMYT1 therapeutic agent to a subject having a cancer comprising a mutation in a gene encoding a biomarker, wherein the biomarker is selected from any one or a combination (e.g., 2, 3, 4, 5 or more) of ATM, MAP2K4, TP53, CDC25A, CACNA1H, CDKN1B, DUSP7, FOXO3, FZD3, JAK1, SMAD2, TGFBR2, MAP3K2, PPP2R1B, and PPP3CC.

In some aspects, the disclosure provides a kit comprising a PKMYT1 therapeutic agent, and a package insert comprising instructions for administering the PKMYT1 therapeutic agent to a subject having a cancer comprising a mutation in a gene encoding a biomarker, wherein the biomarker is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some aspects, the disclosure provides a kit comprising a PKMYT1 therapeutic agent, and a package insert comprising instructions for administering the PKMYT1 therapeutic agent to a subject having a cancer comprising a mutation in a gene encoding a biomarker, wherein the biomarker is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some aspects, the disclosure provides a kit comprising a PKMYT1 therapeutic agent, and a package insert comprising instructions for administering the PKMYT1 therapeutic agent to a subject having a cancer comprising a mutation in a gene encoding a biomarker, wherein the biomarker is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some aspects, the disclosure provides a kit comprising a PKMYT1 therapeutic agent, and a package insert comprising instructions for administering the PKMYT1 therapeutic agent to a subject having a cancer comprising a mutation in a gene encoding a biomarker, wherein the biomarker is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some aspects, the disclosure provides a kit comprising a PKMYT1 therapeutic agent, and a package insert comprising instructions for administering the PKMYT1 therapeutic agent to a subject having a cancer comprising a mutation in a gene encoding a biomarker, wherein the biomarker is selected from any one or any combination (e.g., 2, 3, 4, 5 or more) of the biomarkers listed in Table 1.

In some aspects, the disclosure provides a kit comprising a PKMYT1 therapeutic agent, and a package insert comprising instructions for administering the PKMYT1 therapeutic agent to a subject having a cancer comprising a mutation in a gene encoding a biomarker, wherein the biomarker is selected from any one or any combination (e.g., 2, 3, 4, 5 or more) of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.

In some embodiments, in any of the foregoing or related aspects, the one or more biomarkers is at least 2, 3, 4, or 5 biomarkers selected from ATM, MAP2K4, TP53, CDC25A, CACNA1H, CDKN1B, DUSP7, FOXO3, FZD3, JAK1, SMAD2, TGFBR2, MAP3K2, a PP2 subunit, and PPP3CC. In some embodiments, the PP2 subunit is PPP2R2A. In some embodiments, the PP2 subunit is PPP2R1B.

In some embodiments, in any of the foregoing or related aspects, the one or more biomarkers comprises PPP2R1B. In some aspects, the one or more biomarkers comprises PPP3CC. In some aspects, the one or more biomarkers comprises ATM. In some aspects, the one or more biomarkers comprises CACNA1H. In some aspects, the one or more biomarkers comprises CDC25A. In some aspects, the one or more biomarkers comprises CDKN1B. In some aspects, the one or more biomarkers comprises DUSP7. In some aspects, the one or more biomarkers comprises FOXO3. In some aspects, the one or more biomarkers comprises FZD3. In some aspects, the one or more biomarkers comprises JAK1. In some aspects, the one or more biomarkers comprises MAP2K4. In some aspects, the one or more biomarkers comprises MAP3K2. In some aspects, the one or more biomarkers comprises SMAD2. In some aspects, the one or more biomarkers comprises TGFBR2. In some aspects, the one or more biomarkers comprises TP53.

In some embodiments, in any of the foregoing or related aspects, the one or more biomarkers is at least 2, 3, 4, 5, or more biomarkers selected from BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, CDC16, ATM, MAP2K4, TP53, CDC25A, CACNA1H, CDKN1B, DUSP7, FOXO3, FZD3, JAK1, SMAD2, TGFBR2, MAP3K2, a PP2 subunit, and PPP3CC. In some aspects, the PP2 subunit is PPP2R2A. In some aspects, the PP2 subunit is PPP2R1B.

In any of the foregoing or related aspects, the one or more biomarkers is at least 2, 3, 4, 5, or more biomarkers selected from ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, SARAF, ATM, MAP2K4, TP53, CDC25A, CACNA1H, CDKN1B, DUSP7, FOXO3, FZD3, JAK1, SMAD2, TGFBR2, MAP3K2, a PP2 subunit, and PPP3CC. In some aspects, the PP2 subunit is PPP2R2A. In some aspects, the PP2 subunit is PPP2R1B.

In any of the foregoing or related aspects, the one or more biomarkers is at least 2, 3, 4, 5 or more biomarkers selected from CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, NFIB, ATM, MAP2K4, TP53, CDC25A, CACNA1H, CDKN1B, DUSP7, FOXO3, FZD3, JAK1, SMAD2, TGFBR2, MAP3K2, a PP2 subunit, and PPP3CC. In some aspects, the PP2 subunit is PPP2R2A. In some aspects, the PP2 subunit is PPP2R1B.

In any of the foregoing or related aspects, the one or more biomarkers is at least 2, 3, 4, 5, or more biomarkers selected from SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, DCAF12L1, ATM, MAP2K4, TP53, CDC25A, CACNA1H, CDKN1B, DUSP7, FOXO3, FZD3, JAK1, SMAD2, TGFBR2, MAP3K2, a PP2 subunit, and PPP3CC. In some aspects, the PP2 subunit is PPP2R2A. In some aspects, the PP2 subunit is PPP2R1B.

In any of the foregoing or related aspects, the one or more biomarkers is at least 2, 3, 4, 5, or more biomarkers selected from any one or any combination of the biomarkers listed in Table 1, ATM, MAP2K4, TP53, CDC25A, CACNA1H, CDKN1B, DUSP7, FOXO3, FZD3, JAK1, SMAD2, TGFBR2, MAP3K2, a PP2 subunit, and PPP3CC. In some aspects, the PP2 subunit is PPP2R2A. In some aspects, the PP2 subunit is PPP2R1B.

In any of the foregoing or related aspects, the one or more biomarkers is at least 2, 3, 4, 5, or more biomarkers selected from any one or any combination of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, RAD21, ATM, MAP2K4, TP53, CDC25A, CACNA1H, CDKN1B, DUSP7, FOXO3, FZD3, JAK1, SMAD2, TGFBR2, MAP3K2, a PP2 subunit, and PPP3CC. In some aspects, the PP2 subunit is PPP2R2A. In some aspects, the PP2 subunit is PPP2R1B.

In some embodiments, in any of the foregoing or related aspects, the one or more PKMYT1 therapeutic agents is selected from a small molecule, a peptide, a protein, and a nucleic acid. In some aspects, the one or more PKMYT1 therapeutic agents comprises an anti-PKMYT1 antibody or fragment thereof. In some aspects, the one or more PKMYT1 therapeutic agents comprises an anti-PKMYT1 intrabody or fragment thereof. In some aspects, the one or more PKMYT1 therapeutic agents comprises an RNAi molecule or an aptamer.

In some embodiments, in any of the foregoing or related aspects, the one or more PKMYT1 therapeutic agents comprises a small molecule inhibitor. In some aspects, the small molecule inhibitor is 5-((5-methoxy-2-((4-morpholinophenyl)amino)pyrimidin-4-yl)amino)-2-methylphenol, iV-(2-chloro-6-methylphenyl)-2-((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), A-(5-chlorobenzo[t/][1,3]dioxol-4-yl)-7-(2-(4-methylpiperazin-1-yl)ethoxy)-5-((tetrahydro-2//-pyran-4-yl)oxy)quinazolin-4-amine (saracatinib), (£)-A-(4-((3-chloro-4-fluorophenyl)amino)-3-cyano-7-ethoxyquinolin-6-yl)-4-(dimethylamino)but-2-enamide(pelitinib), A-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-<i]pyrimidin-7(8//)-one (PD-0166285), 6-(2,6-dichlorophenyl)-8-methyl-2-((4-morpholinophenyl)amino)pyrido[2,3-cf]pyrimidin-7(8//)-one (PD-173952), 6-(2,6-dichlorophenyl)-8-methyl-2-((3-(methylthio)phenyl)amino)pyrido[2,3-r/Jpyrimidin-7(8//)-one (PD-173955), or 6-(2,6-dichlorophenyl)-2-((4-fluoro-3-methylphenyl)amino)-8-methylpyrido[2,3-«i]pyrimidin-7(8//)-one (PD-180970).

In some embodiments, in any of the foregoing or related aspects, the one or more PKMYT1 therapeutic agents comprises a gene editing technology for introducing a genetic knockout of the PKMYT1 gene. In some aspects, the gene editing technology comprises CRISPR/Cas9.

In some embodiments, in any of the foregoing or related aspects, the cancer is selected from: acute myeloid leukemia (LAML), adrenocortical carcinoma (ACC), bladder urothelial carcinoma (BLCA), brain lower grade glioma (LGG), breast invasive carcinoma (BRCA), cervical squamous cell carcinoma and endocervical adenocarcinoma (CESC), cholangiocarcinoma (CHOL), chronic myelogenous leukemia (LCML), colon adenocarcinoma (COAD), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), lymphoid neoplasm diffuse large B-cell lymphoma (DLBC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), testicular germ cell tumors (TGCT), thymoma (THYM), thyroid carcinoma (THCA), uterine carcinosarcoma (UCS), uterine corpus endometrial carcinoma (UCEC), and uveal melanoma (UVM).

### BRIEF DESCRIPTION OF THE DRAWINGS

FIG. 1 provides a schematic of an exemplary workflow for validating synthetic lethal pairs of the disclosure, based on a CombiGEM™ assay. Synthetic lethal pairs (gene A and gene B) are identified using computational methods described herein. A barcoded lentiviral library having constructs that encode a single guide RNA (sgRNA) having a target sequence directed to gene A (sgA) and/or gene B (sgB) is transfected into cells of a desired genetic background and barcode proliferation quantified by next generation sequencing is used to determine cell proliferation for a gene A and gene B double knockout relative to gene A knockout alone or gene B knockout alone. Such quantifications are used as a measure of synthetic lethality for a gene A/gene B pair, as further described herein.

FIG. 2 provides a schematic depicting signaling pathways for protein kinase membrane associated tyrosine/threonine 1 (“PKMYT1” or “Kinase MYT1”) and Protein Phosphatase 255 kDa regulatory subunit B alpha (“PPPR2A”).

### DETAILED DESCRIPTION

### Overview

The present disclosure is based, at least in part, on the identification of biomarkers present in one or more human cancers that form a synthetic lethal pair with PKMYT1, wherein an altered (e.g., increased or decreased) expression level and/or activity of the biomarker in a cancer renders it responsive to one or more therapeutic agents that targets PKMYT1 (e.g., a PKMYT1 inhibitory agent). As described herein, computational methods were developed to identify putative biomarkers that are deficient and/or mutated in one or more human cancers, that alone may not substantially impact viability of tumor cells, but when combined with a loss of function of PKMYT1 (e.g., via gene knockout or pharmacological inhibition), result in synthetic lethality to the tumor cells. Moreover, combinatorial screening technologies based on gene-editing (i.e., CRISPR/Cas9) are used to evaluate the predicted biomarkers for a synthetic lethal phenotype following double knockout of the biomarker and PKMYT1. For example, as demonstrated herein, methods of CRISPR-based combinatorial screening described in U.S. Pat. No. 9,315,806B2 were used to generate a double knockout library in a population of cells, wherein each cell comprised a knockout of a biomarker of the disclosure and a knockout of PKMYT1, wherein high throughput screening of cellular fitness provided a readout to validate a synthetic lethal phenotype for synthetic lethal pairs of the disclosure. Through experimental validation, biomarkers are identified that when deficient and/or mutated in a human cancer, combine, or cooperate with a therapeutic agent targeting PKMYT1 to cause tumor cell lethality. Without being bound by theory, an altered (e.g., increased or decreased) expression level and/or activity of one or more biomarkers of the disclosure in a cancer in a subject provides a predictive indicator that the cancer will respond or will likely respond to one or more therapeutic agents for modulating (e.g., decreasing) an expression level and/or activity of PKMYT1, such as one described herein.

Accordingly, the present disclosure provides methods for treating a cancer or cancerous cells thereof having a mutation in, an altered (e.g., increased or decreased) expression level of, and/or an altered activity of, a biomarker described herein, the method comprising administering one or more therapeutic agents targeting PKMYT1, wherein the one or more therapeutic agents results in an altered (e.g., increased or decreased) expression level and/or activity of PKMYT1. In some aspects, the biomarker is identified using a method described herein as forming a synthetic lethal pair with PKMYT1. In some aspects, a decrease in the expression level and/or activity of both the biomarker and PKMYT1 in a tumor cell results in lethality to the tumor cell. In some aspects, the presence of a mutation in the biomarker results in a loss of function (e.g., decreased expression level and/or activity of the biomarker). In some aspects, a decrease in the expression level and/or activity of both the biomarker and PKMYT1 in a tumor cell results in substantially reduced viability of the tumor cell.

In some aspects, the disclosure provides a method for identifying or selecting a subject with cancer to receive one or more therapeutic agents targeting PKMYT1, wherein the method comprises determining the expression level and/or activity of a biomarker described herein in a tumor sample obtained from the subject, wherein an altered (e.g., increased or decreased) expression level and/or activity of the biomarker relative to a reference tissue sample (e.g., a healthy tissue sample) indicates the subject will respond or will likely to respond to treatment with one or more therapeutic agents which modulate (e.g., decrease) the expression level and/or activity of PKMYT1. In some aspects, the tumor sample is a tumor biopsy sample (e.g., fresh or fixed tumor biopsy sample). In some aspects, the tumor sample is a blood sample comprising circulating tumor DNA. In some aspects, a decreased expression level and/or activity of the biomarker indicates the subject will respond or will likely respond following administration of one or more therapeutic agents that decreases the expression level and/or activity of PKMYT1. In some aspects, the one or more therapeutic agents is a therapeutic inhibitor of PKMYT1 (e.g., a pharmacological inhibitor or a gene-editing technology). In some embodiments, the method comprises providing a report predicting the responsiveness of the subject to the treatment based upon detection of an altered (e.g., increased or decreased) expression level and/or activity of the biomarker in the tumor sample obtained from the subject relative to a reference tissue sample (e.g., a healthy tissue sample). In some embodiments, the tumor sample is a tumor biopsy sample (e.g., fresh or fixed tumor biopsy sample). In some embodiments, the tumor sample is a blood sample comprising circulating tumor DNA. In some embodiments, a decreased expression level and/or activity of the biomarker indicates the subject will respond or will likely respond following administration of one or more therapeutic agents that decreases the expression level and/or activity of PKMYT1.

In some aspects, the disclosure provides a method for identifying or selecting a subject with cancer to receive one or more therapeutic agents targeting PKMYT1, wherein the method comprises determining the presence of a mutation in a biomarker described herein in a tumor sample obtained from the subject, wherein the presence of a mutation in the one or more biomarkers indicates the subject will respond or will likely to respond to treatment with one or more therapeutic agents that modulates (e.g., increases or decreases) the expression level and/or activity of PKMYT1. In some aspects, the tumor sample is a tumor biopsy sample (e.g., fresh or fixed tumor biopsy sample). In some aspects, the tumor sample is a blood sample comprising circulating tumor DNA. In some aspects, the presence of a mutation in the one or more biomarkers indicates the subject will respond or will likely respond following administration of one or more therapeutic agents that decreases the expression level and/or activity of PKMYT1. In some aspects, the one or more therapeutic agents is a therapeutic inhibitor of PKMYT1 (e.g., a pharmacological inhibitor or a gene-editing technology).

In some aspects, the mutation in a biomarker is an inactivating mutation or loss of function mutation in a biomarker (such as any inactivating or loss of function mutations described herein or known in the art). In some aspects, the mutation in a biomarker results in a partial loss of function of the biomarker. In some aspects, the mutation in a biomarker results in a complete loss of function of the biomarker. In some aspects, the mutation in a biomarker results in a partial loss of expression and/or activity of the biomarker. In some aspects, the mutation in a biomarker results in a complete loss of expression and/or activity of the biomarker. In some aspects, the mutation is a null mutation (leading to the deletion of the gene encoding the biomarker).

In some aspects, the disclosure provides a method of identifying or selecting a patient to receive one or more therapeutic agents targeting PKMYT1, wherein the method comprises determining the expression level and/or activity of a panel of biomarkers described herein in a tumor sample obtained from the subject, wherein an altered (e.g., increased or decreased) expression level and/or activity of at least one biomarker of the panel relative to a reference tissue sample (e.g., a healthy tissue sample) indicates the subject will respond or will likely to respond to administration of one or more therapeutic agents that manipulates the expression level and/or activity of PKMYT1. In some aspects, the tumor sample is a tumor biopsy sample (e.g., fresh or fixed tumor biopsy sample). In some aspects, the tumor sample is a blood sample comprising circulating tumor DNA. In some aspects, a decreased expression level and/or activity of at least one biomarker of the panel indicates the subject will respond or will likely respond following administration of one or more therapeutic agents that decreases the expression level and/or activity of PKMYT1. In some aspects, the response is reduced tumor progression. In some aspects, the response is reduced tumor burden. In some aspects, the response is reduced risk of metastasis.

In some aspects, the disclosure provides a method of identifying or selecting a patient to receive one or more therapeutic agents targeting PKMYT1, wherein the method comprises determining the presence of a mutation in a panel of biomarkers described herein in a tumor sample obtained from the subject, wherein the presence of a mutation in at least one biomarker of the panel indicates the subject will respond or will likely to respond to administration of one or more therapeutic agents that modulates (e.g., increases or decreases) the expression level and/or activity of PKMYT1. In some aspects, the tumor sample is a tumor biopsy sample (e.g., fresh or fixed tumor biopsy sample). In some aspects, the tumor sample is a blood sample comprising circulating tumor DNA. In some aspects, a mutation in at least one biomarker of the panel indicates the subject will respond or will likely respond following administration of one or more therapeutic agents that decreases the expression level and/or activity of PKMYT1. In some aspects, the mutation results in a loss of function of the at least one biomarker. In some embodiments, the mutation results in a decreased expression level of the at least one biomarker. In some embodiments, the mutation results in a decreased activity of the at least one biomarker. In some aspects, the response is reduced tumor progression. In some aspects, the response is reduced tumor burden. In some aspects, the response is reduced risk of metastasis.

In some embodiments, the one or more therapeutic agents that modulates (e.g., decreases) the expression level and/or activity of PKMYT1 comprises a therapeutic inhibitor of a PKMYT1 gene (e.g., a gene-editing technology). In some embodiments, the one or more therapeutic agents comprises a therapeutic inhibitor of an RNA transcribed from a PKMYT1 gene (e.g., an antisense oligonucleotide or an RNAi molecule targeting a pre-mRNA or mRNA encoding a PKMYT1 polypeptide). In some embodiments, the one or more therapeutic agents comprises a therapeutic inhibitor of a PKMYT1 polypeptide (e.g., a pharmacological inhibitor).

### Synthetic Lethality Biomarkers of the Disclosure

In some embodiments, the disclosure provides biomarkers having altered (e.g., increased or decreased) expression level and/or activity in one or more human cancers, wherein the biomarker forms a synthetic lethal pair with at least one or more target genes. As used herein, a “target gene” refers to a gene or a transcriptional or translational product thereof whose expression level and/or activity in a cell is selectively modulated by a therapeutic agent (e.g., a gene editing technology or a pharmacological inhibitor). In some embodiments, the target gene is PKMYT1.

As used herein, a “synthetic lethal pair” refers to a pair of genes in a cell (e.g., a biomarker and a target gene), wherein an altered (e.g., increased or decreased) expression level and/or activity of both genes, or the transcriptional or translational products thereof, impairs viability of the cell (e.g., substantially reduced cell viability). In some embodiments, an altered (e.g., increased or decreased) expression level and/or activity of one gene of a synthetic lethal pair, but not both, has minimal effect on cell viability. In some embodiments, a cell comprising a decreased expression level and/or activity of both genes of the synthetic lethal pair, or transcriptional or translational products thereof, has substantially reduced viability. In some embodiments, the synthetic lethal pair comprises a biomarker described herein and a target gene. In some embodiments, the synthetic lethal pair comprises a biomarker described herein and PKMYT1.

As used herein, a “biomarker” refers to a gene, or a transcriptional or translational product thereof, whose expression level and/or activity can be detected in a tissue sample obtained from a subject having a disease or disorder (e.g., cancer), wherein an altered (e.g., increased or decreased) expression level and/or activity of the biomarker, e.g., relative to a reference tissue sample, functions as an indicator (e.g., diagnostic, predictive, and/or prognostic indicator). In some embodiments, the biomarker is a predictive indicator, wherein an altered expression level and/or activity of the biomarker in a diseased (e.g., cancerous) tissue sample indicates responsiveness of the disease (e.g., cancer) to a particular therapeutic intervention (e.g., administration of one or more therapeutic agents for modulation (e.g., decrease) of expression level and/or activity of PKMYT1). In some embodiments, the biomarker is a prognostic indicator, wherein an altered expression level and/or activity of the biomarker in a diseased (e.g., cancerous) tissue sample indicates an outcome of the disease or disease progression (e.g., cancer) regardless of therapeutic intervention. In some embodiments, the expression level and/or activity of the biomarker is detected in a tissue sample obtained from a subject having cancer. In some embodiments, an altered (e.g., increased or decreased) expression level and/or activity of the biomarker is a predictive indicator that the subject will respond or will likely respond to therapeutic manipulation of a target gene (e.g., PKMYT1). In some embodiments, a decreased expression level and/or activity of the biomarker is a predictive indicator that the subject will respond or will likely respond to a therapeutic inhibition of a target gene (e.g., PKMYT1).

As used herein, a “tissue sample” refers to a collection of similar cells obtained from a tissue of the subject, e.g., cancer tissue. In some embodiments, the tissue sample is a fresh, frozen, and/or preserved organ, biopsy, and/or aspirate obtained from the subject. In some embodiments, the tissue sample is blood or any blood constituent (e.g., plasma) collected from the subject. In some embodiments, the tissue sample is blood or any blood constituent (e.g., plasma) collected from the subject containing circulating DNA of the diseased tissue (e.g., circulating tumor DNA). In some embodiments, the tissue sample is a bodily fluid (e.g., cerebral spinal fluid, amniotic fluid, peritoneal fluid, or interstitial fluid) obtained from the subject. In some embodiments, the tissue sample is obtained from the diseased tissue or organ (e.g., a cancerous tissue or organ). In some embodiments, the tissue sample comprises non-natural compounds, e.g., preservatives, anticoagulants, buffers, fixatives, nutrients, antibiotics.

As used herein, the term “responsiveness” refers to the degree to which a diseased tissue (e.g., a tumor) in a subject undergoes a desirable therapeutic change upon exposure to an inhibitor of a target gene described herein (e.g., a PKMYT1 gene) or a transcriptional or translation product thereof (e.g., an RNA transcript encoding a PKMYT1 polypeptide or a PKMYT1 polypeptide). In some embodiments, the diseased tissue sample is a tumor and the desirable therapeutic outcome is reduced tumor burden, regression of tumor burden, and/or reduced growth of the tumor.

In some embodiments, an altered (e.g., increased or decreased) expression level of the biomarker in a tissue sample (e.g., cancer sample) obtained from a subject with cancer is a predictive indicator that the subject will respond or will likely respond to therapeutic manipulation of a target gene (e.g., PKMYT1). As used herein, an “altered expression level” refers to an increased or decreased expression level of the biomarker in a diseased tissue sample (e.g., cancer sample) obtained from the subject relative to a reference sample. In some embodiments, a decreased expression level of the biomarker in a diseased tissue sample (e.g., cancer sample) obtained from a subject with cancer is a predictive indicator that the subject will respond or will likely respond to therapeutic inhibition of a target gene (e.g., PKMYT1).

In some embodiments, an altered (e.g., increased or decreased) activity of the biomarker in a tissue sample (e.g., cancer sample) obtained from a subject with cancer is a predictive and/or prognostic indicator that the subject will respond or will likely respond to therapeutic manipulation of a target gene (e.g., PKMYT1). As used herein, an “altered activity level” refers to an increased or decreased activity of the biomarker in a diseased tissue sample (e.g., cancer sample) obtained from the subject relative to a reference sample. In some embodiments, a decreased activity of the biomarker in a tissue sample (e.g., cancer sample) obtained from a subject with cancer is a predictive indicator that the subject will respond or will likely respond to therapeutic inhibition of a target gene (e.g., PKMYT1).

As used herein, a “reference sample,” “reference cell,” “reference tissue,” “control sample,” “control cell,” or “control tissue” each refer to a sample, cell, tissue, standard, or level that is used for comparison to establish whether the expression level and/or activity of the biomarker in a subject having a disease (e.g., cancer) is altered (e.g., increased or decreased) relative to a subject not having the disease (e.g., a non-cancerous subject). For example, in some embodiments, a reference sample is obtained from a subject or subjects lacking the disease or disorder (e.g., a non-cancer subject or subjects). In some embodiments, the reference sample is a non-diseased tissue obtained from the subject having the disease (e.g., cancer).

In some embodiments, the disclosure provides a biomarker comprising one or more mutations (e.g., a loss of function mutation or inactivating mutation resulting in a decreased expression level and/or activity of the biomarker) in one or more human cancers, wherein the biomarker forms a synthetic lethal pair with a PKMYT1 target gene, or a transcriptional or translation product thereof (e.g., an RNA transcript encoding a PKMYT1 polypeptide or a PKMYT1 polypeptide). In some embodiments, the presence of one or more mutations in the biomarker in a diseased (e.g., cancerous) tissue sample indicates responsiveness of the disease (e.g., cancer) to a particular therapeutic intervention directed to the PKMYT1 gene, or a transcriptional or translation product thereof (e.g., administration of one or more therapeutic agents for modulating (e.g., decreasing) an expression level and/or activity of a PKMYT1 polypeptide). In some embodiments, the presence of one or more mutations in the biomarker is detected in a tissue sample obtained from a subject having cancer. In some embodiments, the presence of one or more mutations in the biomarker is a predictive indicator that the subject's cancer will respond or will likely respond to therapeutic manipulation of the PKMYT1 gene, or a transcriptional or translation product thereof. In some embodiments, the presence of one or more mutations in the biomarker that is a loss of function mutation (e.g., results in a decreased expression level and/or activity of the biomarker) is a predictive indicator that the subject's cancer will respond or will likely respond one or more therapeutic agents for modulating the PKMYT1 gene, or a transcriptional or translation product thereof (e.g., an RNA transcript encoding a PKMYT1 polypeptide or a PKMYT1 polypeptide).

In some embodiments, the expression level and/or activity of the biomarker is altered (e.g., increased or decreased) due to one or more mutations. In some embodiments, the one or more mutations occur in the gene encoding the biomarker (e.g., homozygous mutation). In some embodiments, the one or more mutations comprises a nonsynonymous mutation (which results in a change to the encoded protein sequence). In some embodiments, the nonsynonymous mutation occurs adjacent to or proximal to the 5′ end of the open reading frame of the gene encoding the biomarker. Without being bound by theory, a nonsynonymous mutation occurring adjacent to or proximal to the 5′ end of the open reading frame has increased likelihood of generating a loss of function or inactivation of the biomarker. In some embodiments, the one or more mutations (e.g., the nonsynonymous mutation) comprises a missense mutation (point mutation that results in a codon that encodes a different amino acid residue compared to the wild-type or non-mutated amino acid sequence). In some embodiments, the missense mutation occurs adjacent to or proximal to the 5′ end of the open reading frame of the gene encoding the biomarker. In some embodiments, the one or more mutations (e.g., the nonsynonymous mutation) comprises a nonsense mutation (point mutation in the gene sequence that results in a premature stop codon or nonsense codon on the transcribed mRNA that produces a translation product that is a truncated or incomplete). In some embodiments, the nonsense mutation occurs adjacent to or proximal to the 5′ end of the open reading frame of the gene encoding the biomarker. In some embodiments, the one or more mutations (e.g., nonsynonymous mutation) comprises a nonstop mutation (point mutation occurring within translational stop codons that result in continued and inappropriate translation of mRNA transcript into the 3′ untranslated region). In some embodiments, the one or more mutations (e.g., nonsynonymous mutation) comprises an insertion of one or more nucleotides. In some embodiments, the insertion results in a frameshift mutation (change in the open reading frame of the gene encoding the biomarker). In some embodiments, the one or more mutations (e.g., nonsynonymous mutation) comprises a deletion of one or more nucleotides. In some embodiments, the deletion results in a frameshift mutation. In some embodiments, the frameshift mutation results in a gene encoding an altered (e.g., inactivated) protein product. In some embodiments, the one or more mutations comprises an inversion. In some embodiments, the one or more mutations comprises a deletion-insertion. In some embodiments, the one or more mutations comprises a homozygous deletion. In some embodiments, the one or more mutations comprises a missense mutation in the gene encoding the biomarker, wherein the mutated gene is predicted to encode a nonfunctional protein. For example, the mutated gene is predicted to encode a nonfunctional protein using a SIFT algorithm (see, e.g., Nature Protocols (2016) 11:1-9), wherein the SIFT value is equal to or approximately zero. In some embodiments, the one or more mutations comprises a nonsense mutation in the gene encoding the biomarker, wherein the mutated gene encodes a truncated protein. In some embodiments, the one or more mutations comprises a nonstop mutation in the gene encoding the biomarker, wherein the mutated gene encodes a longer (e.g., non-functional or inactivated) protein. In some embodiments, the one or more mutations is a duplication, a deletion, or an insertion. In some embodiments, the duplication, deletion, or insertion results in a frameshift mutation.

In some embodiments, the one or more mutation alters (e.g., increases or decreases) the expression of the gene encoding the biomarker. In some embodiments, the one or more mutations comprises a splice site mutation. In some embodiments, the one or more mutations (e.g., splice site mutation) results in altered splicing of a transcriptional product of the gene encoding the biomarker. In some embodiments, the one or more mutations results in a transcriptional product having impaired nuclear translocation. In some embodiments, the one or more mutations results in a transcriptional product having impaired translation. In some embodiments, the one or more mutations results in a translational product having a non-natural substitution of one amino acid for another. In some embodiments, the one or more mutations results in a translational product having a deletion or an insertion of one or more amino acid residues. In some embodiments, the one or more mutations results in a truncated translational product. In some embodiments, the one or more mutations results in translational product that is a fusion with another protein. In some embodiments, the translational product is inactive or has low activity relative to a translational product expressed from a wild-type gene encoding the biomarker.

In some embodiments, the one or more mutations comprises a nonsynonymous mutation (which results in a change to the encoded protein sequence). In some embodiments, the nonsynonymous mutation occurs adjacent to or proximal to the 5′ end of the open reading frame of the gene encoding the biomarker. Without being bound by theory, a nonsynonymous mutation occurring adjacent to or proximal to the 5′ end of the open reading frame has increased likelihood of generating a loss of function or inactivation of the biomarker.

In some embodiments, the one or more mutations (e.g., the nonsynonymous mutation) comprises a missense mutation (point mutation that results in a codon that encodes a different amino acid residue compared to the wild-type or non-mutated amino acid sequence). In some embodiments, the missense mutation occurs adjacent to or proximal to the 5′ end of the open reading frame of the gene encoding the biomarker.

In some embodiments, the one or more mutations (e.g., the nonsynonymous mutation) comprises a nonsense mutation (point mutation in the gene sequence that results in a premature stop codon or nonsense codon on the transcribed mRNA that produces a translation product that is a truncated or incomplete). In some embodiments, the nonsense mutation occurs adjacent to or proximal to the 5′ end of the open reading frame of the gene encoding the biomarker.

In some embodiments, the one or more mutations (e.g., nonsynonymous mutation) comprises a nonstop mutation (point mutation occurring within translational stop codons that result in continued and inappropriate translation of mRNA transcript into the 3′ untranslated region).

In some embodiments, the one or more mutations (e.g., nonsynonymous mutation) comprises an insertion of one or more nucleotides in the gene encoding the biomarker. In some embodiments, the insertion results in a frameshift mutation (change in the open reading frame of the gene encoding the biomarker).

In some embodiments, the one or more mutations (e.g., nonsynonymous mutation) comprises a deletion of one or more nucleotides in the gene encoding the biomarker. In some embodiments, the deletion results in a frameshift mutation. In some embodiments, the frameshift mutation results in a gene encoding an altered (e.g., inactivated) protein product.

In some embodiments, the one or more mutations comprises an inversion.

In some embodiments, the one or more mutations comprises a deletion-insertion.

In some embodiments, the one or more mutations is a duplication, a deletion, or an insertion in the gene encoding the biomarker. In some embodiments, the duplication, deletion, or insertion results in a frameshift mutation.

In some embodiments, the one or more mutation alters (e.g., increases or decreases) the expression of the gene encoding the biomarker. In some embodiments, the one or more mutations comprises a splice site mutation. In some embodiments, the one or more mutations (e.g., splice site mutation) results in altered splicing of a transcriptional product of the gene encoding the biomarker. In some embodiments, the one or more mutations results in a transcriptional product having impaired nuclear translocation. In some embodiments, the one or more mutations results in a transcriptional product having impaired translation. In some embodiments, the one or more mutations results in a translational product having a non-natural substitution of one amino acid for another. In some embodiments, the one or more mutations results in a translational product having a deletion or an insertion of one or more amino acid residues. In some embodiments, the one or more mutations results in a truncated translational product. In some embodiments, the one or more mutations results in translational product that is a fusion with another protein. In some embodiments, the translational product is inactive or has low activity relative to a translational product expressed from a wild-type gene encoding the biomarker.

In some embodiments, the expression level and/or activity of the biomarker is altered (e.g., increased or decreased) due to one or more deficiency in the biomarker. In some embodiments, the one or more deficiencies are selected from multiple copies of the same gene, hypermethylation, deep deletion, mutation in the gene encoding the biomarker, or a combination thereof.

In some embodiments, the expression level and/or activity of the biomarker is decreased by a mutation in the gene encoding the biomarker. In some embodiments, the mutation is a deletion.

In some embodiments, the presence of a mutation and/or a deficiency in the biomarker is measured in a tissue sample (e.g., cancer sample) obtained from the subject using a method of mutational detection analysis (e.g., next generation sequencing). In some embodiments, the tissue sample is a tumor biopsy sample (e.g., fresh or fixed tumor biopsy sample). In some embodiments, the tissue sample is a blood sample comprising circulating tumor DNA.

In some embodiments, the disclosure provides a biomarker comprising a mutation (e.g., loss of function mutation resulting in a decreased expression level and/or activity of the biomarker) in one or more human cancers, wherein the biomarker forms a synthetic lethal pair with at least one or more target genes. In some embodiments, the presence of a mutation in the biomarker in a diseased (e.g., cancerous) tissue sample indicates responsiveness of the disease (e.g., cancer) to a particular therapeutic intervention (e.g., administration of one or more therapeutic agents for modulation (e.g., decrease) of expression level and/or activity of PKMYT1). In some embodiments, the presence of a mutation in the biomarker is detected in a tissue sample obtained from a subject having cancer. In some embodiments, the presence of a mutation in the biomarker is a predictive indicator that the subject's cancer will respond or will likely respond to therapeutic manipulation of a target gene (e.g., PKMYT1). In some embodiments, the presence of a mutation in the biomarker that results in a loss of function of the biomarker (e.g., decreased expression level and/or activity of the biomarker) is a predictive indicator that the subject's cancer will respond or will likely respond to a therapeutic inhibition of a target gene (e.g., PKMYT1).

In some embodiments, the mutation comprises a deletion of the gene encoding the biomarker (e.g., homozygous deletion). In some embodiments, the mutation is a missense mutation. In some embodiments, the missense mutation results in a gene predicted to encode a nonfunctional protein, optionally wherein the prediction is performed using a SIFT algorithm. In some embodiments, the mutation is a missense mutation resulting in a gene encoding a truncated protein. In some embodiments, the mutation is a nonsense mutation resulting in a gene encoding a truncated protein. In some embodiments, the one or more mutations is a duplication, a deletion, or an insertion. In some embodiments, the duplication, deletion, or insertion results in a frameshift mutation. In some embodiments, the mutation is a splice site mutation. In some embodiments, the mutation results in a loss of function of the biomarker (e.g., decreased expression level and/or activity of the biomarker). In some embodiments, the presence of a mutation in the biomarker is measured in a tissue sample (e.g., cancer sample) obtained from the subject using a method of mutational detection analysis (e.g., next generation sequencing). In some embodiments, the tissue sample is a tumor biopsy sample (e.g., fresh or fixed tumor biopsy sample). In some embodiments, the tissue sample is a blood sample comprising circulating tumor DNA.

In some embodiments, the one or more deficiencies are prevalent in one or more human cancers. As used herein, “prevalent” refers to the frequency in which an altered (e.g., increased or decreased) expression level and/or activity of a biomarker relative to a reference sample occurs in a demographic of subjects affected by a particular type of cancer (e.g., colon adenocarcinoma). In some embodiments, the one or more deficiencies is prevalent (e.g., frequency greater than about 1%, about 2%, about 3%, about 4%, about 5%, about 10%, about 15%, about 20%, about 25%, about 30%, about 35%, about 40%, about 45%, or about 50%) in one or more human cancers selected from acute myeloid leukemia (LAML), adrenocortical carcinoma (ACC), bladder urothelial carcinoma (BLCA), brain lower grade glioma (LGG), breast invasive carcinoma (BRCA), cervical squamous cell carcinoma and endocervical adenocarcinoma (CESC), cholangiocarcinoma (CHOL), chronic myelogenous leukemia (LCML), colon adenocarcinoma (COAD), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), lymphoid neoplasm diffuse large B-cell lymphoma (DLBC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), testicular germ cell tumors (TGCT), thymoma (THYM), thyroid carcinoma (THCA), uterine carcinosarcoma (UCS), uterine corpus endometrial carcinoma (UCEC), and uveal melanoma (UVM).

In some embodiments, the presence of such genetic mutations is identified by assaying tissue-derived cells obtained from a subject. For example, suitable assays for use in the present disclosure include those involving genomic DNA, mRNA, or cDNA. For a nucleic acid-based detection method, genomic DNA is first obtained (using any standard technique) from cells (e.g., ovarian cells) of a subject to be tested. If appropriate, cDNA can be prepared or mRNA can be obtained. In some instances, nucleic acids can be amplified by any known nucleic acid amplification technique (e.g., polymerase chain reaction) to a sufficient quantity and purity, and further analyzed to detect mutations. For example, genomic DNA can be isolated from a sample, and all exonic sequences, and the intron/exon junction regions including the regions required for exon/intron splicing, can be amplified into one or more amplicons and further analyzed for the presence or absence of mutations. In some instances, the assay is a next generation sequencing-based assay, such as FoundationOne®CDx™ or Tempus xT™. In some embodiments, the presence of a mutation in a biomarker of the disclosure is detected using any method of mutational detection analysis that is known in the art. Non-limiting exemplary methods of mutational detection analysis include fluorescence in situ hybridization (FISH), PCR, RT-PCR, gel electrophoresis, DNA microarray, DNA sequencing (e.g., via next generation sequencing or Sanger sequencing), multiplex ligation-dependent probe amplification, fluorescent melting curve analysis, and pyrosequencing.

### Exemplary Synthetic Lethality Biomarkers

In some embodiments, the disclosure provides one or more biomarkers having an altered expression level and/or activity in one or more human cancers, wherein the one or more biomarkers each form a synthetic lethal pair with PKMYT1. In some embodiments, the disclosure provides one or more biomarkers having a loss of function in one or more human cancers, wherein the one or more biomarkers each form a synthetic lethal pair with PKMYT1. In some embodiments, the disclosure provides one or more biomarkers comprising a mutation in one or more human cancers, wherein the one or more biomarkers each form a synthetic lethal pair with PKMYT1. In some embodiments, the disclosure provides one or more biomarkers that are mutated in one or more human cancers, wherein the one or more biomarkers each form a synthetic lethal pair with PKMYT1.

In some embodiments, an altered (e.g., increased or decreased) expression level of the biomarker in a cancer tissue obtained from a subject is a predictive indicator that the subject will or will likely respond to therapeutic manipulation of PKMYT1. In some embodiments, a decreased expression level of the biomarker in a cancer tissue obtained from a subject is a predictive indicator the subject will or will likely respond to therapeutic inhibition of PKMYT1. In some embodiments, a mutation in the biomarker (e.g., a loss of function mutation resulting in a decreased expression level and/or activity of the biomarker) in a cancer tissue obtained from a subject is a predictive indicator that the subject will or will likely respond to therapeutic manipulation of PKMYT1. In some embodiments, the response comprises decreased tumor progression. In some embodiments, the response comprises tumor shrinkage. In some embodiments, the response comprises reduced risk of metastasis.

In some embodiments, the biomarker and PKMYT1 form a synthetic lethal pair, such that inhibition or decreased expression level and/or activity of both the biomarker and PKMYT1 is lethal to the cell (e.g., results in apoptosis, necrosis, inhibition of proliferation, or substantially reduced viability), whereas the inhibition or decreased expression level and/or activity of either gene alone has minimal or no effect on the viability of the cell (e.g., is not sufficient to kill the cell). In some embodiments, a cell or a population of cells having an altered (e.g., decreased or diminished) expression level and/or activity of (i) a biomarker described herein, or (ii) PKMYT1 results in a reduction in viability of the cell or population of cells, but a combination of (i) and (ii) results in a greater reduction in viability of the cell or the population of cells. In some embodiments, a population of cells (e.g., a population of cancer cells) comprising a decreased expression level and/or activity of (i) a biomarker described herein, and (ii) PKMYT1 has a proportion of dead or dying cells that is greater than the sum proportion of the dead or dying cells in a first cell population comprising (i) and a second cell population comprising (ii).

In some embodiments, the biomarker is a protein that is an upstream agonist or antagonist PKMYT1. In some embodiments, PKMYT1 is an upstream agonist or antagonist of the biomarker. In some embodiments, the biomarker is an agonist or antagonist of another gene (or encoded protein) that regulates PKMYT1. In some embodiments, the biomarker and PKMYT1 regulate a subset of the same downstream genes or signaling components. For example, in some embodiments, the biomarker regulates a plurality of downstream genes or signaling components, a subset of which are also regulated by PKMYT1. In some embodiments, the downstream genes or signaling components may affect a cancer-related process, e.g., HIPPO pathway, epithelial-to-mesenchymal transition, PI3K pathway, DNA replication, cell migration, cell metastasis, etc. Alternatively, or in addition to, the biomarker and PKMYT1 may be regulated by a subset of the same genes.

In some embodiments, deficiency in the expression and/or activity level of the biomarker (e.g., via a mutation and/or deletion of the biomarker) enhances modulation (e.g., decrease) of an expression level and/or activity of PKMYT1 by one or more therapeutic agents described herein. In some embodiments, a deficiency in the expression level and/or activity of the biomarker (e.g., via a mutation and/or deletion of the biomarker) is enhanced by an altered (e.g., increased or decreased) expression level and/or activity of PKMYT1 using a therapeutic agent described herein.

PKMYT1 interacts with or regulates CDK1 and thereby affects cell cycle progression. Without being bound by theory, inhibition of PKMYT1 results in uncontrolled cell cycle progression, wherein the presence of damaged DNA will cause cell lethality due to mitotic catastrophe. Accordingly, in some embodiments, a biomarker of the disclosure is involved in regulating DNA damage repair (e.g., as a component or a subunit of a component in a cellular DNA repair pathway). In some embodiments, an altered (e.g., increased or decreased) expression and/or activity of a biomarker that is involved in regulating DNA damage repair (e.g., as a component or a subunit of a component in a cellular DNA repair pathway) results in accumulated damaged DNA. Without being bound by theory, a biomarker that is involved in regulating DNA damage repair (e.g., as a component or a subunit of a component in a cellular DNA repair pathway) is a synthetic lethal pair with PKMYT1 due to accumulation of damaged DNA and uncontrolled cell cycle progression that results in loss of cell viability.

In some embodiments, a biomarker of the disclosure is a subunit of PP2A. In some embodiments, the PP2A subunit is PPP2R1B.

In some embodiments, a biomarker of the disclosure is a protein phosphatase 3 (PP3) subunit. In some embodiments, the PP3 subunit is PPP3CC.

In some embodiments, a biomarker of the disclosure is ATM. In some embodiments, a biomarker of the disclosure is MAP2K4. In some embodiments, a biomarker of the disclosure is TP53. In some embodiments, a biomarker of the disclosure is CDC25A. In some embodiments, a biomarker of the disclosure is CACNA1H. In some embodiments, a biomarker of the disclosure is CDKN1B. In some embodiments, a biomarker of the disclosure is DUSP7. In some embodiments, a biomarker of the disclosure is FOXO3. In some embodiments, a biomarker of the disclosure is FZD3. In some embodiments, a biomarker of the disclosure is JAK1. In some embodiments, a biomarker of the disclosure is SMAD2. In some embodiments, a biomarker of the disclosure is TGFBR2. In some embodiments, a biomarker of the disclosure is MAP3K2.

In some embodiments, a synthetic lethal pair of the disclosure comprises ATM. In some embodiments, a synthetic lethal pair of the disclosure comprises MAP2K4. In some embodiments, a synthetic lethal pair of the disclosure comprises TP53. In some embodiments, a synthetic lethal pair of the disclosure comprises CDC25A. In some embodiments, a synthetic lethal pair of the disclosure comprises CACNA1H. In some embodiments, a synthetic lethal pair of the disclosure comprises CDKN1B. In some embodiments, a synthetic lethal pair of the disclosure comprises DUSP7. In some embodiments, a synthetic lethal pair of the disclosure comprises FOXO3. In some embodiments, a synthetic lethal pair of the disclosure comprises FZD3. In some embodiments, a synthetic lethal pair of the disclosure comprises JAK1. In some embodiments, a synthetic lethal pair of the disclosure comprises SMAD2. In some embodiments, a synthetic lethal pair of the disclosure comprises TGFBR2. In some embodiments, a synthetic lethal pair of the disclosure comprises MAP3K2.

In some embodiments, a synthetic lethal pair of the disclosure comprises PKMYT1 and the biomarker ATM. In some embodiments, a synthetic lethal pair of the disclosure comprises PKMYT1 and the biomarker MAP2K4. In some embodiments, a synthetic lethal pair of the disclosure comprises PKMYT1 and the biomarker TP53. In some embodiments, a synthetic lethal pair of the disclosure comprises PKMYT1 and the biomarker CDC25A. In some embodiments, a synthetic lethal pair of the disclosure comprises PKMYT1 and the biomarker CACNA1H. In some embodiments, a synthetic lethal pair of the disclosure comprises PKMYT1 and the biomarker CDKN1B. In some embodiments, a synthetic lethal pair of the disclosure comprises PKMYT1 and the biomarker DUSP7. In some embodiments, a synthetic lethal pair of the disclosure comprises PKMYT1 and the biomarker FOXO3. In some embodiments, a synthetic lethal pair of the disclosure comprises PKMYT1 and the biomarker FZD3. In some embodiments, a synthetic lethal pair of the disclosure comprises PKMYT1 and the biomarker JAK1. In some embodiments, a synthetic lethal pair of the disclosure comprises PKMYT1 and the biomarker SMAD2. In some embodiments, a synthetic lethal pair of the disclosure comprises PKMYT1 and the biomarker TGFBR2. In some embodiments, a synthetic lethal pair of the disclosure comprises PKMYT1 and the biomarker MAP3K2.

In some embodiments, a biomarker of the disclosure is BIN3. In some embodiments, a biomarker of the disclosure is AGPAT5. In some embodiments, a biomarker of the disclosure is FGF17. In some embodiments, a biomarker of the disclosure is PBK. In some embodiments, a biomarker of the disclosure is NOTCH1. In some embodiments, a biomarker of the disclosure is CNTN5. In some embodiments, a biomarker of the disclosure is IRF2. In some embodiments, a biomarker of the disclosure is ALPK2. In some embodiments, a biomarker of the disclosure is CDH19. In some embodiments, a biomarker of the disclosure is CHKB. In some embodiments, a biomarker of the disclosure is MAPK12. In some embodiments, a biomarker of the disclosure is SLC8A1. In some embodiments, a biomarker of the disclosure is HDAC2. In some embodiments, a biomarker of the disclosure is CDT1. In some embodiments, a biomarker of the disclosure is ADCY2. In some embodiments, a biomarker of the disclosure is SLK. In some embodiments, a biomarker of the disclosure is CDC20B. In some embodiments, a biomarker of the disclosure is RPS6KA3. In some embodiments, a biomarker of the disclosure is STAG1. In some embodiments, a biomarker of the disclosure is CKAP5. In some embodiments, a biomarker of the disclosure is RAD51. In some embodiments, a biomarker of the disclosure is CKS1B. In some embodiments, a biomarker of the disclosure is CCNO. In some embodiments, a biomarker of the disclosure is KCNA2. In some embodiments, a biomarker of the disclosure is MCM4. In some embodiments, a biomarker of the disclosure is PLK4. In some embodiments, a biomarker of the disclosure is CDC16.

In some embodiments, a biomarker of the disclosure is ERICH1. In some embodiments, a biomarker of the disclosure is TNKS. In some embodiments, a biomarker of the disclosure is TDRP. In some embodiments, a biomarker of the disclosure is MTUS1. In some embodiments, a biomarker of the disclosure is TNFRSF10B. In some embodiments, a biomarker of the disclosure is HR. In some embodiments, a biomarker of the disclosure is TNFRSF10D. In some embodiments, a biomarker of the disclosure is DMTN. In some embodiments, a biomarker of the disclosure is ENTPD4. In some embodiments, a biomarker of the disclosure is TNFRSF10C. In some embodiments, a biomarker of the disclosure is PEBP4. In some embodiments, a biomarker of the disclosure is LPL. In some embodiments, a biomarker of the disclosure is LGI3. In some embodiments, a biomarker of the disclosure is SLC7A2. In some embodiments, a biomarker of the disclosure is MTMR9. In some embodiments, a biomarker of the disclosure is MSRA. In some embodiments, a biomarker of the disclosure is PDLIM2. In some embodiments, a biomarker of the disclosure is INTS10. In some embodiments, a biomarker of the disclosure is SH2D4A. In some embodiments, a biomarker of the disclosure is GFRA2. In some embodiments, a biomarker of the disclosure is ZDHHC2. In some embodiments, a biomarker of the disclosure is PDGFRL. In some embodiments, a biomarker of the disclosure is SPAG11B. In some embodiments, a biomarker of the disclosure is PPP1R3B. In some embodiments, a biomarker of the disclosure is SPAG11A. In some embodiments, a biomarker of the disclosure is REEP4. In some embodiments, a biomarker of the disclosure is DEFA5. In some embodiments, a biomarker of the disclosure is DEFB136. In some embodiments, a biomarker of the disclosure is NRG1. In some embodiments, a biomarker of the disclosure is ASAH1. In some embodiments, a biomarker of the disclosure is DEFA3. In some embodiments, a biomarker of the disclosure is EPHX2. In some embodiments, a biomarker of the disclosure is CNOT7. In some embodiments, a biomarker of the disclosure is PNMA2. In some embodiments, a biomarker of the disclosure is TRIM35. In some embodiments, a biomarker of the disclosure is ATRX. In some embodiments, a biomarker of the disclosure is INTS9. In some embodiments, a biomarker of the disclosure is DNAH3. In some embodiments, a biomarker of the disclosure is MAP3K1. In some embodiments, a biomarker of the disclosure is RIMS2. In some embodiments, a biomarker of the disclosure is NSD1. In some embodiments, a biomarker of the disclosure is SARAF.

In some embodiments, a biomarker of the disclosure is CDKN2B. In some embodiments, a biomarker of the disclosure is CSMD3. In some embodiments, a biomarker of the disclosure is LRP1B. In some embodiments, a biomarker of the disclosure is DMRTA1. In some embodiments, a biomarker of the disclosure is PTPRD. In some embodiments, a biomarker of the disclosure is ELAVL2. In some embodiments, a biomarker of the disclosure is FAT1. In some embodiments, a biomarker of the disclosure is CDH1. In some embodiments, a biomarker of the disclosure is NF1. In some embodiments, a biomarker of the disclosure is PPP6R2. In some embodiments, a biomarker of the disclosure is PIM3. In some embodiments, a biomarker of the disclosure is MAPK11. In some embodiments, a biomarker of the disclosure is CDH10. In some embodiments, a biomarker of the disclosure is PCDH15. In some embodiments, a biomarker of the disclosure is ALB. In some embodiments, a biomarker of the disclosure is OR4F21. In some embodiments, a biomarker of the disclosure is LINGO2. In some embodiments, a biomarker of the disclosure is FBN2. In some embodiments, a biomarker of the disclosure is CACNA1E. In some embodiments, a biomarker of the disclosure is LRRC7. In some embodiments, a biomarker of the disclosure is NALCN. In some embodiments, a biomarker of the disclosure is ARID1A. In some embodiments, a biomarker of the disclosure is ADGRB3. In some embodiments, a biomarker of the disclosure is SI. In some embodiments, a biomarker of the disclosure is PKHD1L1. In some embodiments, a biomarker of the disclosure is TBC1D22A. In some embodiments, a biomarker of the disclosure is BNIP3L. In some embodiments, a biomarker of the disclosure is DEFA1. In some embodiments, a biomarker of the disclosure is DEFB103B. In some embodiments, a biomarker of the disclosure is DEFB103A. In some embodiments, a biomarker of the disclosure is HCN1. In some embodiments, a biomarker of the disclosure is RELN. In some embodiments, a biomarker of the disclosure is UNC13C. In some embodiments, a biomarker of the disclosure is XKR5. In some embodiments, a biomarker of the disclosure is CHMP7. In some embodiments, a biomarker of the disclosure is CHRNA2. In some embodiments, a biomarker of the disclosure is CSGALNACT1. In some embodiments, a biomarker of the disclosure is FAM86B2. In some embodiments, a biomarker of the disclosure is EGR3. In some embodiments, a biomarker of the disclosure is XPO7. In some embodiments, a biomarker of the disclosure is TRPS1. In some embodiments, a biomarker of the disclosure is KDM6A.

In some embodiments, a biomarker of the disclosure is NBEA. In some embodiments, a biomarker of the disclosure is VPS37A. In some embodiments, a biomarker of the disclosure is SCN1A. In some embodiments, a biomarker of the disclosure is CSMD2. In some embodiments, a biomarker of the disclosure is GTSE1. In some embodiments, a biomarker of the disclosure is TRMU. In some embodiments, a biomarker of the disclosure is TENM1. In some embodiments, a biomarker of the disclosure is DOCK3. In some embodiments, a biomarker of the disclosure is VPS13B. In some embodiments, a biomarker of the disclosure is RBM10. In some embodiments, a biomarker of the disclosure is RYR2. In some embodiments, a biomarker of the disclosure is SCARA5. In some embodiments, a biomarker of the disclosure is SETBP1. In some embodiments, a biomarker of the disclosure is DYSF. In some embodiments, a biomarker of the disclosure is NLGN4X. In some embodiments, a biomarker of the disclosure is EPHA3. In some embodiments, a biomarker of the disclosure is FBLN1. In some embodiments, a biomarker of the disclosure is ADAMTS20. In some embodiments, a biomarker of the disclosure is IFT74. In some embodiments, a biomarker of the disclosure is KLKB1. In some embodiments, a biomarker of the disclosure is ACVR2A. In some embodiments, a biomarker of the disclosure is ZFHX4. In some embodiments, a biomarker of the disclosure is WWC2. In some embodiments, a biomarker of the disclosure is MOB3B. In some embodiments, a biomarker of the disclosure is DMXL1. In some embodiments, a biomarker of the disclosure is ELAC1. In some embodiments, a biomarker of the disclosure is RBPMS. In some embodiments, a biomarker of the disclosure is ANK1. In some embodiments, a biomarker of the disclosure is CADM2. In some embodiments, a biomarker of the disclosure is C9orf72. In some embodiments, a biomarker of the disclosure is MTNR1A. In some embodiments, a biomarker of the disclosure is PLAA. In some embodiments, a biomarker of the disclosure is NIPBL. In some embodiments, a biomarker of the disclosure is ASPM. In some embodiments, a biomarker of the disclosure is GABRB3. In some embodiments, a biomarker of the disclosure is CTNNA3. In some embodiments, a biomarker of the disclosure is CNTN3. In some embodiments, a biomarker of the disclosure is PPFIA2. In some embodiments, a biomarker of the disclosure is FN1. In some embodiments, a biomarker of the disclosure is HECW1. In some embodiments, a biomarker of the disclosure is DMXL2. In some embodiments, a biomarker of the disclosure is ZFP36L2. In some embodiments, a biomarker of the disclosure is UPK3A. In some embodiments, a biomarker of the disclosure is SMC1B. In some embodiments, a biomarker of the disclosure is SMARCA4. In some embodiments, a biomarker of the disclosure is LRFN5. In some embodiments, a biomarker of the disclosure is TG. In some embodiments, a biomarker of the disclosure is CTNND2. In some embodiments, a biomarker of the disclosure is CHD1. In some embodiments, a biomarker of the disclosure is LSAMP. In some embodiments, a biomarker of the disclosure is PRR5. In some embodiments, a biomarker of the disclosure is NPAP1. In some embodiments, a biomarker of the disclosure is SNTG1. In some embodiments, a biomarker of the disclosure is MDGA2. In some embodiments, a biomarker of the disclosure is BNC2. In some embodiments, a biomarker of the disclosure is SCN2A. In some embodiments, a biomarker of the disclosure is HERC2. In some embodiments, a biomarker of the disclosure is SCN3A. In some embodiments, a biomarker of the disclosure is TRPM1. In some embodiments, a biomarker of the disclosure is FSTL5. In some embodiments, a biomarker of the disclosure is ASH1L. In some embodiments, a biomarker of the disclosure is PRKDC. In some embodiments, a biomarker of the disclosure is TCF4. In some embodiments, a biomarker of the disclosure is SVIL. In some embodiments, a biomarker of the disclosure is CHD4. In some embodiments, a biomarker of the disclosure is PCDH9. In some embodiments, a biomarker of the disclosure is NRXN3. In some embodiments, a biomarker of the disclosure is SNX25. In some embodiments, a biomarker of the disclosure is MPDZ. In some embodiments, a biomarker of the disclosure is TLL1. In some embodiments, a biomarker of the disclosure is EPHA6. In some embodiments, a biomarker of the disclosure is FER. In some embodiments, a biomarker of the disclosure is NFASC. In some embodiments, a biomarker of the disclosure is USP34. In some embodiments, a biomarker of the disclosure is SPEF2. In some embodiments, a biomarker of the disclosure is CHD8. In some embodiments, a biomarker of the disclosure is ABCA12. In some embodiments, a biomarker of the disclosure is ARID2. In some embodiments, a biomarker of the disclosure is KCNIP4. In some embodiments, a biomarker of the disclosure is NFIB.

In some embodiments, a biomarker of the disclosure is SLITRK1. In some embodiments, a biomarker of the disclosure is ZNF521. In some embodiments, a biomarker of the disclosure is CCNB1. In some embodiments, a biomarker of the disclosure is CDK7. In some embodiments, a biomarker of the disclosure is MYT1L. In some embodiments, a biomarker of the disclosure is FZR1. In some embodiments, a biomarker of the disclosure is SERF1A. In some embodiments, a biomarker of the disclosure is GADD45B. In some embodiments, a biomarker of the disclosure is ADGRL2. In some embodiments, a biomarker of the disclosure is TTK. In some embodiments, a biomarker of the disclosure is NRXN2. In some embodiments, a biomarker of the disclosure is UNC13A. In some embodiments, a biomarker of the disclosure is ZBTB7A. In some embodiments, a biomarker of the disclosure is POLD1. In some embodiments, a biomarker of the disclosure is PCDH19. In some embodiments, a biomarker of the disclosure is SLC8A2. In some embodiments, a biomarker of the disclosure is E2F4. In some embodiments, a biomarker of the disclosure is AUTS2. In some embodiments, a biomarker of the disclosure is KCNN2. In some embodiments, a biomarker of the disclosure is CCNH. In some embodiments, a biomarker of the disclosure is FRG2C. In some embodiments, a biomarker of the disclosure is PLK2. In some embodiments, a biomarker of the disclosure is MYO18A. In some embodiments, a biomarker of the disclosure is DCAF12L1.

In some embodiments, a biomarker of the disclosure is any one or any combination (e.g., 2, 3, 4, 5 or more) of the biomarkers listed in Table 1.

TABLE 1
Biomarkers of the Disclosure

| | | | | | |
|---|---|---|---|---|---|
| DCTN6 | DDHD2 | RAB3C | CENPH | APP | KIF3A |
| SETDB2 | VWA5A | PUS3 | PTPN21 | CMC2 | PDIA3 |
| DSEL | DEF8 | MARVELD2 | GTPBP6 | RHOBTB3 | EVI5 |
| F11 | GPR63 | CHRNA6 | TBRG1 | NR2C2 | CAMKK1 |
| SMIM18 | RNF152 | CER1 | GTPBP4 | MAGEB3 | ADPRM |
| UBXN8 | EDNRB | PPP4R2 | PDCD2 | ARSK | CDK11B |
| TTI2 | SCEL | HTRA4 | CHRFAM7A | HSF2 | FSHR |
| IGSF9B | AFG1L | NCKIPSD | CD58 | PLN | POLR2B |
| RCBTB1 | PCNX1 | C16orf95 | ATXN7 | TNFSF12 | YEATS2 |
| HDLBP | NT5DC1 | CCDC73 | GUCY1B1 | RPGR | USP8 |
| CTCF | AKAP12 | KLHL30 | ASF1A | MYO1E | DRC7 |
| DIAPH3 | NEK4 | EEF2 | ZCWPW2 | TENT5A | TPD52L1 |
| TSHZ1 | MANEA | ACADVL | VILL | TMEM25 | NTNG1 |
| TLR3 | VPS26B | MAB21L1 | CALHM4 | TMEM144 | RFX2 |
| HTR2A | ITIH4 | GMPPB | ZNF821 | SERINC5 | ZBTB48 |
| STOX2 | ME1 | ANXA10 | DPAGT1 | SPINK8 | PDE5A |
| PHF11 | ALAS1 | KIF4B | NLRP2 | MCTP2 | VDAC3 |
| DGKH | FECH | NKAIN2 | KLHL2 | PLCD1 | STK11IP |
| THSD1 | CUL5 | CDHR2 | ESAM | SLC22A14 | IFIH1 |
| NAA16 | FBXO31 | SETMAR | IL18 | COMMD6 | HPS1 |
| MYH3 | GLI3 | SMG1 | SLC30A5 | PTPN4 | SLC15A1 |
| INTS6 | DGKI | GFM2 | BAZ1A | DDX53 | NOL9 |
| FRG1 | GRAMD1B | AKAP9 | B3GLCT | HSPA4L | UBE2J2 |
| FHOD3 | DBNDD1 | CD164 | PLA2R1 | CP | GJA3 |
| DLEU7 | RBM15B | ALG9 | SMARCAL1 | UBA3 | FAM234B |
| CDKN2AIP | RXFP2 | ZMYND11 | PIK3R6 | GLB1 | TCEANC |
| CASP8 | NARS1 | LTK | RWDD1 | ZNF501 | ABL2 |
| ALG11 | CCNA1 | PLPP5 | STARD4 | PRRG1 | MGAT5 |
| PDLIM3 | UGGT2 | SLC35A1 | HPSE2 | THUMPD3 | GPR82 |
| CPT1B | APLP2 | ALPG | PDE6B | ZNF286B | LRCH3 |
| PDE10A | UBE3A | C16orf46 | SLC16A1 | ARHGAP11B | KCTD12 |
| DCTD | PHLPP2 | FAM172A | BRD7 | ASAP1 | MSH2 |
| TIAM2 | SMOC2 | TRIP11 | XCR1 | IGSF9 | PRPF40A |
| VEGFC | EPB41L2 | TTLL7 | CCR9 | SLC24A4 | KCNAB2 |
| TDRD3 | LTF | C11orf65 | UCHL3 | SOS1 | TBX22 |
| CBLN2 | UNC93A | EFHC2 | JAML | PUDP | DHX37 |
| NEK3 | RHOA | TKT | SLC17A5 | SYNRG | ELMO3 |
| MED4 | BRF2 | ARHGAP22 | MZT1 | EIF4E3 | ZNF180 |
| PARD6G- | ALG5 | AHI1 | FMR1 | ATXN3L | FAF1 |
| AS1 | | | | | |
| LMO7 | POPDC3 | BARX2 | CTBP2 | CALD1 | VCL |
| EPSTI1 | ROCK1 | KCND3 | DHX15 | CDK5RAP2 | GABARAPL2 |
| HELT | COL10A1 | PGM3 | KCTD19 | TMEM242 | SHOC1 |
| FOXO1 | PRKCD | ECT2L | KCNA5 | ZNF619 | OGA |
| FLNA | SLC38A8 | KIF5C | SACM1L | RBBP7 | TAMM41 |
| NEIL3 | GPR35 | ELOVL4 | FRMD4A | EFTUD2 | SPATA22 |
| GALR1 | BAG4 | SLC6A1 | PFKFB4 | DUSP16 | PDE4B |
| RWDD4 | TUBE1 | WDR82 | DUSP22 | PGBD4 | SCN4B |
| RBFA | AMBRA1 | ITGA10 | UCN2 | TIMM8B | DDX47 |
| GPALPP1 | HAUS6 | HOOK3 | PRAP1 | TMEM94 | PDS5A |
| RNF43 | BORA | MTMR10 | INTU | EPM2AIP1 | RNF31 |
| MTRF1 | KCNJ5 | PPWD1 | PARP14 | MTMR1 | ZNF620 |
| ING2 | SH3GL2 | COTL1 | NAA15 | ETFDH | TRIM45 |
| B2M | CNR1 | FAM9B | LCA5L | TSLP | KCNA1 |
| LACC1 | ARIH2OS | UPF3A | SGTB | ZNF418 | MOCS2 |
| ANKRD37 | CCDC68 | PDGFD | SHPK | POU5F2 | PPP4R1L |
| SLC66A2 | CHRND | ACE2 | FUOM | FAM3D | POLN |
| VPS36 | LAMB1 | PLXND1 | USP48 | ARL8B | NCLN |
| CNMD | POU3F2 | VPS11 | MTSS2 | TMEM171 | MPZL2 |
| ZADH2 | C6orf118 | UMODL1- | LRRC3B | LONP2 | DDX19B |
| | | AS1 | | | |
| NCAPH2 | BOK | ANO5 | MAGEB6 | POLR3A | PDHA1 |
| ARSA | PHF10 | BACE2 | ABHD14A- | PKD2L1 | R3HCC1L |
| | | | ACY1 | | |
| PRKG1 | HMGB2 | NLRP12 | ATP1A3 | MCF2L | FMNL1 |
| DNAH8 | ATG16L1 | WNK4 | ARHGAP18 | LARP1B | GAPVD1 |
| MIOX | SLITRK6 | SECISBP2L | ABL1 | STK32C | MKNK2 |
| CWF19L2 | VGLL4 | SLC16A12 | C11orf1 | GLOD4 | ZMYND15 |
| SIK3 | RNF111 | TGM7 | CCDC112 | GTF2H2C | AMELX |
| INPP5D | DNAJC13 | GJB7 | PIK3C2A | HOMER1 | CPTP |
| OCA2 | TPD52L3 | CCR2 | SUPT16H | TMEM181 | ACOT9 |
| MCTP1 | ANKK1 | WTAP | TBX3 | TMIE | F2RL2 |
| LARP4B | PLCB2 | C3orf62 | ESRP2 | SMIM15 | TNFRSF14 |
| FMN1 | PPIL6 | WAPL | MON2 | MYL3 | OR51I2 |
| UHRF2 | ECEL1 | PLA2G4D | MSH4 | RAD51AP2 | TMEM170A |
| KIF20B | FBXL4 | NEU2 | MANF | HDAC5 | HTR2B |
| GLB1L3 | SUMF1 | NUDT12 | TCAIM | CTSD | SMYD4 |
| ZMYM2 | GABRA5 | TSSK1B | GCSH | SLC35G6 | DCLRE1B |
| SIPA1L2 | MCM9 | C10orf90 | AMY2B | SMC3 | FOXR1 |
| DDX3X | CENPBD1 | HOATZ | ARHGAP33 | GOLGA7 | IFT46 |
| SCAP | SLC22A2 | MSL3 | CBR4 | HEY2 | MPZL3 |
| SASH1 | KAT2B | COG4 | ACOT12 | RRAGA | CSTF2T |
| RASGRF2 | DISP2 | PJA2 | TMEM87A | ADAMTS1 | RER1 |
| PARD3 | STXBP5 | ZPR1 | RAB39A | AKAP7 | BTD |
| PSIP1 | LNPEP | EIF4EBP1 | SPA17 | CDH5 | AURKAIP1 |
| DIPK1C | ELL2 | HAPLN1 | MYBPC1 | SEC24A | ADAM10 |
| MID1 | PTH1R | SMIM8 | TNFRSF9 | DAAM1 | CEMIP |
| FAM120B | ADRB3 | HEPACAM | KDM5B | SYCE1L | PLA2G15 |
| SOX6 | FAM193A | SCAPER | CHAF1A | CPEB4 | CDIN1 |
| MINPP1 | GAN | POU2AF1 | SPRN | PHF14 | LTV1 |
| SERP2 | ATMIN | POGLUT3 | NAF1 | N4BP2L1 | TENT4B |
| SBF2 | OR6T1 | IPO7 | LMOD3 | FRMD5 | DFFB |
| SPIRE2 | IL5RA | DYRK1A | ABHD14B | MYEF2 | SLC35E2A |
| WDR6 | TTC21A | C11orf87 | AASDHPPT | GINS4 | CES2 |
| MBD1 | CCDC88C | NDUFAF4 | ZNF470 | ACAA1 | COPS7B |
| ANKRD22 | CFAP53 | TYRO3 | F2RL1 | CDC34 | RAB9A |
| FAS | UQCRC1 | KCND2 | VAC14 | HK3 | HRH2 |
| HTR1E | ATP10D | SMG6 | TCIM | MAMLD1 | ZNRF1 |
| ITGA2 | ATG5 | TP73 | PDK3 | STIM1 | CXCR5 |
| SMIM2 | ARSH | DVL1 | DYNLRB2 | SLN | REEP6 |
| MYO6 | STARD6 | HPF1 | FBXL3 | PKN2 | POLR2E |
| TBC1D5 | MFAP3L | YBX2 | EPS8 | COLQ | CC2D2B |
| RBM5 | RARB | SLC13A5 | ZDHHC3 | MFAP1 | BANK1 |
| DLEC1 | DUSP28 | ENC1 | OPN4 | NUP88 | ATIC |
| FLT1 | GLT8D1 | C11orf53 | STK10 | NSUN2 | ZNF555 |
| MROH2B | EFNA5 | C11orf45 | GSPT2 | SLC26A1 | FGF22 |
| CD109 | SMCHD1 | ZGRF1 | SLC6A3 | SMPDL3A | RNF170 |
| MTREX | KIF2A | CH25H | HTR1B | IWS1 | FBXO30 |
| CYB5A | MIDEAS | SLC39A12 | SFT2D1 | GLRX3 | PICALM |
| GBE1 | ACAP1 | GCNT4 | ZNF662 | LRRC57 | GOLGA8M |
| JHY | AADAT | ULK2 | NFIC | CDYL | CSRNP1 |
| COPS9 | CASP1 | BCKDHB | TLR2 | ACLY | METTL6 |
| RGCC | KLHL18 | HYLS1 | MICU2 | UBP1 | GOLGA8IP |
| CPNE7 | EIF4A1 | MMP10 | ZFYVE28 | RAPGEF4 | ENO3 |
| ATP8B4 | OR8D2 | THEG | UTP15 | VPS4A | PIK3R4 |
| USP45 | GTF3C6 | TAB2 | ZNFX1 | GATM | TIGD4 |
| KLHDC4 | MCCC2 | CCR5 | ZNF35 | SEC24D | TBX5 |
| KCNG4 | USP2 | SHISA5 | OLFM3 | CLTC | C6orf58 |
| SFMBT1 | CASP5 | PTBP1 | SENP7 | TFB1M | CA5B |
| NEU4 | STAR | INPP5A | GNB1 | ZNF528 | WDR37 |
| CDK10 | GON4L | NUDT7 | UTP4 | KCTD8 | BNC1 |
| STK25 | KIAA0513 | NR1D2 | TMEM62 | AGO2 | DYTN |
| NDUFA10 | P4HTM | GJD2 | ADD1 | CCDC80 | TAOK2 |
| SLC12A6 | SYTL5 | TBCEL | ASB11 | DCLRE1A | CREB3L3 |
| NHLRC3 | MAP7D2 | WDR47 | CRMP1 | ZNF471 | PRICKLE1 |
| RARS2 | SF3B3 | PTPRE | MCF2L2 | IL16 | UGT2A1 |
| MYO5A | PHLDB2 | TP53AIP1 | FDX1 | PPIL4 | RPS6 |
| KDSR | AMT | LARP1 | TRIM69 | TRMT11 | CTTNBP2NL |
| MFSD4B | DYM | PLEKHA5 | TMC1 | SNORC | SEC24C |
| DDX10 | TRPA1 | PLD2 | SLC6A2 | MSMO1 | FAM107A |
| SLC16A10 | SAG | PANK4 | HECA | TIGD1 | APBB1 |
| PPP1R7 | GUCY1A1 | SREK1 | CXCR6 | PPID | FAM124B |
| FHL5 | A2ML1 | SLCO1C1 | CXorf38 | GZMM | SMC6 |
| RPAP1 | SIDT2 | SLC25A46 | EXOSC7 | SGK1 | FAM155A |
| PDCD1 | LIPA | MATK | MLH3 | KLHL15 | CHP1 |
| ING5 | RSBN1 | THRB | CXorf21 | SYN1 | GP1BA |
| USP10 | CLASP1 | INTS11 | FAM122A | KLF13 | OXNAD1 |
| GAS8-AS1 | TUBB8 | ACRV1 | ZFP36L1 | ADAL | JMJD7-PLA2G4B |
| GAL3ST2 | NLGN2 | SUPT6H | CXorf58 | BIRC3 | VAMP3 |
| AGRN | SLC37A2 | SOX15 | ABLIM2 | KIF6 | GREM1 |
| FAN1 | COQ3 | HSPB2 | ACER2 | ASB9 | CDO1 |
| DEPDC1B | FNIP1 | C3orf84 | BICC1 | OR51E2 | ESRRG |
| ERMARD | TWIST2 | TM2D2 | ATP13A3 | UPK2 | SLC9A1 |
| SRPRA | AMIGO3 | CTNNA1 | CELF4 | MAGEB16 | FAM3B |
| BRCA1 | GBX2 | TAGAP | PRKCZ | CSNK1G3 | EFR3A |
| JAM3 | ATP7A | SLC16A14 | TRAPPC13 | BMF | GNA15 |
| ATG4B | PRTG | TCP1 | SLC4A1 | ESM1 | ZRSR2 |
| HPGD | GUCY2D | ERBB3 | ARCN1 | TLCD5 | KLF3 |

In some embodiments, a biomarker of the disclosure is any one or any combination (e.g., 2, 3, 4, 5 or more) of the biomarkers listed in Table 2.

TABLE 2
Biomarkers of the Disclosure

| | | | | |
|---|---|---|---|---|
| OR4F16 | POU4F1 | PRMT5 | E2F2 | CPSF6 |
| BUB1B | UHRF1 | EIF1AX | HDAC1 | NACC1 |
| PLK1 | PPP2R2C | SMPD2 | CCNB2 | WEE2 |
| PAXBP1 | WDR45 | CASP8AP2 | KIF15 | MYC |
| CTR9 | FAM120C | SFN | AGPAT3 | MCM6 |
| AR | BRSK1 | WEE1 | REC8 | ADCY6 |
| EIF3A | EVI5L | ESPL1 | RECQL4 | TPX2 |
| KIF4A | NPAS4 | OTUD5 | ZNF853 | MYBL2 |
| MAGEB10 | MCM10 | DMRTC1B | SRSF4 | CDC23 |
| CHEK1 | SUPT5H | TSSK2 | PPP2R5A | RRM2 |
| CENPM | MCM5 | ANAPC10 | ZBTB12 | MAPK1 |
| AKT1 | GALK2 | FOXM1 | MMP12 | PRKACA |
| ADCY1 | FTSJ1 | EXO1 | KIF2C | DDI2 |
| ATP2B2 | TRAP1 | CHEK2 | HSP90AA1 | MEMO1 |
| HASPIN | PAK3 | KIFC1 | PPP2R2D | IGF1 |
| CTDSPL2 | CENPE | ANKRD52 | CDC7 | SKP1 |
| STAG2 | TPT1 | SPAG5 | NANS | PPIAL4C |
| NCAPG | MAD2L2 | PPP2R2B | MOS | PPIAL4D |
| IGF1R | FBXO5 | ZNF331 | RBX1 | SLC9A6 |
| BLM | CDK16 | PAK1 | MAGED4B | ARPP19 |
| ATR | CDC45 | TNPO2 | KIF23 | NOVA2 |
| AURKB | USP27X | LDB1 | SCML1 | CTAG1B |
| RBL2 | MAPK8 | CDK14 | SPANXA2 | CCNA2 |
| RPS6KA6 | PRR20A | CDC25B | TRIM28 | CDC6 |
| GINS2 | ADCY4 | KCNV1 | SRRM5 | MAGEA9 |
| MAD1L1 | RRM1 | CPEB1 | MAGEA1 | F8A3 |
| ADCY5 | TBR1 | ZNF777 | ACTR3B | ARL17A |
| CHTF18 | PAK2 | RPS6KA1 | EBLN1 | CTAG1A |
| SMC1A | KIF11 | PSG7 | TP53TG3C | MAD2L1 |
| BRSK2 | WDHD1 | CD177 | INS | HSFX1 |
| BRPF3 | MELK | CCNG1 | ORC1 | BNIP3 |
| FOXD4L4 | CHERP | PRAMEF8 | HSP90AB1 | MRGPRG |
| TGIF2LX | CENPF | ZBTB17 | CHAF1B | ANAPC2 |
| SOX5 | BUB1 | CCNF | MCM7 | RAD21 |

In some embodiments, a biomarker of the disclosure is one identified as forming a synthetic lethal pair with PKMYT1 using a method described herein. In some embodiments, a biomarker of the disclosure is any one or any combination (e.g., 2, 3, 4, 5 or more) of the biomarkers listed in Table 3. In some embodiments, a synthetic lethal pair of the disclosure comprises a biomarker selected from Table 3. In some embodiments, a synthetic lethal pair of the disclosure comprises a biomarker selected from Table 3 and PKMYT1. In some embodiments, a biomarker of the disclosure is any one or any combination (e.g., 2, 3, 4, 5 or more) of the biomarkers listed in Table 8 and 9. In some embodiments, a synthetic lethal pair of the disclosure comprises a biomarker selected from Table 8 and 9. In some embodiments, a synthetic lethal pair of the disclosure comprises a biomarker selected from Table 8 and 9 and PKMYT1. In some embodiments, a biomarker of the disclosure is any one or any combination (e.g., 2, 3, 4, 5 or more) of the biomarkers listed in Table 10. In some embodiments, a synthetic lethal pair of the disclosure comprises a biomarker selected from Table 10. In some embodiments, a synthetic lethal pair of the disclosure comprises a biomarker selected from Table 10 and PKMYT1.

Table 3 provides biomarkers that are human genes identified as synthetic lethal pairs with PKMYT1 using a computational method described herein. Table 3 further provides a gene identification number for each human gene that the skilled artisan may use to identify the gene in the National Library of Medicine National Center for Biotechnology Information (NCBI) Gene Database (accessible via the world wide web: ncbi.nlm.nih.gov/). The Gene Database is a searchable database of genes that provides nomenclature, chromosomal localization, gene products, attributes of the gene, associated markers, phenotypes, interactions, links to citations, sequence information, information regarding sequence variants, gene maps, expression reports, homologs, protein domain content, and access to external databases. As is understood by the skilled artisan, the nucleotide sequence corresponding to each gene in Table 3 is accessed by entering the corresponding Gene ID into the NCBI Gene Database and selecting the genomic sequence in the desired format computer-readable formats (e.g., FASTA).

TABLE 3
Biomarkers of the Disclosure

| Biomarker | Gene ID | Biomarker | Gene ID | Biomarker | Gene ID |
|---|---|---|---|---|---|
| ATM | 472 | FOXO3 | 2309 | PPP2R1B | 5519 |
| CACNA1H | 8912 | FZD3 | 7976 | PPP3CC | 5533 |
| CDC25A | 993 | JAK1 | 3716 | SMAD2 | 4087 |
| CDKN1B | 1027 | MAP2K4 | 6416 | TGFBR2 | 7048 |
| DUSP7 | 1849 | MAP3K2 | 10746 | TP53 | 7157 |
| BIN3 | 55909 | C6orf118 | 168090 | TMEM242 | 729515 |
| AGPAT5 | 55326 | BOK | 666 | ZNF619 | 285267 |
| FGF17 | 8822 | PHF10 | 55274 | RBBP7 | 5931 |
| PBK | 55872 | HMGB2 | 3148 | EFTUD2 | 9343 |
| NOTCH1 | 4851 | ATG16L1 | 55054 | DUSP16 | 80824 |
| CNTN5 | 53942 | SLITRK6 | 84189 | PGBD4 | 161779 |
| IRF2 | 3660 | VGLL4 | 9686 | TIMM8B | 26521 |
| ALPK2 | 115701 | RNF111 | 54778 | TMEM94 | 9772 |
| CDH19 | 28513 | DNAJC13 | 23317 | EPM2AIP1 | 9852 |
| CHKB | 1120 | TPD52L3 | 89882 | MTMR1 | 8776 |
| MAPK12 | 6300 | ANKK1 | 255239 | ETFDH | 2110 |
| SLC8A1 | 6546 | PLCB2 | 5330 | TSLP | 85480 |
| HDAC2 | 3066 | PPIL6 | 285755 | ZNF418 | 147686 |
| CDT1 | 81620 | ECEL1 | 9427 | POU5F2 | 134187 |
| ADCY2 | 108 | FBXL4 | 26235 | FAM3D | 131177 |
| SLK | 9748 | SUMF1 | 285362 | ARL8B | 55207 |
| CDC20B | 166979 | GABRA5 | 2558 | TMEM171 | 134285 |
| RPS6KA3 | 6197 | MCM9 | 254394 | LONP2 | 83752 |
| STAG1 | 10274 | CENPBD1 | 92806 | POLR3A | 11128 |
| CKAP5 | 9793 | SLC22A2 | 6582 | PKD2L1 | 9033 |
| RAD51 | 5888 | KAT2B | 8850 | MCF2L | 23263 |
| CKS1B | 1163 | DISP2 | 85455 | LARP1B | 55132 |
| CCNO | 10309 | STXBP5 | 134957 | STK32C | 282974 |
| KCNA2 | 3737 | LNPEP | 4012 | GLOD4 | 51031 |
| MCM4 | 4173 | ELL2 | 22936 | GTF2H2C | 728340 |
| PLK4 | 10733 | PTH1R | 5745 | HOMER1 | 9456 |
| CDC16 | 8881 | ADRB3 | 155 | TMEM181 | 57583 |
| ERICH1 | 157697 | FAM193A | 8603 | TMIE | 259236 |
| TNKS | 8658 | GAN | 8139 | SMIM15 | 643155 |
| TDRP | 157695 | ATMIN | 23300 | MYL3 | 4634 |
| MTUS1 | 57509 | OR6T1 | 219874 | RAD51AP2 | 729475 |
| TNFRSF10B | 8795 | IL5RA | 3568 | HDAC5 | 10014 |
| HR | 55806 | TTC21A | 199223 | CTSD | 1509 |
| TNFRSF10D | 8793 | CCDC88C | 440193 | SLC35G6 | 643664 |
| DMTN | 2039 | CFAP53 | 220136 | SMC3 | 9126 |
| ENTPD4 | 9583 | UQCRC1 | 7384 | GOLGA7 | 51125 |
| TNFRSF10C | 8794 | ATP10D | 57205 | HEY2 | 23493 |
| PEBP4 | 157310 | ATG5 | 9474 | RRAGA | 10670 |
| LPL | 4023 | ARSH | 347527 | ADAMTS1 | 9510 |
| LGI3 | 203190 | STARD6 | 147323 | AKAP7 | 9465 |
| SLC7A2 | 6542 | MFAP3L | 9848 | CDH5 | 1003 |
| MTMR9 | 66036 | RARB | 5915 | SEC24A | 10802 |
| MSRA | 4482 | DUSP28 | 285193 | DAAM1 | 23002 |
| PDLIM2 | 64236 | GLT8D1 | 55830 | SYCE1L | 100130958 |
| INTS10 | 55174 | EFNA5 | 1946 | CPEB4 | 80315 |
| SH2D4A | 63898 | SMCHD1 | 23347 | PHF14 | 9678 |
| GFRA2 | 2675 | KIF2A | 3796 | N4BP2L1 | 90634 |
| ZDHHC2 | 51201 | MIDEAS | 91748 | FRMD5 | 84978 |
| PDGFRL | 5157 | ACAP1 | 9744 | MYEF2 | 50804 |
| SPAG11B | 10407 | AADAT | 51166 | GINS4 | 84296 |
| PPP1R3B | 79660 | CASP1 | 834 | ACAA1 | 30 |
| SPAG11A | 653423 | KLHL18 | 23276 | CDC34 | 997 |
| REEP4 | 80346 | EIF4A1 | 1973 | HK3 | 3101 |
| DEFA5 | 1670 | OR8D2 | 283160 | MAMLD1 | 10046 |
| DEFB136 | 613210 | GTF3C6 | 112495 | STIM1 | 6786 |
| NRG1 | 3084 | MCCC2 | 64087 | SLN | 6588 |
| ASAH1 | 427 | USP2 | 9099 | PKN2 | 5586 |
| DEFA3 | 1668 | CASP5 | 838 | COLQ | 8292 |
| EPHX2 | 2053 | STAR | 6770 | MFAP1 | 4236 |
| CNOT7 | 29883 | GON4L | 54856 | NUP88 | 4927 |
| PNMA2 | 10687 | KIAA0513 | 9764 | NSUN2 | 54888 |
| TRIM35 | 23087 | P4HTM | 54681 | SLC26A1 | 10861 |
| ATRX | 546 | SYTL5 | 94122 | SMPDL3A | 10924 |
| INTS9 | 55756 | MAP7D2 | 256714 | IWS1 | 55677 |
| DNAH3 | 55567 | SF3B3 | 23450 | GLRX3 | 10539 |
| MAP3K1 | 4214 | PHLDB2 | 90102 | LRRC57 | 255252 |
| RIMS2 | 9699 | AMT | 275 | CDYL | 9425 |
| NSD1 | 64324 | DYM | 54808 | ACLY | 47 |
| SARAF | 51669 | TRPA1 | 8989 | UBP1 | 7342 |
| CDKN2B | 1030 | SAG | 6295 | RAPGEF4 | 11069 |
| CSMD3 | 114788 | GUCY1A1 | 2982 | VPS4A | 27183 |
| LRP1B | 53353 | A2ML1 | 144568 | GATM | 2628 |
| DMRTA1 | 63951 | SIDT2 | 51092 | SEC24D | 9871 |
| PTPRD | 5789 | LIPA | 3988 | CLTC | 1213 |
| ELAVL2 | 1993 | RSBN1 | 54665 | TFB1M | 51106 |
| FAT1 | 2195 | CLASP1 | 23332 | ZNF528 | 84436 |
| CDH1 | 999 | TUBB8 | 347688 | KCTD8 | 386617 |
| NF1 | 4763 | NLGN2 | 57555 | AGO2 | 27161 |
| PPP6R2 | 9701 | SLC37A2 | 219855 | CCDC80 | 151887 |
| PIM3 | 415116 | COQ3 | 51805 | DCLRE1A | 9937 |
| MAPK11 | 5600 | FNIP1 | 96459 | ZNF471 | 57573 |
| CDH10 | 1008 | TWIST2 | 117581 | IL16 | 3603 |
| PCDH15 | 65217 | AMIGO3 | 386724 | PPIL4 | 85313 |
| ALB | 213 | GBX2 | 2637 | TRMT11 | 60487 |
| OR4F21 | 441308 | ATP7A | 538 | SNORC | 389084 |
| LINGO2 | 158038 | PRTG | 283659 | MSMO1 | 6307 |
| FBN2 | 2201 | GUCY2D | 3000 | TIGD1 | 200765 |
| CACNA1E | 777 | RAB3C | 115827 | PPID | 5481 |
| LRRC7 | 57554 | PUS3 | 83480 | GZMM | 3004 |
| NALCN | 259232 | MARVELD2 | 153562 | SGK1 | 6446 |
| ARID1A | 8289 | CHRNA6 | 8973 | KLHL15 | 80311 |
| ADGRB3 | 577 | CER1 | 9350 | SYN1 | 6853 |
| SI | 6476 | PPP4R2 | 151987 | KLF13 | 51621 |
| PKHD1L1 | 93035 | HTRA4 | 203100 | ADAL | 161823 |
| TBC1D22A | 25771 | NCKIPSD | 51517 | BIRC3 | 330 |
| BNIP3L | 665 | C16orf95 | 100506581 | KIF6 | 221458 |
| DEFA1 | 1667 | CCDC73 | 493860 | ASB9 | 140462 |
| DEFB103B | 55894 | KLHL30 | 377007 | OR51E2 | 81285 |
| DEFB103A | 414325 | EEF2 | 1938 | UPK2 | 7379 |
| HCN1 | 348980 | ACADVL | 37 | MAGEB16 | 139604 |
| RELN | 5649 | MAB21L1 | 4081 | CSNK1G3 | 1456 |
| UNC13C | 440279 | GMPPB | 29925 | BMF | 90427 |
| XKR5 | 389610 | ANXA10 | 11199 | ESM1 | 11082 |
| CHMP7 | 91782 | KIF4B | 285643 | TLCD5 | 219902 |
| CHRNA2 | 1135 | NKAIN2 | 154215 | KIF3A | 11127 |
| CSGALNACT1 | 55790 | CDHR2 | 54825 | PDIA3 | 2923 |
| FAM86B2 | 653333 | SETMAR | 6419 | EVI5 | 7813 |
| EGR3 | 1960 | SMG1 | 23049 | CAMKK1 | 84254 |
| XPO7 | 23039 | GFM2 | 84340 | ADPRM | 56985 |
| TRPS1 | 7227 | AKAP9 | 10142 | CDK11B | 984 |
| KDM6A | 7403 | CD164 | 8763 | FSHR | 2492 |
| NBEA | 26960 | ALG9 | 79796 | POLR2B | 5431 |
| VPS37A | 137492 | ZMYND11 | 10771 | YEATS2 | 55689 |
| SCN1A | 6323 | LTK | 4058 | USP8 | 9101 |
| CSMD2 | 114784 | PLPP5 | 84513 | DRC7 | 84229 |
| GTSE1 | 51512 | SLC35A1 | 10559 | TPD52L1 | 7164 |
| TRMU | 55687 | ALPG | 251 | NTNG1 | 22854 |
| TENM1 | 10178 | C16orf46 | 123775 | RFX2 | 5990 |
| DOCK3 | 1795 | FAM172A | 83989 | ZBTB48 | 3104 |
| VPS13B | 157680 | TRIP11 | 9321 | PDE5A | 8654 |
| RBM10 | 8241 | TTLL7 | 79739 | VDAC3 | 7419 |
| RYR2 | 6262 | C11orf65 | 160140 | STK11IP | 114790 |
| SCARA5 | 286133 | EFHC2 | 80258 | IFIH1 | 64135 |
| SETBP1 | 26040 | TKT | 7086 | HPS1 | 3257 |
| DYSF | 8291 | ARHGAP22 | 58504 | SLC15A1 | 6564 |
| NLGN4X | 57502 | AHI1 | 54806 | NOL9 | 79707 |
| EPHA3 | 2042 | BARX2 | 8538 | UBE2J2 | 118424 |
| FBLN1 | 2192 | KCND3 | 3752 | GJA3 | 2700 |
| ADAMTS20 | 80070 | PGM3 | 5238 | FAM234B | 57613 |
| IFT74 | 80173 | ECT2L | 345930 | TCEANC | 170082 |
| KLKB1 | 3818 | KIF5C | 3800 | ABL2 | 27 |
| ACVR2A | 92 | ELOVL4 | 6785 | MGAT5 | 4249 |
| ZFHX4 | 79776 | SLC6A1 | 6529 | GPR82 | 27197 |
| WWC2 | 80014 | WDR82 | 80335 | LRCH3 | 84859 |
| MOB3B | 79817 | ITGA10 | 8515 | KCTD12 | 115207 |
| DMXL1 | 1657 | HOOK3 | 84376 | MSH2 | 4436 |
| ELAC1 | 55520 | MTMR10 | 54893 | PRPF40A | 55660 |
| RBPMS | 11030 | PPWD1 | 23398 | KCNAB2 | 8514 |
| ANK1 | 286 | COTL1 | 23406 | TBX22 | 50945 |
| CADM2 | 253559 | FAM9B | 171483 | DHX37 | 57647 |
| C9orf72 | 203228 | UPF3A | 65110 | ELMO3 | 79767 |
| MTNR1A | 4543 | PDGFD | 80310 | ZNF180 | 7733 |
| PLAA | 9373 | ACE2 | 59272 | FAF1 | 11124 |
| NIPBL | 25836 | PLXND1 | 23129 | VCL | 7414 |
| ASPM | 259266 | VPS11 | 55823 | GABARAPL2 | 11345 |
| GABRB3 | 2562 | UMODL1- | 150147 | SHOC1 | 158401 |
| | | AS1 | | | |
| CTNNA3 | 29119 | ANO5 | 203859 | OGA | 10724 |
| CNTN3 | 5067 | BACE2 | 25825 | TAMM41 | 132001 |
| PPFIA2 | 8499 | NLRP12 | 91662 | SPATA22 | 84690 |
| FN1 | 2335 | WNK4 | 65266 | PDE4B | 5142 |
| HECW1 | 23072 | SECISBP2L | 9728 | SCN4B | 6330 |
| DMXL2 | 23312 | SLC16A12 | 387700 | DDX47 | 51202 |
| ZFP36L2 | 678 | TGM7 | 116179 | PDS5A | 23244 |
| UPK3A | 7380 | GJB7 | 375519 | RNF31 | 55072 |
| SMC1B | 27127 | CCR2 | 729230 | ZNF620 | 253639 |
| SMARCA4 | 6597 | WTAP | 9589 | TRIM45 | 80263 |
| LRFN5 | 145581 | C3orf62 | 375341 | KCNA1 | 3736 |
| TG | 7038 | WAPL | 23063 | MOCS2 | 4338 |
| CTNND2 | 1501 | PLA2G4D | 283748 | PPP4R1L | 55370 |
| CHD1 | 1105 | NEU2 | 4759 | POLN | 353497 |
| LSAMP | 4045 | NUDT12 | 83594 | NCLN | 56926 |
| PRR5 | 55615 | TSSK1B | 83942 | MPZL2 | 10205 |
| NPAP1 | 23742 | C10orf90 | 118611 | DDX19B | 11269 |
| SNTG1 | 54212 | HOATZ | 399949 | PDHA1 | 5160 |
| MDGA2 | 161357 | MSL3 | 10943 | R3HCC1L | 27291 |
| BNC2 | 54796 | COG4 | 25839 | FMNL1 | 752 |
| SCN2A | 6326 | PJA2 | 9867 | GAPVD1 | 26130 |
| HERC2 | 8924 | ZPR1 | 8882 | MKNK2 | 2872 |
| SCN3A | 6328 | EIF4EBP1 | 1978 | ZMYND15 | 84225 |
| TRPM1 | 4308 | HAPLN1 | 1404 | AMELX | 265 |
| FSTL5 | 56884 | SMIM8 | 57150 | CPTP | 80772 |
| ASH1L | 55870 | HEPACAM | 220296 | ACOT9 | 23597 |
| PRKDC | 5591 | SCAPER | 49855 | F2RL2 | 2151 |
| TCF4 | 6925 | POU2AF1 | 5450 | TNFRSF14 | 8764 |
| SVIL | 6840 | POGLUT3 | 143888 | OR51I2 | 390064 |
| CHD4 | 1108 | IPO7 | 10527 | TMEM170A | 124491 |
| PCDH9 | 5101 | DYRK1A | 1859 | HTR2B | 3357 |
| NRXN3 | 9369 | C11orf87 | 399947 | SMYD4 | 114826 |
| SNX25 | 83891 | NDUFAF4 | 29078 | DCLRE1B | 64858 |
| MPDZ | 8777 | TYRO3 | 7301 | FOXR1 | 283150 |
| TLL1 | 7092 | KCND2 | 3751 | IFT46 | 56912 |
| EPHA6 | 285220 | SMG6 | 23293 | MPZL3 | 196264 |
| FER | 2241 | TP73 | 7161 | CSTF2T | 23283 |
| NFASC | 23114 | DVL1 | 1855 | RER1 | 11079 |
| USP34 | 9736 | HPF1 | 54969 | BTD | 686 |
| SPEF2 | 79925 | YBX2 | 51087 | AURKAIP1 | 54998 |
| CHD8 | 57680 | SLC13A5 | 284111 | ADAM10 | 102 |
| ABCA12 | 26154 | ENC1 | 8507 | CEMIP | 57214 |
| ARID2 | 196528 | C11orf53 | 341032 | PLA2G15 | 23659 |
| KCNIP4 | 80333 | C11orf45 | 219833 | CDIN1 | 84529 |
| NFIB | 4781 | ZGRF1 | 55345 | LTV1 | 84946 |
| SLITRK1 | 114798 | CH25H | 9023 | TENT4B | 64282 |
| ZNF521 | 25925 | SLC39A12 | 221074 | DFFB | 1677 |
| CCNB1 | 891 | GCNT4 | 51301 | SLC35E2A | 9906 |
| CDK7 | 1022 | ULK2 | 9706 | CES2 | 8824 |
| MYT1L | 23040 | BCKDHB | 594 | COPS7B | 64708 |
| FZR1 | 51343 | HYLS1 | 219844 | RAB9A | 9367 |
| SERF1A | 8293 | MMP10 | 4319 | HRH2 | 3274 |
| GADD45B | 4616 | THEG | 51298 | ZNRF1 | 84937 |
| ADGRL2 | 23266 | TAB2 | 23118 | CXCR5 | 643 |
| TTK | 7272 | CCR5 | 1234 | REEP6 | 92840 |
| NRXN2 | 9379 | SHISA5 | 51246 | POLR2E | 5434 |
| UNC13A | 23025 | PTBP1 | 5725 | CC2D2B | 387707 |
| ZBTB7A | 51341 | INPP5A | 3632 | BANK1 | 55024 |
| POLD1 | 5424 | NUDT7 | 283927 | ATIC | 471 |
| PCDH19 | 57526 | NR1D2 | 9975 | ZNF555 | 148254 |
| SLC8A2 | 6543 | GJD2 | 57369 | FGF22 | 27006 |
| E2F4 | 1874 | TBCEL | 219899 | RNF170 | 81790 |
| AUTS2 | 26053 | WDR47 | 22911 | FBXO30 | 84085 |
| KCNN2 | 3781 | PTPRE | 5791 | PICALM | 8301 |
| CCNH | 902 | TP53AIP1 | 63970 | GOLGA8M | 653720 |
| FRG2C | 100288801 | LARP1 | 23367 | CSRNP1 | 64651 |
| PLK2 | 10769 | PLEKHA5 | 54477 | METTL6 | 131965 |
| MYO18A | 399687 | PLD2 | 5338 | GOLGA8IP | 283796 |
| DCAF12L1 | 139170 | PANK4 | 55229 | ENO3 | 2027 |
| DCTN6 | 10671 | SREK1 | 140890 | PIK3R4 | 30849 |
| SETDB2 | 83852 | SLCO1C1 | 53919 | TIGD4 | 201798 |
| DSEL | 92126 | SLC25A46 | 91137 | TBX5 | 6910 |
| F11 | 2160 | MATK | 4145 | C6orf58 | 352999 |
| SMIM18 | 100507341 | THRB | 7068 | CA5B | 11238 |
| UBXN8 | 7993 | INTS11 | 54973 | WDR37 | 22884 |
| TTI2 | 80185 | ACRV1 | 56 | BNC1 | 646 |
| IGSF9B | 22997 | SUPT6H | 6830 | DYTN | 391475 |
| RCBTB1 | 55213 | SOX15 | 6665 | TAOK2 | 9344 |
| HDLBP | 3069 | HSPB2 | 3316 | CREB3L3 | 84699 |
| CTCF | 10664 | C3orf84 | 646498 | PRICKLE1 | 144165 |
| DIAPH3 | 81624 | TM2D2 | 83877 | UGT2A1 | 10941 |
| TSHZ1 | 10194 | CTNNA1 | 1495 | RPS6 | 6194 |
| TLR3 | 7098 | TAGAP | 117289 | CTTNBP2NL | 55917 |
| HTR2A | 3356 | SLC16A14 | 151473 | SEC24C | 9632 |
| STOX2 | 56977 | TCP1 | 6950 | FAM107A | 11170 |
| PHF11 | 51131 | ERBB3 | 2065 | APBB1 | 322 |
| DGKH | 160851 | CENPH | 64946 | FAM124B | 79843 |
| THSD1 | 55901 | PTPN21 | 11099 | SMC6 | 79677 |
| NAA16 | 79612 | GTPBP6 | 8225 | FAM155A | 728215 |
| MYH3 | 4621 | TBRG1 | 84897 | CHP1 | 11261 |
| INTS6 | 26512 | GTPBP4 | 23560 | GP1BA | 2811 |
| FRG1 | 2483 | PDCD2 | 5134 | OXNAD1 | 92106 |
| FHOD3 | 80206 | CHRFAM7A | 89832 | JMJD7- | 8681 |
| | | | | PLA2G4B | |
| DLEU7 | 220107 | CD58 | 965 | VAMP3 | 9341 |
| CDKN2AIP | 55602 | ATXN7 | 6314 | GREM1 | 26585 |
| CASP8 | 841 | GUCY1B1 | 2983 | CDO1 | 1036 |
| ALG11 | 440138 | ASF1A | 25842 | ESRRG | 2104 |
| PDLIM3 | 27295 | ZCWPW2 | 152098 | SLC9A1 | 6548 |
| CPT1B | 1375 | VILL | 50853 | FAM3B | 54097 |
| PDE10A | 10846 | CALHM4 | 221301 | EFR3A | 23167 |
| DCTD | 1635 | ZNF821 | 55565 | GNA15 | 2769 |
| TIAM2 | 26230 | DPAGT1 | 1798 | ZRSR2 | 8233 |
| VEGFC | 7424 | NLRP2 | 55655 | KLF3 | 51274 |
| TDRD3 | 81550 | KLHL2 | 11275 | OR4F16 | 81399 |
| CBLN2 | 147381 | ESAM | 90952 | BUB1B | 701 |
| NEK3 | 4752 | IL18 | 3606 | PLK1 | 5347 |
| MED4 | 29079 | SLC30A5 | 64924 | PAXBP1 | 94104 |
| PARD6G-AS1 | 100130522 | BAZ1A | 11177 | CTR9 | 9646 |
| LMO7 | 4008 | B3GLCT | 145173 | AR | 367 |
| EPSTI1 | 94240 | PLA2R1 | 22925 | EIF3A | 8661 |
| HELT | 391723 | SMARCAL1 | 50485 | KIF4A | 24137 |
| FOXO1 | 2308 | PIK3R6 | 146850 | MAGEB10 | 139422 |
| FLNA | 2316 | RWDD1 | 51389 | CHEK1 | 1111 |
| NEIL3 | 55247 | STARD4 | 134429 | CENPM | 79019 |
| GALR1 | 2587 | HPSE2 | 60495 | AKT1 | 207 |
| RWDD4 | 201965 | PDE6B | 5158 | ADCY1 | 107 |
| RBFA | 79863 | SLC16A1 | 6566 | ATP2B2 | 491 |
| GPALPP1 | 55425 | BRD7 | 29117 | HASPIN | 83903 |
| RNF43 | 54894 | XCR1 | 2829 | CTDSPL2 | 51496 |
| MTRF1 | 9617 | CCR9 | 10803 | STAG2 | 10735 |
| ING2 | 3622 | UCHL3 | 7347 | NCAPG | 64151 |
| B2M | 567 | JAML | 120425 | IGF1R | 3480 |
| LACC1 | 144811 | SLC17A5 | 26503 | BLM | 641 |
| ANKRD37 | 353322 | MZT1 | 440145 | ATR | 545 |
| SLC66A2 | 80148 | FMR1 | 2332 | AURKB | 9212 |
| VPS36 | 51028 | CTBP2 | 1488 | RBL2 | 5934 |
| CNMD | 11061 | DHX15 | 1665 | RPS6KA6 | 27330 |
| ZADH2 | 284273 | KCTD19 | 146212 | GINS2 | 51659 |
| NCAPH2 | 29781 | KCNA5 | 3741 | MAD1L1 | 8379 |
| ARSA | 410 | SACM1L | 22908 | ADCY5 | 111 |
| PRKG1 | 5592 | FRMD4A | 55691 | CHTF18 | 63922 |
| DNAH8 | 1769 | PFKFB4 | 5210 | SMC1A | 8243 |
| MIOX | 55586 | DUSP22 | 56940 | BRSK2 | 9024 |
| CWF19L2 | 143884 | UCN2 | 90226 | BRPF3 | 27154 |
| SIK3 | 23387 | PRAP1 | 118471 | FOXD4L4 | 349334 |
| INPP5D | 3635 | INTU | 27152 | TGIF2LX | 90316 |
| OCA2 | 4948 | PARP14 | 54625 | SOX5 | 6660 |
| MCTP1 | 79772 | NAA15 | 80155 | POU4F1 | 5457 |
| LARP4B | 23185 | LCA5L | 150082 | UHRF1 | 29128 |
| FMN1 | 342184 | SGTB | 54557 | PPP2R2C | 5522 |
| UHRF2 | 115426 | SHPK | 23729 | WDR45 | 11152 |
| KIF20B | 9585 | FUOM | 282969 | FAM120C | 54954 |
| GLB1L3 | 112937 | USP48 | 84196 | BRSK1 | 84446 |
| ZMYM2 | 7750 | MTSS2 | 92154 | EVI5L | 115704 |
| SIPA1L2 | 57568 | LRRC3B | 116135 | NPAS4 | 266743 |
| DDX3X | 1654 | MAGEB6 | 158809 | MCM10 | 55388 |
| SCAP | 22937 | ABHD14A- | 100526760 | SUPT5H | 6829 |
| | | ACY1 | | | |
| SASH1 | 23328 | ATP1A3 | 478 | MCM5 | 4174 |
| RASGRF2 | 5924 | ARHGAP18 | 93663 | GALK2 | 2585 |
| PARD3 | 56288 | ABL1 | 25 | FTSJ1 | 24140 |
| PSIP1 | 11168 | C11orf1 | 64776 | TRAP1 | 10131 |
| DIPK1C | 125704 | CCDC112 | 153733 | PAK3 | 5063 |
| MID1 | 4281 | PIK3C2A | 5286 | CENPE | 1062 |
| FAM120B | 84498 | SUPT16H | 11198 | TPT1 | 7178 |
| SOX6 | 55553 | TBX3 | 6926 | MAD2L2 | 10459 |
| MINPP1 | 9562 | ESRP2 | 80004 | FBXO5 | 26271 |
| SERP2 | 387923 | MON2 | 23041 | CDK16 | 5127 |
| SBF2 | 81846 | MSH4 | 4438 | CDC45 | 8318 |
| SPIRE2 | 84501 | MANF | 7873 | USP27X | 389856 |
| WDR6 | 11180 | TCAIM | 285343 | MAPK8 | 5599 |
| MBD1 | 4152 | GCSH | 2653 | PRR20A | 122183 |
| ANKRD22 | 118932 | AMY2B | 280 | ADCY4 | 196883 |
| FAS | 355 | ARHGAP33 | 115703 | RRM1 | 6240 |
| HTR1E | 3354 | CBR4 | 84869 | TBR1 | 10716 |
| ITGA2 | 3673 | ACOT12 | 134526 | PAK2 | 5062 |
| SMIM2 | 79024 | TMEM87A | 25963 | KIF11 | 3832 |
| MYO6 | 4646 | RAB39A | 54734 | WDHD1 | 11169 |
| TBC1D5 | 9779 | SPA17 | 53340 | MELK | 9833 |
| RBM5 | 10181 | MYBPC1 | 4604 | CHERP | 10523 |
| DLEC1 | 9940 | TNFRSF9 | 3604 | CENPF | 1063 |
| FLT1 | 2321 | KDM5B | 10765 | BUB1 | 699 |
| MROH2B | 133558 | CHAF1A | 10036 | PRMT5 | 10419 |
| CD109 | 135228 | SPRN | 503542 | EIF1AX | 1964 |
| MTREX | 23517 | NAF1 | 92345 | SMPD2 | 6610 |
| CYB5A | 1528 | LMOD3 | 56203 | CASP8AP2 | 9994 |
| GBE1 | 2632 | ABHD14B | 84836 | SFN | 2810 |
| JHY | 79864 | AASDHPPT | 60496 | WEE1 | 7465 |
| COPS9 | 150678 | ZNF470 | 388566 | ESPL1 | 9700 |
| RGCC | 28984 | F2RL1 | 2150 | OTUD5 | 55593 |
| CPNE7 | 27132 | VAC14 | 55697 | DMRTC1B | 728656 |
| ATP8B4 | 79895 | TCIM | 56892 | TSSK2 | 23617 |
| USP45 | 85015 | PDK3 | 5165 | ANAPC10 | 10393 |
| KLHDC4 | 54758 | DYNLRB2 | 83657 | FOXM1 | 2305 |
| KCNG4 | 93107 | FBXL3 | 26224 | EXO1 | 9156 |
| SFMBT1 | 51460 | EPS8 | 2059 | CHEK2 | 11200 |
| NEU4 | 129807 | ZDHHC3 | 51304 | KIFC1 | 3833 |
| CDK10 | 8558 | OPN4 | 94233 | ANKRD52 | 283373 |
| STK25 | 10494 | STK10 | 6793 | SPAG5 | 10615 |
| NDUFA10 | 4705 | GSPT2 | 23708 | PPP2R2B | 5521 |
| SLC12A6 | 9990 | SLC6A3 | 6531 | ZNF331 | 55422 |
| NHLRC3 | 387921 | HTR1B | 3351 | PAK1 | 5058 |
| RARS2 | 57038 | SFT2D1 | 113402 | TNPO2 | 30000 |
| MYO5A | 4644 | ZNF662 | 389114 | LDB1 | 8861 |
| KDSR | 2531 | NFIC | 4782 | CDK14 | 5218 |
| MFSD4B | 91749 | TLR2 | 7097 | CDC25B | 994 |
| DDX10 | 1662 | MICU2 | 221154 | KCNV1 | 27012 |
| SLC16A10 | 117247 | ZFYVE28 | 57732 | CPEB1 | 64506 |
| PPP1R7 | 5510 | UTP15 | 84135 | ZNF777 | 27153 |
| FHL5 | 9457 | ZNFX1 | 57169 | RPS6KA1 | 6195 |
| RPAP1 | 26015 | ZNF35 | 7584 | PSG7 | 5676 |
| PDCD1 | 5133 | OLFM3 | 118427 | CD177 | 57126 |
| ING5 | 84289 | SENP7 | 57337 | CCNG1 | 900 |
| USP10 | 9100 | GNB1 | 2782 | PRAMEF8 | 391002 |
| GAS8-AS1 | 750 | UTP4 | 84916 | ZBTB17 | 7709 |
| GAL3ST2 | 64090 | TMEM62 | 80021 | CCNF | 899 |
| AGRN | 375790 | ADD1 | 118 | E2F2 | 1870 |
| FAN1 | 22909 | ASB11 | 140456 | HDAC1 | 3065 |
| DEPDC1B | 55789 | CRMP1 | 1400 | CCNB2 | 9133 |
| ERMARD | 55780 | MCF2L2 | 23101 | KIF15 | 56992 |
| SRPRA | 6734 | FDX1 | 2230 | AGPAT3 | 56894 |
| BRCA1 | 672 | TRIM69 | 140691 | REC8 | 9985 |
| JAM3 | 83700 | TMC1 | 117531 | RECQL4 | 9401 |
| ATG4B | 23192 | SLC6A2 | 6530 | ZNF853 | 54753 |
| HPGD | 3248 | HECA | 51696 | SRSF4 | 6429 |
| DDHD2 | 23259 | CXCR6 | 10663 | PPP2R5A | 5525 |
| VWA5A | 4013 | CXorf38 | 159013 | ZBTB12 | 221527 |
| DEF8 | 54849 | EXOSC7 | 23016 | MMP12 | 4321 |
| GPR63 | 81491 | MLH3 | 27030 | KIF2C | 11004 |
| RNF152 | 220441 | CXorf21 | 80231 | HSP90AA1 | 3320 |
| EDNRB | 1910 | FAM122A | 116224 | PPP2R2D | 55844 |
| SCEL | 8796 | ZFP36L1 | 677 | CDC7 | 8317 |
| AFG1L | 246269 | CXorf58 | 254158 | NANS | 54187 |
| PCNX1 | 22990 | ABLIM2 | 84448 | MOS | 4342 |
| NT5DC1 | 221294 | ACER2 | 340485 | RBX1 | 9978 |
| AKAP12 | 9590 | BICC1 | 80114 | MAGED4B | 81557 |
| NEK4 | 6787 | ATP13A3 | 79572 | KIF23 | 9493 |
| MANEA | 79694 | CELF4 | 56853 | SCML1 | 6322 |
| VPS26B | 112936 | PRKCZ | 5590 | SPANXA2 | 728712 |
| ITIH4 | 3700 | TRAPPC13 | 80006 | TRIM28 | 10155 |
| ME1 | 4199 | SLC4A1 | 6521 | SRRM5 | 100170229 |
| ALAS1 | 211 | ARCN1 | 372 | MAGEA1 | 4100 |
| FECH | 2235 | APP | 351 | ACTR3B | 57180 |
| CUL5 | 8065 | CMC2 | 56942 | EBLN1 | 340900 |
| FBXO31 | 79791 | RHOBTB3 | 22836 | TP53TG3C | 653550 |
| GLI3 | 2737 | NR2C2 | 7182 | INS | 3630 |
| DGKI | 9162 | MAGEB3 | 4114 | ORC1 | 4998 |
| GRAMD1B | 57476 | ARSK | 153642 | HSP90AB1 | 3326 |
| DBNDD1 | 79007 | HSF2 | 3298 | CHAF1B | 8208 |
| RBM15B | 29890 | PLN | 5350 | MCM7 | 4176 |
| RXFP2 | 122042 | TNFSF12 | 8742 | CPSF6 | 11052 |
| NARS1 | 4677 | RPGR | 6103 | NACC1 | 112939 |
| CCNA1 | 8900 | MYO1E | 4643 | WEE2 | 494551 |
| UGGT2 | 55757 | TENT5A | 55603 | MYC | 4609 |
| APLP2 | 334 | TMEM25 | 84866 | MCM6 | 4175 |
| UBE3A | 7337 | TMEM144 | 55314 | ADCY6 | 112 |
| PHLPP2 | 23035 | SERINC5 | 256987 | TPX2 | 22974 |
| SMOC2 | 64094 | SPINK8 | 646424 | MYBL2 | 4605 |
| EPB41L2 | 2037 | MCTP2 | 55784 | CDC23 | 8697 |
| LTF | 4057 | PLCD1 | 5333 | RRM2 | 6241 |
| UNC93A | 54346 | SLC22A14 | 9389 | MAPK1 | 5594 |
| RHOA | 387 | COMMD6 | 170622 | PRKACA | 5566 |
| BRF2 | 55290 | PTPN4 | 5775 | DDI2 | 84301 |
| ALG5 | 29880 | DDX53 | 168400 | MEMO1 | 51072 |
| POPDC3 | 64208 | HSPA4L | 22824 | IGF1 | 3479 |
| ROCK1 | 6093 | CP | 1356 | SKP1 | 6500 |
| COL10A1 | 1300 | UBA3 | 9039 | PPIAL4C | 653598 |
| PRKCD | 5580 | GLB1 | 2720 | PPIAL4D | 645142 |
| SLC38A8 | 146167 | ZNF501 | 115560 | SLC9A6 | 10479 |
| GPR35 | 2859 | PRRG1 | 5638 | ARPP19 | 10776 |
| BAG4 | 9530 | THUMPD3 | 25917 | NOVA2 | 4858 |
| TUBE1 | 51175 | ZNF286B | 729288 | CTAG1B | 1485 |
| AMBRA1 | 55626 | ARHGAP11B | 89839 | CCNA2 | 890 |
| HAUS6 | 54801 | ASAP1 | 50807 | CDC6 | 990 |
| BORA | 79866 | IGSF9 | 57549 | MAGEA9 | 4108 |
| KCNJ5 | 3762 | SLC24A4 | 123041 | F8A3 | 474384 |
| SH3GL2 | 6456 | SOS1 | 6654 | ARL17A | 51326 |
| CNR1 | 1268 | PUDP | 8226 | CTAG1A | 246100 |
| ARIH2OS | 646450 | SYNRG | 11276 | MAD2L1 | 4085 |
| CCDC68 | 80323 | EIF4E3 | 317649 | HSFX1 | 100506164 |
| CHRND | 1144 | ATXN3L | 92552 | BNIP3 | 664 |
| LAMB1 | 3912 | CALD1 | 800 | MRGPRG | 386746 |
| POU3F2 | 5454 | CDK5RAP2 | 55755 | ANAPC2 | 29882 |
| RAD21 | 5885 | | | | |

¹Refers to the gene reference number as used in the National Library of Medicine National Center for Biotechnology Information (NCBI) Gene Database (accessible via the world wide web: ncbi.nlm.nih.gov).

### Methods of Identifying Synthetic Lethal Pairs

The present disclosure provides methods for identifying a biomarker that forms a synthetic lethal pair with a target gene (e.g., PKMYT1). In some embodiments, the biomarker has altered (e.g., increased or decreased) expression level and/or activity in one or more human cancers, e.g., due to one or more mutations in the gene encoding the biomarker. In some embodiments, the presence of a mutated biomarker in a human cancer is an indicator (e.g., predictive indicator) that the cancer will respond or will likely respond to one or more therapeutic agents targeting the target gene (e.g., one or more therapeutic agents targeting PKMYT1), such as one or more therapeutic agents that inhibit the target gene or a transcriptional or translational product thereof.

### Computational Approaches

In some embodiments, the disclosure provides one or more biomarkers identified using a computational approach described herein. In some embodiments, one or more biomarkers having altered expression level and/or activity in one or more human cancers that potentially form a synthetic lethal pair with a target gene (e.g., PKMYT1) are identified based on the literature and public data, and candidates identified by, for example, criteria including multi-omics analysis, evaluation of tumor type (e.g., primary tumor), experimental data in relevant cell lines, target tractability, biomarker prevalence, etc.

In some embodiments, a predictive algorithm is applied to a dataset compiled from functional gene interference screens to identify a biomarker of the disclosure. Functional genomic screens based on RNA interference technologies (e.g., short hairpin (shRNA)-based technology) and/or gene-editing technologies (e.g., CRISPR/Cas technology) enable gene-knockout studies to be performed across many different genetic contexts (see, e.g., Huang, et al (2020) Nat. Rev. Drug Disc. 19:23). Several public databases provide catalogs of such data, including, for example, Project DRIVE (see, e.g., McDonald, et al (2017) Cell 170:577); Project Achilles (see, e.g., word wide web: depmap.org/portal/achilles); and Project Score (see, e.g., Behan, et al (2019) Nature 568:511). Predictive algorithms are applied to such large datasets to identify for correlations between target gene silencing, functional outcome (e.g., lethality), and genetic background to identify putative synthetic lethal interactions in human cancer cells. In some embodiments, a predictive algorithm of the disclosure comprises one or more prediction criteria to predict a biomarker that will form a synthetic lethal pair with PKMYT1.

In some embodiments, a predictive algorithm of the disclosure comprises one or more prediction criteria to predict a biomarker that will form a synthetic lethal pair with a target gene described herein (e.g., PKYMT1). In some embodiments, the predictive algorithm comprises performing a statistical test (e.g., a chi-squared test) to determine the association of a loss of function of the biomarker occurring in at least one cell line (e.g., 1, 2, 3, 4 or more cell lines) and sensitivity to perturbation in the target gene (e.g., PKYMT1). In some embodiments, the predictive algorithm comprises performing a statistical test (e.g., a chi-squared test) to determine the association of a loss of function of the biomarker occurring in at least four cell lines and sensitivity to perturbation in the target gene (e.g., PKYMT1). In some embodiments, the one or more prediction criteria comprises a p value determined by the statistical test, wherein a p value of less than about 0.001, about 0.005, or about 0.01 is used to predict a biomarker that forms a synthetic lethal interaction with the target gene (e.g., PKYMT1). In some embodiments, the one or more prediction criteria comprises a p value determined by the statistical test, wherein a p value of less than about 0.001 is used to predict a biomarker that forms a synthetic lethal interaction with the target gene (e.g., PKYMT1). In some embodiments, the predictive algorithm comprises calculating the ratio of (a) the odds of sensitivity to perturbation of the target gene (e.g., PKYMT1) in at least one cell line with a loss of function of the biomarker (e.g., 1, 2, 3, 4 or more cell lines with a loss of function of the biomarker) to (b) the odds of sensitivity to perturbation of the target gene (e.g., PKYMT1) in at least one cell line comprising a wild-type biomarker (e.g., 1, 2, 3, 4, or more cell lines comprising a wild-type biomarker). In some embodiments, the predictive algorithm comprises calculating the ratio of (a) the odds of sensitivity to perturbation of the target gene (e.g., PKYMT1) in at least four cell lines with a loss of function of the biomarker to (b) the odds of sensitivity to perturbation of the target gene (e.g., PKYMT1) in at least four cell lines comprising a wild-type biomarker. In some embodiments, a ratio of greater than about 1.5, about 1.6, about 1.7, about 1.8, about 1.9, about 2.0, about 2.1, about 2.2, about 2.3, about 2.4, or about 2.5 is used to predict a biomarker that forms a synthetic lethal interaction with the target gene (e.g., PKYMT1). In some embodiments, a ratio of greater than about 2 is used to predict a biomarker that forms a synthetic lethal interaction with the target gene (e.g., PKYMT1).

In some embodiments, a machine learning approach is used in conjunction with a database of known synthetic lethal gene interactions to identify a biomarker of the disclosure. In some embodiments, the database comprises comprehensive and rigorously curated information on synthetic lethal interactions collected from publications and/or experimental datasets. In some embodiments, the machine learning algorithm considers one or more different features of the interacting genes based on a genetic interaction database. In some embodiments, the one or more different features are intended to capture the genomics, network and functional relationships between the putative synthetic lethal gene pairs. In some embodiments, a machine learning approach comprises one or more prediction criteria to predict a biomarker that will form a synthetic lethal pair with a target gene described herein (e.g., PKYMT1). In some embodiments, the one or more prediction criteria is a prediction score. In some embodiments, a prediction score of greater than about 0.3 (e.g., about 0.3, about 0.35, about 0.4, about 0.45, about 0.5, about 0.55, or about 0.6) is used to predict a biomarker that forms a synthetic lethal interaction with the target gene (e.g., PKYMT1).

In some embodiments, a biomarker of the disclosure is identified according to a predictive algorithm and/or machine learning algorithm described herein and comprises an inactivating mutation in a gene in a plurality of subjects having a cancer. In some embodiments, the cancer is any one or any combination of human cancers listed in the TCGA (Cancer Genome Atlas Program; see world wide web: cancer.gov/tcga). In some embodiments, the cancer is any one or any combination of colorectal adenocarcinoma (COAD), breast invasive carcinoma (BRCA), lung adenocarcinoma (LUAD), lung squamous cell carcinoma (LUSC), ovarian serous cystadenocarcinoma (OV), and liver hepatocellular carcinoma (LIHC). In some embodiments, the inactivating mutation is a homozygous deletion of a gene. In some embodiments, the inactivating mutation is a missense mutation in a gene predicted to encode a nonfunctional protein. In some embodiments, the inactivating mutation is a missense mutation in a gene predicted to encode a truncated protein. In some embodiments, the inactivating mutation occurs in at least about 1%, about 1.5%, about 2%, about 2.5%, about 3%, about 3.5%, about 4%, about 4.5%, or about 5% subjects having the cancer (e.g., any one or any combination of human cancers listed in the TCGA). In some embodiments, the inactivating mutation occurs in more than about 5% of subjects having the cancer (e.g., any one or any combination of human cancers listed in the TCGA). In some embodiments, the inactivating mutation occurs in at least about 5%, about 6%, about 7%, about 8%, about 9%, about 10%, about 12%, about 15%, or about 20% subjects having the cancer (e.g., any one or any combination of human cancers listed in the TCGA).

In some embodiments, a biomarker of the disclosure (e.g., that forms a synthetic lethal pair with PKMYT1) is identified by one or more computational algorithms described herein. In some embodiments, the biomarker is identified by a predictive algorithm applied to an experimental dataset described herein (e.g., a dataset based on functional genomic screening). In some embodiments, the biomarker is identified by a machine learning algorithm. In some embodiments, the biomarker is identified by a predictive algorithm and a machine learning algorithm. In some embodiments, the predictive algorithm comprises at least one prediction criteria selected from (i) a p value less than about 0.001, wherein the p value is determined by a statistical test (e.g., a chi-squared test) of sensitivity to perturbation of a target gene (e.g., PKYMT1) in at least four or more cell lines having a loss of function of the biomarker; and (ii) an odds ratio of greater than about 2, wherein the odds ratio is a ratio of (a) the sensitivity to perturbation of the target gene (e.g., PKYMT1) in at least four or more cell lines having a loss of function of the biomarker to (b) the odds of sensitivity to perturbation of the target gene (e.g., PKYMT1) in at least four or more cell lines comprising a wild-type biomarker. In some embodiments, the machine learning algorithm comprises a prediction criteria that is a prediction score of greater than about 0.3, about 0.4, or about 0.5. In some embodiments, biomarker comprises an inactivating mutation (e.g., a homozygous deletion, a missense mutation encoding a nonfunctional protein, or a missense mutation encoding a truncated protein) in at least about 1%, about 2%, about 3%, about 4%, about 5%, or more of subjects having a cancer, wherein the cancer is selected from any one or any combination of human cancers listed in the TCGA (e.g., COAD, BRCA, LUAD, LUSC, OV, and/or LIHC).

In some embodiments, a biomarker of the disclosure identified by a predictive algorithm described herein and a machine learning algorithm described herein is selected from any one or any combination (e.g., 2, 3, 4, 5 or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some embodiments, a biomarker of the disclosure is identified by a predictive algorithm described herein, wherein the predictive algorithm comprises at least one prediction criteria selected from (i) a p value less than about 0.001, wherein the p value is determined by a statistical test (e.g., a chi-squared test) of sensitivity to perturbation of a target gene (e.g., PKYMT1) in at least four or more cell lines having a loss of function of the biomarker; and (ii) an odds ratio of greater than about 2, wherein the odds ratio is a ratio of (a) the sensitivity to perturbation of the target gene (e.g., PKYMT1) in at least four or more cell lines having a loss of function of the biomarker to (b) the odds of sensitivity to perturbation of the target gene (e.g., PKYMT1) in at least four or more cell lines comprising a wild-type biomarker, and wherein the biomarker is selected from any one or any combination (e.g., 2, 3, 4, 5, or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some embodiments, a biomarker of the disclosure is identified by a predictive algorithm described herein, wherein the predictive algorithm comprises at least one prediction criteria selected from (i) a p value less than about 0.001, wherein the p value is determined by a statistical test (e.g., a chi-squared test) of sensitivity to perturbation of a target gene (e.g., PKYMT1) in at least four or more cell lines having a loss of function of the biomarker; and (ii) an odds ratio of greater than about 2, wherein the odds ratio is a ratio of (a) the sensitivity to perturbation of the target gene (e.g., PKYMT1) in at least four or more cell lines having a loss of function of the biomarker to (b) the odds of sensitivity to perturbation of the target gene (e.g., PKYMT1) in at least four or more cell lines comprising a wild-type biomarker, wherein the biomarker comprises an inactivating mutation (e.g., a homozygous deletion, a missense mutation encoding a nonfunctional protein, or a missense mutation encoding a truncated protein) in at least about 5% of subjects having a cancer, wherein the cancer is selected from any one or any combination of human cancers listed in the TCGA, and wherein the biomarker is selected from any one or any combination (e.g., 2, 3, 4, 5, or more) of CNTN5, IRF2, ALPK2, CHKB, MAPK12, SLC8A1, CDH19, CDT1, ADCY2, SLK, RPS6KA3, CCNO, HDAC2, CDC20B, STAG1, CKAP5, RAD51, CKS1B, KCNA2, MCM4, PLK4, and CDC16.

In some embodiments, a biomarker of the disclosure is identified by a predictive algorithm described herein, wherein the predictive algorithm comprises at least one prediction criteria selected from (i) a p value less than about 0.001, wherein the p value is determined by a statistical test (e.g., a chi-squared test) of sensitivity to perturbation of a target gene (e.g., PKYMT1) in at least four or more cell lines having a loss of function of the biomarker; and (ii) an odds ratio of greater than about 2, wherein the odds ratio is a ratio of (a) the sensitivity to perturbation of the target gene (e.g., PKYMT1) in at least four or more cell lines having a loss of function of the biomarker to (b) the odds of sensitivity to perturbation of the target gene (e.g., PKYMT1) in at least four or more cell lines comprising a wild-type biomarker, wherein the biomarker comprises an inactivating mutation (e.g., a homozygous deletion, a missense mutation encoding a nonfunctional protein, or a missense mutation encoding a truncated protein) in at least about 3%, about 4%, about 5%, or more of subjects having a cancer, wherein the cancer is selected from any one or any combination of human cancers listed in the TCGA, and wherein the biomarker is selected from any one or any combination (e.g., 2, 3, 4, or 5) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some embodiments, a biomarker of the disclosure is identified by a machine learning algorithm described herein, wherein the machine learning algorithm comprises a prediction criteria that is a prediction score of greater than about 0.3, and wherein the biomarker is selected from any one or any combination (e.g., 2, 3, 4, or 5) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some embodiments, a biomarker of the disclosure is identified by a machine learning algorithm described herein, wherein the machine learning algorithm comprises a prediction criteria that is a prediction score of greater than about 0.3, wherein the biomarker comprises an inactivating mutation (e.g., a homozygous deletion, a missense mutation encoding a nonfunctional protein, or a missense mutation encoding a truncated protein) in at least about 5% of subjects having a cancer, wherein the cancer is selected from any one or any combination of human cancers listed in the TCGA (e.g., COAD, BRCA, LUAD, LUSC, OV, and/or LIHC), and wherein the biomarker is selected from any one or any combination (e.g., 2, 3, 4, or 5) of BIN3, AGPAT5, FGF17, PBK, NOTCH1, CNTN5, IRF2, ALPK2, CHKB, MAPK12, SLC8A1, and CDH19.

In some embodiments, a biomarker of the disclosure is identified by a machine learning algorithm described herein, wherein the machine learning algorithm comprises a prediction criteria that is a prediction score of greater than about 0.5, wherein the biomarker comprises an inactivating mutation (e.g., a homozygous deletion, a missense mutation encoding a nonfunctional protein, or a missense mutation encoding a truncated protein) in at least about 3%, about 4%, or about 5% of subjects having a cancer, wherein the cancer is selected from any one or any combination of human cancers listed in the TCGA (e.g., COAD, BRCA, LUAD, LUSC, OV, and/or LIHC), and wherein the biomarker is selected from any one or any combination (e.g., 2, 3, 4, or 5) of CDT1, ADCY2, SLK, RPS6KA3, and CCNO.

In some embodiments, a biomarker of the disclosure is identified by a machine learning algorithm described herein, wherein the machine learning algorithm comprises a prediction criteria that is a prediction score of greater than about 0.5, wherein the biomarker comprises an inactivating mutation (e.g., a homozygous deletion, a missense mutation encoding a nonfunctional protein, or a missense mutation encoding a truncated protein) in at least about 1%, about 2%, about 3%, about 4%, or about 5% of subjects having a cancer, wherein the cancer is selected from any one or any combination of human cancers listed in the TCGA (e.g., COAD, BRCA, LUAD, LUSC, OV, and/or LIHC), and wherein the biomarker is selected from any one or any combination (e.g., 2, 3, 4, or 5) of CDT1, ADCY2, SLK, RPS6KA3, CCNO, HDAC2, CDC20B, STAG1, CKAP5, RAD51, CKS1B, KCNA2, MCM4, PLK4, and CDC16.

In some embodiments, a biomarker of the disclosure (e.g., that forms a synthetic lethal pair with PKMYT1) is identified by one or more computational algorithms described herein. In some embodiments, the biomarker is identified by a predictive algorithm applied to an experimental dataset as described herein (e.g., a dataset based on functional genomic screening). In some embodiments, the biomarker is identified by a machine learning algorithm. In some embodiments, the biomarker is identified by a predictive algorithm and a machine learning algorithm. In some embodiments, the predictive algorithm comprises at least one prediction criteria selected from (i) a p value less than about 0.001, wherein the p value is determined by a statistical test (e.g., a chi-squared test) of sensitivity to perturbation of a target gene (e.g., PKYMT1) in at least four or more cell lines having a loss of function of the biomarker; and (ii) an odds ratio of greater than about 2, wherein the odds ratio is a ratio of (a) the sensitivity to perturbation of the target gene (e.g., PKYMT1) in at least four or more cell lines having a loss of function of the biomarker to (b) the odds of sensitivity to perturbation of the target gene (e.g., PKYMT1) in at least four or more cell lines comprising a wild-type biomarker. In some embodiments, a biomarker of the disclosure identified by a predictive algorithm described herein is selected from any one or any combination (e.g., 2, 3, 4, 5, or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some embodiments, a biomarker of the disclosure is identified by a predictive algorithm described herein, wherein the predictive algorithm comprises at least one prediction criteria selected from (i) a p value less than about 0.001, wherein the p value is determined by a statistical test (e.g., a chi-squared test) of sensitivity to perturbation of a target gene (e.g., PKYMT1) in at least four or more cell lines having a loss of function of the biomarker; and (ii) an odds ratio of greater than about 2, wherein the odds ratio is a ratio of (a) the sensitivity to perturbation of the target gene (e.g., PKYMT1) in at least four or more cell lines having a loss of function of the biomarker to (b) the odds of sensitivity to perturbation of the target gene (e.g., PKYMT1) in at least four or more cell lines comprising a wild-type biomarker, and wherein the biomarker is selected from any one or any combination (e.g., 2, 3, 4, 5, or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some embodiments, a biomarker of the disclosure is identified by a predictive algorithm described herein, wherein the predictive algorithm comprises at least one prediction criteria selected from (i) a p value less than about 0.001, wherein the p value is determined by a statistical test (e.g., a chi-squared test) of sensitivity to perturbation of a target gene (e.g., PKYMT1) in at least four or more cell lines having a loss of function of the biomarker; and (ii) an odds ratio of greater than about 2, wherein the odds ratio is a ratio of (a) the sensitivity to perturbation of the target gene (e.g., PKYMT1) in at least four or more cell lines having a loss of function of the biomarker to (b) the odds of sensitivity to perturbation of the target gene (e.g., PKYMT1) in at least four or more cell lines comprising a wild-type biomarker, wherein the biomarker comprises an inactivating mutation (e.g., a homozygous deletion, a missense mutation encoding a nonfunctional protein, or a missense mutation encoding a truncated protein) in at least about 3%, about 4%, about 5% of subjects having a cancer, wherein the cancer is selected from any one or any combination of human cancers listed in the TCGA, and wherein the biomarker is selected from any one or any combination (e.g., 2, 3, 4, 5, or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some embodiments, a biomarker of the disclosure is identified by a machine learning algorithm described herein, wherein the machine learning algorithm comprises a prediction criteria that is a prediction score of greater than about 0.3, and wherein the biomarker is selected from CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some embodiments, a biomarker of the disclosure is identified by a machine learning algorithm described herein, wherein the machine learning algorithm comprises a prediction criteria that is a prediction score of greater than about 0.3, wherein the biomarker comprises an inactivating mutation (e.g., a homozygous deletion, a missense mutation encoding a nonfunctional protein, or a missense mutation encoding a truncated protein) in at least about 5% of subjects having a cancer, wherein the cancer is selected from any one or any combination of human cancers listed in the TCGA (e.g., COAD, BRCA, LUAD, LUSC, OV, and/or LIHC), and wherein the biomarker is selected from CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some embodiments, a biomarker of the disclosure is identified by a machine learning algorithm described herein, wherein the machine learning algorithm comprises a prediction criteria that is a prediction score of greater than about 0.5, and wherein the biomarker is selected from any one or any combination (e.g., 2, 3, 4, 5, or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some embodiments, a biomarker of the disclosure is identified by a machine learning algorithm described herein, wherein the machine learning algorithm comprises a prediction criteria that is a prediction score of greater than about 0.5, wherein the biomarker comprises an inactivating mutation (e.g., a homozygous deletion, a missense mutation encoding a nonfunctional protein, or a missense mutation encoding a truncated protein) in at least about 3%, about 4%, or about 5% of subjects having a cancer, and wherein the cancer is selected from any one or any combination of human cancers listed in the TCGA (e.g., COAD, BRCA, LUAD, LUSC, OV, and/or LIHC), and wherein the biomarker is selected from any one or any combination (e.g., 2, 3, 4, 5, or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some embodiments, a biomarker of the disclosure is identified by a predictive algorithm described herein, wherein the predictive algorithm comprises at least one prediction criteria selected from (i) a p value less than about 0.001, wherein the p value is determined by a statistical test (e.g., a chi-squared test) of sensitivity to perturbation of a target gene (e.g., PKYMT1) in at least four or more cell lines having a loss of function of the biomarker; and (ii) an odds ratio of greater than about 2, wherein the odds ratio is a ratio of (a) the sensitivity to perturbation of the target gene (e.g., PKYMT1) in at least four or more cell lines having a loss of function of the biomarker to (b) the odds of sensitivity to perturbation of the target gene (e.g., PKYMT1) in at least four or more cell lines comprising a wild-type biomarker, wherein the cancer is selected from any one or any combination of human cancers listed in the TCGA, and wherein the biomarker is selected from any one or any combination of the biomarkers listed in Table 1.

In some embodiments, a biomarker of the disclosure is identified by a predictive algorithm described herein, wherein the predictive algorithm comprises at least one prediction criteria selected from (i) a p value less than about 0.001, wherein the p value is determined by a statistical test (e.g., a chi-squared test) of sensitivity to perturbation of a target gene (e.g., PKYMT1) in at least four or more cell lines having a loss of function of the biomarker; and (ii) an odds ratio of greater than about 2, wherein the odds ratio is a ratio of (a) the sensitivity to perturbation of the target gene (e.g., PKYMT1) in at least four or more cell lines having a loss of function of the biomarker to (b) the odds of sensitivity to perturbation of the target gene (e.g., PKYMT1) in at least four or more cell lines comprising a wild-type biomarker, wherein the biomarker comprises an inactivating mutation (e.g., a homozygous deletion, a missense mutation encoding a nonfunctional protein, or a missense mutation encoding a truncated protein) in at least about 1%, about 2%, about 3%, about 4%, about 5% or more of subjects having a cancer, wherein the cancer is selected from any one or any combination of human cancers listed in the TCGA, and wherein the biomarker is selected from any one or any combination of the biomarkers listed in Table 1.

In some embodiments, a biomarker of the disclosure is identified by a machine learning algorithm described herein, wherein the machine learning algorithm comprises a prediction criteria that is a prediction score of greater than about 0.5, and wherein the biomarker is selected from any one or any combination (e.g., 2, 3, 4, or 5) of the biomarkers listed in Table 2.

In some embodiments, a biomarker of the disclosure is identified by a machine learning algorithm described herein, wherein the machine learning algorithm comprises a prediction criteria that is a prediction score of greater than about 0.5, wherein the biomarker comprises an inactivating mutation (e.g., a homozygous deletion, a missense mutation encoding a nonfunctional protein, or a missense mutation encoding a truncated protein) in at least about 1%, about 2%, about 3%, about 4%, about 5%, or more of subjects having a cancer, wherein the cancer is selected from any one or any combination of human cancers listed in the TCGA (e.g., COAD, BRCA, LUAD, LUSC, OV, and/or LIHC), and wherein the biomarker is selected from any one or any combination (e.g., 2, 3, 4, or 5) of the biomarkers listed in Table 2.

### Methods of Validating Synthetic Lethal Pairs

### High Throughput Genetic Screening

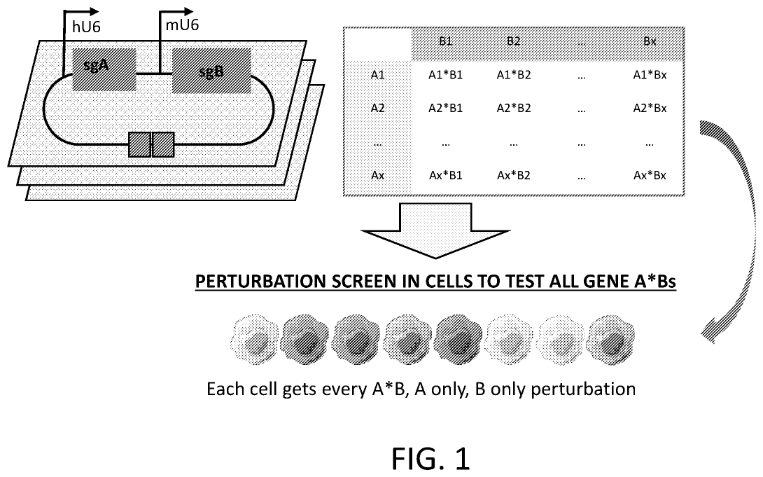

In some embodiments, a biomarker that forms a potential synthetic lethal pair with a target gene (e.g., PKMYT1) identified by one or more computational approaches described herein is further validated using one or more experimental approaches. In some embodiments, the experimental approach comprises a combinatorial genetics en masse (CombiGEM)-CRISPR screen to validate synthetic lethal pairs. Methods of performing CombiGEM screening are described in the art (see, e.g., Wong, et al. (2016) PNAS 113:2544; U.S. Pat. No. 9,315,806, incorporated herein by reference) and further described in the Examples section. In some embodiments, the CombiGEM screen comprises a workflow as depicted schematically in FIG. 1, wherein expression of a first gene encoding the biomarker identified by the one or more computational approaches and a second gene encoding the target gene (e.g., PKMYT1) are knocked down, individually or in combination, in a population of cancer cells using CRISPR/Cas gene editing, and the effect on proliferation is determined.

In some embodiments, a population of cancer cells is contacted with an expression vector (e.g., lentiviral expression vector) comprising a nucleic acid sequence encoding a first gRNA sequence, a second gRNA sequence, a first barcode sequence, and a second barcode sequence. In some embodiments, the first gRNA is directed to a gene encoding the biomarker, wherein the first gRNA comprises a spacer sequence having sequence homology to a target sequence in the gene encoding the biomarker. In some embodiments, the second gRNA is directed to the target genet (e.g., PKMYT1), wherein the second gRNA comprises a spacer sequence having sequence homology to a target sequence in the target gene (e.g., PKMYT1). In some embodiments, the expression vectors is introduced to the population of cancer cells in combination with a site-directed endonuclease (e.g., Cas9), or a nucleic acid encoding a site-directed endonuclease, wherein the first gRNA combines with the site-directed endonuclease to introduce a first genomic cleavage proximal to the target sequences in the gene encoding the biomarker, wherein the second gRNA combines with the site-directed endonuclease to introduce a second genomic cleavage proximal to the target sequence in the target gene (e.g., PKMYT1), and wherein repair of the first and second genomic cleavage by an endogenous DNA repair pathway introduces a mutation (e.g., insertion or deletion) at the sites of genomic cleavage, thereby disrupting expression of the gene encoding the biomarker and the target gene (e.g., PKMYT1). In some embodiments, the first barcode sequence and the second barcode sequence are used to measure integration of the expression vector into genomic DNA using high throughput sequencing (e.g., next generation sequencing).

In some embodiments, a population of cells is contacted with a control expression vector (e.g., lentiviral expression vector). For example, in some embodiments, to determine if a predicted gene pair is synthetically lethal, it is necessary to monitor the effect of disrupting either gene of the predicted synthetic lethal pair individually as well as the combination of the gene pair. Moreover, in some embodiments, it is necessary to monitor the effect of a negative control, in which a control expression vector comprises a nucleic acids sequence encoding an ineffective gRNA e.g., non-specific gRNA, as a “non-cutting” control for one or both genes. In some embodiments, a control expression vector is a vehicle control. In some embodiments, a control expression vector is a positive control. For example, in some embodiments, an expression vector comprises a nucleic acids sequence encoding a gRNA directed to a polymerase (e.g., an RNA polymerase, e.g., POLR2D), which can demonstrate that knockout (and the delivery mechanisms of doing so) of a gene that is essential for cell viability or proliferation results in lethality. In another example of a positive control, knockout of two genes known to be a synthetic lethal pair (e.g., methylthioadenosine phosphorylase (MTAP) and protein arginine methyltransferase 5 (PRMT5)) may be performed, e.g., using expression vectors comprising a nucleic acid sequence encoding a pair of gRNAs directed to each of the known synthetic lethal genes.

In some embodiments, the expression vector library is contacted with at least one population of cancer cells. In some embodiments, the expression vector library is contacted with two or more populations of cancer cells. In some embodiments, the population of cancer cells comprise cells from a primary source (e.g., isolated from a tumor or cancer) or a cell line. In some embodiments, the population of cancer cells belongs to a lineage selected from acute myeloid leukemia (LAML), adrenocortical carcinoma (ACC), bladder urothelial carcinoma (BLCA), brain lower grade glioma (LGG), breast invasive carcinoma (BRCA), cervical squamous cell carcinoma and endocervical adenocarcinoma (CESC), cholangiocarcinoma (CHOL), chronic myelogenous leukemia (LCML), colon adenocarcinoma (COAD), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), lymphoid neoplasm diffuse large B-cell lymphoma (DLBC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), testicular germ cell tumors (TGCT), thymoma (THYM), thyroid carcinoma (THCA), uterine carcinosarcoma (UCS), uterine corpus endometrial carcinoma (UCEC), and uveal melanoma (UVM).

In some embodiments, the population of cancer cells comprise colon adenocarcinoma cells. In some embodiments, the population of cancer cells comprises HT29 cells. In some embodiments, the population of cancer cells comprise LS180 cancer cells. In some embodiments, the population of cancer cells comprise HCT116 cancer cells. In some embodiments, the population of cancer cells comprise hepatocyte carcinoma cancer cells. In some embodiments, the population of cancer cells comprise HepG2 cancer cells. In some embodiments, the population of cancer cells comprise Huh1 cancer cells. In some embodiments, the population of cancer cells comprises Hep3B cancer cells. In some embodiments, the population of cancer cells comprise ovarian adenocarcinoma cancer cells. In some embodiments, the population of cancer cells comprise OVCAR cancer cells. In some embodiments, the population of cancer cells comprise PA1 cancer cells.

In some embodiments, the expression vectors are introduced to the population of cancer cells via transfection (e.g., using a liposome or other nanoparticle) or transduction (e.g., using a virus). In some embodiments, the site-directed endonuclease (e.g., Cas9), or a nucleic acid encoding the site-directed endonuclease (e.g., mRNA or plasmid encoding the site-directed endonuclease or Cas9), is introduced to the population of cancer cells via transfection (e.g., using a liposome or other nanoparticle) or transduction (e.g., using a virus). In some embodiments, the population of cancer cells is engineered to stably express the site-directed endonuclease. In some embodiments, the population of cancer cells is contacted with the expression vectors and/or site-directed endonuclease for a duration that is sufficient to allow for development of synthetic lethal phenotypes. In some embodiments, the contacting is performed for a duration of at least 5-30 days. In some embodiments, the contacting is performed for a duration that is about 7 days, about 14 days, about 21 days, about 28 days, or about 35 days. In some embodiments, proliferation or viability of the population of cancer cells is monitored over the duration. In some embodiments, the viability of the population of cancer cells is normalized or compared to a population of cancer cells contacted with a negative control expression vector or control population of cancer cells that was not treated. Methods of measuring cell viability are known in the art. In some embodiments, the method comprises a PrestoBlue viability assay.

In some embodiments, genomic DNA is harvested from the population of cells and next-generation sequencing is performed to establish the abundance of each of the possible pairs of gRNAs. In some embodiments, segments of the genomic DNA comprising the first barcode sequence and/or the second barcode sequence are amplified (e.g., via PCR) and sequenced (e.g., via NGS). In some embodiments, the number of reads of the first barcode sequence and/or the second barcode sequence are normalized, e.g., per 10⁶ reads of each genomic DNA sample. In some embodiments, the fold change in normalized reads of the first barcode sequence and/or the second barcode sequence is determined across the duration of the contacting compared to reads of the first barcode sequence and/or the second barcode sequence in the library. In some embodiments, the fold change is log transformed to provide a log fold change (LFC), e.g., log₂ fold change. In some embodiments, an LFC of less than zero is for specific expression vector comprising a first and second gRNAs indicates that the combination of gene knockouts induced by the first and second gRNAs has a deleterious effect on the ability of the cells to proliferate.

In some embodiments, the LFC determined for (i) a cell population administered an expression vector comprising the first gRNA and the second gRNA is compared to (ii) the LFC determined for a first control cell population contacted with an expression vector comprising the first gRNA and (iii) the LFC determined for a control cell population contacted with a library of expression vectors comprising the second gRNA. In some embodiments, the LFC for (i), (ii), and (iii) are used to determine a gene interaction score. As used herein, a “gene interaction score” refers to the difference in the observed LFC for the double knockout cell population of (i) as compared to an expected LFC. As used herein, the “expected LFC” refers to an LFC that is the sum of the LFC for the single knockout population of (ii) and the single knockout population of (iii).

In some embodiments, a gene interaction score of less than zero indicates the first gene and the second gene form a synthetic lethal pair. In some embodiments, a gene interaction score of between about −2 and 0 indicates the first gene and the second gene form a synthetic lethal pair. In some embodiments, a gene interaction score of between about −1.5 and 0 indicates the first gene and the second gene form a synthetic lethal pair. In some embodiments, a gene interaction score of between about −2 and −1 indicates the first gene and the second gene form a synthetic lethal pair. In some embodiments, a gene interaction score of between about −1.5 and −0.8 indicates the first gene and the second gene form a synthetic lethal pair. In some embodiments, a gene interaction score of between about −1.5 and −1.0 indicates the first gene and the second gene form a synthetic lethal pair.

In some embodiments, a gene interaction score of less than zero measured in at least one population of cancer cells (e.g., population of cells comprising HT29 cells or LS180 cells) indicates the first gene and the second gene form a synthetic lethal pair. In some embodiments, a gene interaction score of between about −2 and 0 in at least one population of cancer cells (e.g., population of cells comprising HT29 cells or LS180 cells) indicates the first gene and the second gene form a synthetic lethal pair. In some embodiments, a gene interaction score of between about −1.5 and 0 in at least one population of cancer cells (e.g., population of cells comprising HT29 cells or LS180 cells) indicates the first gene and the second gene form a synthetic lethal pair. In some embodiments, a gene interaction score of between about −2 and −1 measured in at least one population of cancer cells (e.g., population of cells comprising HT29 cells or LS180 cells) indicates the first gene and the second gene form a synthetic lethal pair. In some embodiments, a gene interaction score of between about −1.5 and −0.8 measured in at least one population of cancer cells (e.g., population of cells comprising HT29 cells or LS180 cells) indicates the first gene and the second gene form a synthetic lethal pair. In some embodiments, a gene interaction score of between about −1.5 and −1.0 measured in at least one population of cancer cells (e.g., population of cells comprising HT29 cells or LS180 cells) indicates the first gene and the second gene form a synthetic lethal pair.

In some embodiments, a gene interaction score of less than zero measured in at least two populations of cancer cells (e.g., a population of cells comprising HT29 cells and a population of cells comprising LS180 cells) indicates the first gene and the second gene form a synthetic lethal pair. In some embodiments, a gene interaction score of between about −2 and 0 in at least two populations of cancer cells (e.g., population of cells comprising HT29 cells or LS180 cells) indicates the first gene and the second gene form a synthetic lethal pair. In some embodiments, a gene interaction score of between about −1.5 and 0 in at least two populations of cancer cells (e.g., population of cells comprising HT29 cells or LS180 cells) indicates the first gene and the second gene form a synthetic lethal pair. In some embodiments, a gene interaction score of between about −2 and −1 measured in at least two populations of cancer cells (e.g., a population of cells comprising HT29 cells and a population of cells comprising LS180 cells) indicates the first gene and the second gene form a synthetic lethal pair. In some embodiments, a gene interaction score of between about −1.5 and −0.8 measured in at least two populations of cancer cells (e.g., a population of cells comprising HT29 cells and a population of cells comprising LS180 cells) indicates the first gene and the second gene form a synthetic lethal pair. In some embodiments, a gene interaction score of between about −1.5 and −1.0 measured in at least two populations of cancer cells (e.g., a population of cells comprising HT29 cells and a population of cells comprising LS180 cells) indicates the first gene and the second gene form a synthetic lethal pair.

### Exemplary Methods of Validating Synthetic Lethal Pairs

In some embodiments, the biomarkers of the disclosure are validated as a synthetic lethal pair with PKMYT1 using a CombiGEM screen. In some embodiments, the CombiGEM screen comprises contacting a population of cancer cells (e.g., a population of cancer cells comprising HT29 cells or LS180 cells) with an expression vector comprising a nucleic acid sequence encoding from 5′ to 3′: a first gRNA targeting a biomarker gene, a second gRNA targeting PKMYT1, a first barcode sequence, and a second barcode sequence. In some embodiments, the first gRNA comprises a spacer sequence having sequence homology to a target sequence in a gene encoding a biomarker (e.g., a biomarker identified via a computational approach as a putative synthetic lethal pair with PKMYT1). In some embodiments, the second gRNA comprises a spacer sequence having sequence homology to a target sequence in the PKMYT1 gene. In some embodiments, the population of cells comprises a site-directed endonuclease (e.g., Cas9). In some embodiments, the first gRNA combined with the site-directed endonuclease introduces a first genomic cleavage at the target sequence in the gene encoding the biomarker; and the second gRNA combined with the site-directed endonuclease introduces a second genomic cleavage at the target sequence in the PKMYT1 gene; wherein the repair of the first and second genomic cleavage by an endogenous DNA repair pathway introduces a deleterious mutation in the gene encoding the biomarker and the PKMYT1 gene.

In some embodiments, the population of cancer cells (e.g., a population of cancer cells comprising HT29 cells or LS180 cells) is contacted with the expression vector for a duration of at least 15-30 days. In some embodiments, the genomic DNA is harvested from the population of cells, and the number of reads of the first and second DNA barcode sequences is quantified to determine the LFC. In some embodiments, the LFC is determined for (i) a population of cells contacted with an expression vector encoding the first and second gRNAs, (ii) a first control population of cells contacted with an expression vector encoding the first gRNA targeting a biomarker gene, and (iii) a second control population of cells contacted with an expression vector encoding the second gRNA targeting PKMYT1. In some embodiments, the genetic interaction score is quantified as the observed LFC for (i) minus the expected LFC, wherein the expected LFC is the sum of the LFC for (ii) and (iii).

In some embodiments, a biomarker of the disclosure (e.g., that forms a synthetic lethal pair with PKMYT1) is identified by one or more computational algorithms described herein. In some embodiments, the biomarker is identified by a predictive algorithm applied to an experimental dataset (e.g., a dataset based on functional genomic screening). In some embodiments, a biomarker of the disclosure identified by a predictive algorithm described herein is selected from CDKN1B, DUSP7, FOXO3, TP53, and FZD3. In some embodiments, the biomarker is identified by a machine learning algorithm. In some embodiments, a biomarker of the disclosure identified by a machine learning algorithm described herein is selected from ATM, MAP2K4, TP53, and CDC25A. In some embodiments, the biomarker is identified by both a predictive algorithm and a machine learning algorithm. In some embodiments, a biomarker of the disclosure identified by both a predictive algorithm and a machine learning algorithm described herein is TP53.

In some embodiments, a biomarker of the disclosure is validated in a high throughput genetic screen described herein. In some embodiments, the biomarker comprises (i) an LFC of less than about −1 in at least one population of cancer cells (e.g., population of cancer cells comprising HT29 cells or LS180 cells); or (ii) a gene interaction score of less than about −1 in at least one population of cancer cells (e.g., population of cancer cells comprising HT29 cells or LS180 cells). In some embodiments, the biomarker comprises both (i) and (ii). In some embodiments, a biomarker of the disclosure comprises (i) an LFC of less than about −1 in at least two populations of cancer cells (e.g., population of cancer cells comprising HT29 cells and a population of cancer cells comprising LS180 cells); and/or (ii) a gene interaction score of less than about −1 in at least two population of cancer cells (e.g., population of cancer cells comprising HT29 cells and a population of cells comprising LS180 cells). In some embodiments, the biomarker is selected from TP53, CDKN1B, DUSP7, FOXO3, FZD3, SMAD2, ATM, MAP2K4, CACNA1H, JAK1, and TGFBR. In some embodiments, a biomarker of the disclosure comprises (i) an LFC of less than about −1 in at least one populations of cancer cells (e.g., population of cancer cells comprising HT29 cells or a population of cancer cells comprising LS180 cells); and/or (ii) a gene interaction score of less than about −1 in at least one population of cancer cells (e.g., population of cancer cells comprising HT29 cells or a population of cells comprising LS180 cells). In some embodiments, the biomarker is selected from TP53, CDKN1B, DUSP7, FOXO3, FZD3, SMAD2, ATM, MAP2K4, CACNA1H, JAK1, TGFBR, CDC25A, PPP2R1B, PPP3CC and MAP3K2.

In some embodiments, the biomarker is identified by one or more computational algorithms described herein (e.g., a predictive algorithm and/or a machine learning algorithm) and validated in a high throughput genetic screen described herein. In some embodiments, a biomarker of the disclosure is identified by two or more computational algorithms described herein (e.g., a predictive algorithm and a machine learning algorithm) and validated in a high throughput genetic screen described herein by comprising (i) an LFC of less than about −1 in at least two populations of cancer cells (e.g., population of cancer cells comprising HT29 cells and a population of cancer cells comprising LS180 cells); and (ii) a gene interaction score of less than about −1 in at least two population of cancer cells (e.g., population of cancer cells comprising HT29 cells and a population of cells comprising LS180 cells). In some embodiments, the biomarker is TP53.

In some embodiments, a biomarker of the disclosure is identified by one or more computational algorithms described herein (e.g., a predictive algorithm or a machine learning algorithm) and validated in a high throughput genetic screen described herein by comprising (i) an LFC of less than about −1 in at least two populations of cancer cells (e.g., population of cancer cells comprising HT29 cells and/or a population of cancer cells comprising LS180 cells); and (ii) a gene interaction score of less than about −1 in at least two population of cancer cells (e.g., population of cancer cells comprising HT29 cells and/or a population of cells comprising LS180 cells). In some embodiments, the biomarker is TP53. In some embodiments, the biomarker is CDKN1B. In some embodiments, the biomarker is DUSP7. In some embodiments, the biomarker is FOXO3. In some embodiments, the biomarker is FZD3. In some embodiments, the biomarker is SMAD2. In some embodiments, the biomarker is ATM. In some embodiments, the biomarker is MAP2K4.

In some embodiments, a biomarker of the disclosure is identified by one or more computational algorithms described herein (e.g., a predictive algorithm or a machine learning algorithm) and validated in a high throughput genetic screen described herein by comprising (i) an LFC of less than about −1 in at least one populations of cancer cells (e.g., population of cancer cells comprising HT29 cells or a population of cancer cells comprising LS180 cells); and (ii) a gene interaction score of less than about −1 in at least one population of cancer cells (e.g., population of cancer cells comprising HT29 cells or a population of cells comprising LS180 cells). In some embodiments, the biomarker is TP53. In some embodiments, the biomarker is CDKN1B. In some embodiments, the biomarker is DUSP7. In some embodiments, the biomarker is FOXO3. In some embodiments, the biomarker is FZD3. In some embodiments, the biomarker is SMAD2. In some embodiments, the biomarker is ATM. In some embodiments, the biomarker is MAP2K4. In some embodiments, the biomarker is CDC25A.

In some embodiments, a biomarker of the disclosure is validated in a high throughput genetic screen described herein. In some embodiments, the biomarker comprises (i) an LFC of less than about −1 in at least one population of cancer cells (e.g., population of cancer cells comprising HT29 cells or LS180 cells); or (ii) a gene interaction score of less than about −1 in at least one population of cancer cells (e.g., population of cancer cells comprising HT29 cells or LS180 cells). In some embodiments, the biomarker comprises both (i) and (ii).

In some embodiments, a biomarker of the disclosure comprises (i) an LFC of less than about −1 in at least two populations of cancer cells (e.g., population of cancer cells comprising HT29 cells and a population of cancer cells comprising LS180 cells); and/or (ii) a gene interaction score of less than about −1 in at least two population of cancer cells (e.g., population of cancer cells comprising HT29 cells and a population of cells comprising LSI80 cells), wherein the biomarker is selected from any one or any combination (e.g., 2, 3, 4, 5, or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some embodiments, a biomarker of the disclosure comprises (i) an LFC of less than about −1 in at least one populations of cancer cells (e.g., population of cancer cells comprising HT29 cells or a population of cancer cells comprising LS180 cells); and/or (ii) a gene interaction score of less than about −1 in at least one population of cancer cells (e.g., population of cancer cells comprising HT29 cells or a population of cells comprising LS180 cells), wherein the biomarker is selected from any one or any combination (e.g., 2, 3, 4, 5, or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some embodiments, a biomarker of the disclosure comprises (i) an LFC of less than about minus 1 in at least two populations of cancer cells (e.g., population of cancer cells comprising HT29 cells and a population of cancer cells comprising LS180 cells); and/or (ii) a gene interaction score of less than about minus 1 in at least two population of cancer cells (e.g., population of cancer cells comprising HT29 cells and a population of cells comprising LS180 cells), wherein the biomarker is selected from any one or any combination (e.g., 2, 3, 4, 5 or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some embodiments, a biomarker of the disclosure comprises (i) an LFC of less than about minus 1 in at least one populations of cancer cells (e.g., population of cancer cells comprising HT29 cells or a population of cancer cells comprising LS180 cells); and/or (ii) a gene interaction score of less than about minus 1 in at least one population of cancer cells (e.g., population of cancer cells comprising HT29 cells or a population of cells comprising LS180 cells), wherein the biomarker is selected from any one or any combination (e.g., 2, 3, 4, 5, or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some embodiments, a biomarker of the disclosure comprises (i) an LFC of less than about minus 1 in at least two populations of cancer cells (e.g., population of cancer cells comprising HT29 cells and a population of cancer cells comprising LS180 cells); and/or (ii) a gene interaction score of less than about minus 1 in at least two population of cancer cells (e.g., population of cancer cells comprising HT29 cells and a population of cells comprising LS180 cells), wherein the biomarker is selected from any one or any combination (e.g., 2, 3, 4, 5, or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some embodiments, a biomarker of the disclosure comprises (i) an LFC of less than about minus 1 in at least one populations of cancer cells (e.g., population of cancer cells comprising HT29 cells or a population of cancer cells comprising LS180 cells); and/or (ii) a gene interaction score of less than about minus 1 in at least one population of cancer cells (e.g., population of cancer cells comprising HT29 cells or a population of cells comprising LS180 cells), wherein the biomarker is selected from any one or any combination (e.g., 2, 3, 4, 5, or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some embodiments, a biomarker of the disclosure comprises (i) an LFC of less than about minus 1 in at least two populations of cancer cells (e.g., population of cancer cells comprising HT29 cells and a population of cancer cells comprising LS180 cells); and/or (ii) a gene interaction score of less than about minus 1 in at least two population of cancer cells (e.g., population of cancer cells comprising HT29 cells and a population of cells comprising LS180 cells), wherein the biomarker is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some embodiments, a biomarker of the disclosure comprises (i) an LFC of less than about minus 1 in at least one populations of cancer cells (e.g., population of cancer cells comprising HT29 cells or a population of cancer cells comprising LS180 cells); and/or (ii) a gene interaction score of less than about minus 1 in at least one population of cancer cells (e.g., population of cancer cells comprising HT29 cells or a population of cells comprising LS180 cells), wherein the biomarker is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some embodiments, a biomarker of the disclosure comprises (i) an LFC of less than about minus 1 in at least two populations of cancer cells (e.g., population of cancer cells comprising HT29 cells and a population of cancer cells comprising LS180 cells); and/or (ii) a gene interaction score of less than about minus 1 in at least two population of cancer cells (e.g., population of cancer cells comprising HT29 cells and a population of cells comprising LS180 cells), and wherein the biomarker is selected from any one or any combination (e.g., 2, 3, 4, 5 or more) of the biomarkers listed in Table 1.

In some embodiments, a biomarker of the disclosure comprises (i) an LFC of less than about minus 1 in at least one populations of cancer cells (e.g., population of cancer cells comprising HT29 cells or a population of cancer cells comprising LS180 cells); and/or (ii) a gene interaction score of less than about minus 1 in at least one population of cancer cells (e.g., population of cancer cells comprising HT29 cells or a population of cells comprising LS180 cells), and wherein the biomarker is selected from any one or any combination (e.g., 2, 3, 4, 5 or more) of the biomarkers listed in Table 1.

In some embodiments, a biomarker of the disclosure comprises (i) an LFC of less than about minus 1 in at least two populations of cancer cells (e.g., population of cancer cells comprising HT29 cells and a population of cancer cells comprising LS180 cells); and/or (ii) a gene interaction score of less than about minus 1 in at least two population of cancer cells (e.g., population of cancer cells comprising HT29 cells and a population of cells comprising LS180 cells), wherein the biomarker is selected from any one or any combination (e.g., 2, 3, 4, 5 or more) of the biomarkers listed in Table 2.

In some embodiments, a biomarker of the disclosure comprises (i) an LFC of less than about minus 1 in at least one populations of cancer cells (e.g., population of cancer cells comprising HT29 cells or a population of cancer cells comprising LS180 cells); and/or (ii) a gene interaction score of less than about minus 1 in at least one population of cancer cells (e.g., population of cancer cells comprising HT29 cells or a population of cells comprising LS180 cells), wherein the biomarker is selected from any one or any combination (e.g., 2, 3, 4, 5 or more) of the biomarkers listed in Table 2.

In some embodiments, a biomarker of the disclosure comprises (i) an LFC of less than about minus 1 in at least two populations of cancer cells (e.g., population of cancer cells comprising HT29 cells, a population of cancer cells comprising LS180 cells, and/or a population of cancer cells comprising PA1 cells); and/or (ii) a gene interaction score of less than about minus 1 in at least two population of cancer cells (e.g., population of cancer cells comprising HT29 cells, a population of cancer cells comprising LS180 cells, and/or a population of cancer cells comprising PA1 cells), wherein the biomarker is selected from any one or any combination (e.g., 2, 3, 4, 5 or more) of the biomarkers listed in Table 3. In some embodiments, a biomarker of the disclosure comprises (i) an LFC of less than about minus 1 in at least one populations of cancer cells (e.g., population of cancer cells comprising HT29 cells, a population of cancer cells comprising LS180 cells, and/or a population of cancer cells comprising PA1 cells); and/or (ii) a gene interaction score of less than about minus 1 in at least one population of cancer cells (e.g., population of cancer cells comprising HT29 cells, a population of cancer cells comprising LS180 cells, and/or a population of cancer cells comprising PA1 cells), wherein the biomarker is selected from any one or any combination (e.g., 2, 3, 4, 5 or more) of the biomarkers listed in Table 3.

In some embodiments, a biomarker of the disclosure comprises (i) an LFC of less than about minus 1 in at least two populations of cancer cells (e.g., population of cancer cells comprising HT29 cells, a population of cancer cells comprising LS180 cells, and/or a population of cancer cells comprising PA1 cells); and/or (ii) a gene interaction score of less than about minus 1 in at least two population of cancer cells (e.g., population of cancer cells comprising HT29 cells, a population of cancer cells comprising LS180 cells, and/or a population of cancer cells comprising PA1 cells), wherein the biomarker is selected from any one or any combination (e.g., 2, 3, 4, 5 or more) of the biomarkers listed in Table 8 and 9.

In some embodiments, a biomarker of the disclosure comprises (i) an LFC of less than about minus 1 in at least one populations of cancer cells (e.g., population of cancer cells comprising HT29 cells, a population of cancer cells comprising LS180 cells, and/or a population of cancer cells comprising PA1 cells); and/or (ii) a gene interaction score of less than about minus 1 in at least one population of cancer cells (e.g., population of cancer cells comprising HT29 cells, a population of cancer cells comprising LS180 cells, and/or a population of cancer cells comprising PA1 cells), wherein the biomarker is selected from any one or any combination (e.g., 2, 3, 4, 5 or more) of the biomarkers listed in Table 8 and 9.

In some embodiments, a biomarker of the disclosure comprises (i) an LFC of less than about minus 1 in at least two populations of cancer cells (e.g., population of cancer cells comprising HT29 cells, a population of cancer cells comprising LS180 cells, and/or a population of cancer cells comprising PA1 cells); and/or (ii) a gene interaction score of less than about minus 1 in at least two population of cancer cells (e.g., population of cancer cells comprising HT29 cells, a population of cancer cells comprising LS180 cells, and/or a population of cancer cells comprising PA1 cells), wherein the biomarker is selected from any one or any combination (e.g., 2, 3, 4, 5 or more) of the biomarkers listed in Table 10.

In some embodiments, a biomarker of the disclosure comprises (i) an LFC of less than about minus 1 in at least one populations of cancer cells (e.g., population of cancer cells comprising HT29 cells, a population of cancer cells comprising LS180 cells, and/or a population of cancer cells comprising PA1 cells); and/or (ii) a gene interaction score of less than about minus 1 in at least one population of cancer cells (e.g., population of cancer cells comprising HT29 cells, a population of cancer cells comprising LS180 cells, and/or a population of cancer cells comprising PA1 cells), wherein the biomarker is selected from any one or any combination (e.g., 2, 3, 4, 5 or more) of the biomarkers listed in Table 10.

### Methods of Use

The present disclosure provides methods for treating a subject having cancer comprising administering a therapeutic agent described herein that alters (e.g., increase or decrease) the expression and/or activity of a PKMYT1 gene, or a transcriptional or translational product thereof, wherein the subject has a tumor characterized by the presence of a mutation in, an altered expression level of, and/or an altered activity of one or more biomarkers disclosed herein relative to a reference tissue.

The present disclosure provides methods for determining the responsiveness of a subject having cancer to treatment with a therapeutic agent described herein that alters (e.g., increase or decrease) the expression and/or activity of a PKMYT1 gene, or a transcriptional or translational product thereof, the method comprising detecting the presence of a mutation in, an altered expression level of, and/or an altered activity of one or more biomarkers disclosed herein in a cancerous tissue sample obtained from the subject, wherein the presence of a mutation, an altered expression level, and or an altered activity in the cancerous tissue sample relative to a reference tissue indicates the subject will respond or will likely respond to the therapeutic agent.

### Therapeutic Methods

In some embodiments, the present disclosure provides methods for the treatment of cancer (e.g., liver or ovarian cancer). In some embodiments, the cancer is characterized by an altered (e.g., increased or decreased) expression level and/or activity of a biomarker of the disclosure (e.g., a biomarker that forms a synthetic lethal pair with PKMYT1). In some embodiments, the cancer is characterized by the presence of a mutation in a biomarker of the disclosure (e.g., a biomarker that forms a synthetic lethal pair with PKMYT1). In some embodiments, the method comprises administering one or more therapeutic agents for manipulation of the expression level and/or activity of a target gene or a transcriptional or translational product thereof. In some embodiments, the method comprises administering one or more therapeutic agents for modulating the expression level and/or activity of PKMYT1.

In some embodiments, the disclosure provides a method of treating cancer in a subject, comprising administering to the subject one or more therapeutic agents for modulating a PKMYT1 gene, or a transcriptional or translational product thereof, wherein the cancer, or a plurality of cancer cells thereof, comprises one or more mutations in, an altered (e.g., increased or decreased) expression level of, and/or an altered (e.g., increased or decreased) expression level of activity a biomarker described herein, wherein the biomarker forms a synthetic lethal pair with the PKMYT1 gene. In some embodiments, the biomarker is selected from Table 3. In some embodiments, the biomarker is selected from Table 8 and 9. In some embodiments, the biomarker is selected from Table 10.

In some embodiments, the disclosure provides a method of treating cancer in a subject, comprising administering to the subject one or more therapeutic agents for modulating a PKMYT1 gene, or a transcriptional or translational product thereof, wherein the cancer, or a plurality of cancer cells thereof, comprises one or more mutations resulting in a decreased expression level and/or activity of a biomarker selected from Table 3. In some embodiments, the cancer, or a plurality of cancer cells thereof, comprises one or more mutations resulting in a decreased expression level and/or activity of a biomarker selected from Table 8 and 9. In some embodiments, the cancer, or a plurality of cancer cells thereof, comprises one or more mutations resulting in a decreased expression level and/or activity of a biomarker selected from Table 10.

In some embodiments, the disclosure provides a method of treating cancer in a subject, comprising administering to the subject one or more therapeutic agents for decreasing an expression level and/or activity of a PKMYT1 gene, or a transcriptional or translational product thereof, wherein the cancer, or a plurality of cancer cells thereof, comprise a loss of function mutation or an inactivating mutation in a biomarker selected from Table 3. In some embodiments, the cancer, or a plurality of cancer cells thereof, comprise a loss of function mutation or an inactivating mutation in a biomarker selected from Table 8 and 9. In some embodiments, the cancer, or a plurality of cancer cells thereof, comprise a loss of function mutation or an inactivating mutation in a biomarker selected from Table 10.

In some embodiments, the disclosure provides a method of treating cancer in a subject, comprising administering to the subject one or more therapeutic agents for modulating a PKMYT1 gene, or a transcriptional or translational product thereof, wherein the cancer, or a plurality of cancer cells thereof, comprises an altered (e.g., increased or decreased) expression level and/or altered (e.g., increased or decreased) activity of a biomarker selected from Table 3. In some embodiments, the cancer, or a plurality of cancer cells thereof, comprises an altered (e.g., increased or decreased) expression level and/or altered (e.g., increased or decreased) activity of a biomarker selected from Table 8 and 9. In some embodiments, the cancer, or a plurality of cancer cells thereof, comprises an altered (e.g., increased or decreased) expression level and/or altered (e.g., increased or decreased) activity of a biomarker selected from Table 10.

In some embodiments, the disclosure provides a method of promoting tumor regression in a subject, comprising administering to the subject one or more therapeutic agents for modulating a PKMYT1 gene, or a transcriptional or translational product thereof, wherein the tumor, or a plurality of tumor cells thereof, comprises one or more mutations in, an altered (e.g., increased or decreased) expression level of, and/or an altered (e.g., increased or decreased) expression level of activity a biomarker described herein, wherein the biomarker forms a synthetic lethal pair with the PKMYT1 gene. In some embodiments, the biomarker is selected from Table 3. In some embodiments, the biomarker is selected from Table 8 and 9. In some embodiments, the biomarker is selected from Table 10.

In some embodiments, the disclosure provides a method of promoting tumor regression in a subject, comprising administering to the subject one or more therapeutic agents for modulating PKMYT1 gene, or a transcriptional or translational product thereof, wherein the tumor, or a plurality of tumor cells thereof, comprises one or mutations resulting in a decreased expression level and/or activity of a biomarker selected from Table 3. In some embodiments, the tumor, or a plurality of tumor cells thereof, comprise one or more mutations resulting in a decreased expression level and/or activity of a biomarker selected from Table 8 and 9. In some embodiments, the tumor, or a plurality of tumor cells thereof, comprise one or more mutations resulting in a decreased expression level and/or activity of a biomarker selected from Table 10.

In some embodiments, the disclosure provides a method of promoting tumor regression in a subject, comprising administering to the subject one or more therapeutic agents for modulating a PKMYT1 gene, or a transcriptional or translational product thereof, wherein the tumor, or a plurality of tumor cells thereof, comprises a loss of function mutation or an inactivating mutation in a biomarker selected from Table 3. In some embodiments, the tumor, or a plurality of tumor cells thereof, comprises a loss of function mutation or an inactivating mutation in a biomarker selected from Table 8 and 9. In some embodiments, the tumor, or a plurality of tumor cells thereof, comprises a loss of function mutation or an inactivating mutation in a biomarker selected from Table 10.

In some embodiments, the disclosure provides a method of promoting tumor regression in a subject, comprising administering to the subject one or more therapeutic agents for modulating a PKMYT1 gene, or a transcriptional or translational product thereof, wherein the tumor, or a plurality of tumor cells thereof, comprises an altered (e.g., increased or decreased) expression level and/or altered (e.g., increased or decreased) activity of a biomarker selected from Table 3. In some embodiments, the tumor, or a plurality of tumor cells thereof, comprises an altered (e.g., increased or decreased) expression level and/or altered (e.g., increased or decreased) activity of a biomarker selected from Table 8 and 9. In some embodiments, the tumor, or a plurality of tumor cells thereof, comprises an altered (e.g., increased or decreased) expression level and/or altered (e.g., increased or decreased) activity of a biomarker selected from Table 10.

In some embodiments, the disclosure provides a method of promoting or inducing synthetic lethality in a tumor in a subject, comprising administering to the subject one or more therapeutic agents for modulating a PKMYT1 gene, or a transcriptional or translational product thereof, wherein the tumor, or a plurality of tumor cells thereof, comprises one or more mutations in, an altered (e.g., increased or decreased) expression level of, and/or an altered (e.g., increased or decreased) expression level of activity a biomarker described herein, wherein the biomarker forms a synthetic lethal pair with PKMYT1 gene. In some embodiments, the biomarker is selected from Table 3. In some embodiments, the biomarker is selected from Table 8 and 9. In some embodiments, the biomarker is selected from Table 10.

In some embodiments, the disclosure provides a method of promoting or inducing synthetic lethality in a tumor in a subject, comprising administering to the subject one or more therapeutic agents for modulating a PKMYT1 gene, or a transcriptional or translational product thereof, wherein the tumor, or a plurality of tumor cells thereof, comprises one or mutations resulting in a decreased expression level and/or activity of a biomarker selected from Table 3. In some embodiments, wherein the tumor, or a plurality of tumor cells thereof, comprises one or mutations resulting in a decreased expression level and/or activity of a biomarker selected from Table 8 and 9. In some embodiments, wherein the tumor, or a plurality of tumor cells thereof, comprises one or mutations resulting in a decreased expression level and/or activity of a biomarker selected from Table 10.

In some embodiments, the disclosure provides a method of promoting or inducing synthetic lethality in a tumor in a subject, comprising administering to the subject one or more therapeutic agents for modulating a PKMYT1 gene, or a transcriptional or translational product thereof, wherein the tumor, or a plurality of tumor cells thereof, comprises a loss of function mutation or an inactivating mutation in a biomarker selected from Table 3. In some embodiments, the tumor, or a plurality of tumor cells thereof, comprises a loss of function mutation or an inactivating mutation in a biomarker selected from Table 8 and 9. In some embodiments, the tumor, or a plurality of tumor cells thereof, comprises a loss of function mutation or an inactivating mutation in a biomarker selected from Table 10.

In some embodiments, the disclosure provides a method of promoting or inducing synthetic lethality in a tumor in a subject, comprising administering to the subject one or more therapeutic agents for modulating a PKMYT1 gene, or a transcriptional or translational product thereof, wherein the tumor, or a plurality of tumor cells thereof, comprises an altered (e.g., increased or decreased) expression level and/or altered (e.g., increased or decreased) activity of a biomarker selected from Table 3. In some embodiments, the tumor, or a plurality of tumor cells thereof, comprises an altered (e.g., increased or decreased) expression level and/or altered (e.g., increased or decreased) activity of a biomarker selected from Table 8 and 9. In some embodiments, the tumor, or a plurality of tumor cells thereof, comprises an altered (e.g., increased or decreased) expression level and/or altered (e.g., increased or decreased) activity of a biomarker selected from Table 10.

In some embodiments, the one or more mutations is detected in a tissue sample obtained from the subject. In some embodiments, the tissue sample is a tumor biopsy sample (e.g., a fresh or fixed biopsy sample). In some embodiments, the tissue sample is a blood sample or a blood component sample (e.g., plasma) comprising circulating tumor DNA.

In some embodiments, the one or more mutations results in an altered (e.g., increased or decreased) expression level of a biomarker selected from Table 3 in the tumor relative to a reference tissue sample (e.g., healthy control tissue). In some embodiments, the one or more mutations results in an altered (e.g., increased or decreased) expression level of a biomarker selected from Table 8 and 9 in the tumor relative to a reference tissue sample (e.g., healthy control tissue). In some embodiments, the one or more mutations results in an altered (e.g., increased or decreased) expression level of a biomarker selected from Table 10 in the tumor relative to a reference tissue sample (e.g., healthy control tissue). In some embodiments, the one or more mutations results in a decreased expression level of a biomarker selected from Table 3 (e.g., partial or complete loss of expression of the biomarker) in the tumor relative to a reference tissue sample (e.g., healthy control tissue). In some embodiments, the one or more mutations results in a decreased expression level of a biomarker selected from Table 8 and 9 (e.g., partial or complete loss of expression of the biomarker) in the tumor relative to a reference tissue sample (e.g., healthy control tissue). In some embodiments, the one or more mutations results in a decreased expression level of a biomarker selected from Table 10 (e.g., partial or complete loss of expression of the biomarker) in the tumor relative to a reference tissue sample (e.g., healthy control tissue). In some embodiments, the one or more mutations results in a deficient activity (e.g., increased or decreased) of a biomarker selected from Table 3 in the tumor relative to a reference tissue sample (e.g., healthy control tissue). In some embodiments, the one or more mutations results in a deficient activity (e.g., increased or decreased) of a biomarker selected from Table 8 and 9 in the tumor relative to a reference tissue sample (e.g., healthy control tissue). In some embodiments, the one or more mutations results in a deficient activity (e.g., increased or decreased) of a biomarker selected from Table 10 in the tumor relative to a reference tissue sample (e.g., healthy control tissue). In some embodiments, the one or more mutations results in a decreased activity of a biomarker selected from Table 3 (e.g., partial or complete loss of activity of the biomarker) in the tumor relative to a reference tissue sample (e.g., healthy control tissue). In some embodiments, the one or more mutations results in a decreased activity of a biomarker selected from Table 8 and 9 (e.g., partial or complete loss of activity of the biomarker) in the tumor relative to a reference tissue sample (e.g., healthy control tissue). In some embodiments, the one or more mutations results in a decreased activity of a biomarker selected from Table 10 (e.g., partial or complete loss of activity of the biomarker) in the tumor relative to a reference tissue sample (e.g., healthy control tissue).

In some embodiments, the disclosure provides a method of treating cancer in a subject, comprising administering to the subject one or more therapeutic agents for manipulation of a target gene or a transcriptional or translational product thereof (e.g., PKMYT1), wherein the cancer, or a plurality of cancer cells thereof, comprises an altered (e.g., increased or decreased) expression level and/or activity of a biomarker described herein, wherein the biomarker forms a synthetic lethal pair with the target gene. In some embodiments, the expression level of the biomarker is deficient (e.g., under-expressed, mutated, over-expressed) in the cancer or a plurality of cancer cells thereof. In some embodiments, the activity of the biomarker is deficient (e.g., increased or decreased) in the cancer or a plurality of cancer cells thereof.

In some embodiments, the cancer comprises a decreased expression level of the biomarker. In some embodiments, a plurality of cancer cells comprises a decreased expression level of the biomarker, wherein the plurality of cancer cells is at least about 5% of the total number of cancer cells in the subject. In some embodiments, the plurality of cancer cells is at least about 10% of the total number of cancer cells in the subject. In some embodiments, the plurality of cancer cells is about 10%, about 20%, about 30%, about 40%, about 50%, about 60%, about 70%, about 80%, about 90%, or about 100% of the total number of cancer cells in the subject. In some embodiments, the cancer comprises a decreased activity of the biomarker. In some embodiments, a plurality of cancer cells comprises a decreased activity of the biomarker, wherein the plurality of cancer cells is at least 5% of the total number of cancer cells in the subject. In some embodiments, the expression level of the biomarker is at least 1.1-fold, 1.5-fold, 2-fold, 3-fold, 5-fold, 10-fold lower in the cancer compared to a reference tissue (e.g., healthy control tissue). In some embodiments, the activity of the biomarker is at least 1.1-fold, 1.5-fold, 2-fold, 3-fold, 5-fold, 10-fold lower in the cancer compared to a reference tissue (e.g., healthy control tissue).

In some embodiments, the disclosure provides a method of treating cancer in a subject, comprising administering to the subject one or more therapeutic agents for manipulation of a target gene or a transcriptional or translational product thereof (e.g., PKMYT1), wherein the cancer, or a plurality of cancer cells thereof, comprises a mutation in the biomarker. In some embodiments, the mutation is a loss of function mutation described herein resulting in a decreased expression level and/or activity of the biomarker. In some embodiments, the mutation is detected in a tissue sample obtained from the subject. In some embodiments, the tissue sample is a tumor biopsy sample (e.g., a fresh or fixed biopsy sample). In some embodiments, the tissue sample is a blood sample or a blood component sample (e.g., plasma) comprising circulating tumor DNA.

In some embodiments, the disclosure provides a method of promoting tumor regression in a subject, comprising administering to the subject one or more therapeutic agents for manipulation of a target gene or a transcriptional or translational product thereof (e.g., PKMYT1), wherein the tumor, or a plurality of tumor cells thereof, comprises an altered (e.g., increased or decreased) expression level and/or activity of a biomarker described herein, and wherein the biomarker forms a synthetic lethal pair with the target gene. In some embodiments, the disclosure provides a method of promoting or inducing synthetic lethality in a tumor in a subject, comprising administering to the subject one or more therapeutic agents for manipulation of a target gene or a transcriptional or translational product thereof (e.g., PKMYT1), wherein the tumor, or a plurality of tumor cells thereof, comprises an altered (e.g., increased or decreased) expression level and/or activity of a biomarker described herein, and wherein the biomarker forms a synthetic lethal pair with the target gene. In some embodiments, the expression level of the biomarker is deficient (e.g., under-expressed, mutated, over-expressed) in the tumor or a plurality of tumor cells thereof. In some embodiments, the activity of the biomarker is deficient (e.g., increased or decreased) in the tumor or a plurality of tumor cells thereof. In some embodiments, a plurality of tumor cells comprises a decreased expression level of the biomarker, wherein the plurality of tumor cells is at least 5% of the total number of tumor cells in the subject. In some embodiments, the tumor comprises a decreased activity of the biomarker. In some embodiments, a plurality of tumor cells comprises a decreased activity of the biomarker, wherein the plurality of tumor cells is at least 5% of the total number of cancer cells in the subject. In some embodiments, the expression level of the biomarker is at least 1.1-fold, 1.5-fold, 2-fold, 3-fold, 5-fold, 10-fold lower in the tumor compared to a reference tissue (e.g., healthy control tissue). In some embodiments, the activity of the biomarker is at least 1.1-fold, 1.5-fold, 2-fold, 3-fold, 5-fold, 10-fold lower in the tumor compared to a reference tissue (e.g., healthy control tissue).

In some embodiments, the disclosure provides a method of promoting tumor regression in a subject, comprising administering to the subject one or more therapeutic agents for manipulation of a target gene or a transcriptional or translational product thereof (e.g., PKMYT1), wherein the tumor, or a plurality of tumor cells thereof, comprise a mutation in a biomarker described herein (e.g., a loss of function mutation resulting in a decreased expression level and/or activity of the biomarker), and wherein the biomarker forms a synthetic lethal pair with the target gene (e.g., PKMYT1).

In some embodiments, the disclosure provides a method of promoting or inducing synthetic lethality in a tumor in a subject, comprising administering to the subject one or more therapeutic agents for manipulation of a target gene or a transcriptional or translational product thereof (e.g., PKMYT1), wherein the tumor, or a plurality of tumor cells thereof, comprise a mutation in a biomarker described herein (e.g., a loss of function mutation resulting in a decreased expression level and/or activity of the biomarker), and wherein the biomarker forms a synthetic lethal pair with the target gene (e.g., PKMYT1).

In some embodiments, the mutation results in an altered (e.g., increased or decreased) expression level of the biomarker in the tumor relative to a reference tissue sample (e.g., healthy control tissue). In some embodiments, the mutation results in a decreased expression level of the biomarker (e.g., partial or complete loss of expression of the biomarker) in the tumor relative to a reference tissue sample (e.g., healthy control tissue). In some embodiments, the mutation results in a deficient activity (e.g., increased or decreased) of the biomarker in the tumor relative to a reference tissue sample (e.g., healthy control tissue). In some embodiments, the mutation results in a decreased activity of the biomarker (e.g., partial or complete loss of activity of the biomarker) in the tumor relative to a reference tissue sample (e.g., healthy control tissue).

In some embodiments, the biomarker is selected from any one or any combination (e.g., 2, 3, 4, 5, or more) of biomarkers listed in Table 3. In some embodiments, the biomarker is selected from any one or any combination (e.g., 2, 3, 4, 5, or more) of biomarkers listed in Table 8 and 9. In some embodiments, the biomarker is selected from any one or any combination (e.g., 2, 3, 4, 5, or more) of biomarkers listed in Table 10.

In some embodiments, the biomarker is selected from ATM, MAP2K4, TP53, CDC25A, CACNA1H, CDKN1B, DUSP7, FOXO3, FZD3, JAK1, SMAD2, TGFBR2, MAP3K2, a protein phosphatase 3 (PP3) subunit, a protein phosphatase 2 (PP2A) subunit, and any combination thereof (e.g., 2, 3, 4, 5 or more thereof). In some embodiments, the biomarker is a subunit of PP2A. In some embodiments, the biomarker is PPP2R1B. In some embodiments, the biomarker is a protein phosphatase 3 (PP3) subunit. In some embodiments, the PP3 subunit is PPP3CC. In some embodiments, the biomarker is ATM. In some embodiments, the biomarker is MAP2K4. In some embodiments, the biomarker is TP53. In some embodiments, the biomarker is CDC25A. In some embodiments, the biomarker is CACNA1H. In some embodiments, the biomarker is CDKN1B. In some embodiments, the biomarker is DUSP7. In some embodiments, the biomarker is FOXO3. In some embodiments, the biomarker is FZD3. In some embodiments, the biomarker is JAK1. In some embodiments, the biomarker is SMAD2. In some embodiments, the biomarker is TGFBR2. In some embodiments, the biomarker is MAP3K2.

In some embodiments, the biomarker is selected from any one or any combination (e.g., 2, 3, 4, 5, or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16. In some embodiments, the biomarker is BIN3. In some embodiments, the biomarker is AGPAT5. In some embodiments, the biomarker is FGF17. In some embodiments, the biomarker is PBK. In some embodiments, the biomarker is NOTCH1. In some embodiments, the biomarker is CNTN5. In some embodiments, the biomarker is IRF2. In some embodiments, the biomarker is ALPK2. In some embodiments, the biomarker is CDH19. In some embodiments, the biomarker is CHKB. In some embodiments, the biomarker is MAPK12. In some embodiments, the biomarker is SLC8A1. In some embodiments, the biomarker is HDAC2. In some embodiments, the biomarker is CDT1. In some embodiments, the biomarker is ADCY2. In some embodiments, the biomarker is SLK. In some embodiments, the biomarker is CDC20B. In some embodiments, the biomarker is RPS6KA3. In some embodiments, the biomarker is STAG1. In some embodiments, the biomarker is CKAP5. In some embodiments, the biomarker is RAD51. In some embodiments, the biomarker is CKS1B. In some embodiments, the biomarker is CCNO. In some embodiments, the biomarker is KCNA2. In some embodiments, the biomarker is MCM4. In some embodiments, the biomarker is PLK4. In some embodiments, the biomarker is CDC16.

In some embodiments, the biomarker is selected from any one or any combination (e.g., 2, 3, 4, 5, or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF. In some embodiments, the biomarker is ERICH1. In some embodiments, the biomarker is TNKS. In some embodiments, the biomarker is TDRP. In some embodiments, the biomarker is MTUS1. In some embodiments, the biomarker is TNFRSF10B. In some embodiments, the biomarker is HR. In some embodiments, the biomarker is TNFRSF10D. In some embodiments, the biomarker is DMTN. In some embodiments, the biomarker is ENTPD4. In some embodiments, the biomarker is TNFRSF10C. In some embodiments, the biomarker is PEBP4. In some embodiments, the biomarker is LPL. In some embodiments, the biomarker is LGI3. In some embodiments, the biomarker is SLC7A2. In some embodiments, the biomarker is MTMR9. In some embodiments, the biomarker is MSRA. In some embodiments, the biomarker is PDLIM2. In some embodiments, the biomarker is INTS10. In some embodiments, the biomarker is SH2D4A. In some embodiments, the biomarker is GFRA2. In some embodiments, the biomarker is ZDHHC2. In some embodiments, the biomarker is PDGFRL. In some embodiments, the biomarker is SPAG11B. In some embodiments, the biomarker is PPP1R3B. In some embodiments, the biomarker is SPAG11A. In some embodiments, the biomarker is REEP4. In some embodiments, the biomarker is DEFA5. In some embodiments, the biomarker is DEFB136. In some embodiments, the biomarker is NRG1. In some embodiments, the biomarker is ASAH1. In some embodiments, the biomarker is DEFA3. In some embodiments, the biomarker is EPHX2. In some embodiments, the biomarker is CNOT7. In some embodiments, the biomarker is PNMA2. In some embodiments, the biomarker is TRIM35. In some embodiments, the biomarker is ATRX. In some embodiments, the biomarker is INTS9. In some embodiments, the biomarker is DNAH3. In some embodiments, the biomarker is MAP3K1. In some embodiments, the biomarker is RIMS2. In some embodiments, the biomarker is NSD1. In some embodiments, the biomarker is SARAF.

In some embodiments, the biomarker is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some embodiments, the biomarker is selected from any one or any combination (e.g., 2, 3, 4, 5, or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1. In some embodiments, the biomarker is SLITRK1. In some embodiments, the biomarker is ZNF521. In some embodiments, the biomarker is CCNB1. In some embodiments, the biomarker is CDK7. In some embodiments, the biomarker is MYT1L. In some embodiments, the biomarker is FZR1. In some embodiments, the biomarker is SERF1A. In some embodiments, the biomarker is GADD45B. In some embodiments, the biomarker is ADGRL2. In some embodiments, the biomarker is TTK. In some embodiments, the biomarker is NRXN2. In some embodiments, the biomarker is UNC13A. In some embodiments, the biomarker is ZBTB7A. In some embodiments, the biomarker is POLD1. In some embodiments, the biomarker is PCDH19. In some embodiments, the biomarker is SLC8A2. In some embodiments, the biomarker is E2F4. In some embodiments, the biomarker is AUTS2. In some embodiments, the biomarker is KCNN2. In some embodiments, the biomarker is CCNH. In some embodiments, the biomarker is FRG2C. In some embodiments, the biomarker is PLK2. In some embodiments, the biomarker is MYO18A. In some embodiments, the biomarker is DCAF12L1.

In some embodiments, the biomarker is selected from any one or any combination (e.g., 2, 3, 4, 5, or more) of the biomarkers listed in Table 1.

In some embodiments, the biomarker is selected from any one or any combination (e.g., 2, 3, 4, 5, or more) of the biomarkers listed in Table 2.

In some embodiments, the method comprises administering a therapeutically effective amount of the one or more therapeutic agents. In some embodiments, administering a therapeutically effective amount of the one or more therapeutic agents alters (e.g., increases or decreases) the expression level of the target gene or a transcriptional or translational product thereof (e.g., PKMYT1). In some embodiments, administering a therapeutically effective amount of the one or more therapeutic agents alters (e.g., increases or decreases) the activity of the target gene or a transcriptional or translational product thereof (e.g., PKMYT1). In some embodiments, the administration of the one or more therapeutic agents result in the inhibition or death of cancer cells comprising an altered (e.g., increased or decreased) expression level and/or activity of the biomarker that forms a synthetic lethal pair with the target gene.

In some cases, the cancerous tissue is breast tissue, pancreatic tissue, uterine tissue, bladder tissue, colorectal tissue, prostate tissue, liver tissue, or ovarian tissue. In some cases, the cancerous tissue is liver tissue. In some case, the cancerous tissue is ovarian tissue.

In some embodiments, the inhibition of expression level and/or activity (e.g., via genetic manipulation resulting in a knock down or knock out or via pharmacological inhibition) of the target gene (e.g., PKMYT1) in a cancer cell or a population thereof having an altered (e.g., increased or decreased) expression level and/or activity of the biomarker is lethal to the cancer cell or population thereof, but non-toxic or non-lethal to a control cell or population thereof (e.g., a healthy cell or healthy population of cells) having a normal expression level and/or activity of the biomarker. In some embodiments, a method of treating a subject having a cancer, which cancer comprises a deficiency in expression level and/or activity of the biomarker, using a single inhibitor (e.g., a therapeutically effective amount of a therapeutic agent that causes a decrease in expression level and/or activity of PKMYT1) is beneficial for reducing tumor progression, while having minimal toxicity to normal cells of the subject.

### Diagnostic Methods

In some embodiments, the disclosure provides a method for determining whether a subject with cancer will respond or will likely respond to one or more therapeutic agents for manipulation of a target gene or a transcriptional or translational product thereof (e.g., PKMYT1), wherein the cancer has an altered (e.g., increased or decreased) expression level and/or activity of a biomarker described herein. In some embodiments, the method comprises determining the expression level and/or activity of the biomarker in a cancer sample obtained from the subject, wherein an altered (e.g., increased or decreased) expression level and/or activity relative to a reference tissue sample indicates the subject will respond or will likely respond to the one or more therapeutic agents. In some embodiments, the method comprises obtaining a cancer sample from the subject and detecting a mutation in a biomarker, wherein the presence of a mutation indicates the subject will respond or will likely respond to the one or more therapeutic agents.

In some embodiments, the disclosure provides a method for determining whether a subject with cancer will respond or will likely respond to one or more therapeutic agents for modulating a PKMYT1 gene, or a transcriptional or translational product thereof, wherein the cancer comprises an altered (e.g., increased or decreased) expression level and/or altered (e.g., increased or decreased) activity of a biomarker selected from Table 3. In some embodiments, the cancer comprises an altered (e.g., increased or decreased) expression level and/or altered (e.g., increased or decreased) activity of a biomarker selected from Table 8 and 9. In some embodiments, the cancer comprises an altered (e.g., increased or decreased) expression level and/or altered (e.g., increased or decreased) activity of a biomarker selected from Table 10.

In some embodiments, the disclosure provides a method for determining whether a subject with cancer will respond or will likely respond to one or more therapeutic agents for modulating a PKMYT1 gene, or a transcriptional or translational product thereof, wherein the cancer comprises one or more mutations resulting in a decreased expression level and/or activity of a biomarker selected from Table 3. In some embodiments, the cancer comprises one or more mutations resulting in a decreased expression level and/or activity of a biomarker selected from Table 8 and 9. In some embodiments, the cancer comprises one or more mutations resulting in a decreased expression level and/or activity of a biomarker selected from Table 10.

In some embodiments, the disclosure provides a method for determining whether a subject with cancer will respond or will likely respond to one or more therapeutic agents for modulating a PKMYT1 gene, or a transcriptional or translational product thereof, wherein the cancer comprises a loss of function mutation or an inactivating mutation in a biomarker selected from Table 3. In some embodiments, the cancer comprises a loss of function mutation or an inactivating mutation in a biomarker selected from Table 8 and 9. In some embodiments, the cancer comprises a loss of function mutation or an inactivating mutation in a biomarker selected from Table 10.

In some embodiments, the disclosure provides a method for selecting a subject having cancer to receive one or more therapeutic agents for manipulation of a target gene or a transcriptional or translational product thereof (e.g., PKMYT1), wherein the cancer has an altered (e.g., increased or decreased) expression level and/or activity of a biomarker described herein. In some embodiments, the method comprises determining the expression level and/or activity of the biomarker in a cancer sample obtained from the subject, wherein an altered (e.g., increased or decreased) expression level and/or activity relative to a reference tissue sample is used to select the subject to receive the one or more therapeutic agents. In some embodiments, the method comprises obtaining a cancer sample from the subject and detecting a mutation in a biomarker, wherein the presence of a mutation is used to select the subject to receive the one or more therapeutic agents.

In some embodiments, the disclosure provides a method for selecting a subject having cancer to receive one or more therapeutic agents for modulating a PKMYT1 gene, or a transcriptional or translational product thereof, wherein the cancer comprises an altered (e.g., increased or decreased) expression level and/or altered (e.g., increased or decreased) activity of a biomarker selected from Table 3. In some embodiments, the cancer comprises an altered (e.g., increased or decreased) expression level and/or altered (e.g., increased or decreased) activity of a biomarker selected from Table 8 and 9. In some embodiments, the cancer comprises an altered (e.g., increased or decreased) expression level and/or altered (e.g., increased or decreased) activity of a biomarker selected from Table 10.

In some embodiments, the disclosure provides a method for selecting a subject having cancer to receive one or more therapeutic agents for modulating a PKMYT1 gene, or a transcriptional or translational product thereof, wherein the cancer comprises one or more mutations resulting in a decreased expression level and/or activity of a biomarker selected from Table 3. In some embodiments, the cancer comprises one or more mutations resulting in a decreased expression level and/or activity of a biomarker selected from Table 8 and 9. In some embodiments, the cancer comprises one or more mutations resulting in a decreased expression level and/or activity of a biomarker selected from Table 10.

In some embodiments, the disclosure provides a method for selecting a subject having cancer to receive one or more therapeutic agents for modulating a PKMYT1 gene, or a transcriptional or translational product thereof, wherein the cancer comprises a loss of function mutation or an inactivating mutation in a biomarker selected from Table 3. In some embodiments, the cancer comprises a loss of function mutation or an inactivating mutation in a biomarker selected from Table 8 and 9. In some embodiments, the cancer comprises a loss of function mutation or an inactivating mutation in a biomarker selected from Table 10.

In some embodiments, the disclosure provides a method for predicting responsiveness of a subject having cancer to one or more therapeutic agents for manipulation of a target gene or a transcriptional or translational product thereof (e.g., PKMYT1), wherein the cancer has an altered (e.g., increased or decreased) expression level and/or activity of a biomarker described herein. In some embodiments, the method comprises determining the expression level and/or activity of the biomarker in a cancer sample obtained from the subject, wherein an altered (e.g., increased or decreased) expression level and/or activity relative to a reference tissue sample indicates the subject's cancer will respond or will likely respond to the one or more therapeutic agents. In some embodiments, the method comprises obtaining a cancer sample from the subject and detecting a mutation in a biomarker, wherein the presence of a mutation indicates the subject's cancer will respond or will likely respond to the one or more therapeutic agents.

In some embodiments, the disclosure provides a method for predicting responsiveness of a subject having cancer to one or more therapeutic agents for modulating a PKMYT1 gene, or a transcriptional or translational product thereof, wherein the cancer comprises an altered (e.g., increased or decreased) expression level and/or altered (e.g., increased or decreased) activity of a biomarker selected from Table 3. In some embodiments, wherein the cancer comprises an altered (e.g., increased or decreased) expression level and/or altered (e.g., increased or decreased) activity of a biomarker selected from Table 8 and 9. In some embodiments, wherein the cancer comprises an altered (e.g., increased or decreased) expression level and/or altered (e.g., increased or decreased) activity of a biomarker selected from Table 10.

In some embodiments, the disclosure provides a method for predicting responsiveness of a subject having cancer to one or more therapeutic agents for modulating a PKMYT1 gene, or a transcriptional or translational product thereof, wherein the cancer comprises one or more mutations resulting in a decreased expression level and/or activity of a biomarker selected from Table 3. In some embodiments, the cancer comprises one or more mutations resulting in a decreased expression level and/or activity of a biomarker selected from Table 8 and 9. In some embodiments, the cancer comprises one or more mutations resulting in a decreased expression level and/or activity of a biomarker selected from Table 10.

In some embodiments, the disclosure provides a method for predicting responsiveness of a subject having cancer to one or more therapeutic agents for modulating a PKMYT1 gene, or a transcriptional or translational product thereof, wherein the cancer comprises a loss of function mutation or an inactivating mutation in a biomarker selected from Table 3. In some embodiments, the cancer comprises a loss of function mutation or an inactivating mutation in a biomarker selected from Table 8 and 9. In some embodiments, the cancer comprises a loss of function mutation or an inactivating mutation in a biomarker selected from Table 10.

In some embodiments, the method comprises obtaining a cancer sample from the subject and detecting a mutation in a biomarker, wherein the presence of a mutation indicates the subject will respond or will likely respond to the one or more therapeutic agents for modulating PKMYT1 gene, or a transcriptional or translational product thereof.

In some embodiments, the method comprises determining the expression level and/or activity of the biomarker in a cancer sample obtained from the subject, wherein an altered (e.g., increased or decreased) expression level and/or activity relative to a reference tissue sample indicates the subject will respond or will likely respond to the one or more therapeutic agents for modulating PKMYT1 gene, or a transcriptional or translational product thereof.

In some embodiments, the biomarker is selected from any one or any combination (e.g., 2, 3, 4, 5, or more) of biomarkers listed in Table 3. In some embodiments, the biomarker is selected from any one or any combination (e.g., 2, 3, 4, 5, or more) of biomarkers listed in Table 8 and 9. In some embodiments, the biomarker is selected from any one or any combination (e.g., 2, 3, 4, 5, or more) of biomarkers listed in Table 10.

In some embodiments, the biomarkers is selected from MAP2K4, TP53, CDC25A, CACNA1H, CDKN1B, DUSP7, FOXO3, FZD3, JAK1, SMAD2, TGFBR2, MAP3K2, a PP3 subunit, a PP2A subunit, and any combination thereof (e.g., 2, 3, 4, 5, or more thereof). In some embodiments, the PP2A subunit is PPP2R1B. In some embodiments, the PP3 subunit is PPP3CC.

In some embodiments, the biomarker is selected from any one or any combination (e.g., 2, 3, 4, 5, or more) of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.

In some embodiments, the biomarker is selected from any one or any combination (e.g., 2, 3, 4, 5, or more) of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.

In some embodiments, the biomarker is selected from any one or any combination (e.g., 2, 3, 4, 5 or more) of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.

In some embodiments, the biomarker is selected from any one or any combination (e.g., 2, 3, 4, 5 or more) of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.

In some embodiments, the biomarker is selected from any one or any combination (e.g., 2, 3, 4, 5 or more) of the biomarkers listed in Table 1.

In some embodiments, the biomarker is selected from any one or any combination (e.g., 2, 3, 4, 5 or more) of the biomarkers listed in Table 2.

In some embodiments, the disclosure provides a method for determining whether a subject with cancer will respond or will likely respond to one or more therapeutic agents for manipulation of a target gene or a transcriptional or translational product thereof (e.g., PKMYT1), comprising determining the expression level and/or activity of a panel of biomarkers in a cancer sample obtained from the subject, wherein the panel comprises at least 1, 2, 3, 4, 5, 6, 7, 8, 9, 10, 11, 12, 13, 14, or 15 biomarkers described herein, wherein an altered (e.g., increased or decreased) expression level and/or activity relative to a reference tissue sample of at least one biomarker of the panel indicates the subject will respond or will likely respond to the one or more therapeutic agents. In some embodiments, the method comprises obtaining a cancer sample from the subject and detecting a mutation in at least one biomarker of the panel, wherein the presence of the mutation indicates the subject will respond or will likely respond to the one or more therapeutic agents.

In some embodiments, the disclosure provides a method for determining whether a subject with cancer will respond or will likely respond to one or more therapeutic agents for manipulation of a target gene or a transcriptional or translational product thereof (e.g., PKMYT1), comprising determining the presence of a mutation in a panel of biomarkers in a cancer sample obtained from the subject, wherein the panel comprises at least 1, 2, 3, 4, 5, 6, 7, 8, 9, 10, 11, 12, 13, 14, or 15 biomarkers described herein, wherein the presence of a mutation in at least one biomarker of the panel indicates the subject will respond or will likely respond to the one or more therapeutic agents.

In some embodiments, the disclosure provides a method for selecting a subject having cancer to receive one or more therapeutic agents for manipulation of a target gene or a transcriptional or translational product thereof (e.g., PKMYT1), comprising determining the expression level and/or activity of a panel of biomarkers in a cancer sample obtained from the subject, wherein the panel comprises at least 1, 2, 3, 4, 5, 6, 7, 8, 9, 10, 11, 12, 13, 14, or 15 biomarkers described herein, wherein an altered (e.g., increased or decreased) expression level and/or activity relative to a reference tissue sample of at least one biomarker of the panel is used to select the subject to receive the one or more therapeutic agents. In some embodiments, the method comprises obtaining a cancer sample from the subject and detecting a mutation in at least one biomarker of the panel, wherein the presence of a mutation is used to select the subject to receive the one or more therapeutic agents.

In some embodiments, the disclosure provides a method for selecting a subject having cancer to receive one or more therapeutic agents for manipulation of a target gene or a transcriptional or translational product thereof (e.g., PKMYT1), comprising determining the presence of a mutation in a panel of biomarkers in a cancer sample obtained from the subject, wherein the panel comprises at least 1, 2, 3, 4, 5, 6, 7, 8, 9, 10, 11, 12, 13, 14, or 15 biomarkers described herein, wherein the presence of a mutation in at least one biomarker of the panel is used to select the subject to receive the one or more therapeutic agents.

In some embodiments, the disclosure provides a method for predicting responsiveness of a subject having cancer to one or more therapeutic agents for manipulation of a target gene or a transcriptional or translational product thereof (e.g., PKMYT1), comprising determining the expression level and/or activity of a panel of biomarkers in a cancer sample obtained from the subject, wherein the panel comprises at least 1, 2, 3, 4, 5, 6, 7, 8, 9, 10, 11, 12, 13, 14, or 15 biomarkers described herein, wherein an altered (e.g., increased or decreased) expression level and/or activity relative to a reference tissue sample of at least one biomarker of the panel indicates the subject's cancer will respond or will likely respond to the one or more therapeutic agents. In some embodiments, the method comprises obtaining a cancer sample from the subject and detecting a mutation in at least one biomarker of the panel, wherein the presence of a mutation indicates the subject's cancer will respond or will likely respond to the one or more therapeutic agents.

In some embodiments, the disclosure provides a method for predicting responsiveness of a subject having cancer to one or more therapeutic agents for manipulation of a target gene or a transcriptional or translational product thereof (e.g., PKMYT1), comprising determining the presence of a mutation in a panel of biomarkers in a cancer sample obtained from the subject, wherein the panel comprises at least 1, 2, 3, 4, 5, 6, 7, 8, 9, 10, 11, 12, 13, 14, or 15 biomarkers described herein, wherein the presence of a mutation in at least one biomarker of the panel indicates the subject will respond or will likely respond to the one or more therapeutic agents.

In some embodiments, the panel of biomarkers comprises at least one biomarker selected from any one of the biomarkers listed in Table 3. In some embodiments, the panel of biomarkers comprises at least two biomarkers selected from any one or any combination of the biomarkers listed in Table 3. In some embodiments, the panel of biomarkers comprises at least three biomarkers selected from any one or any combination of the biomarkers listed in Table 3. In some embodiments, the panel of biomarkers comprises at least four biomarkers selected from any one or any combination of the biomarkers listed in Table 3. In some embodiments, the panel of biomarkers comprises at least five biomarkers selected from any one or any combination of the biomarkers listed in Table 3. In some embodiments, the panel of biomarkers comprises at least six biomarkers selected from any one or any combination of the biomarkers listed in Table 3. In some embodiments, the panel of biomarkers comprises at least seven biomarkers selected from any one or any combination of the biomarkers listed in Table 3. In some embodiments, the panel of biomarkers comprises at least eight biomarkers selected from any one or any combination of the biomarkers listed in Table 3. In some embodiments, the panel of biomarkers comprises at least nine biomarkers selected from any one or any combination of the biomarkers listed in Table 3. In some embodiments, the panel of biomarkers comprises at least ten or more biomarkers selected from any one or any combination of the biomarkers listed in Table 3.

In some embodiments, the panel of biomarkers comprises at least one biomarker selected from any one of the biomarkers listed in Table 8 and 9. In some embodiments, the panel of biomarkers comprises at least two biomarkers selected from any one or any combination of the biomarkers listed in Table 8 and 9. In some embodiments, the panel of biomarkers comprises at least three biomarkers selected from any one or any combination of the biomarkers listed in Table 8 and 9. In some embodiments, the panel of biomarkers comprises at least four biomarkers selected from any one or any combination of the biomarkers listed in Table 8 and 9. In some embodiments, the panel of biomarkers comprises at least five biomarkers selected from any one or any combination of the biomarkers listed in Table 8 and 9. In some embodiments, the panel of biomarkers comprises at least six biomarkers selected from any one or any combination of the biomarkers listed in Table 8 and 9. In some embodiments, the panel of biomarkers comprises at least seven biomarkers selected from any one or any combination of the biomarkers listed in Table 8 and 9. In some embodiments, the panel of biomarkers comprises at least eight biomarkers selected from any one or any combination of the biomarkers listed in Table 8 and 9. In some embodiments, the panel of biomarkers comprises at least nine biomarkers selected from any one or any combination of the biomarkers listed in Table 8 and 9. In some embodiments, the panel of biomarkers comprises at least ten or more biomarkers selected from any one or any combination of the biomarkers listed in Table 8 and 9.

In some embodiments, the panel of biomarkers comprises at least one biomarker selected from any one of the biomarkers listed in Table 10. In some embodiments, the panel of biomarkers comprises at least two biomarkers selected from any one or any combination of the biomarkers listed in Table 10. In some embodiments, the panel of biomarkers comprises at least three biomarkers selected from any one or any combination of the biomarkers listed in Table 10. In some embodiments, the panel of biomarkers comprises at least four biomarkers selected from any one or any combination of the biomarkers listed in Table 10. In some embodiments, the panel of biomarkers comprises at least five biomarkers selected from any one or any combination of the biomarkers listed in Table 10. In some embodiments, the panel of biomarkers comprises at least six biomarkers selected from any one or any combination of the biomarkers listed in Table 10. In some embodiments, the panel of biomarkers comprises at least seven biomarkers selected from any one or any combination of the biomarkers listed in Table 10. In some embodiments, the panel of biomarkers comprises at least eight biomarkers selected from any one or any combination of the biomarkers listed in Table 10. In some embodiments, the panel of biomarkers comprises at least nine biomarkers selected from any one or any combination of the biomarkers listed in Table 10. In some embodiments, the panel of biomarkers comprises at least ten or more biomarkers selected from any one or any combination of the biomarkers listed in Table 10.

In some embodiments, the panel of biomarker comprises ATM and at least one (e.g., 1, 2, 3, 4, 5, 6, 7, 8, 9, 10, 11, 12, 13, or 14) other biomarker described herein. In some embodiments, the panel of biomarker comprises MAP2K4 and at least one (e.g., 1, 2, 3, 4, 5, 6, 7, 8, 9, 10, 11, 12, 13, or 14) other biomarker described herein. In some embodiments, the panel of biomarker comprises TP53 and at least one (e.g., 1, 2, 3, 4, 5, 6, 7, 8, 9, 10, 11, 12, 13, or 14) other biomarker described herein. In some embodiments, the panel of biomarker comprises CDC25A and at least one (e.g., 1, 2, 3, 4, 5, 6, 7, 8, 9, 10, 11, 12, 13, or 14) other biomarker described herein. In some embodiments, the panel of biomarker comprises CACNA1H and at least one (e.g., 1, 2, 3, 4, 5, 6, 7, 8, 9, 10, 11, 12, 13, or 14) other biomarker described herein. In some embodiments, the panel of biomarker comprises CDKN1B and at least one (e.g., 1, 2, 3, 4, 5, 6, 7, 8, 9, 10, 11, 12, 13, or 14) other biomarker described herein. In some embodiments, the panel of biomarker comprises DUSP7 and at least one (e.g., 1, 2, 3, 4, 5, 6, 7, 8, 9, 10, 11, 12, 13, or 14) other biomarker described herein. In some embodiments, the panel of biomarker comprises FOXO3 and at least one (e.g., 1, 2, 3, 4, 5, 6, 7, 8, 9, 10, 11, 12, 13, or 14) other biomarker described herein. In some embodiments, the panel of biomarker comprises FZD3 and at least one (e.g., 1, 2, 3, 4, 5, 6, 7, 8, 9, 10, 11, 12, 13, or 14) other biomarker described herein. In some embodiments, the panel of biomarker comprises JAK1 and at least one (e.g., 1, 2, 3, 4, 5, 6, 7, 8, 9, 10, 11, 12, 13, or 14) other biomarker described herein. In some embodiments, the panel of biomarker comprises SMAD2 and at least one (e.g., 1, 2, 3, 4, 5, 6, 7, 8, 9, 10, 11, 12, 13, or 14) other biomarker described herein. In some embodiments, the panel of biomarker comprises TGFBR2 and at least one (e.g., 1, 2, 3, 4, 5, 6, 7, 8, 9, 10, 11, 12, 13, or 14) other biomarker described herein. In some embodiments, the panel of biomarker comprises MAP3K2 and at least one (e.g., 1, 2, 3, 4, 5, 6, 7, 8, 9, 10, 11, 12, 13, or 14) other biomarker described herein. In some embodiments, the panel of biomarker comprises PPP2R1B and at least one (e.g., 1, 2, 3, 4, 5, 6, 7, 8, 9, 10, 11, 12, 13, or 14) other biomarker described herein. In some embodiments, the panel of biomarker comprises a PP3 subunit and at least one (e.g., 1, 2, 3, 4, 5, 6, 7, 8, 9, 10, 11, 12, 13, or 14) other biomarker described herein. In some embodiments, the panel of biomarker comprises PPP3CC and at least one (e.g., 1, 2, 3, 4, 5, 6, 7, 8, 9, 10, 11, 12, 13, or 14) other biomarker described herein. In some embodiments, the panel of biomarker comprises PP2A subunit and at least one (e.g., 1, 2, 3, 4, 5, 6, 7, 8, 9, 10, 11, 12, 13, or 14) other biomarker described herein. In some embodiments, the panel of biomarker comprises PP2R1B subunit and at least one (e.g., 1, 2, 3, 4, 5, 6, 7, 8, 9, 10, 11, 12, 13, or 14) other biomarker described herein.

In some embodiments, the panel of biomarkers comprises at least one biomarker selected from MAP2K4, TP53, CDC25A, CACNA1H, CDKN1B, DUSP7, FOXO3, FZD3, JAK1, SMAD2, TGFBR2, MAP3K2, a PP3 subunit, a PP2A subunit, and any combination thereof. In some embodiments, the PP2A subunit is PPP2R1B. In some embodiments, the PP3 subunit is PPP3CC.

In some embodiments, the panel of biomarkers comprises BIN3 and one or more (e.g., 1, 2, 3, 4, 5, or more) additional biomarkers described herein. In some embodiments, the panel of biomarkers comprises AGPAT5 and one or more (e.g., 1, 2, 3, 4, 5, or more) additional biomarkers described herein. In some embodiments, the panel of biomarkers comprises FGF17 and one or more (e.g., 1, 2, 3, 4, 5, or more) additional biomarkers described herein. In some embodiments, the panel of biomarkers comprises PBK and one or more (e.g., 1, 2, 3, 4, 5, or more) additional biomarkers described herein. In some embodiments, the panel of biomarkers comprises NOTCH1 and one or more (e.g., 1, 2, 3, 4, 5, or more) additional biomarkers described herein. In some embodiments, the panel of biomarkers comprises CNTN5 and one or more (e.g., 1, 2, 3, 4, 5, or more) additional biomarkers described herein. In some embodiments, the panel of biomarkers comprises IRF2 and one or more (e.g., 1, 2, 3, 4, 5, or more) additional biomarkers described herein. In some embodiments, the panel of biomarkers comprises ALPK2 and one or more (e.g., 1, 2, 3, 4, 5, or more) additional biomarkers described herein. In some embodiments, the panel of biomarkers comprises CDH19 and one or more (e.g., 1, 2, 3, 4, 5, or more) additional biomarkers described herein. In some embodiments, the panel of biomarkers comprises CHKB and one or more (e.g., 1, 2, 3, 4, 5, or more) additional biomarkers described herein. In some embodiments, the panel of biomarkers comprises MAPK12 and one or more (e.g., 1, 2, 3, 4, 5, or more) additional biomarkers described herein. In some embodiments, the panel of biomarkers comprises SLC8A1 and one or more (e.g., 1, 2, 3, 4, 5, or more) additional biomarkers described herein. In some embodiments, the panel of biomarkers comprises HDAC2 and one or more (e.g., 1, 2, 3, 4, 5, or more) additional biomarkers described herein. In some embodiments, the panel of biomarkers comprises CDT1 and one or more (e.g., 1, 2, 3, 4, 5, or more) additional biomarkers described herein. In some embodiments, the panel of biomarkers comprises ADCY2 and one or more (e.g., 1, 2, 3, 4, 5, or more) additional biomarkers described herein. In some embodiments, the panel of biomarkers comprises SLK and one or more (e.g., 1, 2, 3, 4, 5, or more) additional biomarkers described herein. In some embodiments, the panel of biomarkers comprises CDC20B and one or more (e.g., 1, 2, 3, 4, 5, or more) additional biomarkers described herein. In some embodiments, the panel of biomarkers comprises RPS6KA3 and one or more (e.g., 1, 2, 3, 4, 5, or more) additional biomarkers described herein. In some embodiments, the panel of biomarkers comprises STAG1 and one or more (e.g., 1, 2, 3, 4, 5, or more) additional biomarkers described herein. In some embodiments, the panel of biomarkers comprises CKAP5 and one or more (e.g., 1, 2, 3, 4, 5, or more) additional biomarkers described herein. In some embodiments, the panel of biomarkers comprises RAD51 and one or more (e.g., 1, 2, 3, 4, 5, or more) additional biomarkers described herein. In some embodiments, the panel of biomarkers comprises CKS1B and one or more (e.g., 1, 2, 3, 4, 5, or more) additional biomarkers described herein. In some embodiments, the panel of biomarkers comprises CCNO and one or more (e.g., 1, 2, 3, 4, 5, or more) additional biomarkers described herein. In some embodiments, the panel of biomarkers comprises KCNA2 and one or more (e.g., 1, 2, 3, 4, 5, or more) additional biomarkers described herein. In some embodiments, the panel of biomarkers comprises MCM4 and one or more (e.g., 1, 2, 3, 4, 5, or more) additional biomarkers described herein. In some embodiments, the panel of biomarkers comprises PLK4 and one or more (e.g., 1, 2, 3, 4, 5, or more) additional biomarkers described herein. In some embodiments, the panel of biomarkers comprises CDC16 and one or more (e.g., 1, 2, 3, 4, 5, or more) additional biomarkers described herein.

In some embodiments, the panel of biomarkers comprises ERICH1 and at least one (e.g., 1, 2, 3, 4, 5 or more) additional biomarkers described herein. In some embodiments, the panel of biomarkers comprises TNKS and at least one (e.g., 1, 2, 3, 4, 5 or more) additional biomarkers described herein. In some embodiments, the panel of biomarkers comprises TDRP and at least one (e.g., 1, 2, 3, 4, 5 or more) additional biomarkers described herein. In some embodiments, the panel of biomarkers comprises MTUS1 and at least one (e.g., 1, 2, 3, 4, 5 or more) additional biomarkers described herein. In some embodiments, the panel of biomarkers comprises TNFRSF10B and at least one (e.g., 1, 2, 3, 4, 5 or more) additional biomarkers described herein. In some embodiments, the panel of biomarkers comprises HR and at least one (e.g., 1, 2, 3, 4, 5 or more) additional biomarkers described herein. In some embodiments, the panel of biomarkers comprises TNFRSF10D and at least one (e.g., 1, 2, 3, 4, 5 or more) additional biomarkers described herein. In some embodiments, the panel of biomarkers comprises DMTN and at least one (e.g., 1, 2, 3, 4, 5 or more) additional biomarkers described herein. In some embodiments, the panel of biomarkers comprises ENTPD4 and at least one (e.g., 1, 2, 3, 4, 5 or more) additional biomarkers described herein. In some embodiments, the panel of biomarkers comprises TNFRSF10C and at least one (e.g., 1, 2, 3, 4, 5 or more) additional biomarkers described herein. In some embodiments, the panel of biomarkers comprises PEBP4 and at least one (e.g., 1, 2, 3, 4, 5 or more) additional biomarkers described herein. In some embodiments, the panel of biomarkers comprises LPL and at least one (e.g., 1, 2, 3, 4, 5 or more) additional biomarkers described herein. In some embodiments, the panel of biomarkers comprises LGI3 and at least one (e.g., 1, 2, 3, 4, 5 or more) additional biomarkers described herein. In some embodiments, the panel of biomarkers comprises SLC7A2 and at least one (e.g., 1, 2, 3, 4, 5 or more) additional biomarkers described herein. In some embodiments, the panel of biomarkers comprises MTMR9 and at least one (e.g., 1, 2, 3, 4, 5 or more) additional biomarkers described herein. In some embodiments, the panel of biomarkers comprises MSRA and at least one (e.g., 1, 2, 3, 4, 5 or more) additional biomarkers described herein. In some embodiments, the panel of biomarkers comprises PDLIM2 and at least one (e.g., 1, 2, 3, 4, 5 or more) additional biomarkers described herein. In some embodiments, the panel of biomarkers comprises INTS10 and at least one (e.g., 1, 2, 3, 4, 5 or more) additional biomarkers described herein. In some embodiments, the panel of biomarkers comprises SH2D4A and at least one (e.g., 1, 2, 3, 4, 5 or more) additional biomarkers described herein. In some embodiments, the panel of biomarkers comprises GFRA2 and at least one (e.g., 1, 2, 3, 4, 5 or more) additional biomarkers described herein. In some embodiments, the panel of biomarkers comprises ZDHHC2 and at least one (e.g., 1, 2, 3, 4, 5 or more) additional biomarkers described herein. In some embodiments, the panel of biomarkers comprises PDGFRL and at least one (e.g., 1, 2, 3, 4, 5 or more) additional biomarkers described herein. In some embodiments, the panel of biomarkers comprises SPAG11B and at least one (e.g., 1, 2, 3, 4, 5 or more) additional biomarkers described herein. In some embodiments, the panel of biomarkers comprises PPP1R3B and at least one (e.g., 1, 2, 3, 4, 5 or more) additional biomarkers described herein. In some embodiments, the panel of biomarkers comprises SPAG11A and at least one (e.g., 1, 2, 3, 4, 5 or more) additional biomarkers described herein. In some embodiments, the panel of biomarkers comprises REEP4 and at least one (e.g., 1, 2, 3, 4, 5 or more) additional biomarkers described herein. In some embodiments, the panel of biomarkers comprises DEFA5 and at least one (e.g., 1, 2, 3, 4, 5 or more) additional biomarkers described herein. In some embodiments, the panel of biomarkers comprises DEFB136 and at least one (e.g., 1, 2, 3, 4, 5 or more) additional biomarkers described herein. In some embodiments, the panel of biomarkers comprises NRG1 and at least one (e.g., 1, 2, 3, 4, 5 or more) additional biomarkers described herein. In some embodiments, the panel of biomarkers comprises ASAH1 and at least one (e.g., 1, 2, 3, 4, 5 or more) additional biomarkers described herein. In some embodiments, the panel of biomarkers comprises DEFA3 and at least one (e.g., 1, 2, 3, 4, 5 or more) additional biomarkers described herein. In some embodiments, the panel of biomarkers comprises EPHX2 and at least one (e.g., 1, 2, 3, 4, 5 or more) additional biomarkers described herein. In some embodiments, the panel of biomarkers comprises CNOT7 and at least one (e.g., 1, 2, 3, 4, 5 or more) additional biomarkers described herein. In some embodiments, the panel of biomarkers comprises PNMA2 and at least one (e.g., 1, 2, 3, 4, 5 or more) additional biomarkers described herein. In some embodiments, the panel of biomarkers comprises TRIM35 and at least one (e.g., 1, 2, 3, 4, 5 or more) additional biomarkers described herein. In some embodiments, the panel of biomarkers comprises ATRX and at least one (e.g., 1, 2, 3, 4, 5 or more) additional biomarkers described herein. In some embodiments, the panel of biomarkers comprises INTS9 and at least one (e.g., 1, 2, 3, 4, 5 or more) additional biomarkers described herein. In some embodiments, the panel of biomarkers comprises DNAH3 and at least one (e.g., 1, 2, 3, 4, 5 or more) additional biomarkers described herein. In some embodiments, the panel of biomarkers comprises MAP3K1 and at least one (e.g., 1, 2, 3, 4, 5 or more) additional biomarkers described herein. In some embodiments, the panel of biomarkers comprises RIMS2 and at least one (e.g., 1, 2, 3, 4, 5 or more) additional biomarkers described herein. In some embodiments, the panel of biomarkers comprises NSD1 and at least one (e.g., 1, 2, 3, 4, 5 or more) additional biomarkers described herein. In some embodiments, the panel of biomarkers comprises SARAF and at least one (e.g., 1, 2, 3, 4, 5 or more) other biomarkers described herein.

In some embodiments, the panel of biomarkers comprises CDKN2B and one or more (e.g., 1, 2, 3, 4, 5, or more) additional biomarkers described herein. In some embodiments, the panel of biomarkers comprises CSMD3 and one or more (e.g., 1, 2, 3, 4, 5, or more) additional biomarkers described herein. In some embodiments, the panel of biomarkers comprises LRP1B and one or more (e.g., 1, 2, 3, 4, 5, or more) additional biomarkers described herein. In some embodiments, the panel of biomarkers comprises DMRTA1 and one or more (e.g., 1, 2, 3, 4, 5, or more) additional biomarkers described herein. In some embodiments, the panel of biomarkers comprises PTPRD and one or more (e.g., 1, 2, 3, 4, 5, or more) additional biomarkers described herein. In some embodiments, the panel of biomarkers comprises ELAVL2 and one or more (e.g., 1, 2, 3, 4, 5, or more) additional biomarkers described herein. In some embodiments, the panel of biomarkers comprises FAT1 and one or more (e.g., 1, 2, 3, 4, 5, or more) additional biomarkers described herein. In some embodiments, the panel of biomarkers comprises CDH1 and one or more (e.g., 1, 2, 3, 4, 5, or more) additional biomarkers described herein. In some embodiments, the panel of biomarkers comprises NF1 and one or more (e.g., 1, 2, 3, 4, 5, or more) additional biomarkers described herein. In some embodiments, the panel of biomarkers comprises PPP6R2 and one or more (e.g., 1, 2, 3, 4, 5, or more) additional biomarkers described herein. In some embodiments, the panel of biomarkers comprises PIM3 and one or more (e.g., 1, 2, 3, 4, 5, or more) additional biomarkers described herein. In some embodiments, the panel of biomarkers comprises MAPK11 and one or more (e.g., 1, 2, 3, 4, 5, or more) additional biomarkers described herein. In some embodiments, the panel of biomarkers comprises CDH10 and one or more (e.g., 1, 2, 3, 4, 5, or more) additional biomarkers described herein. In some embodiments, the panel of biomarkers comprises PCDH15 and one or more (e.g., 1, 2, 3, 4, 5, or more) additional biomarkers described herein. In some embodiments, the panel of biomarkers comprises ALB and one or more (e.g., 1, 2, 3, 4, 5, or more) additional biomarkers described herein. In some embodiments, the panel of biomarkers comprises OR4F21 and one or more (e.g., 1, 2, 3, 4, 5, or more) additional biomarkers described herein. In some embodiments, the panel of biomarkers comprises LINGO2 and one or more (e.g., 1, 2, 3, 4, 5, or more) additional biomarkers described herein. In some embodiments, the panel of biomarkers comprises FBN2 and one or more (e.g., 1, 2, 3, 4, 5, or more) additional biomarkers described herein. In some embodiments, the panel of biomarkers comprises CACNA1E and one or more (e.g., 1, 2, 3, 4, 5, or more) additional biomarkers described herein. In some embodiments, the panel of biomarkers comprises LRRC7 and one or more (e.g., 1, 2, 3, 4, 5, or more) additional biomarkers described herein. In some embodiments, the panel of biomarkers comprises NALCN and one or more (e.g., 1, 2, 3, 4, 5, or more) additional biomarkers described herein. In some embodiments, the panel of biomarkers comprises ARID1A and one or more (e.g., 1, 2, 3, 4, 5, or more) additional biomarkers described herein. In some embodiments, the panel of biomarkers comprises ADGRB3 and one or more (e.g., 1, 2, 3, 4, 5, or more) additional biomarkers described herein. In some embodiments, the panel of biomarkers comprises SI and one or more (e.g., 1, 2, 3, 4, 5, or more) additional biomarkers described herein. In some embodiments, the panel of biomarkers comprises PKHD1L1 and one or more (e.g., 1, 2, 3, 4, 5, or more) additional biomarkers described herein. In some embodiments, the panel of biomarkers comprises TBC1D22A and one or more (e.g., 1, 2, 3, 4, 5, or more) additional biomarkers described herein. In some embodiments, the panel of biomarkers comprises BNIP3L and one or more (e.g., 1, 2, 3, 4, 5, or more) additional biomarkers described herein. In some embodiments, the panel of biomarkers comprises DEFA1 and one or more (e.g., 1, 2, 3, 4, 5, or more) additional biomarkers described herein. In some embodiments, the panel of biomarkers comprises DEFB103B and one or more (e.g., 1, 2, 3, 4, 5, or more) additional biomarkers described herein. In some embodiments, the panel of biomarkers comprises DEFB103A and one or more (e.g., 1, 2, 3, 4, 5, or more) additional biomarkers described herein. In some embodiments, the panel of biomarkers comprises HCN1 and one or more (e.g., 1, 2, 3, 4, 5, or more) additional biomarkers described herein. In some embodiments, the panel of biomarkers comprises RELN and one or more (e.g., 1, 2, 3, 4, 5, or more) additional biomarkers described herein. In some embodiments, the panel of biomarkers comprises UNC13C and one or more (e.g., 1, 2, 3, 4, 5, or more) additional biomarkers described herein. In some embodiments, the panel of biomarkers comprises XKR5 and one or more (e.g., 1, 2, 3, 4, 5, or more) additional biomarkers described herein. In some embodiments, the panel of biomarkers comprises CHMP7 and one or more (e.g., 1, 2, 3, 4, 5, or more) additional biomarkers described herein. In some embodiments, the panel of biomarkers comprises CHRNA2 and one or more (e.g., 1, 2, 3, 4, 5, or more) additional biomarkers described herein. In some embodiments, the panel of biomarkers comprises CSGALNACT1 and one or more (e.g., 1, 2, 3, 4, 5, or more) additional biomarkers described herein. In some embodiments, the panel of biomarkers comprises FAM86B2 and one or more (e.g., 1, 2, 3, 4, 5, or more) additional biomarkers described herein. In some embodiments, the panel of biomarkers comprises EGR3 and one or more (e.g., 1, 2, 3, 4, 5, or more) additional biomarkers described herein. In some embodiments, the panel of biomarkers comprises XPO7 and one or more (e.g., 1, 2, 3, 4, 5, or more) additional biomarkers described herein. In some embodiments, the panel of biomarkers comprises TRPS1 and one or more (e.g., 1, 2, 3, 4, 5, or more) additional biomarkers described herein. In some embodiments, the panel of biomarkers comprises KDM6A and one or more (e.g., 1, 2, 3, 4, 5, or more) additional biomarkers described herein. In some embodiments, the panel of biomarkers comprises NBEA and one or more (e.g., 1, 2, 3, 4, 5, or more) additional biomarkers described herein. In some embodiments, the panel of biomarkers comprises VPS37A and one or more (e.g., 1, 2, 3, 4, 5, or more) additional biomarkers described herein. In some embodiments, the panel of biomarkers comprises SCN1A and one or more (e.g., 1, 2, 3, 4, 5, or more) additional biomarkers described herein. In some embodiments, the panel of biomarkers comprises CSMD2 and one or more (e.g., 1, 2, 3, 4, 5, or more) additional biomarkers described herein. In some embodiments, the panel of biomarkers comprises GTSE1 and one or more (e.g., 1, 2, 3, 4, 5, or more) additional biomarkers described herein. In some embodiments, the panel of biomarkers comprises TRMU and one or more (e.g., 1, 2, 3, 4, 5, or more) additional biomarkers described herein. In some embodiments, the panel of biomarkers comprises TENM1 and one or more (e.g., 1, 2, 3, 4, 5, or more) additional biomarkers described herein. In some embodiments, the panel of biomarkers comprises DOCK3 and one or more (e.g., 1, 2, 3, 4, 5, or more) additional biomarkers described herein. In some embodiments, the panel of biomarkers comprises VPS13B and one or more (e.g., 1, 2, 3, 4, 5, or more) additional biomarkers described herein. In some embodiments, the panel of biomarkers comprises RBM10 and one or more (e.g., 1, 2, 3, 4, 5, or more) additional biomarkers described herein. In some embodiments, the panel of biomarkers comprises RYR2 and one or more (e.g., 1, 2, 3, 4, 5, or more) additional biomarkers described herein. In some embodiments, the panel of biomarkers comprises SCARA5 and one or more (e.g., 1, 2, 3, 4, 5, or more) additional biomarkers described herein. In some embodiments, the panel of biomarkers comprises SETBP1 and one or more (e.g., 1, 2, 3, 4, 5, or more) additional biomarkers described herein. In some embodiments, the panel of biomarkers comprises DYSF and one or more (e.g., 1, 2, 3, 4, 5, or more) additional biomarkers described herein. In some embodiments, the panel of biomarkers comprises NLGN4X and one or more (e.g., 1, 2, 3, 4, 5, or more) additional biomarkers described herein. In some embodiments, the panel of biomarkers comprises EPHA3 and one or more (e.g., 1, 2, 3, 4, 5, or more) additional biomarkers described herein. In some embodiments, the panel of biomarkers comprises FBLN1 and one or more (e.g., 1, 2, 3, 4, 5, or more) additional biomarkers described herein. In some embodiments, the panel of biomarkers comprises ADAMTS20 and one or more (e.g., 1, 2, 3, 4, 5, or more) additional biomarkers described herein. In some embodiments, the panel of biomarkers comprises IFT74 and one or more (e.g., 1, 2, 3, 4, 5, or more) additional biomarkers described herein. In some embodiments, the panel of biomarkers comprises KLKB1 and one or more (e.g., 1, 2, 3, 4, 5, or more) additional biomarkers described herein. In some embodiments, the panel of biomarkers comprises ACVR2A and one or more (e.g., 1, 2, 3, 4, 5, or more) additional biomarkers described herein. In some embodiments, the panel of biomarkers comprises ZFHX4 and one or more (e.g., 1, 2, 3, 4, 5, or more) additional biomarkers described herein. In some embodiments, the panel of biomarkers comprises WWC2 and one or more (e.g., 1, 2, 3, 4, 5, or more) additional biomarkers described herein. In some embodiments, the panel of biomarkers comprises MOB3B and one or more (e.g., 1, 2, 3, 4, 5, or more) additional biomarkers described herein. In some embodiments, the panel of biomarkers comprises DMXL1 and one or more (e.g., 1, 2, 3, 4, 5, or more) additional biomarkers described herein. In some embodiments, the panel of biomarkers comprises ELAC1 and one or more (e.g., 1, 2, 3, 4, 5, or more) additional biomarkers described herein. In some embodiments, the panel of biomarkers comprises RBPMS and one or more (e.g., 1, 2, 3, 4, 5, or more) additional biomarkers described herein. In some embodiments, the panel of biomarkers comprises ANK1 and one or more (e.g., 1, 2, 3, 4, 5, or more) additional biomarkers described herein. In some embodiments, the panel of biomarkers comprises CADM2 and one or more (e.g., 1, 2, 3, 4, 5, or more) additional biomarkers described herein. In some embodiments, the panel of biomarkers comprises C9orf72 and one or more (e.g., 1, 2, 3, 4, 5, or more) additional biomarkers described herein. In some embodiments, the panel of biomarkers comprises MTNR1A and one or more (e.g., 1, 2, 3, 4, 5, or more) additional biomarkers described herein. In some embodiments, the panel of biomarkers comprises PLAA and one or more (e.g., 1, 2, 3, 4, 5, or more) additional biomarkers described herein. In some embodiments, the panel of biomarkers comprises NIPBL and one or more (e.g., 1, 2, 3, 4, 5, or more) additional biomarkers described herein. In some embodiments, the panel of biomarkers comprises ASPM and one or more (e.g., 1, 2, 3, 4, 5, or more) additional biomarkers described herein. In some embodiments, the panel of biomarkers comprises GABRB3 and one or more (e.g., 1, 2, 3, 4, 5, or more) additional biomarkers described herein. In some embodiments, the panel of biomarkers comprises CTNNA3 and one or more (e.g., 1, 2, 3, 4, 5, or more) additional biomarkers described herein. In some embodiments, the panel of biomarkers comprises CNTN3 and one or more (e.g., 1, 2, 3, 4, 5, or more) additional biomarkers described herein. In some embodiments, the panel of biomarkers comprises PPFIA2 and one or more (e.g., 1, 2, 3, 4, 5, or more) additional biomarkers described herein. In some embodiments, the panel of biomarkers comprises FN1 and one or more (e.g., 1, 2, 3, 4, 5, or more) additional biomarkers described herein. In some embodiments, the panel of biomarkers comprises HECW1 and one or more (e.g., 1, 2, 3, 4, 5, or more) additional biomarkers described herein. In some embodiments, the panel of biomarkers comprises DMXL2 and one or more (e.g., 1, 2, 3, 4, 5, or more) additional biomarkers described herein. In some embodiments, the panel of biomarkers comprises ZFP36L2 and one or more (e.g., 1, 2, 3, 4, 5, or more) additional biomarkers described herein. In some embodiments, the panel of biomarkers comprises UPK3A and one or more (e.g., 1, 2, 3, 4, 5, or more) additional biomarkers described herein. In some embodiments, the panel of biomarkers comprises SMC1B and one or more (e.g., 1, 2, 3, 4, 5, or more) additional biomarkers described herein. In some embodiments, the panel of biomarkers comprises SMARCA4 and one or more (e.g., 1, 2, 3, 4, 5, or more) additional biomarkers described herein. In some embodiments, the panel of biomarkers comprises LRFN5 and one or more (e.g., 1, 2, 3, 4, 5, or more) additional biomarkers described herein. In some embodiments, the panel of biomarkers comprises TG and one or more (e.g., 1, 2, 3, 4, 5, or more) additional biomarkers described herein. In some embodiments, the panel of biomarkers comprises CTNND2 and one or more (e.g., 1, 2, 3, 4, 5, or more) additional biomarkers described herein. In some embodiments, the panel of biomarkers comprises CHD1 and one or more (e.g., 1, 2, 3, 4, 5, or more) additional biomarkers described herein. In some embodiments, the panel of biomarkers comprises LSAMP and one or more (e.g., 1, 2, 3, 4, 5, or more) additional biomarkers described herein. In some embodiments, the panel of biomarkers comprises PRR5 and one or more (e.g., 1, 2, 3, 4, 5, or more) additional biomarkers described herein. In some embodiments, the panel of biomarkers comprises NPAP1 and one or more (e.g., 1, 2, 3, 4, 5, or more) additional biomarkers described herein. In some embodiments, the panel of biomarkers comprises SNTG1 and one or more (e.g., 1, 2, 3, 4, 5, or more) additional biomarkers described herein. In some embodiments, the panel of biomarkers comprises MDGA2 and one or more (e.g., 1, 2, 3, 4, 5, or more) additional biomarkers described herein. In some embodiments, the panel of biomarkers comprises BNC2 and one or more (e.g., 1, 2, 3, 4, 5, or more) additional biomarkers described herein. In some embodiments, the panel of biomarkers comprises SCN2A and one or more (e.g., 1, 2, 3, 4, 5, or more) additional biomarkers described herein. In some embodiments, the panel of biomarkers comprises HERC2 and one or more (e.g., 1, 2, 3, 4, 5, or more) additional biomarkers described herein. In some embodiments, the panel of biomarkers comprises SCN3A and one or more (e.g., 1, 2, 3, 4, 5, or more) additional biomarkers described herein. In some embodiments, the panel of biomarkers comprises TRPM1 and one or more (e.g., 1, 2, 3, 4, 5, or more) additional biomarkers described herein. In some embodiments, the panel of biomarkers comprises FSTL5 and one or more (e.g., 1, 2, 3, 4, 5, or more) additional biomarkers described herein. In some embodiments, the panel of biomarkers comprises ASH1L and one or more (e.g., 1, 2, 3, 4, 5, or more) additional biomarkers described herein. In some embodiments, the panel of biomarkers comprises PRKDC and one or more (e.g., 1, 2, 3, 4, 5, or more) additional biomarkers described herein. In some embodiments, the panel of biomarkers comprises TCF4 and one or more (e.g., 1, 2, 3, 4, 5, or more) additional biomarkers described herein. In some embodiments, the panel of biomarkers comprises SVIL and one or more (e.g., 1, 2, 3, 4, 5, or more) additional biomarkers described herein. In some embodiments, the panel of biomarkers comprises CHD4 and one or more (e.g., 1, 2, 3, 4, 5, or more) additional biomarkers described herein. In some embodiments, the panel of biomarkers comprises PCDH9 and one or more (e.g., 1, 2, 3, 4, 5, or more) additional biomarkers described herein. In some embodiments, the panel of biomarkers comprises NRXN3 and one or more (e.g., 1, 2, 3, 4, 5, or more) additional biomarkers described herein. In some embodiments, the panel of biomarkers comprises SNX25 and one or more (e.g., 1, 2, 3, 4, 5, or more) additional biomarkers described herein. In some embodiments, the panel of biomarkers comprises MPDZ and one or more (e.g., 1, 2, 3, 4, 5, or more) additional biomarkers described herein. In some embodiments, the panel of biomarkers comprises TLL1 and one or more (e.g., 1, 2, 3, 4, 5, or more) additional biomarkers described herein. In some embodiments, the panel of biomarkers comprises EPHA6 and one or more (e.g., 1, 2, 3, 4, 5, or more) additional biomarkers described herein. In some embodiments, the panel of biomarkers comprises FER and one or more (e.g., 1, 2, 3, 4, 5, or more) additional biomarkers described herein. In some embodiments, the panel of biomarkers comprises NFASC and one or more (e.g., 1, 2, 3, 4, 5, or more) additional biomarkers described herein. In some embodiments, the panel of biomarkers comprises USP34 and one or more (e.g., 1, 2, 3, 4, 5, or more) additional biomarkers described herein. In some embodiments, the panel of biomarkers comprises SPEF2 and one or more (e.g., 1, 2, 3, 4, 5, or more) additional biomarkers described herein. In some embodiments, the panel of biomarkers comprises CHD8 and one or more (e.g., 1, 2, 3, 4, 5, or more) additional biomarkers described herein. In some embodiments, the panel of biomarkers comprises ABCA12 and one or more (e.g., 1, 2, 3, 4, 5, or more) additional biomarkers described herein. In some embodiments, the panel of biomarkers comprises ARID2 and one or more (e.g., 1, 2, 3, 4, 5, or more) additional biomarkers described herein. In some embodiments, the panel of biomarkers comprises KCNIP4 and one or more (e.g., 1, 2, 3, 4, 5, or more) additional biomarkers described herein. In some embodiments, the panel of biomarkers comprises NFIB and at least one (e.g., at least 1, 2, 3, 4, 5, or more) other biomarker described herein.

In some embodiments, the panel of biomarkers comprises SLITRK1 and one or more (e.g., 1, 2, 3, 4, 5, or more) additional biomarkers described herein. In some embodiments, the panel of biomarkers comprises ZNF521 and one or more (e.g., 1, 2, 3, 4, 5, or more) additional biomarkers described herein. In some embodiments, the panel of biomarkers comprises CCNB1 and one or more (e.g., 1, 2, 3, 4, 5, or more) additional biomarkers described herein. In some embodiments, the panel of biomarkers comprises CDK7 and one or more (e.g., 1, 2, 3, 4, 5, or more) additional biomarkers described herein. In some embodiments, the panel of biomarkers comprises MYT1L and one or more (e.g., 1, 2, 3, 4, 5, or more) additional biomarkers described herein. In some embodiments, the panel of biomarkers comprises FZR1 and one or more (e.g., 1, 2, 3, 4, 5, or more) additional biomarkers described herein. In some embodiments, the panel of biomarkers comprises SERF1A and one or more (e.g., 1, 2, 3, 4, 5, or more) additional biomarkers described herein. In some embodiments, the panel of biomarkers comprises GADD45B and one or more (e.g., 1, 2, 3, 4, 5, or more) additional biomarkers described herein. In some embodiments, the panel of biomarkers comprises ADGRL2 and one or more (e.g., 1, 2, 3, 4, 5, or more) additional biomarkers described herein. In some embodiments, the panel of biomarkers comprises TTK and one or more (e.g., 1, 2, 3, 4, 5, or more) additional biomarkers described herein. In some embodiments, the panel of biomarkers comprises NRXN2 and one or more (e.g., 1, 2, 3, 4, 5, or more) additional biomarkers described herein. In some embodiments, the panel of biomarkers comprises UNC13A and one or more (e.g., 1, 2, 3, 4, 5, or more) additional biomarkers described herein. In some embodiments, the panel of biomarkers comprises ZBTB7A and one or more (e.g., 1, 2, 3, 4, 5, or more) additional biomarkers described herein. In some embodiments, the panel of biomarkers comprises POLD1 and one or more (e.g., 1, 2, 3, 4, 5, or more) additional biomarkers described herein. In some embodiments, the panel of biomarkers comprises PCDH19 and one or more (e.g., 1, 2, 3, 4, 5, or more) additional biomarkers described herein. In some embodiments, the panel of biomarkers comprises SLC8A2 and one or more (e.g., 1, 2, 3, 4, 5, or more) additional biomarkers described herein. In some embodiments, the panel of biomarkers comprises E2F4 and one or more (e.g., 1, 2, 3, 4, 5, or more) additional biomarkers described herein. In some embodiments, the panel of biomarkers comprises AUTS2 and one or more (e.g., 1, 2, 3, 4, 5, or more) additional biomarkers described herein. In some embodiments, the panel of biomarkers comprises KCNN2 and one or more (e.g., 1, 2, 3, 4, 5, or more) additional biomarkers described herein. In some embodiments, the panel of biomarkers comprises CCNH and one or more (e.g., 1, 2, 3, 4, 5, or more) additional biomarkers described herein. In some embodiments, the panel of biomarkers comprises FRG2C and one or more (e.g., 1, 2, 3, 4, 5, or more) additional biomarkers described herein. In some embodiments, the panel of biomarkers comprises PLK2 and one or more (e.g., 1, 2, 3, 4, 5, or more) additional biomarkers described herein. In some embodiments, the panel of biomarkers comprises MYO18A and one or more (e.g., 1, 2, 3, 4, 5, or more) additional biomarkers described herein. In some embodiments, the panel of biomarkers comprises DCAF12L1 and one or more (e.g., 1, 2, 3, 4, 5, or more) additional biomarkers described herein.

In some embodiments, the panel of biomarkers comprises at least one biomarker selected from any one of the biomarkers listed in Table 1. In some embodiments, the panel of biomarkers comprises at least two biomarkers selected from any one or any combination of the biomarkers listed in Table 1. In some embodiments, the panel of biomarkers comprises at least three biomarkers selected from any one or any combination of the biomarkers listed in Table 1. In some embodiments, the panel of biomarkers comprises at least four biomarkers selected from any one or any combination of the biomarkers listed in Table 1. In some embodiments, the panel of biomarkers comprises at least five biomarkers selected from any one or any combination of the biomarkers listed in Table 1. In some embodiments, the panel of biomarkers comprises at least six biomarkers selected from any one or any combination of the biomarkers listed in Table 1. In some embodiments, the panel of biomarkers comprises at least seven biomarkers selected from any one or any combination of the biomarkers listed in Table 1. In some embodiments, the panel of biomarkers comprises at least eight biomarkers selected from any one or any combination of the biomarkers listed in Table 1. In some embodiments, the panel of biomarkers comprises at least nine biomarkers selected from any one or any combination of the biomarkers listed in Table 1. In some embodiments, the panel of biomarkers comprises at least ten or more biomarkers selected from any one or any combination of the biomarkers listed in Table 1.

In some embodiments, the panel of biomarkers comprises at least one biomarker selected from any one of the biomarkers listed in Table 2. In some embodiments, the panel of biomarkers comprises at least two biomarkers selected from any one or any combination of the biomarkers listed in Table 2. In some embodiments, the panel of biomarkers comprises at least three biomarkers selected from any one or any combination of the biomarkers listed in Table 2. In some embodiments, the panel of biomarkers comprises at least four biomarkers selected from any one or any combination of the biomarkers listed in Table 2. In some embodiments, the panel of biomarkers comprises at least five biomarkers selected from any one or any combination of the biomarkers listed in Table 2. In some embodiments, the panel of biomarkers comprises at least six biomarkers selected from any one or any combination of the biomarkers listed in Table 2. In some embodiments, the panel of biomarkers comprises at least seven biomarkers selected from any one or any combination of the biomarkers listed in Table 2. In some embodiments, the panel of biomarkers comprises at least eight biomarkers selected from any one or any combination of the biomarkers listed in Table 2. In some embodiments, the panel of biomarkers comprises at least nine biomarkers selected from any one or any combination of the biomarkers listed in Table 2. In some embodiments, the panel of biomarkers comprises at least ten or more biomarkers selected from any one or any combination of the biomarkers listed in Table 2.

In some embodiments, the panel of biomarkers comprises OR4F16 and at least one (e.g., 1, 2, 3, 4, 5, or more) additional biomarker described herein. In some embodiments, the panel of biomarkers comprises BUB1B and at least one (e.g., 1, 2, 3, 4, 5, or more) additional biomarker described herein. In some embodiments, the panel of biomarkers comprises PLK1 and at least one (e.g., 1, 2, 3, 4, 5, or more) additional biomarker described herein. In some embodiments, the panel of biomarkers comprises PAXBP1 and at least one (e.g., 1, 2, 3, 4, 5, or more) additional biomarker described herein. In some embodiments, the panel of biomarkers comprises CTR9 and at least one (e.g., 1, 2, 3, 4, 5, or more) additional biomarker described herein. In some embodiments, the panel of biomarkers comprises AR and at least one (e.g., 1, 2, 3, 4, 5, or more) additional biomarker described herein. In some embodiments, the panel of biomarkers comprises EIF3A and at least one (e.g., 1, 2, 3, 4, 5, or more) additional biomarker described herein. In some embodiments, the panel of biomarkers comprises KIF4A and at least one (e.g., 1, 2, 3, 4, 5, or more) additional biomarker described herein. In some embodiments, the panel of biomarkers comprises MAGEB10 and at least one (e.g., 1, 2, 3, 4, 5, or more) additional biomarker described herein. In some embodiments, the panel of biomarkers comprises CHEK1 and at least one (e.g., 1, 2, 3, 4, 5, or more) additional biomarker described herein. In some embodiments, the panel of biomarkers comprises CENPM and at least one (e.g., 1, 2, 3, 4, 5, or more) additional biomarker described herein. In some embodiments, the panel of biomarkers comprises AKT1 and at least one (e.g., 1, 2, 3, 4, 5, or more) additional biomarker described herein. In some embodiments, the panel of biomarkers comprises ADCY1 and at least one (e.g., 1, 2, 3, 4, 5, or more) additional biomarker described herein. In some embodiments, the panel of biomarkers comprises ATP2B2 and at least one (e.g., 1, 2, 3, 4, 5, or more) additional biomarker described herein. In some embodiments, the panel of biomarkers comprises HASPIN and at least one (e.g., 1, 2, 3, 4, 5, or more) additional biomarker described herein. In some embodiments, the panel of biomarkers comprises CTDSPL2 and at least one (e.g., 1, 2, 3, 4, 5, or more) additional biomarker described herein. In some embodiments, the panel of biomarkers comprises STAG2 and at least one (e.g., 1, 2, 3, 4, 5, or more) additional biomarker described herein. In some embodiments, the panel of biomarkers comprises NCAPG and at least one (e.g., 1, 2, 3, 4, 5, or more) additional biomarker described herein. In some embodiments, the panel of biomarkers comprises IGF1R and at least one (e.g., 1, 2, 3, 4, 5, or more) additional biomarker described herein. In some embodiments, the panel of biomarkers comprises BLM and at least one (e.g., 1, 2, 3, 4, 5, or more) additional biomarker described herein. In some embodiments, the panel of biomarkers comprises ATR and at least one (e.g., 1, 2, 3, 4, 5, or more) additional biomarker described herein. In some embodiments, the panel of biomarkers comprises AURKB and at least one (e.g., 1, 2, 3, 4, 5, or more) additional biomarker described herein. In some embodiments, the panel of biomarkers comprises RBL2 and at least one (e.g., 1, 2, 3, 4, 5, or more) additional biomarker described herein. In some embodiments, the panel of biomarkers comprises RPS6KA6 and at least one (e.g., 1, 2, 3, 4, 5, or more) additional biomarker described herein. In some embodiments, the panel of biomarkers comprises GINS2 and at least one (e.g., 1, 2, 3, 4, 5, or more) additional biomarker described herein. In some embodiments, the panel of biomarkers comprises MAD1L1 and at least one (e.g., 1, 2, 3, 4, 5, or more) additional biomarker described herein. In some embodiments, the panel of biomarkers comprises ADCY5 and at least one (e.g., 1, 2, 3, 4, 5, or more) additional biomarker described herein. In some embodiments, the panel of biomarkers comprises CHTF18 and at least one (e.g., 1, 2, 3, 4, 5, or more) additional biomarker described herein. In some embodiments, the panel of biomarkers comprises SMC1A and at least one (e.g., 1, 2, 3, 4, 5, or more) additional biomarker described herein. In some embodiments, the panel of biomarkers comprises BRSK2 and at least one (e.g., 1, 2, 3, 4, 5, or more) additional biomarker described herein. In some embodiments, the panel of biomarkers comprises BRPF3 and at least one (e.g., 1, 2, 3, 4, 5, or more) additional biomarker described herein. In some embodiments, the panel of biomarkers comprises FOXD4L4 and at least one (e.g., 1, 2, 3, 4, 5, or more) additional biomarker described herein. In some embodiments, the panel of biomarkers comprises TGIF2LX and at least one (e.g., 1, 2, 3, 4, 5, or more) additional biomarker described herein. In some embodiments, the panel of biomarkers comprises SOX5 and at least one (e.g., 1, 2, 3, 4, 5, or more) additional biomarker described herein. In some embodiments, the panel of biomarkers comprises POU4F1 and at least one (e.g., 1, 2, 3, 4, 5, or more) additional biomarker described herein. In some embodiments, the panel of biomarkers comprises UHRF1 and at least one (e.g., 1, 2, 3, 4, 5, or more) additional biomarker described herein. In some embodiments, the panel of biomarkers comprises PPP2R2C and at least one (e.g., 1, 2, 3, 4, 5, or more) additional biomarker described herein. In some embodiments, the panel of biomarkers comprises WDR45 and at least one (e.g., 1, 2, 3, 4, 5, or more) additional biomarker described herein. In some embodiments, the panel of biomarkers comprises FAM120C and at least one (e.g., 1, 2, 3, 4, 5, or more) additional biomarker described herein. In some embodiments, the panel of biomarkers comprises BRSK1 and at least one (e.g., 1, 2, 3, 4, 5, or more) additional biomarker described herein. In some embodiments, the panel of biomarkers comprises EVI5L and at least one (e.g., 1, 2, 3, 4, 5, or more) additional biomarker described herein. In some embodiments, the panel of biomarkers comprises NPAS4 and at least one (e.g., 1, 2, 3, 4, 5, or more) additional biomarker described herein. In some embodiments, the panel of biomarkers comprises MCM10 and at least one (e.g., 1, 2, 3, 4, 5, or more) additional biomarker described herein. In some embodiments, the panel of biomarkers comprises SUPT5H and at least one (e.g., 1, 2, 3, 4, 5, or more) additional biomarker described herein. In some embodiments, the panel of biomarkers comprises MCM5 and at least one (e.g., 1, 2, 3, 4, 5, or more) additional biomarker described herein. In some embodiments, the panel of biomarkers comprises GALK2 and at least one (e.g., 1, 2, 3, 4, 5, or more) additional biomarker described herein. In some embodiments, the panel of biomarkers comprises FTSJ1 and at least one (e.g., 1, 2, 3, 4, 5, or more) additional biomarker described herein. In some embodiments, the panel of biomarkers comprises TRAP1 and at least one (e.g., 1, 2, 3, 4, 5, or more) additional biomarker described herein. In some embodiments, the panel of biomarkers comprises PAK3 and at least one (e.g., 1, 2, 3, 4, 5, or more) additional biomarker described herein. In some embodiments, the panel of biomarkers comprises CENPE and at least one (e.g., 1, 2, 3, 4, 5, or more) additional biomarker described herein. In some embodiments, the panel of biomarkers comprises TPT1 and at least one (e.g., 1, 2, 3, 4, 5, or more) additional biomarker described herein. In some embodiments, the panel of biomarkers comprises MAD2L2 and at least one (e.g., 1, 2, 3, 4, 5, or more) additional biomarker described herein. In some embodiments, the panel of biomarkers comprises FBXO5 and at least one (e.g., 1, 2, 3, 4, 5, or more) additional biomarker described herein. In some embodiments, the panel of biomarkers comprises CDK16 and at least one (e.g., 1, 2, 3, 4, 5, or more) additional biomarker described herein. In some embodiments, the panel of biomarkers comprises CDC45 and at least one (e.g., 1, 2, 3, 4, 5, or more) additional biomarker described herein. In some embodiments, the panel of biomarkers comprises USP27X and at least one (e.g., 1, 2, 3, 4, 5, or more) additional biomarker described herein. In some embodiments, the panel of biomarkers comprises MAPK8 and at least one (e.g., 1, 2, 3, 4, 5, or more) additional biomarker described herein. In some embodiments, the panel of biomarkers comprises PRR20A and at least one (e.g., 1, 2, 3, 4, 5, or more) additional biomarker described herein. In some embodiments, the panel of biomarkers comprises ADCY4 and at least one (e.g., 1, 2, 3, 4, 5, or more) additional biomarker described herein. In some embodiments, the panel of biomarkers comprises RRM1 and at least one (e.g., 1, 2, 3, 4, 5, or more) additional biomarker described herein. In some embodiments, the panel of biomarkers comprises TBR1 and at least one (e.g., 1, 2, 3, 4, 5, or more) additional biomarker described herein. In some embodiments, the panel of biomarkers comprises PAK2 and at least one (e.g., 1, 2, 3, 4, 5, or more) additional biomarker described herein. In some embodiments, the panel of biomarkers comprises KIF11 and at least one (e.g., 1, 2, 3, 4, 5, or more) additional biomarker described herein. In some embodiments, the panel of biomarkers comprises WDHD1 and at least one (e.g., 1, 2, 3, 4, 5, or more) additional biomarker described herein. In some embodiments, the panel of biomarkers comprises MELK and at least one (e.g., 1, 2, 3, 4, 5, or more) additional biomarker described herein. In some embodiments, the panel of biomarkers comprises CHERP and at least one (e.g., 1, 2, 3, 4, 5, or more) additional biomarker described herein. In some embodiments, the panel of biomarkers comprises CENPF and at least one (e.g., 1, 2, 3, 4, 5, or more) additional biomarker described herein. In some embodiments, the panel of biomarkers comprises BUB1 and at least one (e.g., 1, 2, 3, 4, 5, or more) additional biomarker described herein. In some embodiments, the panel of biomarkers comprises PRMT5 and at least one (e.g., 1, 2, 3, 4, 5, or more) additional biomarker described herein. In some embodiments, the panel of biomarkers comprises EIF1AX and at least one (e.g., 1, 2, 3, 4, 5, or more) additional biomarker described herein. In some embodiments, the panel of biomarkers comprises SMPD2 and at least one (e.g., 1, 2, 3, 4, 5, or more) additional biomarker described herein. In some embodiments, the panel of biomarkers comprises CASP8AP2 and at least one (e.g., 1, 2, 3, 4, 5, or more) additional biomarker described herein. In some embodiments, the panel of biomarkers comprises SFN and at least one (e.g., 1, 2, 3, 4, 5, or more) additional biomarker described herein. In some embodiments, the panel of biomarkers comprises WEE1 and at least one (e.g., 1, 2, 3, 4, 5, or more) additional biomarker described herein. In some embodiments, the panel of biomarkers comprises ESPL1 and at least one (e.g., 1, 2, 3, 4, 5, or more) additional biomarker described herein. In some embodiments, the panel of biomarkers comprises OTUD5 and at least one (e.g., 1, 2, 3, 4, 5, or more) additional biomarker described herein. In some embodiments, the panel of biomarkers comprises DMRTC1B and at least one (e.g., 1, 2, 3, 4, 5, or more) additional biomarker described herein. In some embodiments, the panel of biomarkers comprises TSSK2 and at least one (e.g., 1, 2, 3, 4, 5, or more) additional biomarker described herein. In some embodiments, the panel of biomarkers comprises ANAPC10 and at least one (e.g., 1, 2, 3, 4, 5, or more) additional biomarker described herein. In some embodiments, the panel of biomarkers comprises FOXM1 and at least one (e.g., 1, 2, 3, 4, 5, or more) additional biomarker described herein. In some embodiments, the panel of biomarkers comprises EXO1 and at least one (e.g., 1, 2, 3, 4, 5, or more) additional biomarker described herein. In some embodiments, the panel of biomarkers comprises CHEK2 and at least one (e.g., 1, 2, 3, 4, 5, or more) additional biomarker described herein. In some embodiments, the panel of biomarkers comprises KIFC1 and at least one (e.g., 1, 2, 3, 4, 5, or more) additional biomarker described herein. In some embodiments, the panel of biomarkers comprises ANKRD52 and at least one (e.g., 1, 2, 3, 4, 5, or more) additional biomarker described herein. In some embodiments, the panel of biomarkers comprises SPAG5 and at least one (e.g., 1, 2, 3, 4, 5, or more) additional biomarker described herein. In some embodiments, the panel of biomarkers comprises PPP2R2B and at least one (e.g., 1, 2, 3, 4, 5, or more) additional biomarker described herein. In some embodiments, the panel of biomarkers comprises ZNF331 and at least one (e.g., 1, 2, 3, 4, 5, or more) additional biomarker described herein. In some embodiments, the panel of biomarkers comprises PAK1 and at least one (e.g., 1, 2, 3, 4, 5, or more) additional biomarker described herein. In some embodiments, the panel of biomarkers comprises TNPO2 and at least one (e.g., 1, 2, 3, 4, 5, or more) additional biomarker described herein. In some embodiments, the panel of biomarkers comprises LDB1 and at least one (e.g., 1, 2, 3, 4, 5, or more) additional biomarker described herein. In some embodiments, the panel of biomarkers comprises CDK14 and at least one (e.g., 1, 2, 3, 4, 5, or more) additional biomarker described herein. In some embodiments, the panel of biomarkers comprises CDC25B and at least one (e.g., 1, 2, 3, 4, 5, or more) additional biomarker described herein. In some embodiments, the panel of biomarkers comprises KCNV1 and at least one (e.g., 1, 2, 3, 4, 5, or more) additional biomarker described herein. In some embodiments, the panel of biomarkers comprises CPEB1 and at least one (e.g., 1, 2, 3, 4, 5, or more) additional biomarker described herein. In some embodiments, the panel of biomarkers comprises ZNF777 and at least one (e.g., 1, 2, 3, 4, 5, or more) additional biomarker described herein. In some embodiments, the panel of biomarkers comprises RPS6KA1 and at least one (e.g., 1, 2, 3, 4, 5, or more) additional biomarker described herein.

In some embodiments, the panel of biomarkers comprises PSG7 and at least one (e.g., 1, 2, 3, 4, 5, or more) additional biomarker described herein. In some embodiments, the panel of biomarkers comprises CD177 and at least one (e.g., 1, 2, 3, 4, 5, or more) additional biomarker described herein. In some embodiments, the panel of biomarkers comprises CCNG1 and at least one (e.g., 1, 2, 3, 4, 5, or more) additional biomarker described herein. In some embodiments, the panel of biomarkers comprises PRAMEF8 and at least one (e.g., 1, 2, 3, 4, 5, or more) additional biomarker described herein. In some embodiments, the panel of biomarkers comprises ZBTB17 and at least one (e.g., 1, 2, 3, 4, 5, or more) additional biomarker described herein. In some embodiments, the panel of biomarkers comprises CCNF and at least one (e.g., 1, 2, 3, 4, 5, or more) additional biomarker described herein. In some embodiments, the panel of biomarkers comprises E2F2 and at least one (e.g., 1, 2, 3, 4, 5, or more) additional biomarker described herein. In some embodiments, the panel of biomarkers comprises HDAC1 and at least one (e.g., 1, 2, 3, 4, 5, or more) additional biomarker described herein. In some embodiments, the panel of biomarkers comprises CCNB2 and at least one (e.g., 1, 2, 3, 4, 5, or more) additional biomarker described herein. In some embodiments, the panel of biomarkers comprises KIF15 and at least one (e.g., 1, 2, 3, 4, 5, or more) additional biomarker described herein. In some embodiments, the panel of biomarkers comprises AGPAT3 and at least one (e.g., 1, 2, 3, 4, 5, or more) additional biomarker described herein. In some embodiments, the panel of biomarkers comprises REC8 and at least one (e.g., 1, 2, 3, 4, 5, or more) additional biomarker described herein. In some embodiments, the panel of biomarkers comprises RECQL4 and at least one (e.g., 1, 2, 3, 4, 5, or more) additional biomarker described herein. In some embodiments, the panel of biomarkers comprises ZNF853 and at least one (e.g., 1, 2, 3, 4, 5, or more) additional biomarker described herein. In some embodiments, the panel of biomarkers comprises SRSF4 and at least one (e.g., 1, 2, 3, 4, 5, or more) additional biomarker described herein. In some embodiments, the panel of biomarkers comprises PPP2R5A and at least one (e.g., 1, 2, 3, 4, 5, or more) additional biomarker described herein. In some embodiments, the panel of biomarkers comprises ZBTB12 and at least one (e.g., 1, 2, 3, 4, 5, or more) additional biomarker described herein. In some embodiments, the panel of biomarkers comprises MMP12 and at least one (e.g., 1, 2, 3, 4, 5, or more) additional biomarker described herein. In some embodiments, the panel of biomarkers comprises KIF2C and at least one (e.g., 1, 2, 3, 4, 5, or more) additional biomarker described herein. In some embodiments, the panel of biomarkers comprises HSP90AA1 and at least one (e.g., 1, 2, 3, 4, 5, or more) additional biomarker described herein. In some embodiments, the panel of biomarkers comprises PPP2R2D and at least one (e.g., 1, 2, 3, 4, 5, or more) additional biomarker described herein. In some embodiments, the panel of biomarkers comprises CDC7 and at least one (e.g., 1, 2, 3, 4, 5, or more) additional biomarker described herein. In some embodiments, the panel of biomarkers comprises NANS and at least one (e.g., 1, 2, 3, 4, 5, or more) additional biomarker described herein. In some embodiments, the panel of biomarkers comprises MOS and at least one (e.g., 1, 2, 3, 4, 5, or more) additional biomarker described herein. In some embodiments, the panel of biomarkers comprises RBX1 and at least one (e.g., 1, 2, 3, 4, 5, or more) additional biomarker described herein. In some embodiments, the panel of biomarkers comprises MAGED4B and at least one (e.g., 1, 2, 3, 4, 5, or more) additional biomarker described herein. In some embodiments, the panel of biomarkers comprises KIF23 and at least one (e.g., 1, 2, 3, 4, 5, or more) additional biomarker described herein. In some embodiments, the panel of biomarkers comprises SCML1 and at least one (e.g., 1, 2, 3, 4, 5, or more) additional biomarker described herein. In some embodiments, the panel of biomarkers comprises SPANXA2 and at least one (e.g., 1, 2, 3, 4, 5, or more) additional biomarker described herein. In some embodiments, the panel of biomarkers comprises TRIM28 and at least one (e.g., 1, 2, 3, 4, 5, or more) additional biomarker described herein. In some embodiments, the panel of biomarkers comprises SRRM5 and at least one (e.g., 1, 2, 3, 4, 5, or more) additional biomarker described herein. In some embodiments, the panel of biomarkers comprises MAGEA1 and at least one (e.g., 1, 2, 3, 4, 5, or more) additional biomarker described herein. In some embodiments, the panel of biomarkers comprises ACTR3B and at least one (e.g., 1, 2, 3, 4, 5, or more) additional biomarker described herein. In some embodiments, the panel of biomarkers comprises EBLN1 and at least one (e.g., 1, 2, 3, 4, 5, or more) additional biomarker described herein. In some embodiments, the panel of biomarkers comprises TP53TG3C and at least one (e.g., 1, 2, 3, 4, 5, or more) additional biomarker described herein. In some embodiments, the panel of biomarkers comprises INS and at least one (e.g., 1, 2, 3, 4, 5, or more) additional biomarker described herein. In some embodiments, the panel of biomarkers comprises ORC1 and at least one (e.g., 1, 2, 3, 4, 5, or more) additional biomarker described herein. In some embodiments, the panel of biomarkers comprises HSP90AB1 and at least one (e.g., 1, 2, 3, 4, 5, or more) additional biomarker described herein. In some embodiments, the panel of biomarkers comprises CHAF1B and at least one (e.g., 1, 2, 3, 4, 5, or more) additional biomarker described herein. In some embodiments, the panel of biomarkers comprises MCM7 and at least one (e.g., 1, 2, 3, 4, 5, or more) additional biomarker described herein. In some embodiments, the panel of biomarkers comprises CPSF6 and at least one (e.g., 1, 2, 3, 4, 5, or more) additional biomarker described herein. In some embodiments, the panel of biomarkers comprises NACC1 and at least one (e.g., 1, 2, 3, 4, 5, or more) additional biomarker described herein. In some embodiments, the panel of biomarkers comprises WEE2 and at least one (e.g., 1, 2, 3, 4, 5, or more) additional biomarker described herein. In some embodiments, the panel of biomarkers comprises MYC and at least one (e.g., 1, 2, 3, 4, 5, or more) additional biomarker described herein. In some embodiments, the panel of biomarkers comprises MCM6 and at least one (e.g., 1, 2, 3, 4, 5, or more) additional biomarker described herein. In some embodiments, the panel of biomarkers comprises ADCY6 and at least one (e.g., 1, 2, 3, 4, 5, or more) additional biomarker described herein. In some embodiments, the panel of biomarkers comprises TPX2 and at least one (e.g., 1, 2, 3, 4, 5, or more) additional biomarker described herein. In some embodiments, the panel of biomarkers comprises MYBL2 and at least one (e.g., 1, 2, 3, 4, 5, or more) additional biomarker described herein. In some embodiments, the panel of biomarkers comprises CDC23 and at least one (e.g., 1, 2, 3, 4, 5, or more) additional biomarker described herein. In some embodiments, the panel of biomarkers comprises RRM2 and at least one (e.g., 1, 2, 3, 4, 5, or more) additional biomarker described herein. In some embodiments, the panel of biomarkers comprises MAPK1 and at least one (e.g., 1, 2, 3, 4, 5, or more) additional biomarker described herein. In some embodiments, the panel of biomarkers comprises PRKACA and at least one (e.g., 1, 2, 3, 4, 5, or more) additional biomarker described herein. In some embodiments, the panel of biomarkers comprises DDI2 and at least one (e.g., 1, 2, 3, 4, 5, or more) additional biomarker described herein. In some embodiments, the panel of biomarkers comprises MEMO1 and at least one (e.g., 1, 2, 3, 4, 5, or more) additional biomarker described herein. In some embodiments, the panel of biomarkers comprises IGF1 and at least one (e.g., 1, 2, 3, 4, 5, or more) additional biomarker described herein. In some embodiments, the panel of biomarkers comprises SKP1 and at least one (e.g., 1, 2, 3, 4, 5, or more) additional biomarker described herein. In some embodiments, the panel of biomarkers comprises PPIAL4C and at least one (e.g., 1, 2, 3, 4, 5, or more) additional biomarker described herein. In some embodiments, the panel of biomarkers comprises PPIAL4D and at least one (e.g., 1, 2, 3, 4, 5, or more) additional biomarker described herein. In some embodiments, the panel of biomarkers comprises SLC9A6 and at least one (e.g., 1, 2, 3, 4, 5, or more) additional biomarker described herein. In some embodiments, the panel of biomarkers comprises ARPP19 and at least one (e.g., 1, 2, 3, 4, 5, or more) additional biomarker described herein. In some embodiments, the panel of biomarkers comprises NOVA2 and at least one (e.g., 1, 2, 3, 4, 5, or more) additional biomarker described herein. In some embodiments, the panel of biomarkers comprises CTAG1B and at least one (e.g., 1, 2, 3, 4, 5, or more) additional biomarker described herein. In some embodiments, the panel of biomarkers comprises CCNA2 and at least one (e.g., 1, 2, 3, 4, 5, or more) additional biomarker described herein. In some embodiments, the panel of biomarkers comprises CDC6 and at least one (e.g., 1, 2, 3, 4, 5, or more) additional biomarker described herein. In some embodiments, the panel of biomarkers comprises MAGEA9 and at least one (e.g., 1, 2, 3, 4, 5, or more) additional biomarker described herein. In some embodiments, the panel of biomarkers comprises F8A3 and at least one (e.g., 1, 2, 3, 4, 5, or more) additional biomarker described herein. In some embodiments, the panel of biomarkers comprises ARL17A and at least one (e.g., 1, 2, 3, 4, 5, or more) additional biomarker described herein. In some embodiments, the panel of biomarkers comprises CTAG1A and at least one (e.g., 1, 2, 3, 4, 5, or more) additional biomarker described herein. In some embodiments, the panel of biomarkers comprises MAD2L1 and at least one (e.g., 1, 2, 3, 4, 5, or more) additional biomarker described herein. In some embodiments, the panel of biomarkers comprises HSFX1 and at least one (e.g., 1, 2, 3, 4, 5, or more) additional biomarker described herein. In some embodiments, the panel of biomarkers comprises BNIP3 and at least one (e.g., 1, 2, 3, 4, 5, or more) additional biomarker described herein. In some embodiments, the panel of biomarkers comprises MRGPRG and at least one (e.g., 1, 2, 3, 4, 5, or more) additional biomarker described herein. In some embodiments, the panel of biomarkers comprises ANAPC2 and at least one (e.g., 1, 2, 3, 4, 5, or more) additional biomarker described herein. In some embodiments, the panel of biomarkers comprises RAD21 and at least one (e.g., 1, 2, 3, 4, 5, or more) additional biomarker described herein.

In some embodiments, the additional biomarker is selected from any one or any combination (e.g., 2, 3, 4, 5, or more) of MAP2K4, TP53, CDC25A, CACNA1H, CDKN1B, DUSP7, FOXO3, FZD3, JAK1, SMAD2, TGFBR2, MAP3K2, a PP3 subunit, and a PP2A subunit. In some embodiments, the PP2A subunit is PPP2R1B. In some embodiments, the PP3 subunit is PPP3CC. In some embodiments, the PP2A subunit is selected from: 65 kDa regulatory subunit A alpha (PPP2R1A), 65 kDa regulatory subunit A beta (PPP2R1B), 55 kDa regulatory subunit B alpha (PPP2R2A), 55 kDa regulatory subunit B beta (PPP2R2B), 55 kDa regulatory subunit B gamma (PPP2R2C), 55 kDa regulatory subunit B delta (PPP2R2D), 72/130 kDa regulatory subunit B (PPP2R3A), 48 kDa regulatory subunit B (PPP2R3B), regulatory subunit B″ subunit gamma (PPP2R3C), regulatory subunit B′ (PPP2R4), 56 kDa regulatory subunit alpha (PPP2R5A), 56 kDa regulatory subunit beta (PPP2R5B), 56 kDa regulatory subunit gamma (PPP2R5C), 56 kDa regulatory subunit delta (PPP2R5D), 56 kDa regulatory subunit epsilon (PPP2R5E), catalytic subunit alpha (PPP2CA), and catalytic subunit beta (PPP2CB). In some embodiments, the PP2A subunit is PPP2R2A.

### Therapeutic Agents

In some embodiments, the disclosure provides a method of treating cancer in a subject, comprising administering to the subject a therapeutically effective amount of one or more therapeutic agents that alter (e.g., increase or decrease) the expression and/or activity of PKMYT1.

In some embodiments, the one or more therapeutic agent used to alter (e.g., decrease or increase) expression level and/or activity of PKMYT1 comprises a small molecule (e.g., a molecule having a molecular weight of less than 900 Daltons), a protein, an intrabody, a peptide, a ribonucleic acid (RNA) molecule, a deoxyribonucleic acid (DNA) construct, or a combination thereof (e.g., a protein-nucleic acid complex).

In some embodiments, the one or more therapeutic agents comprises a protein-nucleic acid complex, e.g., an endonuclease complex and a nucleic acid construct. In some cases, the endonuclease complex comprises a clustered regularly interspaced short palindromic repeat (CRISPR) associated (Cas) protein or variant thereof (e.g., an engineered variant) or a nucleic acid encoding the Cas protein or variant thereof. In some embodiments, the endonuclease complex comprises a clustered regularly interspaced short palindromic repeat (CRISPR) associated (Cas) protein or variant thereof (e.g., an engineered variant). In some embodiments, the nucleic construct is co-administered with the endonuclease complex. In some embodiments, the nucleic acid comprises an endonuclease gene. In some embodiments, the nucleic acid comprises a gene encoding a Cas protein or variant thereof (e.g., an engineered variant). In some embodiments, the nucleic acid is transcribed and translated by the cell using the cell's own machinery (e.g., polymerases, ribosomes, etc.) once the nucleic acid is introduced or delivered to a cell (e.g., cancer cell).

In some embodiments, the endonuclease complex comprises an endonuclease, e.g., a Cas protein, or other nucleic acid-interacting enzyme (e.g., ligase, helicase, reverse transcriptase, transcriptase, polymerase, etc.). In some embodiments, the Cas protein comprises any Cas type (e.g., Cas I, Cas IA, Cas IB, Cas IC, Cas ID, Cas IE, Cas IF, Cas IU, Cas III, Cas IIIA, Cas IIIB, Cas IIIC, Cas HID, Cas IV, Cas IVA, Cas IVB, Cas II, Cas IIA, Cas IIB, Cas IIC, Cas V, Cas VI). In some embodiments, the Cas protein comprises other proteins (e.g., a fusion protein). In some embodiments, the Cas protein comprises an additional enzyme that associates with a nucleic acid molecule (e.g., ligase, transcriptase, transposase, nuclease, endonuclease, reverse transcriptase, polymerase, helicase, etc.). In some embodiments, the endonuclease complex is delivered exogenously or is encoded in the nucleic acid construct for transcription and translation within the cell.

In some embodiments, the one or more therapeutic agents comprises a small molecule inhibitor (e.g., a molecule having a molecular weight of less than 900 Daltons). In some embodiments, the small molecule is configured to decrease the expression level and/or activity level of PKMYT1, or the small molecule is configured to decrease the expression level and/or activity level of PKMYT1 in combination with a deficiency or mutation in the gene encoding the biomarker. In some embodiments, the small molecule may directly interact with both the first gene and the second gene. For example, the small molecule may inhibit the protein or proteins encoded by one or both of the first gene and the second gene, respectively. Alternatively or in addition to, the small molecule may inhibit an upstream effector or downstream protein in a signaling pathway in which one or both of the genes interact.

In some embodiments, the small molecule inhibitor comprises a PKMYT1 inhibitor. Non-limiting examples of PKMYT1 inhibitor include 5-((5-methoxy-2-((4-morpholinophenyl)amino)pyrimidin-4-yl)amino)-2-methylphenol, N-(2-chloro-6-methylphenyl)-2-((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), N-(5-chlorobenzo[d][1,3]dioxol-4-yl)-7-(2-(4-methylpiperazin-1-yl)ethoxy)-5-((tetrahydro-2H-pyran-4-yl)oxy)quinazolin-4-amine (saracatinib), (E)-N-(4-((3-chloro-4-fluorophenyl)amino)-3-cyano-7-ethoxyquinolin-6-yl)-4-(dimethylamino)but-2-enamide (pelitinib), N-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-d]pyrimidin-7(8H)-one (PD-0166285), 6-(2,6-dichlorophenyl)-8-methyl-2-((4-morpholinophenyl)amino)pyrido[2,3-d]pyrimidin-7(8H)-one (PD-173952), 6-(2,6-dichlorophenyl)-8-methyl-2-((3-(methylthio)phenyl)amino)pyrido[2,3-d]pyrimidin-7(8H)-one (PD-173955), and 6-(2,6-dichlorophenyl)-2-((4-fluoro-3-methylphenyl)amino)-8-methylpyrido[2,3-d]pyrimidin-7(8H)-one (PD-180970). In some embodiments, the PKMYT1 inhibitor is dasatinib, saracatinib, pelitinib, tyrphostin AG 1478, PD-0166285, PD-173952, PD-173955, or PD-180970.

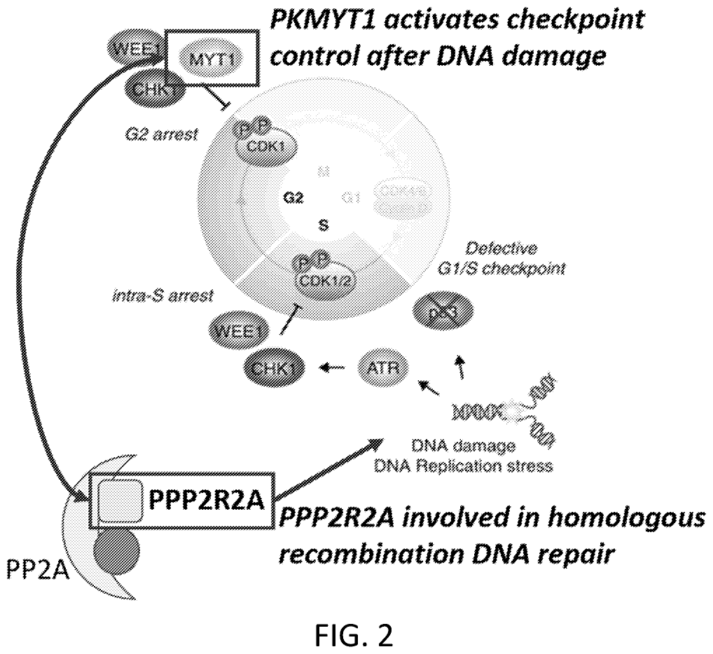

In some embodiments, the small molecule inhibitor is configured to inhibit or decrease the expression of PKMYT1 gene or the activity of PKMYT1 (a protein derived from the PKMYT1 gene), either directly or indirectly. In some embodiments, the small molecule inhibitor inhibits PKMYT1 by binding to the PKMYT1 kinase domain. In some embodiments, the small molecule inhibitor is an allosteric inhibitor of PKMYT1. In some embodiments, the small molecule inhibitor inhibits a protein upstream or downstream of PKMYT1 in a signaling pathway, such as, but not limited to, those shown in FIG. 2. In some embodiments, the small molecule inhibitor inhibits or otherwise decreases the expression or activity level of WEE1, CHK1, CDK1, CDK2, PPP2R2A, FOXM1, PLK1, and/or EZH2.

In some embodiments, the small molecule inhibitor comprises a combination of small molecule inhibitors or derivatives thereof. For example, in some embodiments, a small molecule inhibitor is engineered or modified for dual specificity, wherein the small molecule inhibitor decreases expression level and/or activity of PKMYT1 and the biomarker. In some embodiments, a combination of small molecule inhibitors (e.g., a small molecule “cocktail”) is used to decrease expression level and/or activity of PKMYT1 alone or both PKMYT1 and the biomarker.

In some embodiments, the small molecule inhibitor is administered in any useful concentration.

For example, in some embodiments, the small molecule is administered at a concentration of about 0.5 nanomolar (nM), about 1 nM, about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 micromolar (μM), about 2 μM, about 3 μM, about 4 μM, about 5 μM, about 6 μM, about 7 μM, about 8 μM, about 9 μM, about 10 μM. In some embodiments, the small molecule inhibitor is administered at a concentration of at least about 0.5 nanomolar (nM), at least about 1 nM, at least about 10 nM, at least about 20 nM, at least about 30 nM, at least about 40 nM, at least about 50 nM, at least about 60 nM, at least about 70 nM, at least about 80 nM, at least about 90 nM, at least about 100 nM, at least about 200 nM, at least about 300 nM, at least about 400 nM, at least about 500 nM, at least about 600 nM, at least about 700 nM, at least about 800 nM, at least about 900 nM, at least about 1 micromolar (μM), at least about 2 μM, at least about 3 μM, at least about 4 μM, at least about 5 μM, at least about 6 μM, at least about 7 μM, at least about 8 μM, at least about 9 μM, at least about 10 μM. In some embodiments, the small molecule inhibitor is administered at a concentration of not more than about 10 μM, at most about 9 μM, at most about 8 μM, at most about 7 μM, at most about 6 μM, at most about 5 μM, at most about 4 μM, at most about 3 μM, at most about 2 μM, at most about 1 μM, at most about 900 nM, at most about 800 nM, at most about 700 nM, at most about 600 nM, at most about 500 nM, at most about 400 nM, at most about 300 nM, at most about 200 nM, at most about 100 nM, at most about 90 nM, at most about 80 nM, at most about 70 nM, at most about 60 nM, at most about 50 nM, at most about 40 nM, at most about 30 nM, at most about 20 nM, at most about 10 nM, at most about 1 nM, at most about 0.5 nM, etc. A range of concentrations may be used, e.g., between 22 nM-1 μM. Wherein more than one small molecule is used, the concentrations may be the same of different for each small molecule used.

In some embodiments, the administration of the one or more therapeutic agents in a subject having a cancer with a mutation or deficiency in a biomarker described herein requires a lower concentration or dosage to achieve therapeutic efficacy. For example, in some embodiments, a lower dosage of PKMYT1 inhibitor is sufficient to kill cancer cells comprising a deficiency and/or mutation in a gene encoding a biomarker described herein that is a synthetic lethal pair with PKMYT1, as compared to control cells (e.g., non-cancer cells) that do not have the biomarker deficiency and/or mutation. Without being bound by theory, as higher dosages or concentrations of PKMYT1 inhibition in a subject may increase toxicity, administration of a lower concentration or dosage of PKMYT1 inhibitor in selected or pre-screened cancer types (e.g., cancers comprising the deficiency and/or mutation in a biomarker described herein that is a synthetic lethal pair with PKMYT1) is advantageous to reduce toxicity and side effects to the subject.

In some embodiments, the one or more therapeutic agents comprises a protein or peptide. For example, in some embodiments, the one or more therapeutic agents comprises an antibody, an antibody fragment, a hormone, a ligand, or an immunoglobulin. In some embodiments, the protein or peptide is naturally occurring or is synthetic. In some embodiments, the protein comprises an engineered variant of a protein (e.g., recombinant protein), or fragment thereof. In some embodiments, the protein is subjected to other modifications, e.g., post-translational modifications, including but not limited to: glycosylation, acylation, prenylation, lipoylation, alkylation, amidation, acetylation, methylation, formylation, butyrylation, carboxylation, phosphorylation, malonylation, hydroxylation, iodination, propionylation, S-nitrosylation, S-glutationylation, succinylation, sulfation, glycation, carbamylation, carbonylation, biotinylation, carbamylation, oxidation, pegylation, sumoylation, ubiquitination, ubiquitylation, racemization, etc. One or more modifications may be made to the protein or peptide.

In some embodiments, the one or more therapeutic agents comprises a nucleic acid molecule, e.g., an RNA molecule. In some embodiments, the RNA molecule comprises any suitable RNA molecule and size sufficient to decrease the expression level and/or activity of PKMYT1. In some embodiments, the RNA molecule comprises a small hairpin RNA (shRNA) molecule, a small interfering RNA (siRNA), a microRNA (miRNA), or other useful RNA molecule. In some embodiments, the RNA molecule comprises a messenger RNA (mRNA), transfer RNA (tRNA), ribosomal RNAs (rRNA), small nuclear RNA (snRNA), piwi-interacting (piRNA), non-coding RNA (ncRNA), long non-coding RNA, (lncRNA), and fragments of any of the foregoing. In some embodiments, the RNA molecule is single-stranded, double-stranded, or partially single- or double-stranded.

It will be appreciated that one or more therapeutic agents (e.g., peptides, RNA molecules, protein-nucleic acid complexes) are listed as examples and that a combination of therapeutic agent types may be used to treat the subject. For example, in some embodiments, administering one or more different types of therapeutic agents may be used to alter (e.g., increase or decrease) the expression level and/or activity of PKMYT1. For example, in some embodiments, a protein or peptide co-administered with a small molecule (e.g., a molecule having a molecular weight of less than 900 Daltons), an RNA molecule, a DNA molecule, or a complexed molecule (e.g., protein-nucleic acid molecule) is used to alter (e.g., increase or decrease) the expression level and/or activity of PKMYT1. In some embodiments, an RNA molecule is co-administered with a small molecule, a DNA molecule, or a complexed molecule to alter (e.g., increase or decrease) the expression level and/or activity of PKMYT1. In some embodiments, a small molecule is co-administered with a DNA molecule or a complexed molecule to alter (e.g., increase or decrease) the expression level and/or activity of PKMYT1. Any of these combinations may be used to alter (e.g., increase or decrease) the expression level and/or activity of PKMYT1 in a cell comprising a mutation and/or deficiency in a biomarker described herein (e.g., a biomarker forming a synthetic lethal pair with PKMYT1). These combinations are non-limiting examples of different combinations of agents that may be used to treat the subject having or suspected of having cancer (e.g., liver or ovarian cancer).

### Administration

In some embodiments, the present disclosure provides methods and compositions for delivery, administration of, or exposure to one or more therapeutic agents described herein. In some embodiments, one or more therapeutic agents are delivered to a subject (e.g., in vivo), or to a cell or population of cells from a subject (e.g., ex vivo or in vivo). In some embodiments, the one or more therapeutic agents are delivered to a subject in one or more delivery vesicles, such as a nanoparticle. In some embodiments, the nanoparticle is any suitable nanoparticle and may be a solid, semi-solid, semi-liquid or a gel. In some embodiments, the nanoparticle is a lipophilic or amphiphilic particle. For example, a nanoparticle may comprise a micelle, liposome, exosome, or other lipid-containing vesicle. In some embodiments, the nanoparticle is configured for targeted delivery to a certain cell or cell type (e.g., cancer cell). In such cases, the nanoparticle is decorated with any number of ligands, e.g., antibodies, nucleic acid molecules (e.g., ribonucleic acid (RNA) molecules or deoxyribonucleic acid (DNA) molecules), proteins, peptides, which may specifically bind to a certain cell or cell type (e.g., cancer cell).

In some embodiments, the one or more therapeutic agents are delivered using viral approaches. For example, in some embodiments, the one or more therapeutic agents is administered using a viral vector. In such cases, the one or more therapeutic agents is encapsulated in a virus for delivery to a cell, population of cells, or the subject. In some embodiments, the virus is an adeno-associated virus (AAV), a retrovirus, a lentivirus, a herpes simplex virus, or other useful virus. In some embodiments, the virus is engineered or naturally occurring.

In some embodiments, the one or more therapeutic agents is delivered to a subject (e.g., human patient) systemically or locally (e.g., at the tumor site) using a single or variety of approaches. For example, in some embodiments, the one or more therapeutic agents is delivered or administered orally, intravenously, intraperitoneally, intratumorally, subcutaneously, topically, transdermally, transmucosally, or through another administration approach.

In some embodiments, the one or more therapeutic agents is delivered to the subject enterally. For example, in some embodiments, the one or more therapeutic agents is administered to the subject orally, nasally, rectally, sublingually, sub-labially, buccally, topically, or through an enema. In some embodiments, the one or more therapeutic agents is formulated into a tablet, capsule, drop or other formulation. In some embodiments, the formulation is configured to be delivered enterally.

In some embodiments, the one or more therapeutic agents is delivered to the subject parenterally. For example, in some embodiments, the one or more therapeutic agents is administered via systemic or local injection. In some embodiments, the local injection comprises administration to the central nervous system (e.g., epidurally, intracerebrally, intracerebroventricularly). In some embodiments, the local injection comprise administration to the skin (e.g., epicutaneously). In some embodiments, the one or more therapeutic agents are formulated in a transdermal patch, wherein the one or more therapeutic agents are delivered to the skin of the subject. In some embodiments, the one or more therapeutic agents is delivered sublingually and/or bucally, extra-amniotically, nasally, intra-arterially, intra-articularly, intravavernously, intracardiacally, intradermally, intralesionally, intramuscularly, intraocularly, intraosseously, intraperitoneally, intrathecally, intrauterinely, intravaginally, intravenously, intravesically, intravitreally, subcutaneously, trans-dermally, perivascularly, transmucosally, or through another route of administration. In some embodiments, the one or more therapeutic agents is delivered topically.

In some embodiments, the one or more therapeutic agents is delivered to the subject using a targeted delivery approach (e.g., for targeted delivery to the tumor site) or using a delivery approach to increase uptake of a cell of the one or more therapeutic agents. In some embodiments, the delivery approach comprises magnetic drug delivery (e.g., magnetic nanoparticle-based drug delivery), an acoustic targeted drug delivery approach, a self-microemulsifying drug delivery system, or other delivery approach.

### Pharmaceutical Compositions

In some embodiments, the disclosure provides a pharmaceutical composition for treating a cancer (e.g., liver or ovarian cancer), comprising (i) one or more therapeutic agents and (ii) a pharmaceutically acceptable carrier. In some embodiments, the one or more therapeutic agents is present in an amount that is effective to alter (e.g., increase or decrease) expression level and/or activity of PKMYT1 following administration or exposure to the subject. In some embodiments, the pharmaceutically acceptable carrier stabilizes the one or more therapeutic agents or provides therapeutic enhancement of the one or more therapeutic agents following administration to the subject as compared to the one or more therapeutic agents administered in the absence of the pharmaceutically acceptable carrier.

In some embodiments, pharmaceutically acceptable carrier comprises a substance, which substance may be used to confer a property to the one or more therapeutic agents used to alter (e.g., increase or decrease) the expression level and/or activity of PKMYT1. For example, in some embodiments, the pharmaceutically acceptable carrier comprises a substance for stabilization of the one or more therapeutic agents. In some embodiments, the pharmaceutically acceptable carrier comprises a substance for bulking up a solid, liquid, or gel formulation of the one or more therapeutic agents. In some embodiments, the substance confers a therapeutic enhancement to the one or more therapeutic agents (e.g., by enhancing solubility). In some embodiments, the substance is used to alter a property of the pharmaceutical composition, such as the viscosity. In some embodiments, the substance is used to alter a property of the one or more therapeutic agent, e.g., bioavailability, absorption, hydrophilicity, hydrophobicity, pharmacokinetics, etc.

In some embodiments, the pharmaceutically acceptable carrier comprises a binding agent, anti-adherent agent, a coating, a disintegrant, a glidant (e.g., silica gel, talc, magnesium carbonate), a lubricant, a preservative, a sorbent, a sweetener, a vehicle, or a combination thereof. For example, in some embodiments, the pharmaceutically acceptable carrier comprises a powder, a mineral, a metal, a sugar (e.g. saccharide or polysaccharide), a sugar alcohol, a naturally occurring polymer (e.g., cellulose, methylcellulose) synthetic polymer (e.g., polyethylene glycol or polyvinylpyrrolidone), an alcohol, a thickening agent, a starch, a macromolecule (e.g., lipid, protein, carbohydrate, nucleic acid molecule), etc.

In some embodiments, the one or more therapeutic agents is formulated into an aerosol, pill, tablet, capsule (e.g., asymmetric membrane capsule), pastille, elixir, emulsion, powder, solution, suspension, tincture, liquid, gel, dry powder, vapor, droplet, ointment, patch, or a combination thereof. In some embodiments, the one or more therapeutic agents is formulated in a gel or polymer and delivered via a thin film.

In some embodiments, the one or more therapeutic agents is formulated for targeted delivery or for increased uptake by a cell. For example, in some embodiments, the one or more therapeutic agents is formulated with another agent, which may improve the solubility, hydrophobicity, hydrophilicity, absorbability, half-life, bioavailability, release profile, or other property of the one or more therapeutic agents. For example, in some embodiments, the one or more therapeutic agents is formulated with a polymer which enables a controlled release profile (e.g., slow release). In some embodiments, the one or more therapeutic agents is formulated as a coating or with a coating (e.g., bovine submaxillary mucin coatings, polymer coatings, etc.) to alter a property of the one or more therapeutic agents (e.g., bioavailability, pharmacokinetics, etc.).

In some embodiments, the one or more therapeutic agents is formulated using a retro-metabolic drug design. In such embodiments, the one or more therapeutic agents is assessed for metabolic effects in a cell, and a new formulation comprising a derivative (e.g., chemically synthesized alternative or engineered variant) is prepared to change a property of the one or more therapeutic agents (e.g., to increase efficacy, minimize undesirable side effects, alter bioavailability, etc.).

### Kits

In some embodiments, the disclosure provides a kit comprising one or more therapeutic agent described herein. In some embodiments, the kit further comprises a package insert comprising instructions for using the one or more therapeutic agents described herein for treating or delaying progression of cancer in a subject. In some embodiments, the kit further comprises a package insert comprising instructions for using the one or more therapeutic agents described herein for treating or delaying progression of cancer in a subject, wherein the cancer has altered (e.g., increased or decreased) expression level and/or activity of a biomarker described herein. In some embodiments, the kit further comprises a package insert comprising instructions for using the one or more therapeutic agents described herein for treating or delaying progression of cancer in a subject, wherein the cancer comprises a mutation in a biomarker described herein (e.g., loss of function mutation resulting a decreased expression level and/or activity of the biomarker). In some embodiments, the kit further comprises materials desirable from a commercial and user standpoint, such as other buffers, diluents, filters, needles, and syringes. Suitable containers for the one or more therapeutic agent include, for example, bottles, vials, bags and syringes.

In some embodiments, the disclosure provides a kit comprising one or more therapeutic agents described herein for altering the expression level and/or activity of PKMYT1, and a package insert comprising instructions for treating or delaying progression of cancer in a subject, wherein the cancer has altered (e.g., increased or decreased) expression level and/or activity of a biomarker described herein. In some embodiments, the disclosure provides a kit comprising one or more therapeutic agents described herein for inhibiting PKMYT1, and a package insert comprising instructions for treating or delaying progression of cancer in a subject, wherein the cancer has a decreased expression level and/or activity of a biomarker described herein.

In some embodiments, the disclosure provides a kit comprising one or more therapeutic agents described herein for altering the expression level and/or activity of PKMYT1, and a package insert comprising instructions for treating or delaying progression of cancer in a subject, wherein the cancer comprises a mutation in a biomarker described herein (e.g., loss of function mutation resulting a decreased expression level and/or activity of the biomarker). In some embodiments, the disclosure provides a kit comprising one or more therapeutic agents described herein for inhibiting PKMYT1, and a package insert comprising instructions for treating or delaying progression of cancer in a subject, wherein the cancer comprises a mutation in a biomarker described herein (e.g., loss of function mutation resulting a decreased expression level and/or activity of the biomarker).

In some embodiments, the disclosure provides a kit comprising one or more therapeutic agents described herein for altering the expression level and/or activity of PKMYT1, and a package insert comprising instructions for treating or delaying progression of cancer in a subject, wherein the cancer has a loss of function or an inactivating mutation in a biomarker described herein. In some embodiments, the disclosure provides a kit comprising one or more therapeutic agents described herein for inhibiting PKMYT1, and a package insert comprising instructions for treating or delaying progression of cancer in a subject, wherein the cancer has a loss of function or an inactivating mutation in a biomarker described herein.

In some embodiments, the disclosure provides a kit comprising one or more therapeutic agents described herein for altering the expression level and/or activity of PKMYT1, and a package insert comprising instructions for treating or delaying progression of cancer in a subject, wherein the cancer has an altered (e.g., increased or decreased) expression level and/or activity of any one or any combination of biomarkers listed in Table 3. In some embodiments, the disclosure provides a kit comprising one or more therapeutic agents described herein for inhibiting PKMYT1, and a package insert comprising instructions for treating or delaying progression of cancer in a subject, wherein the cancer has a decreased expression level and/or activity of any one or any combination of biomarkers listed in Table 3.

In some embodiments, the disclosure provides a kit comprising one or more therapeutic agents described herein for altering the expression level and/or activity of PKMYT1, and a package insert comprising instructions for treating or delaying progression of cancer in a subject, wherein the cancer has one or more mutations in any one or any combination of biomarkers listed in Table 3. In some embodiments, the disclosure provides a kit comprising one or more therapeutic agents described herein for inhibiting PKMYT1, and a package insert comprising instructions for treating or delaying progression of cancer in a subject, wherein the cancer has one or more mutations in any one or any combination of biomarkers listed in Table 3.

In some embodiments, the disclosure provides a kit comprising one or more therapeutic agents described herein for altering the expression level and/or activity of PKMYT1, and a package insert comprising instructions for treating or delaying progression of cancer in a subject, wherein the cancer has a loss of function or an inactivating mutation in any one or any combination of biomarkers listed in Table 3. In some embodiments, the disclosure provides a kit comprising one or more therapeutic agents described herein for inhibiting PKMYT1, and a package insert comprising instructions for treating or delaying progression of cancer in a subject, wherein the cancer has a loss of function or an inactivating mutation in any one or any combination of biomarkers listed in Table 3.

In some embodiments, the disclosure provides a kit comprising one or more therapeutic agents described herein for altering the expression level and/or activity of PKMYT1, and a package insert comprising instructions for reducing tumor burden in a subject, wherein the cancer has altered (e.g., increased or decreased) expression level and/or activity of a biomarker described herein. In some embodiments, the disclosure provides a kit comprising one or more therapeutic agents described herein for inhibiting PKMYT1, and a package insert comprising instructions for reducing tumor burden in a subject, wherein the cancer has a decreased expression level and/or activity of a biomarker described herein.

In some embodiments, the disclosure provides a kit comprising one or more therapeutic agents described herein for altering the expression level and/or activity of PKMYT1, and a package insert comprising instructions for reducing tumor burden in a subject, wherein the cancer comprises a mutation in a biomarker described herein (e.g., loss of function mutation resulting a decreased expression level and/or activity of the biomarker). In some embodiments, the disclosure provides a kit comprising one or more therapeutic agents described herein for inhibiting PKMYT1, and a package insert comprising instructions for reducing tumor burden in a subject, wherein the cancer comprises a mutation in a biomarker described herein (e.g., loss of function mutation resulting a decreased expression level and/or activity of the biomarker).

In some embodiments, the disclosure provides a kit comprising one or more therapeutic agents described herein for altering the expression level and/or activity of PKMYT1, and a package insert comprising instructions for reducing tumor burden in a subject, wherein the cancer has a loss of function or an inactivating mutation in a biomarker described herein. In some embodiments, the disclosure provides a kit comprising one or more therapeutic agents described herein for inhibiting PKMYT1, and a package insert comprising instructions for reducing tumor burden in a subject, wherein the cancer has a loss of function or an inactivating mutation in a biomarker described herein.

In some embodiments, the disclosure provides a kit comprising one or more therapeutic agents described herein for altering the expression level and/or activity of PKMYT1, and a package insert comprising instructions for reducing tumor burden in a subject, wherein the cancer has altered (e.g., increased or decreased) expression level and/or activity of any one or any combination of biomarkers listed in Table 3. In some embodiments, the disclosure provides a kit comprising one or more therapeutic agents described herein for inhibiting PKMYT1, and a package insert comprising instructions for reducing tumor burden in a subject, wherein the cancer has a decreased expression level and/or activity of any one or any combination of biomarkers listed in Table 3.

In some embodiments, the disclosure provides a kit comprising one or more therapeutic agents described herein for altering the expression level and/or activity of PKMYT1, and a package insert comprising instructions for reducing tumor burden in a subject, wherein the cancer has one or more mutations in any one or any combination of biomarkers listed in Table 3. In some embodiments, the disclosure provides a kit comprising one or more therapeutic agents described herein for inhibiting PKMYT1, and a package insert comprising instructions for reducing tumor burden in a subject, wherein the cancer has one or more mutations in any one or any combination of biomarkers listed in Table 3.

In some embodiments, the disclosure provides a kit comprising one or more therapeutic agents described herein for altering the expression level and/or activity of PKMYT1, and a package insert comprising instructions for reducing tumor burden in a subject, wherein the cancer has a loss of function or an inactivating mutation in any one or any combination of biomarkers listed in Table 3. In some embodiments, the disclosure provides a kit comprising one or more therapeutic agents described herein for inhibiting PKMYT1, and a package insert comprising instructions for reducing tumor burden in a subject, wherein the cancer has a loss of function or an inactivating mutation in any one or any combination of biomarkers listed in Table 3.

### Definitions

While various embodiments of the invention have been shown and described herein, it will be obvious to those skilled in the art that such embodiments are provided by way of example only. Numerous variations, changes, and substitutions may occur to those skilled in the art without departing from the invention. It will be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “subject,” as used herein, generally refers to an animal, such as a mammal (e.g., human), reptile, or avian (e.g., bird), or other organism, such as a plant. For example, the subject can be a vertebrate, a mammal, a rodent (e.g., a mouse), a primate, a simian or a human. The subject can be a healthy individual, an individual that is asymptomatic with respect to a disease (e.g., liver or ovarian cancer), an individual that has or is suspected of having the disease (e.g., liver or ovarian cancer) or a pre-disposition to the disease, or an individual that is symptomatic with respect to the disease. The subject may be in need of therapy. The subject can be a patient undergoing monitoring or treatment by a healthcare provider, such as a treating physician.

As used herein, the term “patient” refers to a human subject having a disease or condition in need of treatment. In some embodiments, a patient to be treated or tested for responsiveness to a treatment according to the methods described herein is one who has been diagnosed with a cancer, such as any cancer described herein. Diagnosis may be performed by any method or technique known in the art, such as x-ray, MRI, or biopsy, and may also be confirmed by a physician. To minimize exposure of a patient to drug treatments that may not be therapeutic, the patient may be determined to be either responsive or non-responsive to a cancer treatment, such as a PKMYT1 therapeutic agent described herein, according to the methods described herein prior to treatment.

The term “genome,” as used herein, generally refers to genomic information from a subject, which may be, for example, at least a portion or an entirety of a subject's hereditary information. A genome can be encoded in a deoxyribonucleic acid (DNA) molecule (s) and may be expressed in a ribonucleic acid (RNA) molecule(s). A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The term “contacting” as used herein means establishing a physical connection between two or more entities. Methods of contacting cells with external entities both in vivo, in vitro, and ex vivo are well known in the biological arts. In exemplary embodiments of the disclosure, the step of contacting a mammalian cell with a composition (e.g., a composition comprising a therapeutic agent described herein) is performed in vivo. For example, contacting a composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a composition comprising a therapeutic agent described herein) and a cell may be contacted, for example, by adding the composition to the culture medium of the cell and may involve or result in transfection. Moreover, more than one cell may be contacted by the composition.

As used herein the terms “cancer” and “cancerous” refer to or describe the physiological condition in mammals (e.g., humans) that is typically characterized by unregulated cell proliferation. Examples of cancer include, but are not limited to, brain cancer (e.g., astrocytoma, glioblastoma multiforme, and craniopharyngioma), metastatic cancer (e.g., breast cancer that has metastasized to the brain), breast cancer (e.g., an estrogen receptor-positive (ERpos) breast cancer or a metastatic form of breast cancer), prostate cancer, ovarian cancer (e.g., ovarian adenocarcinoma or embryonal carcinoma), liver cancer (e.g., hepatocellular carcinoma (HCC) or hepatoma), myeloma (e.g., multiple myeloma), colorectal cancer (e.g., colon cancer and rectal cancer), leukemia (e.g., acute myeloid leukemia, acute lymphoid leukemia, chronic myeloid leukemia, chronic lymphocytic leukemia, acute myeloblastic leukemia, acute promyelocytic leukemia, acute myelomonocytic leukemia, acute monocytic leukemia, acute erythroleukemia, and chronic leukemia), myelodysplastic syndrome, lymphoma (e.g., diffuse large B-cell lymphoma, cutaneous T-cell lymphoma, peripheral T-cell lymphoma, Hodgkin's lymphoma, non-Hodgkin's lymphoma, Waldenstrom's macroglobulinemia, and lymphocytic lymphoma), cervical cancer, esophageal cancer, melanoma, glioma (e.g., oligodendroglioma), pancreatic cancer (e.g., adenosquamous carcinoma, signet ring cell carcinoma, hepatoid carcinoma, colloid carcinoma, islet cell carcinoma, and pancreatic neuroendocrine carcinoma), gastrointestinal stromal tumor, sarcoma (e.g., fibrosarcoma, myxosarcoma, liposarcoma, chondrosarcoma, osteogenic sarcoma, angiosarcoma, endotheliosarcoma, lymphangiosarcoma, lymphangioendotheliosarcoma, leiomyosarcoma, Ewing's sarcoma, and rhabdomyosarcoma), breast cancer (e.g., medullary carcinoma), bladder cancer, head and neck cancer (e.g., squamous cell carcinoma of the head and neck), lung cancer (e.g., non-small cell lung carcinoma, large cell carcinoma, bronchogenic carcinoma, and papillary adenocarcinoma), oral cavity cancer, uterine cancer, testicular cancer (e.g., seminoma and embryonal carcinoma), skin cancer (e.g., squamous cell carcinoma and basal cell carcinoma), thyroid cancer (e.g., papillary carcinoma and medullary carcinoma), stomach cancer, intra-epithelial cancer, bone cancer, biliary tract cancer, eye cancer, larynx cancer, kidney cancer (e.g., renal cell carcinoma and Wilms tumor), gastric cancer, blastoma (e.g., nephroblastoma, medulloblastoma, hemangioblastoma, neuroblastoma, and retinoblastoma), polycythemia vera, chordoma, synovioma, mesothelioma, adenocarcinoma, sweat gland carcinoma, sebaceous gland carcinoma, cystadenocarcinoma, bile duct carcinoma, choriocarcinoma, epithelial carcinoma, ependymoma, pinealoma, acoustic neuroma, schwannoma, meningioma, pituitary adenoma, nerve sheath tumor, cancer of the small intestine, cancer of the endocrine system, cancer of the penis, cancer of the urethra, cutaneous or intraocular melanoma, a gynecologic tumor, solid tumors of childhood, and neoplasms of the central nervous system. The term cancer includes solid tumors (e.g., breast cancer or brain cancer) and hematological cancers (e.g., cancer of the blood, such as lymphoma (e.g., diffuse large B-cell lymphoma (DLBCL), cutaneous T-cell lymphoma (CTCL), peripheral T-cell lymphoma (PTCL), and Hodgkin's lymphoma)).

Whenever a gene is referred to herein, it will be understood that a single gene can be referred to by different names. For example, “protein kinase, membrane associated tyrosine/threonine 1” and “membrane-associated tyrosine- and threonine-specific cdc2-inhibitory kinase” both refer to the same gene, PKMYT1. As another example, “protein phosphatase 2 regulatory subunit B alpha” and “serine/threonine-protein phosphatase 2A 55 kDa regulatory subunit B alpha isoform” both refer to the same gene, PPP2R2A.

### Other Embodiments

The disclosure relates to the following embodiments. Throughout this section, the term embodiment is abbreviated as “E” followed by an ordinal. For example, EA-1 is equivalent to Embodiment A-1.
Embodiment 1. A method of identifying a subject having a disease or disorder for treatment with one or more PKMYT1 therapeutic agents, the method comprising determining the expression level and/or activity of one or more biomarkers in a diseased tissue sample obtained from the subject, wherein the one or more biomarkers is selected from any one or a combination of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.
Embodiment 2. A method of determining responsiveness of a subject having a disease or disorder to one or more PKMYT1 therapeutic agents, the method comprising determining the expression level and/or activity of one or more biomarkers in a diseased tissue sample obtained from the subject, wherein the one or more biomarkers is selected from any one or a combination of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.
Embodiment 3. The method of E-1 or E-2, wherein the diseased tissue sample comprises an altered expression level and/or activity of the one or more biomarkers relative to a reference tissue sample.
Embodiment 4. The method of any one of E-1 to E-3, wherein the expression level and/or activity of the one or more biomarkers is reduced relative to a reference tissue sample.
Embodiment 5. The method of any one of E-1 to E-4, wherein the diseased tissue comprises a mutation in the one or more biomarkers.
Embodiment 6. The method of E-5, wherein the mutation is a deletion.
Embodiment 7. The method of E-5 or E-6, wherein the mutation is detected by sequencing genomic DNA in the diseased tissue sample, optionally via next generation sequencing.
Embodiment 8. The method of any one of E-1 to E-7, wherein the subject has a tumor, and wherein the diseased tissue sample comprises a tumor sample, a circulating tumor DNA sample, a tumor biopsy sample, or a fixed tumor sample.
Embodiment 9. The method of E-8, wherein the tumor comprises a plurality of tumor cells comprising the mutation.
Embodiment 10. The method of any one of E-1 to E-9, wherein the one or more biomarkers comprises 2, 3, 4, or 5 biomarkers selected from ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment 11. The method of E-10, wherein the PP2 subunit is PPP2R1B.
Embodiment 12. The method of E-10 or E-11, wherein the PP2 subunit is PPP2R2A.
Embodiment 13. The method of any one of E-1 to E-12, further comprising administering one or more PKMYT1 therapeutic agents to the subject.
Embodiment 14. The method of E-13, wherein the administering results in a reduced expression level and/or activity of PKMYT1 in a tumor of the subject.
Embodiment 15. The method of E-14, wherein the reduced expression level and/or activity of PKMYT1 induces synthetic lethality in the tumor.
Embodiment 16. The method of E-15, wherein the synthetic lethality promotes tumor regression.
Embodiment 17. A method of treating a cancer or promoting tumor regression in a subject having a tumor comprising an altered expression level and/or activity of one or more biomarkers, wherein the one or more biomarkers is selected from any one or a combination of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B, the method comprising: administering to the subject a therapeutically effective amount of one or more protein kinase, membrane associated tyrosine/threonine 1 (PKMYT1) therapeutic agents.
Embodiment 18. The method of E-17, wherein the tumor comprises a reduced expression level and/or activity of the one or more biomarkers as measured in a tumor sample obtained from the subject relative to a reference tissue sample.
Embodiment 19. A method of identifying a cancer subject to receive one or more PKMYT1 therapeutic agents, comprising
(i) determining the expression level and/or activity of one or more biomarkers in a tumor sample obtained from the subject, wherein the one or more biomarkers are selected from any one or a combination of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B; and
(ii) administering one or more PKMYT1 therapeutic agents to the subject based on an expression level and/or activity of the one or more biomarkers that is reduced relative to a healthy control.
Embodiment 20. The method of E-18 or E-19, wherein the tumor sample is a circulating tumor DNA sample, a tumor biopsy sample, or a fixed tumor sample.
Embodiment 21. The method of E-17 to E-20, wherein the tumor comprises a mutation in the one or more biomarkers.
Embodiment 22. The method of E-21, wherein the mutation is a deletion.
Embodiment 23. The method of E-21 or E-22, wherein the mutation is detected by sequencing genomic tumor DNA, optionally via next generation sequencing.
Embodiment 24. The method of any one of E-17 to E-23, wherein the tumor comprises a plurality of tumor cells comprising the mutation.
Embodiment 25. The method of any one of E-17 to E-24, wherein the one or more biomarkers comprises 2, 3, 4, or 5 biomarkers selected from ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment 26. The method of E-25, wherein the PP2 subunit is PPP2R1B.
Embodiment 27. The method of E-25 or E-26, wherein the PP2 subunit is PPP2R2A.
Embodiment 28. The method of any one of E-17 to E-27, wherein the administering results in a reduced expression level and/or activity of PKMYT1 in a tumor of the subject.
Embodiment 29. The method of E-28, wherein the reduced expression level and/or activity of PKMYT1 induces synthetic lethality in the tumor.
Embodiment 30. The method of E-29, wherein the synthetic lethality promotes tumor regression.
Embodiment 31. The method of any one of E-1 to E-30, wherein the one or more biomarkers comprises PPP2R1B.
Embodiment 32. The method of any one of E-1 to E-30, wherein the one or more biomarkers comprises PPP3CC.
Embodiment 33. The method of any one of E-1 to E-30, wherein the one or more biomarkers comprises ATM.
Embodiment 34. The method of any one of E-1 to E-30, wherein the one or more biomarkers comprises CACNA1H.
Embodiment 35. The method of any one of E-1 to E-30, wherein the one or more biomarkers comprises CDC25A.
Embodiment 36. The method of any one of E-1 to E-30, wherein the one or more biomarkers comprises CDKN1B.
Embodiment 37. The method of any one of E-1 to E-30, wherein the one or more biomarkers comprises DUSP7.
Embodiment 38. The method of any one of E-1 to E-30, wherein the one or more biomarkers comprises FOXO3.
Embodiment 39. The method of any one of E-1 to E-30, wherein the one or more biomarkers comprises FZD3.
Embodiment 40. The method of any one of E-1 to E-30, wherein the one or more biomarkers comprises JAK1.
Embodiment 41. The method of any one of E-1 to E-30, wherein the one or more biomarkers comprises MAP2K4.
Embodiment 42. The method of any one of E-1 to E-30, wherein the one or more biomarkers comprises MAP3K2.
Embodiment 43. The method of any one of E-1 to E-30, wherein the one or more biomarkers comprises SMAD2.
Embodiment 44. The method of any one of E-1 to E-30, wherein the one or more biomarkers comprises TGFBR2.
Embodiment 45. The method of any one of E-1 to E-30, wherein the one or more biomarkers comprises TP53.
Embodiment 46. The method of any one of E-1 to E-45, wherein the one or more PKMYT1 therapeutic agents is selected from a small molecule, a peptide, a protein, and a nucleic acid.
Embodiment 47. The method of E-46, wherein the one or more PKMYT1 therapeutic agents comprises an anti-PKMYT1 antibody or fragment thereof.
Embodiment 48. The method of E-46, wherein the one or more PKMYT1 therapeutic agents comprises an anti-PKMYT1 intrabody or fragment thereof.
Embodiment 49. The method of E-46, wherein the one or more PKMYT1 therapeutic agents comprises an RNAi molecule or an aptamer.
Embodiment 50. The method of E-46, wherein the one or more PKMYT1 therapeutic agents comprises a small molecule inhibitor.
Embodiment 51. The method of E-50, wherein the small molecule inhibitor is 5-((5-methoxy-2-((4-morpholinophenyl)amino)pyrimidin-4-yl)amino)-2-methylphenol, iV-(2-chloro-6-methylphenyl)-2-((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), A-(5-chlorobenzo[t/][1,3]dioxol-4-yl)-7-(2-(4-methylpiperazin-1-yl)ethoxy)-5-((tetrahydro-2//-pyran-4-yl)oxy)quinazolin-4-amine (saracatinib), (£)-A-(4-((3-chloro-4-fluorophenyl)amino)-3-cyano-7-ethoxyquinolin-6-yl)-4-(dimethylamino)but-2-enamide(pelitinib), A-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-<i]pyrimidin-7(8//)-one (PD-0166285), 6-(2,6-dichlorophenyl)-8-methyl-2-((4-morpholinophenyl)amino)pyrido[2,3-cf]pyrimidin-7(8//)-one (PD-173952), 6-(2,6-dichlorophenyl)-8-methyl-2-((3-(methylthio)phenyl)amino)pyrido[2,3-r/Jpyrimidin-7(8//)-one (PD-173955), or 6-(2,6-dichlorophenyl)-2-((4-fluoro-3-methylphenyl)amino)-8-methylpyrido[2,3-«i]pyrimidin-7(8//)-one (PD-180970).
Embodiment 52. The method of E-46, wherein the one or more PKMYT1 therapeutic agents comprises a gene editing technology for introducing a genetic knockout of the PKMYT1 gene.
Embodiment 53. The method of E-52, wherein the gene editing technology comprises CRISPR/Cas9.
Embodiment 54. The method of any one of E-8 to E-53, wherein the tumor is selected from acute myeloid leukemia (LAML), adrenocortical carcinoma (ACC), bladder urothelial carcinoma (BLCA), brain lower grade glioma (LGG), breast invasive carcinoma (BRCA), cervical squamous cell carcinoma and endocervical adenocarcinoma (CESC), cholangiocarcinoma (CHOL), chronic myelogenous leukemia (LCML), colon adenocarcinoma (COAD), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), lymphoid neoplasm diffuse large B-cell lymphoma (DLBC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), testicular germ cell tumors (TGCT), thymoma (THYM), thyroid carcinoma (THCA), uterine carcinosarcoma (UCS), uterine corpus endometrial carcinoma (UCEC), and uveal melanoma (UVM).
Embodiment 55. Use of one or more PKMYT1 therapeutic agents for treating a cancer or promoting tumor regression in a subject, wherein the subject has been identified based on an altered expression level and/or activity of one or more biomarkers, wherein the one or more biomarkers is selected from any one or a combination of ATM, MAP2K4, TP53, CDC25A, CACNA1H, CDKN1B, DUSP7, FOXO3, FZD3, JAK1, SMAD2, TGFBR2, MAP3K2, PPP2R1B and PPP3CC.
Embodiment 56. Use of one or more PKMYT1 therapeutic agents in the manufacture of a medicament for treating a cancer or promoting tumor regression in a subject, wherein the subject has been identified based on an altered expression level and/or activity of one or more biomarkers, wherein the one or more biomarkers is selected from any one or a combination of ATM, MAP2K4, TP53, CDC25A, CACNA1H, CDKN1B, DUSP7, FOXO3, FZD3, JAK1, SMAD2, TGFBR2, MAP3K2, PPP3CC, and PPP2R1B.
Embodiment 57. A kit comprising a PKMYT1 therapeutic agent, and a package insert comprising instructions for administering the PKMYT1 therapeutic agent to a subject having a cancer comprising an altered expression level and/or activity of one or more biomarkers, wherein the one or more biomarkers is selected from any one or a combination of ATM, MAP2K4, TP53, CDC25A, CACNA1H, CDKN1B, DUSP7, FOXO3, FZD3, JAK1, SMAD2, TGFBR2, MAP3K2, PPP2R1B, and PPP3CC.
Embodiment A-1. A method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.
Embodiment A-2. A method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.
Embodiment A-3. The method of EA-1 or EA-2, wherein the diseased tissue sample comprises an altered expression level and/or activity of the one or more biomarkers relative to a reference tissue sample.
Embodiment A-4. The method of EA-3, wherein the expression level and/or activity of the one or more biomarkers is reduced relative to a reference tissue sample.
Embodiment A-5. The method of any one of claims 1-4, wherein the diseased tissue sample comprises a mutation in the one or more biomarkers relative to a reference tissue sample.
Embodiment A-6. The method of EA-5, wherein the mutation is a loss of function mutation, optionally wherein the loss of function mutation is a deletion of the gene encoding the biomarker.
Embodiment A-7. The method of EA-5 or EA-6, wherein the mutation is detected by sequencing genomic DNA in the diseased tissue sample, optionally via next generation sequencing.
Embodiment A-8. The method of any one of EA-1 to EA-7, wherein the subject has a tumor, and wherein the diseased tissue sample comprises a tumor sample, a circulating tumor DNA sample, a tumor biopsy sample, or a fixed tumor sample.
Embodiment A-9. The method of EA-8, wherein the tumor comprises a plurality of tumor cells comprising the mutation.
Embodiment A-10. The method of any one of EA-1 to EA-9, wherein the one or more biomarkers comprises 2, 3, 4, 5, or more biomarkers selected from BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, CDC16, ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment A-11. The method of EA-10, wherein the PP2 subunit is PPP2R1B.
Embodiment A-12. The method of EA-10 or EA-11, wherein the PP2 subunit is PPP2R2A.
Embodiment A-13. The method of any one of EA-1 to EA-12, further comprising administering one or more PKMYT1 therapeutic agents to the subject.
Embodiment A-14. The method of EA-13, wherein the administering results in a reduced expression level and/or activity of PKMYT1 in a tumor of the subject.
Embodiment A-15. The method of EA-14, wherein the reduced expression level and/or activity of PKMYT1 induces synthetic lethality in the tumor.
Embodiment A-16. The method of EA-15, wherein the synthetic lethality promotes tumor regression.
Embodiment A-17. A method of treating a cancer or promoting tumor regression in a subject having a tumor comprising a mutation in, an altered expression level of, and/or an altered activity of one or more biomarkers, wherein the one or more biomarkers is selected from any one or any combination of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16, the method comprising: administering to the subject a therapeutically effective amount of one or more protein kinase, membrane associated tyrosine/threonine 1 (PKMYT1) therapeutic agents.
Embodiment A-18. The method of EA-17, wherein the tumor comprises a loss of function mutation in, a reduced expression level of, and/or a reduced activity of the one or more biomarkers as measured in a tumor sample obtained from the subject relative to a reference tissue sample.
Embodiment A-19. A method of identifying a cancer subject to receive one or more PKMYT1 therapeutic agents, comprising
(i) determining the presence of a mutation in, the expression level of, and/or the activity of one or more biomarkers in a tumor sample obtained from the subject, wherein the one or more biomarkers are selected from any one or any combination of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16; and
(ii) administering one or more PKMYT1 therapeutic agents to the subject based on presence of a mutation in, a reduced expression level of, and/or a reduced activity of the one or more biomarkers relative to a healthy control.
Embodiment A-20. The method of EA-18 or EA-19, wherein the tumor sample is a circulating tumor DNA sample, a tumor biopsy sample, or a fixed tumor sample.
Embodiment A-21. The method of any one of EA-18 to EA-20, wherein the tumor sample comprises a mutation in the one or more biomarkers.
Embodiment A-22. The method of EA-21, wherein the mutation is a loss of function mutation, optionally wherein the loss of function mutation is a deletion of the gene encoding the biomarker.
Embodiment A-23. The method of EA-21 or EA-22, wherein the mutation is detected by sequencing genomic tumor DNA, optionally via next generation sequencing.
Embodiment A-24. The method of any one of EA-18 to EA-23, wherein the tumor sample comprises a plurality of tumor cells comprising the mutation.
Embodiment A-25. The method of any one of EA-17 to EA-24, wherein the one or more biomarkers comprises 2, 3, 4, 5, or more biomarkers selected from BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, CDC16, ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment A-26. The method of EA-25, wherein the PP2 subunit is PPP2R1B.
Embodiment A-27. The method of EA-25 or EA-26, wherein the PP2 subunit is PPP2R2A.
Embodiment A-28. The method of any one of EA-17 to EA-27, wherein the administering results in a reduced expression level and/or activity of PKMYT1 in a tumor of the subject.
Embodiment A-29. The method of EA-28, wherein the reduced expression level and/or activity of PKMYT1 induces synthetic lethality in the tumor.
Embodiment A-30. The method of EA-29, wherein the synthetic lethality promotes tumor regression.
Embodiment A-31. The method of any one of EA-1 to EA-30, wherein the one or more PKMYT1 therapeutic agents is selected from a small molecule, a peptide, a protein, and a nucleic acid.
Embodiment A-32. The method of EA-31, wherein the one or more PKMYT1 therapeutic agents comprises an anti-PKMYT1 antibody or fragment thereof.
Embodiment A-33. The method of EA-31, wherein the one or more PKMYT1 therapeutic agents comprises an anti-PKMYT1 intrabody or fragment thereof.
Embodiment A-34. The method of EA-31, wherein the one or more PKMYT1 therapeutic agents comprises an RNAi molecule or an aptamer.
Embodiment A-35. The method of EA-31, wherein the one or more PKMYT1 therapeutic agents comprises a small molecule inhibitor.
Embodiment A-36. The method of EA-35, wherein the small molecule inhibitor is selected from 5-((5-methoxy-2-((4-morpholinophenyl)amino)pyrimidin-4-yl)amino)-2-methylphenol, iV-(2-chloro-6-methylphenyl)-2-((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), A-(5-chlorobenzo[t/][1,3]dioxol-4-yl)-7-(2-(4-methylpiperazin-1-yl)ethoxy)-5-((tetrahydro-2//-pyran-4-yl)oxy)quinazolin-4-amine (saracatinib), (£)-A-(4-((3-chloro-4-fluorophenyl)amino)-3-cyano-7-ethoxyquinolin-6-yl)-4-(dimethylamino)but-2-enamide(pelitinib), A-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-<i]pyrimidin-7(8//)-one (PD-0166285), 6-(2,6-dichlorophenyl)-8-methyl-2-((4-morpholinophenyl)amino)pyrido[2,3-cf]pyrimidin-7(8//)-one (PD-173952), 6-(2,6-dichlorophenyl)-8-methyl-2-((3-(methylthio)phenyl)amino)pyrido[2,3-r/Jpyrimidin-7(8//)-one (PD-173955), and 6-(2,6-dichlorophenyl)-2-((4-fluoro-3-methylphenyl)amino)-8-methylpyrido[2,3-«i]pyrimidin-7(8//)-one (PD-180970).
Embodiment A-37. The method of EA-31, wherein the one or more PKMYT1 therapeutic agents comprises a gene editing technology for introducing a genetic knockout of the PKMYT1 gene.
Embodiment A-38. The method of EA-37, wherein the gene editing technology comprises CRISPR/Cas9.
Embodiment A-39. The method of any one of EA-8 to EA-38, wherein the cancer is selected from: acute myeloid leukemia (LAML), adrenocortical carcinoma (ACC), bladder urothelial carcinoma (BLCA), brain lower grade glioma (LGG), breast invasive carcinoma (BRCA), cervical squamous cell carcinoma and endocervical adenocarcinoma (CESC), cholangiocarcinoma (CHOL), chronic myelogenous leukemia (LCML), colon adenocarcinoma (COAD), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), lymphoid neoplasm diffuse large B-cell lymphoma (DLBC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), testicular germ cell tumors (TGCT), thymoma (THYM), thyroid carcinoma (THCA), uterine carcinosarcoma (UCS), uterine corpus endometrial carcinoma (UCEC), and uveal melanoma (UVM).
Embodiment A-40. Use of one or more PKMYT1 therapeutic agents for treating a cancer or promoting tumor regression in a subject, wherein the subject has been identified based on the presence of a mutation in, an altered expression level and/or altered activity of one or more biomarkers, wherein the one or more biomarkers is selected from any one or any combination of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.
Embodiment A-41. Use of one or more PKMYT1 therapeutic agents in the manufacture of a medicament for treating a cancer or promoting tumor regression in a subject, wherein the subject has been identified based on the presence of a mutation in, an altered expression level and/or altered activity of one or more biomarkers, wherein the one or more biomarkers is selected from any one or any combination of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.
Embodiment A-42. A kit comprising a PKMYT1 therapeutic agent, and a package insert comprising instructions for administering the PKMYT1 therapeutic agent to a subject having a cancer comprising a mutation in, an altered expression level and/or altered activity of one or more biomarkers, wherein the one or more biomarkers is selected from any one or any combination of BIN3, AGPAT5, FGF17, PBK, NOTCH1, CNTN5, IRF2, ALPK2, CDH19, CHKB, MAPK12, SLC8A1, HDAC2, CDT1, ADCY2, SLK, CDC20B, RPS6KA3, STAG1, CKAP5, RAD51, CKS1B, CCNO, KCNA2, MCM4, PLK4, and CDC16.
Embodiment B-1. A method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.
Embodiment B-2. A method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.
Embodiment B-3. The method of EB-1 or EB-2, wherein the diseased tissue sample comprises an altered expression level and/or activity of the one or more biomarkers relative to a reference tissue sample.
Embodiment B-4. The method of any one of EB-1 to EB-3, wherein the expression level and/or activity of the one or more biomarkers is reduced relative to a reference tissue sample.
Embodiment B-5. The method of any one of EB-1 to EB-4, wherein the diseased tissue sample comprises a mutation in the one or more biomarkers relative to a reference tissue sample.
Embodiment B-6. The method of EB-5, wherein the mutation is a loss of function mutation, optionally wherein the loss of function mutation is a deletion of the gene encoding the biomarker.
Embodiment B-7. The method of EB-5 or EB-6, wherein the mutation is detected by sequencing genomic DNA in the diseased tissue sample, optionally via next generation sequencing.
Embodiment B-8. The method of any one of EB-1 to EB-7, wherein the subject has a tumor, and wherein the diseased tissue sample comprises a tumor sample, a circulating tumor DNA sample, a tumor biopsy sample, or a fixed tumor sample.
Embodiment B-9. The method of EB-8, wherein the tumor comprises a plurality of tumor cells comprising the mutation.
Embodiment B-10. The method of any one of EB-1 to EB-9, wherein the one or more biomarkers comprises 2, 3, 4, 5, or more biomarkers selected from ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, SARAF, ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment B-11. The method of EB-10, wherein the PP2 subunit is PPP2R1B.
Embodiment B-12. The method of EB-10 or EB-11, wherein the PP2 subunit is PPP2R2A.
Embodiment B-13. The method of any one of claims EB-1 to EB-12, further comprising administering one or more PKMYT1 therapeutic agents to the subject.
Embodiment B-14. The method of EB-13, wherein the administering results in a reduced expression level and/or activity of PKMYT1 in a tumor of the subject.
Embodiment B-15. The method of EB-14, wherein the reduced expression level and/or activity of PKMYT1 induces synthetic lethality in the tumor.
Embodiment B-16. The method of EB-15, wherein the synthetic lethality promotes tumor regression.
Embodiment B-17. A method of treating a cancer or promoting tumor regression in a subject having a tumor comprising a mutation in, an altered expression level of, and/or an altered activity of one or more biomarkers, wherein the one or more biomarkers is selected from any one or any combination of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF, the method comprising: administering to the subject a therapeutically effective amount of one or more protein kinase, membrane associated tyrosine/threonine 1 (PKMYT1) therapeutic agents.
Embodiment B-18. The method of EB-17, wherein the tumor comprises a loss of function mutation in, a reduced expression level of, and/or a reduced activity of the one or more biomarkers as measured in a tumor sample obtained from the subject relative to a reference tissue sample.
Embodiment B-19. A method of identifying a cancer subject to receive one or more PKMYT1 therapeutic agents, comprising
(i) determining the presence of a mutation in, the expression level of, and/or the activity of one or more biomarkers in a tumor sample obtained from the subject, wherein the one or more biomarkers are selected from any one or any combination of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF; and
(ii) administering one or more PKMYT1 therapeutic agents to the subject based on presence of a mutation in, a reduced expression level of, and/or a reduced activity of the one or more biomarkers relative to a healthy control.
Embodiment B-20. The method of EB-18 or EB-19, wherein the tumor sample is a circulating tumor DNA sample, a tumor biopsy sample, or a fixed tumor sample.
Embodiment B-21. The method of any one of EB-18 to EB-20, wherein the tumor sample comprises a mutation in the one or more biomarkers.
Embodiment B-22. The method of EB-21, wherein the mutation is a loss of function mutation, optionally wherein the loss of function mutation is a deletion of the gene encoding the biomarker.
Embodiment B-23. The method of EB-21 or EB-22, wherein the mutation is detected by sequencing genomic tumor DNA, optionally via next generation sequencing.
Embodiment B-24. The method of any one of EB-18 to EB-23, wherein the tumor sample comprises a plurality of tumor cells comprising the mutation.
Embodiment B-25. The method of any one of claims EB-17 to EB-24, wherein the one or more biomarkers comprises 2, 3, 4, 5, or more biomarkers selected from ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, SARAF, ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment B-26. The method of EB-25, wherein the PP2 subunit is PPP2R1B.
Embodiment B-27. The method of EB-25 or EB-26, wherein the PP2 subunit is PPP2R2A.
Embodiment B-28. The method of any one of claims B-17 to B-27, wherein the administering results in a reduced expression level and/or activity of PKMYT1 in a tumor of the subject.
Embodiment B-29. The method of EB-28, wherein the reduced expression level and/or activity of PKMYT1 induces synthetic lethality in the tumor.
Embodiment B-30. The method of EB-29, wherein the synthetic lethality promotes tumor regression.
Embodiment B-31. The method of any one of claims 1-30, wherein the one or more PKMYT1 therapeutic agents is selected from a small molecule, a peptide, a protein, and a nucleic acid.
Embodiment B-32. The method of EB-31, wherein the one or more PKMYT1 therapeutic agents comprises an anti-PKMYT1 antibody or fragment thereof.
Embodiment B-33. The method of EB-31, wherein the one or more PKMYT1 therapeutic agents comprises an anti-PKMYT1 intrabody or fragment thereof.
Embodiment B-34. The method of EB-31, wherein the one or more PKMYT1 therapeutic agents comprises an RNAi molecule or an aptamer.
Embodiment B-35. The method of EB-31, wherein the one or more PKMYT1 therapeutic agents comprises a small molecule inhibitor.
Embodiment B-36. The method of EB-35, wherein the small molecule inhibitor is selected from 5-((5-methoxy-2-((4-morpholinophenyl)amino)pyrimidin-4-yl)amino)-2-methylphenol, iV-(2-chloro-6-methylphenyl)-2-((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), A-(5-chlorobenzo[t/][1,3]dioxol-4-yl)-7-(2-(4-methylpiperazin-1-yl)ethoxy)-5-((tetrahydro-2//-pyran-4-yl)oxy)quinazolin-4-amine (saracatinib), (£)-A-(4-((3-chloro-4-fluorophenyl)amino)-3-cyano-7-ethoxyquinolin-6-yl)-4-(dimethylamino)but-2-enamide(pelitinib), A-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-<i]pyrimidin-7(8//)-one (PD-0166285), 6-(2,6-dichlorophenyl)-8-methyl-2-((4-morpholinophenyl)amino)pyrido[2,3-cf]pyrimidin-7(8//)-one (PD-173952), 6-(2,6-dichlorophenyl)-8-methyl-2-((3-(methylthio)phenyl)amino)pyrido[2,3-r/Jpyrimidin-7(8//)-one (PD-173955), and 6-(2,6-dichlorophenyl)-2-((4-fluoro-3-methylphenyl)amino)-8-methylpyrido[2,3-«i]pyrimidin-7(8//)-one (PD-180970).
Embodiment B-37. The method of EB-31, wherein the one or more PKMYT1 therapeutic agents comprises a gene editing technology for introducing a genetic knockout of the PKMYT1 gene.
Embodiment B-38. The method of EB-37, wherein the gene editing technology comprises CRISPR/Cas9.
Embodiment B-39. The method of any one of EB-8 to EB-38, wherein the cancer is selected from: acute myeloid leukemia (LAML), adrenocortical carcinoma (ACC), bladder urothelial carcinoma (BLCA), brain lower grade glioma (LGG), breast invasive carcinoma (BRCA), cervical squamous cell carcinoma and endocervical adenocarcinoma (CESC), cholangiocarcinoma (CHOL), chronic myelogenous leukemia (LCML), colon adenocarcinoma (COAD), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), lymphoid neoplasm diffuse large B-cell lymphoma (DLBC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), testicular germ cell tumors (TGCT), thymoma (THYM), thyroid carcinoma (THCA), uterine carcinosarcoma (UCS), uterine corpus endometrial carcinoma (UCEC), and uveal melanoma (UVM).
Embodiment B-40. Use of one or more PKMYT1 therapeutic agents for treating a cancer or promoting tumor regression in a subject, wherein the subject has been identified based on the presence of a mutation in, an altered expression level and/or altered activity of one or more biomarkers, wherein the one or more biomarkers is selected from any one or any combination of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.
Embodiment B-41. Use of one or more PKMYT1 therapeutic agents in the manufacture of a medicament for treating a cancer or promoting tumor regression in a subject, wherein the subject has been identified based on the presence of a mutation in, an altered expression level and/or altered activity of one or more biomarkers, wherein the one or more biomarkers is selected from any one or any combination of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.
Embodiment B-42. A kit comprising a PKMYT1 therapeutic agent, and a package insert comprising instructions for administering the PKMYT1 therapeutic agent to a subject having a cancer comprising a mutation in, an altered expression level and/or altered activity of one or more biomarkers, wherein the one or more biomarkers is selected from any one or any combination of ERICH1, TNKS, TDRP, MTUS1, TNFRSF10B, HR, TNFRSF10D, DMTN, ENTPD4, TNFRSF10C, PEBP4, LPL, LGI3, SLC7A2, MTMR9, MSRA, PDLIM2, INTS10, SH2D4A, GFRA2, ZDHHC2, PDGFRL, SPAG11B, PPP1R3B, SPAG11A, REEP4, DEFA5, DEFB136, NRG1, ASAH1, DEFA3, EPHX2, CNOT7, PNMA2, TRIM35, ATRX, INTS9, DNAH3, MAP3K1, RIMS2, NSD1, and SARAF.
Embodiment C-1. A method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.
Embodiment C-2. A method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.
Embodiment C-3. The method of EC-1 or EC-2, wherein the diseased tissue sample comprises an altered expression level and/or activity of the one or more biomarkers relative to a reference tissue sample.
Embodiment C-4. The method of EC-3, wherein the expression level and/or activity of the one or more biomarkers is reduced relative to a reference tissue sample.
Embodiment C-5. The method of any one of EC-1 to EC-4, wherein the diseased tissue sample comprises a mutation in the one or more biomarkers relative to a reference tissue sample.
Embodiment C-6. The method of EC-5, wherein the mutation is a loss of function mutation, optionally wherein the loss of function mutation is a deletion of the gene encoding the biomarker.
Embodiment C-7. The method of EC-5 or EC-6, wherein the mutation is detected by sequencing genomic DNA in the diseased tissue sample, optionally via next generation sequencing.
Embodiment C-8. The method of any one of EC-1 to EC-7, wherein the subject has a tumor, and wherein the diseased tissue sample comprises a tumor sample, a circulating tumor DNA sample, a tumor biopsy sample, or a fixed tumor sample.
Embodiment C-9. The method of EC-8, wherein the tumor comprises a plurality of tumor cells comprising the mutation.
Embodiment C-10. The method of any one of EC-1 to EC-9, wherein the one or more biomarkers comprises 2, 3, 4, 5 or more biomarkers selected from CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, NFIB, ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment C-11. The method of EC-10, wherein the PP2 subunit is PPP2R1B.
Embodiment C-12. The method of EC-10 or EC-11, wherein the PP2 subunit is PPP2R2A.
Embodiment C-13. The method of any one of EC-1 to EC-12, further comprising administering one or more PKMYT1 therapeutic agents to the subject.
Embodiment C-14. The method of EC-13, wherein the administering results in a reduced expression level and/or activity of PKMYT1 in a tumor of the subject.
Embodiment C-15. The method of EC-14, wherein the reduced expression level and/or activity of PKMYT1 induces synthetic lethality in the tumor.
Embodiment C-16. The method of EC-15, wherein the synthetic lethality promotes tumor regression.
Embodiment C-17. A method of treating a cancer or promoting tumor regression in a subject having a tumor comprising a mutation in, an altered expression level of, and/or an altered activity of one or more biomarkers, wherein the one or more biomarkers is selected from any one or any combination of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB, the method comprising: administering to the subject a therapeutically effective amount of one or more protein kinase, membrane associated tyrosine/threonine 1 (PKMYT1) therapeutic agents.
Embodiment C-18. The method of EC-17, wherein the tumor comprises a loss of function mutation in, a reduced expression level of, and/or a reduced activity of the one or more biomarkers as measured in a tumor sample obtained from the subject relative to a reference tissue sample.
Embodiment C-19. A method of identifying a cancer subject to receive one or more PKMYT1 therapeutic agents, comprising
(i) determining the presence of a mutation in, the expression level of, and/or the activity of one or more biomarkers in a tumor sample obtained from the subject, wherein the one or more biomarkers are selected from any one or any combination of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB; and
(ii) administering one or more PKMYT1 therapeutic agents to the subject based on presence of a mutation in, a reduced expression level of, and/or a reduced activity of the one or more biomarkers relative to a healthy control.
Embodiment C-20. The method of EC-18 or EC-19, wherein the tumor sample is a circulating tumor DNA sample, a tumor biopsy sample, or a fixed tumor sample.
Embodiment C-21. The method of any one of EC-18 to EC-20, wherein the tumor sample comprises a mutation in the one or more biomarkers.
Embodiment C-22. The method of EC-21, wherein the mutation is a loss of function mutation, optionally wherein the loss of function mutation is a deletion of the gene encoding the biomarker.
Embodiment C-23. The method of EC-21 or EC-22, wherein the mutation is detected by sequencing genomic tumor DNA, optionally via next generation sequencing.
Embodiment C-24. The method of any one of EC-18 to EC-23, wherein the tumor sample comprises a plurality of tumor cells comprising the mutation.
Embodiment C-25. The method of any one of EC-17 to EC-24, wherein the one or more biomarkers comprises 2, 3, 4, 5, or more biomarkers selected from CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, NFIB, ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment C-26. The method of EC-25, wherein the PP2 subunit is PPP2R1B.
Embodiment C-27. The method of EC-25 or EC-26, wherein the PP2 subunit is PPP2R2A.
Embodiment C-28. The method of any one of EC-17 to EC-27, wherein the administering results in a reduced expression level and/or activity of PKMYT1 in a tumor of the subject.
Embodiment C-29. The method of EC-28, wherein the reduced expression level and/or activity of PKMYT1 induces synthetic lethality in the tumor.
Embodiment C-30. The method of EC-29, wherein the synthetic lethality promotes tumor regression.
Embodiment C-31. The method of any one of EC-1 to EC-30, wherein the one or more PKMYT1 therapeutic agents is selected from a small molecule, a peptide, a protein, and a nucleic acid.
Embodiment C-32. The method of EC-31, wherein the one or more PKMYT1 therapeutic agents comprises an anti-PKMYT1 antibody or fragment thereof.
Embodiment C-33. The method of EC-31, wherein the one or more PKMYT1 therapeutic agents comprises an anti-PKMYT1 intrabody or fragment thereof.
Embodiment C-34. The method of EC-31, wherein the one or more PKMYT1 therapeutic agents comprises an RNAi molecule or an aptamer.
Embodiment C-35. The method of EC-31, wherein the one or more PKMYT1 therapeutic agents comprises a small molecule inhibitor.
Embodiment C-36. The method of EC-35, wherein the small molecule inhibitor is selected from 5-((5-methoxy-2-((4-morpholinophenyl)amino)pyrimidin-4-yl)amino)-2-methylphenol, iV-(2-chloro-6-methylphenyl)-2-((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), A-(5-chlorobenzo[t/][1,3]dioxol-4-yl)-7-(2-(4-methylpiperazin-1-yl)ethoxy)-5-((tetrahydro-2//-pyran-4-yl)oxy)quinazolin-4-amine (saracatinib), (£)-A-(4-((3-chloro-4-fluorophenyl)amino)-3-cyano-7-ethoxyquinolin-6-yl)-4-(dimethylamino)but-2-enamide(pelitinib), A-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-<i]pyrimidin-7(8//)-one (PD-0166285), 6-(2,6-dichlorophenyl)-8-methyl-2-((4-morpholinophenyl)amino)pyrido[2,3-cf]pyrimidin-7(8//)-one (PD-173952), 6-(2,6-dichlorophenyl)-8-methyl-2-((3-(methylthio)phenyl)amino)pyrido[2,3-r/Jpyrimidin-7(8//)-one (PD-173955), and 6-(2,6-dichlorophenyl)-2-((4-fluoro-3-methylphenyl)amino)-8-methylpyrido[2,3-«i]pyrimidin-7(8//)-one (PD-180970).
Embodiment C-37. The method of EC-31, wherein the one or more PKMYT1 therapeutic agents comprises a gene editing technology for introducing a genetic knockout of the PKMYT1 gene.
Embodiment C-38. The method of EC-37, wherein the gene editing technology comprises CRISPR/Cas9.
Embodiment C-39. The method of any one of EC-8 to EC-38, wherein the cancer is selected from: acute myeloid leukemia (LAML), adrenocortical carcinoma (ACC), bladder urothelial carcinoma (BLCA), brain lower grade glioma (LGG), breast invasive carcinoma (BRCA), cervical squamous cell carcinoma and endocervical adenocarcinoma (CESC), cholangiocarcinoma (CHOL), chronic myelogenous leukemia (LCML), colon adenocarcinoma (COAD), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), lymphoid neoplasm diffuse large B-cell lymphoma (DLBC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), testicular germ cell tumors (TGCT), thymoma (THYM), thyroid carcinoma (THCA), uterine carcinosarcoma (UCS), uterine corpus endometrial carcinoma (UCEC), and uveal melanoma (UVM).
Embodiment C-40. Use of one or more PKMYT1 therapeutic agents for treating a cancer or promoting tumor regression in a subject, wherein the subject has been identified based on the presence of a mutation in, an altered expression level and/or altered activity of one or more biomarkers, wherein the one or more biomarkers is selected from any one or any combination of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.
Embodiment C-41. Use of one or more PKMYT1 therapeutic agents in the manufacture of a medicament for treating a cancer or promoting tumor regression in a subject, wherein the subject has been identified based on the presence of a mutation in, an altered expression level and/or altered activity of one or more biomarkers, wherein the one or more biomarkers is selected from any one or any combination of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.
Embodiment C-42. A kit comprising a PKMYT1 therapeutic agent, and a package insert comprising instructions for administering the PKMYT1 therapeutic agent to a subject having a cancer comprising a mutation in, an altered expression level and/or altered activity of one or more biomarkers, wherein the one or more biomarkers is selected from any one or any combination of CDKN2B, CSMD3, LRP1B, DMRTA1, PTPRD, ELAVL2, FAT1, CDH1, NF1, PPP6R2, PIM3, MAPK11, CDH10, PCDH15, ALB, OR4F21, LINGO2, FBN2, CACNA1E, LRRC7, NALCN, ARID1A, ADGRB3, SI, PKHD1L1, TBC1D22A, BNIP3L, DEFA1, DEFB103B, DEFB103A, HCN1, RELN, UNC13C, XKR5, CHMP7, CHRNA2, CSGALNACT1, FAM86B2, EGR3, XPO7, TRPS1, KDM6A, NBEA, VPS37A, SCN1A, CSMD2, GTSE1, TRMU, TENM1, DOCK3, VPS13B, RBM10, RYR2, SCARA5, SETBP1, DYSF, NLGN4X, EPHA3, FBLN1, ADAMTS20, IFT74, KLKB1, ACVR2A, ZFHX4, WWC2, MOB3B, DMXL1, ELAC1, RBPMS, ANK1, CADM2, C9orf72, MTNR1A, PLAA, NIPBL, ASPM, GABRB3, CTNNA3, CNTN3, PPFIA2, FN1, HECW1, DMXL2, ZFP36L2, UPK3A, SMC1B, SMARCA4, LRFN5, TG, CTNND2, CHD1, LSAMP, PRR5, NPAP1, SNTG1, MDGA2, BNC2, SCN2A, HERC2, SCN3A, TRPM1, FSTL5, ASH1L, PRKDC, TCF4, SVIL, CHD4, PCDH9, NRXN3, SNX25, MPDZ, TLL1, EPHA6, FER, NFASC, USP34, SPEF2, CHD8, ABCA12, ARID2, KCNIP4, and NFIB.
Embodiment D-1. A method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.
Embodiment D-2. A method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.
Embodiment D-3. The method of ED-1 or ED-2, wherein the diseased tissue sample comprises an altered expression level and/or activity of the one or more biomarkers relative to a reference tissue sample.
Embodiment D-4. The method of ED-3, wherein the expression level and/or activity of the one or more biomarkers is reduced relative to a reference tissue sample.
Embodiment D-5. The method of any one of ED-1 to ED-4, wherein the diseased tissue sample comprises a mutation in the one or more biomarkers relative to a reference tissue sample.
Embodiment D-6. The method of ED-5, wherein the mutation is a loss of function mutation, optionally wherein the loss of function mutation is a deletion of the gene encoding the biomarker.
Embodiment D-7. The method of ED-5 or ED-6, wherein the mutation is detected by sequencing genomic DNA in the diseased tissue sample, optionally via next generation sequencing.
Embodiment D-8. The method of any one of ED-1 to ED-7, wherein the subject has a tumor, and wherein the diseased tissue sample comprises a tumor sample, a circulating tumor DNA sample, a tumor biopsy sample, or a fixed tumor sample.
Embodiment D-9. The method of ED-8, wherein the tumor comprises a plurality of tumor cells comprising the mutation.
Embodiment D-10. The method of any one of ED-1 to ED-9, wherein the one or more biomarkers comprises 2, 3, 4, 5 or more biomarkers selected from SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, DCAF12L1, ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment D-11. The method of ED-10, wherein the PP2 subunit is PPP2R1B.
Embodiment D-12. The method of ED-10 or ED-11, wherein the PP2 subunit is PPP2R2A.
Embodiment D-13. The method of any one of ED-1 to ED-12, further comprising administering one or more PKMYT1 therapeutic agents to the subject.
Embodiment D-14. The method of ED-13, wherein the administering results in a reduced expression level and/or activity of PKMYT1 in a tumor of the subject.
Embodiment D-15. The method of ED-14, wherein the reduced expression level and/or activity of PKMYT1 induces synthetic lethality in the tumor.
Embodiment D-16. The method of ED-15, wherein the synthetic lethality promotes tumor regression.
Embodiment D-17. A method of treating a cancer or promoting tumor regression in a subject having a tumor comprising a mutation in, an altered expression level of, and/or an altered activity of one or more biomarkers, wherein the one or more biomarkers is selected from any one or any combination of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1, the method comprising: administering to the subject a therapeutically effective amount of one or more protein kinase, membrane associated tyrosine/threonine 1 (PKMYT1) therapeutic agents.
Embodiment D-18. The method of ED-17, wherein the tumor comprises a loss of function mutation in, a reduced expression level of, and/or a reduced activity of the one or more biomarkers as measured in a tumor sample obtained from the subject relative to a reference tissue sample.
Embodiment D-19. A method of identifying a cancer subject to receive one or more PKMYT1 therapeutic agents, comprising
(i) determining the presence of a mutation in, the expression level of, and/or the activity of one or more biomarkers in a tumor sample obtained from the subject, wherein the one or more biomarkers are selected from any one or any combination of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1; and
(ii) administering one or more PKMYT1 therapeutic agents to the subject based on presence of a mutation in, a reduced expression level of, and/or a reduced activity of the one or more biomarkers relative to a healthy control.
Embodiment D-20. The method of ED-18 or ED-19, wherein the tumor sample is a circulating tumor DNA sample, a tumor biopsy sample, or a fixed tumor sample.
Embodiment D-21. The method of any one of ED-18 to ED-20, wherein the tumor sample comprises a mutation in the one or more biomarkers.
Embodiment D-22. The method of ED-21, wherein the mutation is a loss of function mutation, optionally wherein the loss of function mutation is a deletion of the gene encoding the biomarker.
Embodiment D-23. The method of ED-21 or ED-22, wherein the mutation is detected by sequencing genomic tumor DNA, optionally via next generation sequencing.
Embodiment D-24. The method of any one of ED-18 to ED-23, wherein the tumor sample comprises a plurality of tumor cells comprising the mutation.
Embodiment D-25. The method of any one of ED-17 to ED-24, wherein the one or more biomarkers comprises 2, 3, 4, 5 or more biomarkers selected from SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, DCAF12L1, ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment D-26. The method of ED-25, wherein the PP2 subunit is PPP2R1B.
Embodiment D-27. The method of ED-25 or ED-26, wherein the PP2 subunit is PPP2R2A.
Embodiment D-28. The method of any one of ED-17 to ED-27, wherein the administering results in a reduced expression level and/or activity of PKMYT1 in a tumor of the subject.
Embodiment D-29. The method of ED-28, wherein the reduced expression level and/or activity of PKMYT1 induces synthetic lethality in the tumor.
Embodiment D-30. The method of ED-29, wherein the synthetic lethality promotes tumor regression.
Embodiment D-31. The method of any one of ED-1 to ED-30, wherein the one or more PKMYT1 therapeutic agents is selected from a small molecule, a peptide, a protein, and a nucleic acid.
Embodiment D-32. The method of ED-31, wherein the one or more PKMYT1 therapeutic agents comprises an anti-PKMYT1 antibody or fragment thereof.
Embodiment D-33. The method of ED-31, wherein the one or more PKMYT1 therapeutic agents comprises an anti-PKMYT1 intrabody or fragment thereof.
Embodiment D-34. The method of ED-31, wherein the one or more PKMYT1 therapeutic agents comprises an RNAi molecule or an aptamer.
Embodiment D-35. The method of ED-31, wherein the one or more PKMYT1 therapeutic agents comprises a small molecule inhibitor.
Embodiment D-36. The method of ED-35, wherein the small molecule inhibitor is selected from 5-((5-methoxy-2-((4-morpholinophenyl)amino)pyrimidin-4-yl)amino)-2-methylphenol, iV-(2-chloro-6-methylphenyl)-2-((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), A-(5-chlorobenzo[t/][1,3]dioxol-4-yl)-7-(2-(4-methylpiperazin-1-yl)ethoxy)-5-((tetrahydro-2//-pyran-4-yl)oxy)quinazolin-4-amine (saracatinib), (£)-A-(4-((3-chloro-4-fluorophenyl)amino)-3-cyano-7-ethoxyquinolin-6-yl)-4-(dimethylamino)but-2-enamide(pelitinib), A-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-<i]pyrimidin-7(8//)-one (PD-0166285), 6-(2,6-dichlorophenyl)-8-methyl-2-((4-morpholinophenyl)amino)pyrido[2,3-cf]pyrimidin-7(8//)-one (PD-173952), 6-(2,6-dichlorophenyl)-8-methyl-2-((3-(methylthio)phenyl)amino)pyrido[2,3-r/Jpyrimidin-7(8//)-one (PD-173955), and 6-(2,6-dichlorophenyl)-2-((4-fluoro-3-methylphenyl)amino)-8-methylpyrido[2,3-«i]pyrimidin-7(8//)-one (PD-180970).
Embodiment D-37. The method of ED-31, wherein the one or more PKMYT1 therapeutic agents comprises a gene editing technology for introducing a genetic knockout of the PKMYT1 gene.
Embodiment D-38. The method of ED-37, wherein the gene editing technology comprises CRISPR/Cas9.
Embodiment D-39. The method of any one of ED-8 to ED-38, wherein the cancer is selected from: acute myeloid leukemia (LAML), adrenocortical carcinoma (ACC), bladder urothelial carcinoma (BLCA), brain lower grade glioma (LGG), breast invasive carcinoma (BRCA), cervical squamous cell carcinoma and endocervical adenocarcinoma (CESC), cholangiocarcinoma (CHOL), chronic myelogenous leukemia (LCML), colon adenocarcinoma (COAD), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), lymphoid neoplasm diffuse large B-cell lymphoma (DLBC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), testicular germ cell tumors (TGCT), thymoma (THYM), thyroid carcinoma (THCA), uterine carcinosarcoma (UCS), uterine corpus endometrial carcinoma (UCEC), and uveal melanoma (UVM).
Embodiment D-40. Use of one or more PKMYT1 therapeutic agents for treating a cancer or promoting tumor regression in a subject, wherein the subject has been identified based on the presence of a mutation in, an altered expression level and/or altered activity of one or more biomarkers, wherein the one or more biomarkers is selected from any one or any combination of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.
Embodiment D-41. Use of one or more PKMYT1 therapeutic agents in the manufacture of a medicament for treating a cancer or promoting tumor regression in a subject, wherein the subject has been identified based on the presence of a mutation in, an altered expression level and/or altered activity of one or more biomarkers, wherein the one or more biomarkers is selected from any one or any combination of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.
Embodiment D-42. A kit comprising a PKMYT1 therapeutic agent, and a package insert comprising instructions for administering the PKMYT1 therapeutic agent to a subject having a cancer comprising a mutation in, an altered expression level and/or altered activity of one or more biomarkers, wherein the one or more biomarkers is selected from any one or any combination of SLITRK1, ZNF521, CCNB1, CDK7, MYT1L, FZR1, SERF1A, GADD45B, ADGRL2, TTK, NRXN2, UNC13A, ZBTB7A, POLD1, PCDH19, SLC8A2, E2F4, AUTS2, KCNN2, CCNH, FRG2C, PLK2, MYO18A, and DCAF12L1.
Embodiment E-1. A method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of the biomarkers listed in Table 1.
Embodiment E-2. A method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of the biomarkers listed in Table 1.
Embodiment E-3. The method of EE-1 or EE-2, wherein the diseased tissue sample comprises an altered expression level and/or activity of the one or more biomarkers relative to a reference tissue sample.
Embodiment E-4. The method of EE-3, wherein the expression level and/or activity of the one or more biomarkers is reduced relative to a reference tissue sample.
Embodiment E-5. The method of any one of EE-1 to EE-4, wherein the diseased tissue sample comprises a mutation in the one or more biomarkers relative to a reference tissue sample.
Embodiment E-6. The method of EE-5, wherein the mutation is a loss of function mutation, optionally wherein the loss of function mutation is a deletion of the gene encoding the biomarker.
Embodiment E-7. The method of EE-5 or EE-6, wherein the mutation is detected by sequencing genomic DNA in the diseased tissue sample, optionally via next generation sequencing.
Embodiment E-8. The method of any one of EE-1 to EE-7, wherein the subject has a tumor, and wherein the diseased tissue sample comprises a tumor sample, a circulating tumor DNA sample, a tumor biopsy sample, or a fixed tumor sample.
Embodiment E-9. The method of EE-8, wherein the tumor comprises a plurality of tumor cells comprising the mutation.
Embodiment E-10. The method of any one of EE-1 to EE-9, wherein the one or more biomarkers comprises 2, 3, 4, 5 or more biomarkers selected from the biomarkers listed in Table 1, ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment E-11. The method of EE-10, wherein the PP2 subunit is PPP2R1B.
Embodiment E-12. The method of EE-10 or EE-11, wherein the PP2 subunit is PPP2R2A.
Embodiment E-13. The method of any one of EE-1 to EE-12, further comprising administering one or more PKMYT1 therapeutic agents to the subject.
Embodiment E-14. The method of EE-13, wherein the administering results in a reduced expression level and/or activity of PKMYT1 in a tumor of the subject.
Embodiment E-15. The method of EE-14, wherein the reduced expression level and/or activity of PKMYT1 induces synthetic lethality in the tumor.
Embodiment E-16. The method of EE-15, wherein the synthetic lethality promotes tumor regression.
Embodiment E-17. A method of treating a cancer or promoting tumor regression in a subject having a tumor comprising a mutation in, an altered expression level of, and/or an altered activity of one or more biomarkers, wherein the one or more biomarkers is selected from any one or any combination of the biomarkers listed in Table 1, the method comprising: administering to the subject a therapeutically effective amount of one or more protein kinase, membrane associated tyrosine/threonine 1 (PKMYT1) therapeutic agents.
Embodiment E-18. The method of EE-17, wherein the tumor comprises a loss of function mutation in, a reduced expression level of, and/or a reduced activity of the one or more biomarkers as measured in a tumor sample obtained from the subject relative to a reference tissue sample.
Embodiment E-19. A method of identifying a cancer subject to receive one or more PKMYT1 therapeutic agents, comprising
(i) determining the presence of a mutation in, the expression level of, and/or the activity of one or more biomarkers in a tumor sample obtained from the subject, wherein the one or more biomarkers are selected from any one or any combination of the biomarkers listed in Table 1; and
(ii) administering one or more PKMYT1 therapeutic agents to the subject based on presence of a mutation in, a reduced expression level of, and/or a reduced activity of the one or more biomarkers relative to a healthy control.
Embodiment E-20. The method of EE-18 or EE-19, wherein the tumor sample is a circulating tumor DNA sample, a tumor biopsy sample, or a fixed tumor sample.
Embodiment E-21. The method of any one of claims 18-20, wherein the tumor sample comprises a mutation in the one or more biomarkers.
Embodiment E-22. The method of EE-21, wherein the mutation is a loss of function mutation, optionally wherein the loss of function mutation is a deletion of the gene encoding the biomarker.
Embodiment E-23. The method of EE-21 or EE-22, wherein the mutation is detected by sequencing genomic tumor DNA, optionally via next generation sequencing.
Embodiment E-24. The method of any one of EE-18 to EE-23, wherein the tumor sample comprises a plurality of tumor cells comprising the mutation.
Embodiment E-25. The method of any one of EE-17 to EE-24, wherein the one or more biomarkers comprises 2, 3, 4, 5 or more biomarkers selected from the biomarkers listed in Table 1, ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment E-26. The method of EE-25, wherein the PP2 subunit is PPP2R1B.
Embodiment E-27. The method of EE-25 or 26, wherein the PP2 subunit is PPP2R2A.
Embodiment E-28. The method of any one of claims 17-27, wherein the administering results in a reduced expression level and/or activity of PKMYT1 in a tumor of the subject.
Embodiment E-29. The method of EE-28, wherein the reduced expression level and/or activity of PKMYT1 induces synthetic lethality in the tumor.
Embodiment E-30. The method of EE-29, wherein the synthetic lethality promotes tumor regression.
Embodiment E-31. The method of any one of EE-1 to EE-30, wherein the one or more PKMYT1 therapeutic agents is selected from a small molecule, a peptide, a protein, and a nucleic acid.
Embodiment E-32. The method of EE-31, wherein the one or more PKMYT1 therapeutic agents comprises an anti-PKMYT1 antibody or fragment thereof.
Embodiment E-33. The method of EE-31, wherein the one or more PKMYT1 therapeutic agents comprises an anti-PKMYT1 intrabody or fragment thereof.
Embodiment E-34. The method of EE-31, wherein the one or more PKMYT1 therapeutic agents comprises an RNAi molecule or an aptamer.
Embodiment E-35. The method of EE-31, wherein the one or more PKMYT1 therapeutic agents comprises a small molecule inhibitor.
Embodiment E-36. The method of EE-35, wherein the small molecule inhibitor is selected from 5-((5-methoxy-2-((4-morpholinophenyl)amino)pyrimidin-4-yl)amino)-2-methylphenol, iV-(2-chloro-6-methylphenyl)-2-((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), A-(5-chlorobenzo[t/][1,3]dioxol-4-yl)-7-(2-(4-methylpiperazin-1-yl)ethoxy)-5-((tetrahydro-2//-pyran-4-yl)oxy)quinazolin-4-amine (saracatinib), (£)-A-(4-((3-chloro-4-fluorophenyl)amino)-3-cyano-7-ethoxyquinolin-6-yl)-4-(dimethylamino)but-2-enamide(pelitinib), A-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-<i]pyrimidin-7(8//)-one (PD-0166285), 6-(2,6-dichlorophenyl)-8-methyl-2-((4-morpholinophenyl)amino)pyrido[2,3-cf]pyrimidin-7(8//)-one (PD-173952), 6-(2,6-dichlorophenyl)-8-methyl-2-((3-(methylthio)phenyl)amino)pyrido[2,3-r/Jpyrimidin-7(8//)-one (PD-173955), and 6-(2,6-dichlorophenyl)-2-((4-fluoro-3-methylphenyl)amino)-8-methylpyrido[2,3-«i]pyrimidin-7(8//)-one (PD-180970).
Embodiment E-37. The method of EE-31, wherein the one or more PKMYT1 therapeutic agents comprises a gene editing technology for introducing a genetic knockout of the PKMYT1 gene.
Embodiment E-38. The method of EE-37, wherein the gene editing technology comprises CRISPR/Cas9.
Embodiment E-39. The method of any one of EE-8 to EE-38, wherein the cancer is selected from: acute myeloid leukemia (LAML), adrenocortical carcinoma (ACC), bladder urothelial carcinoma (BLCA), brain lower grade glioma (LGG), breast invasive carcinoma (BRCA), cervical squamous cell carcinoma and endocervical adenocarcinoma (CESC), cholangiocarcinoma (CHOL), chronic myelogenous leukemia (LCML), colon adenocarcinoma (COAD), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), lymphoid neoplasm diffuse large B-cell lymphoma (DLBC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), testicular germ cell tumors (TGCT), thymoma (THYM), thyroid carcinoma (THCA), uterine carcinosarcoma (UCS), uterine corpus endometrial carcinoma (UCEC), and uveal melanoma (UVM).
Embodiment E-40. Use of one or more PKMYT1 therapeutic agents for treating a cancer or promoting tumor regression in a subject, wherein the subject has been identified based on the presence of a mutation in, an altered expression level and/or altered activity of one or more biomarkers, wherein the one or more biomarkers is selected from any one or any combination of the biomarkers listed in Table 1.
Embodiment E-41. Use of one or more PKMYT1 therapeutic agents in the manufacture of a medicament for treating a cancer or promoting tumor regression in a subject, wherein the subject has been identified based on the presence of a mutation in, an altered expression level and/or altered activity of one or more biomarkers, wherein the one or more biomarkers is selected from any one or any combination of the biomarkers listed in Table 1.
Embodiment E-42. A kit comprising a PKMYT1 therapeutic agent, and a package insert comprising instructions for administering the PKMYT1 therapeutic agent to a subject having a cancer comprising a mutation in, an altered expression level and/or altered activity of one or more biomarkers, wherein the one or more biomarkers is selected from any one or any combination of the biomarkers listed in Table 1.
Embodiment F-1. A method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.
Embodiment F-2. A method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.
Embodiment F-3. The method of EF-1 or EF-2, wherein the diseased tissue sample comprises an altered expression level and/or activity of the one or more biomarkers relative to a reference tissue sample.
Embodiment F-4. The method of EF-3, wherein the expression level and/or activity of the one or more biomarkers is reduced relative to a reference tissue sample.
Embodiment F-5. The method of any one of EF-1 to EF-4, wherein the diseased tissue sample comprises a mutation in the one or more biomarkers relative to a reference tissue sample.
Embodiment F-6. The method of EF-5, wherein the mutation is a loss of function mutation, optionally wherein the loss of function mutation is a deletion of the gene encoding the biomarker.
Embodiment F-7. The method of EF-5 or EF-6, wherein the mutation is detected by sequencing genomic DNA in the diseased tissue sample, optionally via next generation sequencing.
Embodiment F-8. The method of any one of EF-1 to EF-7, wherein the subject has a tumor, and wherein the diseased tissue sample comprises a tumor sample, a circulating tumor DNA sample, a tumor biopsy sample, or a fixed tumor sample.
Embodiment F-9. The method of EF-8, wherein the tumor comprises a plurality of tumor cells comprising the mutation.
Embodiment F-10. The method of any one of EF-1 to EF-9, wherein the one or more biomarkers comprises 2, 3, 4, 5 or more biomarkers selected from OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, RAD21, ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment F-11. The method of EF-10, wherein the PP2 subunit is PPP2R1B.
Embodiment F-12. The method of EF-10 or EF-11, wherein the PP2 subunit is PPP2R2A.
Embodiment F-13. The method of any one of EF-1 to EF-12, further comprising administering one or more PKMYT1 therapeutic agents to the subject.
Embodiment F-14. The method of EF-13, wherein the administering results in a reduced expression level and/or activity of PKMYT1 in a tumor of the subject.
Embodiment F-15. The method of EF-14, wherein the reduced expression level and/or activity of PKMYT1 induces synthetic lethality in the tumor.
Embodiment F-16. The method of EF-15, wherein the synthetic lethality promotes tumor regression.
Embodiment F-17. A method of treating a cancer or promoting tumor regression in a subject having a tumor comprising a mutation in, an altered expression level of, and/or an altered activity of one or more biomarkers, wherein the one or more biomarkers is selected from any one or any combination of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21, the method comprising: administering to the subject a therapeutically effective amount of one or more protein kinase, membrane associated tyrosine/threonine 1 (PKMYT1) therapeutic agents.
Embodiment F-18. The method of EF-17, wherein the tumor comprises a loss of function mutation in, a reduced expression level of, and/or a reduced activity of the one or more biomarkers as measured in a tumor sample obtained from the subject relative to a reference tissue sample.
Embodiment F-19. A method of identifying a cancer subject to receive one or more PKMYT1 therapeutic agents, comprising
(i) determining the presence of a mutation in, the expression level of, and/or the activity of one or more biomarkers in a tumor sample obtained from the subject, wherein the one or more biomarkers are selected from any one or any combination of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21; and
(ii) administering one or more PKMYT1 therapeutic agents to the subject based on presence of a mutation in, a reduced expression level of, and/or a reduced activity of the one or more biomarkers relative to a healthy control.
Embodiment F-20. The method of EF-18 or EF-19, wherein the tumor sample is a circulating tumor DNA sample, a tumor biopsy sample, or a fixed tumor sample.
Embodiment F-21. The method of any one of claims 18-20, wherein the tumor sample comprises a mutation in the one or more biomarkers.
Embodiment F-22. The method of EF-21, wherein the mutation is a loss of function mutation, optionally wherein the loss of function mutation is a deletion of the gene encoding the biomarker.
Embodiment F-23. The method of EF-21 or EF-22, wherein the mutation is detected by sequencing genomic tumor DNA, optionally via next generation sequencing.
Embodiment F-24. The method of any one of EF-18 to EF-23, wherein the tumor sample comprises a plurality of tumor cells comprising the mutation.
Embodiment F-25. The method of any one of EF-17 to EF-24, wherein the one or more biomarkers comprises 2, 3, 4, 5 or more biomarkers selected from OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, RAD21, ATM, MAP2K4, TP53, CDC25A, CACNA1H, CDKN1B, DUSP7, FOXO3, FZD3, JAK1, SMAD2, TGFBR2, MAP3K2, PPP3CC, and a PP2 subunit.
Embodiment F-26. The method of EF-25, wherein the PP2 subunit is PPP2R1B.
Embodiment F-27. The method of EF-25 or EF-26, wherein the PP2 subunit is PPP2R2A.
Embodiment F-28. The method of any one of EF-17 to EF-27, wherein the administering results in a reduced expression level and/or activity of PKMYT1 in a tumor of the subject.
Embodiment F-29. The method of EF-28, wherein the reduced expression level and/or activity of PKMYT1 induces synthetic lethality in the tumor.
Embodiment F-30. The method of EF-29, wherein the synthetic lethality promotes tumor regression.
Embodiment F-31. The method of any one of EF-1 to EF-30, wherein the one or more PKMYT1 therapeutic agents is selected from a small molecule, a peptide, a protein, and a nucleic acid.
Embodiment F-32. The method of EF-31, wherein the one or more PKMYT1 therapeutic agents comprises an anti-PKMYT1 antibody or fragment thereof.
Embodiment F-33. The method of EF-31, wherein the one or more PKMYT1 therapeutic agents comprises an anti-PKMYT1 intrabody or fragment thereof.
Embodiment F-34. The method of EF-31, wherein the one or more PKMYT1 therapeutic agents comprises an RNAi molecule or an aptamer.
Embodiment F-35. The method of EF-31, wherein the one or more PKMYT1 therapeutic agents comprises a small molecule inhibitor.
Embodiment F-36. The method of EF-35, wherein the small molecule inhibitor is selected from 5-((5-methoxy-2-((4-morpholinophenyl)amino)pyrimidin-4-yl)amino)-2-methylphenol, iV-(2-chloro-6-methylphenyl)-2-((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), A-(5-chlorobenzo[t/][1,3]dioxol-4-yl)-7-(2-(4-methylpiperazin-1-yl)ethoxy)-5-((tetrahydro-2//-pyran-4-yl)oxy)quinazolin-4-amine (saracatinib), (£)-A-(4-((3-chloro-4-fluorophenyl)amino)-3-cyano-7-ethoxyquinolin-6-yl)-4-(dimethylamino)but-2-enamide(pelitinib), A-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-<i]pyrimidin-7(8//)-one (PD-0166285), 6-(2,6-dichlorophenyl)-8-methyl-2-((4-morpholinophenyl)amino)pyrido[2,3-cf]pyrimidin-7(8//)-one (PD-173952), 6-(2,6-dichlorophenyl)-8-methyl-2-((3-(methylthio)phenyl)amino)pyrido[2,3-r/Jpyrimidin-7(8//)-one (PD-173955), and 6-(2,6-dichlorophenyl)-2-((4-fluoro-3-methylphenyl)amino)-8-methylpyrido[2,3-«i]pyrimidin-7(8//)-one (PD-180970).
Embodiment F-37. The method of EF-31, wherein the one or more PKMYT1 therapeutic agents comprises a gene editing technology for introducing a genetic knockout of the PKMYT1 gene.
Embodiment F-38. The method of EF-37, wherein the gene editing technology comprises CRISPR/Cas9.
Embodiment F-39. The method of any one of EF-8 to EF-38, wherein the cancer is selected from: acute myeloid leukemia (LAML), adrenocortical carcinoma (ACC), bladder urothelial carcinoma (BLCA), brain lower grade glioma (LGG), breast invasive carcinoma (BRCA), cervical squamous cell carcinoma and endocervical adenocarcinoma (CESC), cholangiocarcinoma (CHOL), chronic myelogenous leukemia (LCML), colon adenocarcinoma (COAD), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), lymphoid neoplasm diffuse large B-cell lymphoma (DLBC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), testicular germ cell tumors (TGCT), thymoma (THYM), thyroid carcinoma (THCA), uterine carcinosarcoma (UCS), uterine corpus endometrial carcinoma (UCEC), and uveal melanoma (UVM).
Embodiment F-40. Use of one or more PKMYT1 therapeutic agents for treating a cancer or promoting tumor regression in a subject, wherein the subject has been identified based on the presence of a mutation in, an altered expression level and/or altered activity of one or more biomarkers, wherein the one or more biomarkers is selected from any one or any combination of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.
Embodiment F-41. Use of one or more PKMYT1 therapeutic agents in the manufacture of a medicament for treating a cancer or promoting tumor regression in a subject, wherein the subject has been identified based on the presence of a mutation in, an altered expression level and/or altered activity of one or more biomarkers, wherein the one or more biomarkers is selected from any one or any combination of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.
Embodiment F-42. A kit comprising a PKMYT1 therapeutic agent, and a package insert comprising instructions for administering the PKMYT1 therapeutic agent to a subject having a cancer comprising a mutation in, an altered expression level and/or altered activity of one or more biomarkers, wherein the one or more biomarkers is selected from any one or any combination of OR4F16, BUB1B, PLK1, PAXBP1, CTR9, AR, EIF3A, KIF4A, MAGEB10, CHEK1, CENPM, AKT1, ADCY1, ATP2B2, HASPIN, CTDSPL2, STAG2, NCAPG, IGF1R, BLM, ATR, AURKB, RBL2, RPS6KA6, GINS2, MAD1L1, ADCY5, CHTF18, SMC1A, BRSK2, BRPF3, FOXD4L4, TGIF2LX, SOX5, POU4F1, UHRF1, PPP2R2C, WDR45, FAM120C, BRSK1, EVI5L, NPAS4, MCM10, SUPT5H, MCM5, GALK2, FTSJ1, TRAP1, PAK3, CENPE, TPT1, MAD2L2, FBXO5, CDK16, CDC45, USP27X, MAPK8, PRR20A, ADCY4, RRM1, TBR1, PAK2, KIF11, WDHD1, MELK, CHERP, CENPF, BUB1, PRMT5, EIF1AX, SMPD2, CASP8AP2, SFN, WEE1, ESPL1, OTUD5, DMRTC1B, TSSK2, ANAPC10, FOXM1, EXO1, CHEK2, KIFC1, ANKRD52, SPAG5, PPP2R2B, ZNF331, PAK1, TNPO2, LDB1, CDK14, CDC25B, KCNV1, CPEB1, ZNF777, RPS6KA1, PSG7, CD177, CCNG1, PRAMEF8, ZBTB17, CCNF, E2F2, HDAC1, CCNB2, KIF15, AGPAT3, REC8, RECQL4, ZNF853, SRSF4, PPP2R5A, ZBTB12, MMP12, KIF2C, HSP90AA1, PPP2R2D, CDC7, NANS, MOS, RBX1, MAGED4B, KIF23, SCML1, SPANXA2, TRIM28, SRRM5, MAGEA1, ACTR3B, EBLN1, TP53TG3C, INS, ORC1, HSP90AB1, CHAF1B, MCM7, CPSF6, NACC1, WEE2, MYC, MCM6, ADCY6, TPX2, MYBL2, CDC23, RRM2, MAPK1, PRKACA, DDI2, MEMO1, IGF1, SKP1, PPIAL4C, PPIAL4D, SLC9A6, ARPP19, NOVA2, CTAG1B, CCNA2, CDC6, MAGEA9, F8A3, ARL17A, CTAG1A, MAD2L1, HSFX1, BNIP3, MRGPRG, ANAPC2, and RAD21.

### EXAMPLES

### Example 1—Prediction of Biomarkers Forming a Synthetic Lethal Pair with PKMYT1

A computational approach was used to identify genes that are inactivated (e.g., via mutation or deletion) in tumor cells that when combined with loss of function of a target gene (e.g., by genetic knockout using CRISPR/Cas9 or by pharmacological inhibition) generate a synthetic lethal phenotype. As used herein, “gene A” or a “gene A biomarker” each refer to a gene in the genetic background of a tumor (e.g., primary tumor) of one or more human cancers that when inactivated by deletion or mutation (e.g., homozygous deletion, missense mutation that is deleterious to protein function, or missense mutation rendering an open reading frame that encodes a truncated protein) has little effect on cell viability on its own, but when combined with loss of function of the target gene, referred to herein as “gene B,” results in synthetic lethality. This example is based on prediction of gene A biomarkers that function as synthetic lethal pairs with the human protein kinase PKMYT1. The computational approaches taken to identify the biomarkers involved mining public and proprietary datasets using unbiased, orthogonal algorithms. Described in this example are the computational methods and criteria that were used to prioritize predicted gene A biomarkers for further validation.

A first algorithm (referred to herein as “algorithm A”) was developed to evaluate one or more publicly available databases. Suitable databases catalog data generated from genetic knockout-libraries (e.g., RNAi or CRISPR/Cas9 libraries) screened for lethality across many genetic contexts. Algorithm A enables analysis of the lethality of specific gene knockouts across the different genetic backgrounds to identify putative synthetic lethal pairs. For example, algorithm A considers the gene mutations present in a given genetic background and mines the gene-knockout screen for potential targets that when suppressed (e.g., using a gene-knockout tool such as CRISPR/Cas9 or an inhibitory drug), result in synthetic lethality. Algorithm A was implemented with certain prediction criteria to predict gene A biomarkers that form synthetic lethal pairs with PKMYT1. The prediction criteria used in conjunction with algorithm A to select predicted gene A biomarkers include (a)(i) a P value of less than 0.001 or (a)(ii) odds ratio of greater than 2; and (b) gene inactivation in at least 4 or more cell lines. The P value was derived from a chi-squared test of association of the biomarker mutation and sensitivity to perturbation in PKYMT1. The odds ratio was defined as the ratio of the odds of sensitivity to PKYMT1 perturbation in cells with a mutation in the biomarker to the odds of sensitivity to PKYMT1 perturbation in cells that are wild-type for the biomarker. Predicted biomarkers that met these prediction criteria were further tiered based on prevalence. Prevalence was determined as the frequency of inactivating mutations (i.e., homozygous deletion, missense mutation that are deleterious, or missense mutations that are protein truncating variants) across all 28 cancer types listed in TCGA (Cancer Genome Atlas Program; see world wide web: cancer.gov/tcga). Predicted biomarkers with (i) prevalence >5% were categorized as “algorithm A Tier 1”; (ii) prevalence of ≥3% and <5% were categorized as “algorithm A Tier 2”; and (iii) prevalence of ≥1% and <3% were categorized as “algorithm A Tier 3.” According to these prediction criteria, 1 biomarker was identified as algorithm A Tier 1 biomarker, 49 biomarkers were identified as algorithm A Tier 2 biomarkers, and 896 biomarkers were identified as algorithm A Tier 3 biomarkers.

A second algorithm (referred to herein as “algorithm B”) is based on a machine-learning model. A large database featuring synthetic lethal pairs was developed from internally-generated functional genomic and experimental data and externally sourced and/or publicly-available datasets. The database was used to train the machine learning model to identify gene interactions that could function as synthetic lethal pairs. Algorithm B was implemented with prediction criteria to predict gene A biomarkers that form a synthetic lethal pair with PKMYT1. The prediction criteria included a prediction score and prevalence data. The prediction score ranged between 0 and 1, wherein a gene A biomarker with a higher prediction score has a stronger probability of forming a synthetic lethal pair with a given target gene B (e.g., PKYMT1). A prediction score of 0.3 was used as an estimated cutoff that maximizes the sum of sensitivity and specificity based on the algorithm B training data. Predicted biomarkers that met the prediction criteria were tiered based on prevalence across six human cancer types listed in the TCGA, including: colorectal adenocarcinoma (COAD), breast invasive carcinoma (BRCA), lung adenocarcinoma (LUAD), lung squamous cell carcinoma (LUSC), ovarian serous cystadenocarcinoma (OV), and liver hepatocellular carcinoma (LIHC). Biomarkers having (i) a prediction score of greater than 0.3 and a prevalence of >5% were categorized as “algorithm B Tier 1”; (ii) a prediction score of greater than 0.5 and a prevalence of ≥3% and <5% were categorized as “algorithm B Tier 2”; and (iii) a prediction score of greater than 0.5 and a prevalence of ≥1% and <3% were categorized as “algorithm B Tier 3.” According to these prediction criteria, 138 biomarkers were identified as algorithm B Tier 1 biomarkers, 29 biomarkers were identified as algorithm B Tier 2 biomarkers, and 181 biomarkers were identified as algorithm B Tier 3 biomarkers.

Of the biomarkers that were identified based on the prediction criteria for algorithm A and B, the biomarkers selected for further validation as forming synthetic lethal pairs with PKYMT1 are indicated below:
(1) As shown in Table 4, ATM, MAP2K4, and TP53 were predicted as loss of function biomarkers that form synthetic lethal pairs with PKMYT1 according to the algorithm B Tier 1 prediction criteria; and CDC25A was predicted as a loss of function biomarker according to the algorithm B Tier 3 prediction criteria. Furthermore, TP53 was predicted as a loss of function biomarker that forms a synthetic lethal pair with PKMYT1 according to prediction criteria for both algorithm A Tier 1 and algorithm B Tier 1. Without being bound by theory, a biomarker identified by both algorithm A and algorithm B as a predicted synthetic lethal pair with PKMYT1 is expected to have a higher likelihood of being validated as a synthetic lethal pair with PKMYT1. This rationale is based, at least in part, on the understanding that algorithm A and algorithm B are orthogonal approaches to identify putative synthetic lethal pairs, with algorithm A being a predictive algorithm that provides for analysis of experimental datasets and algorithm B a machine-learning model, and they are not expected to yield an extensive overlap of false positive LOF biomarkers;
(2) biomarkers identified by both algorithm A and algorithm B that include the 27 biomarkers identified in Table 4 (CNTN5, IRF2, ALPK2, CHKB, MAPK12, SLC8A1, CDH19, CDT1, ADCY2, SLK, RPS6KA3, CCNO, HDAC2, CDC20B, STAG1, CKAP5, RAD51, CKS1B, KCNA2, MCM4, PLK4, CDC16, BIN3, AGPAT5, FGF17, PBK, and NOTCH1);

TABLE 4
Gene A biomarkers identified based on Algorithm
A and Algorithm B prediction criteria

| Algorithm A | Algorithm B | Biomarker |
|---|---|---|
| Algorithm A Tier 1 | — | CDKN1B, DUSP7, |
| | | FOXO3, |
| Algorithm A Tier 2 | — | FZD3 |
| — | Algorithm B Tier 1 | ATM, MAP2K4 |
| — | Algorithm B Tier 3 | CDC25A |

Algorithm A Tier 1 + Algorithm B Tier 1

| | | |
|---|---|---|
| Algorithm A Tier 1 | Algorithm B Tier 1 | CNTN5 |
| Algorithm A Tier 1 | Algorithm B Tier 1 | IRF2 |
| Algorithm A Tier 1 | Algorithm B Tier 1 | ALPK2 |
| Algorithm A Tier 1 | Algorithm B Tier 1 | CHKB |
| Algorithm A Tier 1 | Algorithm B Tier 1 | MAPK12 |
| Algorithm A Tier 1 | Algorithm B Tier 1 | SLC8A1 |
| Algorithm A Tier 1 | Algorithm B Tier 1 | CDH19 |
| Algorithm A Tier 1 | Algorithm B Tier 1 | TP53 |

Algorithm A Tier 1 + Algorithm B Tier 2

| | | |
|---|---|---|
| Algorithm A Tier 1 | Algorithm B Tier 2 | CDT1 |
| Algorithm A Tier 1 | Algorithm B Tier 2 | ADCY2 |
| Algorithm A Tier 1 | Algorithm B Tier 2 | SLK |
| Algorithm A Tier 1 | Algorithm B Tier 2 | RPS6KA3 |
| Algorithm A Tier 1 | Algorithm B Tier 2 | CCNO |

Algorithm A Tier 1 + Algorithm B Tier 3

| | | |
|---|---|---|
| Algorithm A Tier 1 | Algorithm B Tier 3 | HDAC2 |
| Algorithm A Tier 1 | Algorithm B Tier 3 | CDC20B |
| Algorithm A Tier 1 | Algorithm B Tier 3 | STAG1 |
| Algorithm A Tier 1 | Algorithm B Tier 3 | CKAP5 |
| Algorithm A Tier 1 | Algorithm B Tier 3 | RAD51 |
| Algorithm A Tier 1 | Algorithm B Tier 3 | CKS1B |
| Algorithm A Tier 1 | Algorithm B Tier 3 | KCNA2 |
| Algorithm A Tier 1 | Algorithm B Tier 3 | MCM4 |
| Algorithm A Tier 1 | Algorithm B Tier 3 | PLK4 |
| Algorithm A Tier 1 | Algorithm B Tier 3 | CDC16 |

Algorithm A Tier 2 + Algorithm B Tier 1

| | | |
|---|---|---|
| Algorithm A Tier 2 | Algorithm B Tier 1 | BIN3 |
| Algorithm A Tier 2 | Algorithm B Tier 1 | AGPAT5 |
| Algorithm A Tier 2 | Algorithm B Tier 1 | FGF17 |
| Algorithm A Tier 2 | Algorithm B Tier 1 | PBK |
| Algorithm A Tier 2 | Algorithm B Tier 1 | NOTCH1 |

(3) biomarkers identified by algorithm A as Tier 1 or Tier 2 biomarkers, listed in Table 5;

TABLE 5
Gene A biomarkers identified based
on Algorithm A prediction criteria

| | | |
|---|---|---|
| ERICH1 | MTMR9 | NRG1 |
| TNKS | MSRA | ASAH1 |
| TDRP | PDLIM2 | DEFA3 |
| MTUS1 | INTS10 | EPHX2 |
| TNFRSF10B | SH2D4A | CNOT7 |
| HR | GFRA2 | PNMA2 |
| TNFRSF10D | ZDHHC2 | TRIM35 |
| DMTN | PDGFRL | ATRX |
| ENTPD4 | SPAG11B | INTS9 |
| TNFRSF10C | PPP1R3B | DNAH3 |
| PEBP4 | SPAG11A | MAP3K1 |
| LPL | REEP4 | RIMS2 |
| LGI3 | DEFA5 | NSD1 |
| SLC7A2 | DEFB136 | SARAF |

(4) biomarkers identified according to algorithm B Tier 1 prediction criteria and listed in Table 6;

TABLE 6
Gene A biomarkers identified based on Algorithm B Tier 1 prediction criteria

| | | | | | |
|---|---|---|---|---|---|
| CDKN2B | ARID1A | NBEA | ZFHX4 | UPK3A | SVIL |
| CSMD3 | ADGRB3 | VPS37A | WWC2 | SMC1B | CHD4 |
| LRP1B | SI | SCN1A | MOB3B | SMARCA4 | PCDH9 |
| DMRTA1 | PKHD1L1 | CSMD2 | DMXL1 | LRFN5 | NRXN3 |
| PTPRD | TBC1D22A | GTSE1 | ELAC1 | TG | SNX25 |
| ELAVL2 | BNIP3L | TRMU | RBPMS | CTNND2 | MPDZ |
| FAT1 | DEFA1 | TENM1 | ANK1 | CHD1 | TLL1 |
| CDH1 | DEFB103B | DOCK3 | CADM2 | LSAMP | EPHA6 |
| NF1 | DEFB103A | VPS13B | C9orf72 | PRR5 | FER |
| PPP6R2 | HCN1 | RBM10 | MTNR1A | NPAP1 | NFASC |
| PIM3 | RELN | RYR2 | PLAA | SNTG1 | USP34 |
| MAPK11 | UNC13C | SCARA5 | NIPBL | MDGA2 | SPEF2 |
| CDH10 | XKR5 | SETBP1 | ASPM | BNC2 | CHD8 |
| PCDH15 | CHMP7 | DYSF | GABRB3 | SCN2A | ABCA12 |
| ALB | CHRNA2 | NLGN4X | CTNNA3 | HERC2 | ARID2 |
| OR4F21 | CSGALNACT1 | EPHA3 | CNTN3 | SCN3A | KCNIP4 |
| LINGO2 | FAM86B2 | FBLN1 | PPFIA2 | TRPM1 | NFIB |
| FBN2 | EGR3 | ADAMTS20 | FN1 | FSTL5 | — |
| CACNA1E | XPO7 | IFT74 | HECW1 | ASH1L | — |
| LRRC7 | TRPS1 | KLKB1 | DMXL2 | PRKDC | — |
| NALCN | KDM6A | ACVR2A | ZFP36L2 | TCF4 | — |

(5) biomarkers identified according to the algorithm B Tier 2 prediction criteria and listed in Table 7;

TABLE 7
Gene A biomarkers identified based on
Algorithm B Tier 2 prediction criteria

| | | | |
|---|---|---|---|
| SLITRK1 | SERF1A | ZBTB7A | KCNN2 |
| ZNF521 | GADD45B | POLD1 | CCNH |
| CCNB1 | ADGRL2 | PCDH19 | FRG2C |
| CDK7 | TTK | SLC8A2 | PLK2 |
| MYT1L | NRXN2 | E2F4 | MYO18A |
| FZR1 | UNC13A | AUTS2 | DCAF12L1 |

(6) biomarkers identified as algorithm A Tier 3 biomarkers and listed in Table 1; and
(7) biomarkers identified as algorithm B Tier 3 biomarkers and listed in Table 2.

### Example 2—CombiGEM Methods for Identifying Synthetic Lethality Pairs

A combinatorial genetic en masse (CombiGEM™) screen is used to validate synthetic lethal pairs identified by the computational methods described in Example 1. Methods of performing CombiGEM™ are known in the art and described in U.S. Pat. No. 9,315,806; Bari, et al. (2017) Scientific Reports 7:6993; Wong, et al (2016) PNAS 113:2544-2549, each of which are incorporated by reference in their entirety. This example provides an overview of the pooled screening method to validate predicted synthetic lethal pairs of gene A biomarkers and gene B targets.

Briefly, oligonucleotide synthesis is used to generate a library of barcoded gRNA target sequences directed to gene A biomarkers (“gene A gRNA library”) and a library of barcoded gRNA target sequences directed to gene B targets (“gene B gRNA library”). The libraries are pooled and cloned into a storage vector downstream a promoter (e.g., a human U6 (hU6) promoter for the gene A gRNA library and a murine U6 (mU6) promoter for the gene B gRNA library). The gRNA backbone sequence is then inserted into the storage vector libraries in a single-pot ligation reaction to create the gene A and gene B barcoded sgRNA libraries. Within the sgRNA construct, there are “internal” restriction sites positioned between the gRNA sequence and its barcode (e.g., BamHI and EcoRI sites) and “external” restriction sites positioned at the ends (e.g., Bg1II and MfeI sites).

A first “gene A sgRNA lentiviral library” is generated by digesting the gene A sgRNA constructs at the external restriction sites and inserting into a destination lentiviral backbone having compatible overhangs that are generated by digestion, thereby generating a barcoded sgRNA lentiviral library for generating loss-of-function in gene A.

A second “gene B sgRNA lentiviral library” is generated by digesting the gene B sgRNA constructs at the external restriction sites and inserting into a destination lentiviral backbone having compatible overhangs that are generated by digestion, thereby generating a barcoded sgRNA lentiviral library for generating loss-of-function in gene B.

A third “gene A*B sgRNA lentiviral library” is generated by digesting the gene A sgRNA constructs at the external restriction sites and inserting into a destination lentiviral backbone having compatible overhangs that are generated by digestion. Subsequently, the gene B sgRNA construct is digested at its external restrictions sites and inserted into the lentiviral construct at a site generated by digestion of the internal restriction sites positioned adjacent to the gene A sgRNA sequences. The resulting library of lentiviral vectors contains 5′ to 3′: (i) a gene A-targeting sgRNA, (ii) a gene B-targeting sgRNA, and (iii) concatenated gene A sgRNA and gene B sgRNA barcodes that enable tracking of individual combinatorial members within pooled populations via next generation sequencing. FIG. 1 provides a schematic depicting the constructs of the gene A*B sgRNA lentiviral library.

The barcoded sgRNA lentiviral libraries are evaluated in Cas9-expressing cancer cell lines (e.g., HT29 and/or LS180 cell lines). Briefly, the lentiviruses are produced and packaged, e.g., in HEK293T cells. The cell culture are transduced with either the gene A sgRNA lentiviral library, the gene B sgRNA lentiviral library, or the gene A*B sgRNA lentiviral library. A low multiplicity of infection is used to ensure single copy integration in most cells. Transfected cells are cultured, e.g., for a period of 20-30 days. Genomic DNA is harvested and used for quantification of the integrated barcodes using a combination of barcode amplification by PCR and sequencing by NGS. The proliferation rate of a particular clone is based on the relative frequency of its barcode with regard to the whole population. The barcode reads are normalized per million reads for each sample. To measure cell proliferation, barcode count ratios of normalized barcode reads were calculated as fold changes. The calculated fold change was log transformed to give the log₂ fold-change (LFC). Pro-proliferation and anti-proliferation phenotypes have an LFC of greater than zero and less than zero respectively.

The LFC values are determined for cells transfected using gene A sgRNA sequences (gene A knockout), the gene B sgRNA sequences (gene B knockout), and the geneA*B sgRNA sequences (gene A*B double knockout). The gene interaction (GI) score is calculated, which is the difference between the observed LFC of the gene A*B double knockout and the expected LFC of the gene A*B double knockout that would be obtained by simply adding gene A knockout LFC+gene B knockout LFC. Synthetic lethal pairs are then selected as those having at least one cell line with (i) a gene A*B double knockout LFC of less than −1 and (II) a GI score of less than −1.

The foregoing CombiGEM methods are used to validate predicted biomarkers as synthetic lethal pairs with PKMYT1. A gene A sgRNA lentiviral library and a gene A*B sgRNA lentiviral library is developed, in which the gene A sgRNA library targets the predicted biomarkers identified in Example 1 and gene B sgRNA targets PKMYT1. The barcoded sgRNA lentiviral libraries are evaluated in Cas9-expressing cancer cell lines (e.g., HT29 or LS180). Lentiviral constructs encoding the PKMYT1 sgRNA are also evaluated in Cas9-expressing cancer cell lines (e.g., HT29 or LS180 cell lines). Following culture, the cells are harvested and LFC and GI values are determined as described above.

### Example 3—Validation of Predicted Biomarkers and PKMYT1 as Synthetic Lethality Pairs

The CombiGEM methods described in Example 2 were used to validate putative biomarkers as synthetic lethal pairs with PKMYT1. A gene A sgRNA lentiviral library and a gene A*B sgRNA lentiviral library were developed, in which the gene A sgRNA library targets the putative biomarkers identified in Example 1 and gene B sgRNA targets PKMYT1. 15 biomarkers were evaluated as synthetic lethal pairs with PKMYT1 by CombiGEM, with biomarkers predicted by the computational approaches described in Example 1 (CDKN1B, DUSP7, FOXO3, TP53, FZD3, ATM, MAP2K4, CDC25A) or predicted based on mechanistic hypothesis. The barcoded sgRNA lentiviral libraries were evaluated in Cas9-expressing colon cancer cell lines (HT29 and LS180). Lentiviral constructs encoding the PKMYT1 sgRNA were also evaluated in Cas9-expressing HT29 and LS180 cell lines. Following culture, the cells were harvested and LFC and GI values were determined as described in Example 2.

The LFC values for HT29 and LS180 cells transfected with PKMYT1-targeting sgRNA were −0.12 and −1.47 respectively. The 15 biomarkers were validated as being synthetic lethal pairs with PKMYT1 based on the validation criteria indicated in Example 2 (i.e., the gene A*B double knockout has (i) an LFC less than −1, and (ii) a GI score of less than −1 in at least one cell line). Shown in Table 8 are gene A biomarkers that were validated as synthetic lethal pairs with PKMYT1 in HT29 and/or LS180 cell lines.

TABLE 8
15 Gene A Biomarkers Satisfying CombiGEM Validation
Criteria in at least one cancer cell line

| | | LFC | | | | | | | | |
|---|---|---|---|---|---|---|---|---|---|---|
| | Cell | Gene | Gene | | Prevalence* | | | | | |
| Biomarker | line | A | A*B | GI | COAD | BRCA | LUAD | LUSC | OV | LIHC |
| ATM | HT29 | −0.01 | −1.64 | −1.51 | 10.04 | 2.93 | 6.30 | 3.57 | 1.53 | 2.12 |
| | LS180 | −0.38 | −2.86 | −1.01 | | | | | | |
| CACNA1H | HT29 | 0.12 | −1.37 | −1.37 | 6.99 | 0.64 | 1.93 | 2.58 | 1.02 | 1.33 |
| | LS180 | −0.24 | −2.76 | −1.05 | | | | | | |
| CDC25A | HT29 | −0.18 | −1.5 | −1.2 | 1.53 | 0.64 | 0.18 | 0.79 | 1.02 | 0.27 |
| | LS180 | −0.67 | −2.94 | −0.8 | | | | | | |
| CDKN1B | HT29 | 0.3 | −1.25 | −1.43 | 0.87 | 1.74 | 2.63 | 0.20 | 0.51 | 0.80 |
| | LS180 | 0.13 | −2.45 | −1.1 | | | | | | |
| DUSP7 | HT29 | 0.38 | −1.27 | −1.54 | 0.66 | 0.82 | 0.35 | 1.59 | 0.51 | 0.27 |
| | LS180 | 0.05 | −2.67 | −1.25 | | | | | | |
| FOXO3 | HT29 | 0.39 | −1.25 | −1.53 | 1.09 | 0.82 | 1.75 | 0.60 | 1.19 | 1.86 |
| | LS180 | 0.25 | −2.67 | −1.45 | | | | | | |
| FZD3 | HT29 | 0.28 | −1.19 | −1.36 | 6.33 | 5.30 | 5.08 | 5.16 | 6.29 | 6.63 |
| | LS180 | −0.21 | −2.68 | −1.01 | | | | | | |
| JAK1 | HT29 | 0.69 | −1.18 | −1.75 | 2.18 | 1.19 | 1.58 | 1.79 | 0.85 | 1.06 |
| | LS180 | 0.35 | −2.85 | −1.73 | | | | | | |
| MAP2K4 | HT29 | 0.27 | −1.22 | −1.38 | 6.77 | 6.58 | 2.63 | 1.19 | 3.74 | 2.92 |
| | LS180 | 0.12 | −2.57 | −1.22 | | | | | | |
| MAP3K2 | HT29 | −0.14 | −1.48 | −1.23 | 1.09 | 0.18 | 0.53 | 0.20 | 1.19 | 0.53 |
| | LS180 | −0.83 | −2.97 | −0.67 | | | | | | |
| PPP2R1B | HT29 | 0.20 | −1.33 | −1.41 | 0.66 | 1.65 | 1.05 | 1.59 | 1.19 | 0.80 |
| | LS180 | −0.71 | −3.13 | −0.95 | | | | | | |
| PPP3CC | HT29 | 0.15 | −1.09 | −1.12 | 5.90 | 5.94 | 5.08 | 6.15 | 7.14 | 7.43 |
| | LS180 | −0.37 | −2.47 | −0.63 | | | | | | |
| SMAD2 | HT29 | 0.20 | −1.25 | −1.33 | 6.11 | 0.73 | 2.10 | 1.79 | 2.04 | 0.27 |
| | LS180 | −0.24 | −2.74 | −1.03 | | | | | | |
| TGFBR2 | HT29 | 0.28 | −1.48 | −1.65 | 3.71 | 0.46 | 0.88 | 2.18 | 0.85 | 0.27 |
| | LS180 | 0.24 | −2.71 | −1.48 | | | | | | |
| TP53 | HT29 | 0.44 | −1.25 | −1.57 | 37.55 | 27.97 | 45.18 | 70.83 | 56.29 | 26.79 |
| | LS180 | 1.66 | −1.44 | −1.62 | | | | | | |

Based on the data shown in Table 8, several biomarkers that were validated in the HT29 and/or LS180 cell lines were predicted by algorithm 1 and/or algorithm 2:
(i) TP53 was predicted as an algorithm 1 Tier 1 biomarker and an algorithm 2 Tier 1 biomarker and validated as a synthetic lethal pair with PKYMT1 in both HT29 and LS180 cell lines;
(ii) CDKN1B, DUSP7, FOXO3, FZD3, and SMAD2 were predicted as algorithm 1 Tier 1 or algorithm 1 Tier 2 biomarkers and were validated in both HT29 and LS180 cell lines;
(iii) ATM and MAP2K4 were predicted as an algorithm 2 Tier 1 biomarker and validated in both HT29 and LS180 cell lines; and
(iv) CDC25A was predicted as an algorithm 2 Tier 3 biomarker and validated in the HT29 cell line.

Additional biomarkers identified in Table 8 include:
(i) CACNA1H, JAK1, and TGFBR, which were validated in the HT29 and LS180 cell lines; and
(ii) PPP2R1B, PPP3CC and MAP3K2, which were validated in the HT29 cell line.

Table 8 also show the prevalence of gene A biomarker mutations across colorectal adenocarcinoma (COAD), breast invasive carcinoma (BRCA), lung adenocarcinoma (LUAD), lung squamous cell carcinoma (LUSC), ovarian serous cystadenocarcinoma (OV), and liver hepatocellular carcinoma (LIHC) primary human tumors. Without being bound by theory, a gene A biomarker with a higher prevalence for inactivation (e.g., via mutation or deletion) in a single cancer will provide a larger patient subset that will receive a therapeutic benefit when administered a therapy that induces a PKMYT1 loss of function (e.g., via genetic knockout using CRISPR Cas9 or pharmacological inhibition). Additionally, and without being bound by theory, a gene A biomarker that is prevalent in more than one cancer type will enable use of a therapy that induces a PKMYT1 loss of function (e.g., via genetic knockout using CRISPR Cas9 or pharmacological inhibition) for treatment of multiple cancers.

The 15 validated biomarkers were also considered for potential mechanisms of synthetic lethality with PKMYT1. As previously described in PCT/US2021/25230, which is herein incorporated by reference, protein phosphatase 2 regulatory subunit A alpha (PPP2R2A) was identified as a gene A biomarker that is a synthetic lethal pair with PKMYT1. As shown in FIG. 2, PKMYT1 is a checkpoint kinase involved in cell cycle regulation and PPP2R2A is part of a complex that is involved in DNA repair. Without being bound by theory, loss of PPP2R2A function is expected to enhance the effect of PKMYT1 inhibition. Additionally, and without being bound by theory, loss of PPP2R2A function is expected to increase the level of DNA damage in tumor cells, which will synergize with a PKMYT1 loss of function by driving damaged cells into cell cycle and mitotic catastrophe, resulting in cell death. Similar or distinct mechanisms of synthetic lethality are anticipated for loss of function in gene A biomarkers following inhibition of PKMYT1 function. Interestingly, PPP2R1B, PPP3CC, and ATM identified in Table 8 are gene A biomarkers that are involved in DNA repair. Without being bound by theory, mutations in these biomarkers are expected to increase the level of DNA damage in tumor cells, resulting in uncontrolled cell cycle progression subsequent to the loss of function of PKMYT1 (e.g., by genetic knockout or pharmacological inhibition).

### Example 4—Validation of Predicted Biomarkers and PKMYT1 as Synthetic Lethality Pairs

The CombiGEM methods described in Example 2 were used to validate putative biomarkers as synthetic lethal pairs with PKMYT1. A gene A sgRNA lentiviral library and a gene A*B sgRNA lentiviral library were developed, in which the gene A sgRNA library targets certain biomarkers identified in Example 1 and gene B sgRNA targets PKMYT1. The barcoded sgRNA lentiviral libraries were evaluated in a Cas9-expressing colon cancer cell line (LS180) or in a Cas9-expressing ovarian cancer cell line (PA1). Lentiviral constructs encoding the PKMYT1 sgRNA were also evaluated in Cas9-expressing HT29 and LS180 cell lines. Following culture, the cells were harvested and LFC and GI values were determined as described in Example 2.

The LFC values for LS180 cells transfected with PKMYT1-targeting sgRNA were −0.12 and −1.47 respectively. Shown in Tables 9 and 10 are the LFC values for a double-knockout of the gene A biomarker and PKMYT1 and the GI score as measured in LS180 cells or PA1 cells respectively. Also included as a gene A biomarker was PPP2R2A (as previously described in PCT/US2021/25230, for forming a synthetic lethal pair with PKMYT1). PPP2R2A was observed to have a LFC (gene A*B) and GI respectively of −3.41 and −1.02 in LS180 cells and of −3.48 and −2.19 in PA1 cells.

TABLE 9
Effect of Biomarker Knockdown
in PKMYT1 Knockout LS180 Cells

| Biomarker | LFC (gene A*B) | GI |
|---|---|---|
| CSMD3 | −1.82 | −1.08 |
| ATM | −2.26 | −1.00 |
| ARID1A | −2.82 | −1.41 |
| PCDH15 | −1.81 | −0.97 |
| UNC13C | −1.75 | −1.13 |
| CSGALNACT1 | −1.86 | −1.02 |
| EGR3 | −1.79 | −1.05 |
| LRP1B | −1.67 | −1.05 |
| CACNA1E | −1.77 | −1.32 |
| VPS13B | −1.83 | −1.06 |
| DOCK3 | −1.69 | −1.00 |
| CHMP7 | −4.13 | −1.21 |
| SETBP1 | −2.13 | −1.00 |
| DYSF | −2.00 | −1.26 |
| XPO7 | −1.82 | −1.08 |
| VPS37A | −2.63 | −1.15 |
| FAM86B2 | −2.10 | −0.97 |
| ACVR2A | −1.96 | −1.12 |
| XKR5 | −1.84 | −1.03 |
| SCN1A | −1.71 | −1.07 |
| TRPS1 | −1.65 | −0.99 |
| DEFA1 | −2.36 | −1.43 |
| DEFB103B | −2.17 | −1.16 |
| NIPBL | −2.80 | −1.12 |
| ASPM | −1.98 | −1.16 |
| TENM1 | −1.86 | −1.01 |
| DMXL2 | −2.10 | −1.26 |
| FN1 | −1.43 | −1.00 |
| CHD1 | −2.37 | −1.14 |
| CDH10 | −2.08 | −1.12 |
| LRRC7 | −1.67 | −0.95 |
| PRKDC | −3.27 | −1.26 |
| ASH1L | −2.50 | −1.33 |
| CHD4 | −2.46 | −1.10 |
| SVIL | −1.76 | −1.09 |
| NALCN | −1.70 | −1.05 |
| ARID2 | −2.64 | −1.19 |
| CHD8 | −2.48 | −0.97 |
| FBN2 | −2.21 | −1.25 |
| FER | −2.20 | −1.08 |
| CHRNA2 | −1.96 | −1.15 |
| USP34 | −1.76 | −1.02 |
| ABCA12 | −1.69 | −0.97 |
| INTS10 | −3.49 | −1.14 |
| TNKS | −1.53 | −1.22 |
| MTUS1 | −1.87 | −1.11 |
| SLC7A2 | −1.58 | −1.04 |
| RNF43 | −1.58 | −1.01 |
| ERICH1 | −1.72 | −0.98 |
| ASAH1 | −1.67 | −1.00 |
| ZDHHC2 | −1.92 | −1.25 |
| CNOT7 | −1.57 | −0.99 |
| ENTPD4 | −1.85 | −0.95 |
| SH2D4A | −1.82 | −0.99 |
| MTMR9 | −2.28 | −1.09 |
| FHOD3 | −1.86 | −1.02 |
| AGPAT5 | −1.72 | −1.22 |
| SPAG11A | −1.68 | −0.99 |
| PDLIM2 | −2.15 | −1.21 |
| GFRA2 | −1.88 | −1.07 |
| MSRA | −1.82 | −1.06 |
| LGI3 | −2.26 | −1.33 |
| REEP4 | −2.19 | −1.24 |
| PDGFRL | −1.73 | −1.11 |
| HR | −1.72 | −1.00 |
| TNFRSF10D | −2.17 | −1.15 |
| DMTN | −2.08 | −1.27 |
| PEBP4 | −2.02 | −1.15 |
| FZD3 | −2.00 | −1.23 |
| TNFRSF10B | −1.89 | −1.16 |
| DEFB136 | −1.71 | −1.10 |
| PPP1R3B | −1.56 | −1.08 |
| SMAD2 | −1.98 | −1.03 |
| DNAH3 | −1.71 | −0.99 |
| DEFA5 | −1.67 | −1.07 |
| EPHX2 | −1.93 | −1.09 |
| TNFRSF10C | −1.71 | −1.03 |
| PNMA2 | −1.87 | −1.04 |
| LARP4B | −1.46 | −1.10 |
| TRIM35 | −2.31 | −1.33 |
| INTS9 | −2.32 | −1.00 |
| CDH19 | −1.75 | −1.05 |
| MYH3 | −1.73 | −1.05 |
| CSMD2 | −2.39 | −1.13 |
| ZFHX4 | −2.20 | −1.29 |
| SARAF | −2.13 | −1.15 |
| PBK | −2.02 | −1.24 |
| RELN | −1.92 | −1.19 |
| PRKG1 | −2.01 | −1.03 |
| UBXN8 | −1.99 | −1.03 |
| EPHA3 | −1.97 | −1.18 |
| FSTL5 | −1.91 | −1.10 |
| CALD1 | −1.88 | −1.04 |
| SMIM18 | −1.84 | −1.05 |
| FAT1 | −1.78 | −0.95 |
| WWC2 | −1.76 | −1.00 |
| ADAMTS20 | −1.76 | −1.14 |
| KLKB1 | −1.63 | −0.96 |
| OCA2 | −1.35 | −1.06 |
| TLL1 | −1.98 | −1.14 |
| CCDC73 | −1.98 | −1.29 |
| TSSK1B | −2.41 | −1.57 |
| DNAH8 | −2.21 | −1.34 |
| MAP3K1 | −2.14 | −1.31 |
| AKAP9 | −2.07 | −1.24 |
| PLXND1 | −1.98 | −1.24 |
| AKAP12 | −1.98 | −1.25 |
| GABRA5 | −1.81 | −0.95 |
| PCDH9 | −2.16 | −1.32 |
| ROCK1 | −2.09 | −1.20 |
| SMCHD1 | −2.03 | −1.35 |
| SCN2A | −1.89 | −1.10 |
| TLR3 | −1.75 | −1.09 |
| STOX2 | −1.53 | −1.09 |
| MIDEAS | −1.44 | −0.98 |
| HERC2 | −2.09 | −1.04 |
| MCTP1 | −2.03 | −1.26 |
| NRXN2 | −2.00 | −1.00 |
| GALR1 | −1.93 | −1.35 |
| DNAJC13 | −1.83 | −1.18 |
| RAD51AP2 | −1.78 | −0.95 |
| TG | −1.78 | −1.23 |
| NF1 | −1.74 | −1.27 |
| SNX25 | −1.69 | −1.25 |
| CWF19L2 | −2.93 | −1.05 |
| FAN1 | −2.57 | −1.56 |
| ANK1 | −2.43 | −1.10 |
| TRIP11 | −2.23 | −1.57 |
| KIF20B | −2.23 | −1.21 |
| RNF111 | −2.09 | −1.19 |
| MBD1 | −2.07 | −1.26 |
| PCDH19 | −2.06 | −1.22 |
| PTPRD | −2.04 | −1.21 |
| ADGRB3 | −1.74 | −1.16 |
| SLC8A2 | −1.60 | −1.38 |
| PLD2 | −1.45 | −0.97 |
| GLI3 | −1.31 | −1.09 |
| ULK2 | −2.53 | −1.47 |
| KDSR | −2.39 | −1.07 |
| SLC25A46 | −2.16 | −1.28 |
| DYM | −2.12 | −1.01 |
| CD58 | −2.12 | −1.03 |
| CFAP53 | −2.04 | −1.36 |
| ADGRL2 | −2.01 | −1.18 |
| TP73 | −1.90 | −1.23 |
| SLC12A6 | −1.89 | −0.98 |
| MYO5A | −1.85 | −1.13 |
| MON2 | −1.81 | −1.18 |
| KCNA5 | −1.77 | −1.01 |
| KCNN2 | −1.75 | −1.12 |
| SLC16A1 | −1.69 | −1.01 |
| FAM193A | −1.67 | −1.03 |
| CDKN2AIP | −1.67 | −1.06 |
| CNTN5 | −1.58 | −0.95 |
| TSLP | −2.38 | −1.47 |
| ZADH2 | −2.19 | −1.32 |
| GABRB3 | −2.15 | −1.10 |
| CTNNA1 | −2.09 | −1.05 |
| TGM7 | −2.05 | −0.96 |
| NUDT12 | −2.03 | −1.05 |
| PCNX1 | −2.02 | −1.40 |
| MYO18A | −1.98 | −1.07 |
| FRMD4A | −1.96 | −1.31 |
| SHPK | −1.94 | −1.21 |
| LSAMP | −1.94 | −1.11 |
| SLC66A2 | −1.92 | −1.31 |
| ADCY2 | −1.90 | −1.06 |
| CCDC68 | −1.73 | −1.08 |
| CTNNA3 | −1.72 | −1.12 |
| TRPA1 | −1.59 | −1.07 |
| PPFIA2 | −1.51 | −1.02 |
| SLK | −1.48 | −1.01 |
| PLK1 | −4.22 | −0.99 |
| PAXBP1 | −2.98 | −1.23 |
| ELL2 | −2.38 | −1.31 |
| EFNA5 | −2.16 | −1.24 |
| ENC1 | −2.16 | −1.22 |
| LTK | −2.14 | −1.29 |
| UBE3A | −2.11 | −0.99 |
| WNK4 | −2.07 | −1.15 |
| HTR1E | −2.06 | −1.24 |
| RBM10 | −2.03 | −0.99 |
| PLA2G4D | −1.98 | −1.11 |
| ADRB3 | −1.97 | −1.28 |
| DDHD2 | −1.96 | −1.07 |
| CTR9 | −1.95 | −1.14 |
| CELF4 | −1.92 | −1.00 |
| ATP10D | −1.92 | −1.34 |
| RNF152 | −1.90 | −1.02 |
| ACAP1 | −1.89 | −1.12 |
| RWDD4 | −1.86 | −1.14 |
| BAZ1A | −1.79 | −1.11 |
| CLASP1 | −1.79 | −1.02 |
| PIK3R6 | −1.78 | −1.07 |
| GUCY1A1 | −1.76 | −1.17 |
| NRXN3 | −1.76 | −1.19 |
| MSH2 | −1.75 | −1.17 |
| AR | −1.73 | −1.01 |
| SLC8A1 | −1.61 | −0.95 |
| STARD4 | −1.57 | −1.05 |
| TTK | −3.32 | −1.30 |
| DHX15 | −2.84 | −1.09 |
| HASPIN | −2.51 | −1.26 |
| EPHA6 | −2.48 | −1.11 |
| SUPT16H | −2.40 | −1.54 |
| SNTG1 | −2.39 | −1.13 |
| ANKRD37 | −2.26 | −1.34 |
| SLITRK1 | −2.23 | −1.40 |
| SCAPER | −2.16 | −1.26 |
| ATP2B2 | −2.14 | −1.30 |
| MLH3 | −2.13 | −1.29 |
| PLA2R1 | −2.06 | −1.14 |
| RASGRF2 | −2.05 | −1.01 |
| GCNT4 | −2.04 | −1.31 |
| ACADVL | −2.02 | −1.33 |
| GP1BA | −2.02 | −1.19 |
| NSD1 | −2.00 | −1.07 |
| ARHGAP22 | −1.99 | −1.13 |
| TMEM94 | −1.95 | −1.23 |
| BNC1 | −1.94 | −1.20 |
| CKAP5 | −1.93 | −1.03 |
| PHLDB2 | −1.86 | −1.03 |
| ITGA10 | −1.86 | −1.13 |
| ADAM10 | −1.85 | −1.01 |
| MCF2L | −1.82 | −1.12 |
| KCND3 | −1.80 | −1.19 |
| HELT | −1.76 | −1.17 |
| CBLN2 | −1.76 | −0.97 |
| PJA2 | −1.75 | −1.43 |
| ENO3 | −1.75 | −0.95 |
| CCDC88C | −1.73 | −1.11 |
| MTNR1A | −1.66 | −1.03 |
| TBX3 | −1.58 | −1.00 |
| ATR | −3.31 | −1.23 |
| POLR2B | −2.67 | −0.96 |
| BAG4 | −2.36 | −1.34 |
| MINPP1 | −2.28 | −1.29 |
| EFR3A | −2.21 | −1.30 |
| DISP2 | −2.18 | −1.35 |
| ZNFX1 | −2.14 | −1.00 |
| DLEC1 | −2.12 | −1.29 |
| KIF3A | −2.10 | −1.26 |
| UNC13A | −2.07 | −1.03 |
| GON4L | −2.05 | −1.13 |
| PDLIM3 | −1.99 | −1.16 |
| CCDC80 | −1.98 | −1.00 |
| GUCY1B1 | −1.95 | −1.15 |
| JHY | −1.88 | −1.12 |
| ZFYVE28 | −1.86 | −1.10 |
| HRH2 | −1.84 | −1.01 |
| MARVELD2 | −1.83 | −1.03 |
| PLCB2 | −1.82 | −0.98 |
| HPSE2 | −1.82 | −1.06 |
| TMEM62 | −1.81 | −1.09 |
| KCND2 | −1.80 | −0.98 |
| PHF14 | −1.76 | −1.08 |
| MYT1L | −1.68 | −1.12 |
| MTMR10 | −1.67 | −1.01 |
| DAAM1 | −1.64 | −0.96 |
| HPS1 | −1.64 | −1.02 |
| SASH1 | −1.63 | −1.04 |
| FRG1 | −1.58 | −0.95 |
| MCF2L2 | −1.57 | −1.17 |
| CC2D2B | −1.54 | −1.21 |
| CRMP1 | −1.42 | −0.99 |
| USP48 | −2.87 | −1.09 |
| SOS1 | −2.55 | −1.19 |
| TM2D2 | −2.53 | −1.07 |
| CD109 | −2.52 | −1.24 |
| EPSTI1 | −2.48 | −1.63 |
| ADCY5 | −2.30 | −0.97 |
| CHTF18 | −2.28 | −1.14 |
| SMG6 | −2.22 | −1.22 |
| ERBB3 | −2.18 | −1.11 |
| BRSK2 | −2.12 | −1.21 |
| NLGN2 | −2.08 | −1.34 |
| VEGFC | −2.05 | −1.22 |
| ADAMTS1 | −2.03 | −1.09 |
| TYRO3 | −2.01 | −1.35 |
| TRIM69 | −1.99 | −1.31 |
| PLPP5 | −1.99 | −1.08 |
| UPF3A | −1.95 | −1.55 |
| MFSD4B | −1.94 | −1.04 |
| FNIP1 | −1.94 | −1.25 |
| UHRF2 | −1.92 | −1.08 |
| ZNF418 | −1.92 | −0.95 |
| LRFN5 | −1.91 | −1.11 |
| GJD2 | −1.91 | −1.04 |
| PARP14 | −1.88 | −1.06 |
| R3HCC1L | −1.86 | −1.37 |
| APBB1 | −1.85 | −1.03 |
| EVI5 | −1.82 | −1.03 |
| MYEF2 | −1.78 | −1.00 |
| IL16 | −1.78 | −1.07 |
| MCTP2 | −1.76 | −1.05 |
| HTRA4 | −1.75 | −1.28 |
| BRPF3 | −1.73 | −1.10 |
| HDLBP | −1.73 | −0.95 |
| RAB3C | −1.72 | −0.95 |
| ITGA2 | −1.65 | −0.96 |
| FRMD5 | −1.64 | −1.16 |
| IFIH1 | −1.59 | −1.34 |
| CYB5A | −1.58 | −1.11 |
| CP | −1.53 | −1.22 |
| MCM10 | −3.45 | −1.07 |
| SUPT5H | −2.92 | −1.38 |
| MATK | −2.76 | −1.49 |
| PLK2 | −2.65 | −1.11 |
| KLF3 | −2.55 | −1.04 |
| ADD1 | −2.46 | −1.33 |
| HDAC2 | −2.41 | −1.35 |
| ARHGAP33 | −2.23 | −1.08 |
| PTPN4 | −2.21 | −1.34 |
| HSPA4L | −2.21 | −1.33 |
| SYNRG | −2.20 | −1.37 |
| ZMYM2 | −2.16 | −1.07 |
| BMF | −2.10 | −1.14 |
| AMBRA1 | −2.05 | −1.02 |
| TMEM144 | −2.04 | −1.09 |
| SENP7 | −2.04 | −1.23 |
| EVI5L | −2.02 | −1.18 |
| SPATA22 | −2.02 | −1.07 |
| TRIM45 | −1.97 | −1.21 |
| FAM172A | −1.95 | −1.38 |
| KIF2A | −1.95 | −1.17 |
| CDO1 | −1.94 | −1.06 |
| KCNG4 | −1.93 | −1.14 |
| BRSK1 | −1.92 | −1.09 |
| SLC15A1 | −1.92 | −1.09 |
| OPN4 | −1.92 | −1.33 |
| POU3F2 | −1.88 | −1.12 |
| POU5F2 | −1.88 | −1.04 |
| HECA | −1.87 | −1.16 |
| DUSP16 | −1.85 | −1.02 |
| SLC4A1 | −1.83 | −1.13 |
| MCCC2 | −1.83 | −0.98 |
| VWA5A | −1.76 | −1.16 |
| TTLL7 | −1.74 | −1.28 |
| MID1 | −1.73 | −1.05 |
| HTR1B | −1.72 | −1.09 |
| LAMB1 | −1.69 | −1.06 |
| CSNK1G3 | −1.64 | −1.12 |
| ADAL | −1.63 | −1.09 |
| RHOBTB3 | −1.60 | −1.17 |
| KLF13 | −1.60 | −1.03 |
| DVL1 | −1.59 | −1.03 |
| MFAP1 | −4.34 | −1.04 |
| MTREX | −3.68 | −0.97 |
| BUB1B | −3.25 | −1.24 |
| IPO7 | −3.25 | −0.98 |
| WDHD1 | −3.21 | −1.04 |
| UTP4 | −2.92 | −1.17 |
| CHP1 | −2.76 | −1.33 |
| USP45 | −2.37 | −1.17 |
| NAA15 | −2.31 | −1.10 |
| GAN | −2.23 | −1.10 |
| RSBN1 | −2.19 | −1.30 |
| KCNA2 | −2.19 | −1.29 |
| ACOT12 | −2.17 | −1.34 |
| KIF6 | −2.15 | −1.28 |
| NLRP12 | −2.15 | −1.15 |
| STK10 | −2.11 | −1.30 |
| HCN1 | −2.10 | −1.26 |
| SMG1 | −2.09 | −1.10 |
| KIF5C | −2.08 | −1.29 |
| PKD2L1 | −2.05 | −1.21 |
| RBL2 | −2.05 | −1.06 |
| GATM | −2.01 | −1.18 |
| WDR6 | −2.01 | −1.10 |
| ASB11 | −2.00 | −1.27 |
| MELK | −2.00 | −1.18 |
| KCTD8 | −1.97 | −1.14 |
| TMC1 | −1.96 | −1.20 |
| CXorf58 | −1.86 | −1.09 |
| GREM1 | −1.86 | −1.11 |
| CEMIP | −1.86 | −1.22 |
| HDAC5 | −1.84 | −1.01 |
| FLT1 | −1.84 | −1.00 |
| ANO5 | −1.83 | −1.37 |
| CHERP | −1.82 | −1.18 |
| NEIL3 | −1.81 | −0.98 |
| YBX2 | −1.81 | −0.97 |
| MTMR1 | −1.78 | −1.42 |
| KLHL2 | −1.77 | −1.07 |
| KCNAB2 | −1.77 | −0.97 |
| NUDT7 | −1.72 | −1.11 |
| GAPVD1 | −1.70 | −1.09 |
| SMYD4 | −1.70 | −1.05 |
| NLRP2 | −1.69 | −1.09 |
| MAMLD1 | −1.67 | −1.04 |
| SHOC1 | −1.64 | −1.09 |
| CSTF2T | −1.62 | −1.09 |
| TCEANC | −1.56 | −1.00 |
| KIF4A | −3.66 | −1.19 |
| PDS5A | −3.40 | −1.45 |
| DUSP22 | −3.03 | −1.25 |
| LARP1 | −2.98 | −1.14 |
| NUP88 | −2.90 | −1.06 |
| SPAG5 | −2.86 | −1.12 |
| MYO6 | −2.62 | −1.57 |
| AKT1 | −2.56 | −1.17 |
| ANKRD52 | −2.53 | −1.17 |
| CDC20B | −2.42 | −1.01 |
| NSUN2 | −2.41 | −1.42 |
| GBE1 | −2.40 | −1.35 |
| HAPLN1 | −2.34 | −1.38 |
| NAA16 | −2.32 | −1.22 |
| ALAS1 | −2.25 | −1.04 |
| ARSA | −2.23 | −1.28 |
| TNFRSF9 | −2.19 | −1.02 |
| PTPN21 | −2.18 | −1.18 |
| SLC16A10 | −2.18 | −1.17 |
| TRAPPC13 | −2.14 | −1.16 |
| RBM15B | −2.13 | −1.12 |
| NFIB | −2.12 | −1.39 |
| BCKDHB | −2.11 | −1.69 |
| SMOC2 | −2.10 | −1.33 |
| CDK11B | −2.10 | −1.35 |
| TUBB8 | −2.09 | −1.10 |
| KIFC1 | −2.09 | −1.30 |
| PPP2R2B | −2.08 | −1.24 |
| CPEB4 | −2.08 | −1.24 |
| CCNA1 | −2.08 | −1.23 |
| KLHDC4 | −2.07 | −1.30 |
| INPP5A | −2.07 | −1.23 |
| PUDP | −2.05 | −1.12 |
| DEPDC1B | −2.05 | −1.15 |
| TNPO2 | −2.04 | −1.10 |
| MICU2 | −1.99 | −1.21 |
| PDE4B | −1.99 | −1.32 |
| CDC25A | −1.99 | −1.01 |
| TBR1 | −1.98 | −1.22 |
| GLT8D1 | −1.95 | −1.18 |
| LRCH3 | −1.95 | −1.11 |
| GSPT2 | −1.93 | −1.11 |
| GALK2 | −1.93 | −1.13 |
| RPGR | −1.91 | −1.27 |
| SLITRK6 | −1.90 | −1.07 |
| TENT5A | −1.90 | −1.15 |
| NPAP1 | −1.89 | −1.29 |
| SLC6A2 | −1.87 | −1.14 |
| TIGD4 | −1.86 | −1.14 |
| HPGD | −1.86 | −1.05 |
| EIF4A1 | −1.86 | −1.01 |
| PRPF40A | −1.85 | −1.18 |
| ZNF528 | −1.81 | −1.29 |
| ARHGAP18 | −1.81 | −1.06 |
| NEK4 | −1.79 | −1.31 |
| VAMP3 | −1.77 | −0.99 |
| VCL | −1.76 | −1.36 |
| PDE5A | −1.76 | −1.05 |
| ZNF180 | −1.72 | −1.12 |
| DGKI | −1.71 | −1.34 |
| CDK14 | −1.65 | −0.95 |
| TAB2 | −1.64 | −1.08 |
| MKNK2 | −1.63 | −0.98 |
| TNFSF12 | −1.60 | −1.01 |
| TMEM25 | −1.59 | −1.07 |
| ATXN3L | −1.56 | −0.98 |
| MYBPC1 | −1.55 | −0.98 |
| MANF | −1.54 | −1.00 |
| CDH1 | −1.50 | −1.05 |
| CENPH | −3.10 | −1.27 |
| OTUD5 | −3.00 | −1.06 |
| ZBTB7A | −2.92 | −1.31 |
| PDHA1 | −2.78 | −1.03 |
| ADCY4 | −2.76 | −0.97 |
| CCNF | −2.54 | −1.30 |
| MAD1L1 | −2.45 | −1.27 |
| IWS1 | −2.41 | −1.05 |
| REC8 | −2.40 | −1.20 |
| NFIC | −2.37 | −1.39 |
| CCNB2 | −2.36 | −1.06 |
| NAF1 | −2.35 | −1.37 |
| HTR2A | −2.31 | −0.99 |
| ECEL1 | −2.31 | −1.15 |
| PIK3C2A | −2.31 | −1.47 |
| KIF15 | −2.28 | −1.03 |
| MSH4 | −2.24 | −1.28 |
| TRMU | −2.19 | −1.15 |
| KLHL15 | −2.19 | −1.03 |
| DYRK1A | −2.16 | −1.25 |
| CH25H | −2.16 | −1.32 |
| PRKCZ | −2.15 | −1.44 |
| PRKCD | −2.15 | −1.03 |
| GPR63 | −2.15 | −1.16 |
| TCIM | −2.11 | −1.33 |
| GBX2 | −2.09 | −1.07 |
| CAMKK1 | −2.07 | −1.36 |
| CADM2 | −2.07 | −1.31 |
| TBC1D22A | −2.07 | −0.97 |
| MMP10 | −2.06 | −1.23 |
| ING5 | −2.05 | −1.20 |
| BICC1 | −2.04 | −1.25 |
| HEY2 | −2.04 | −1.18 |
| SEC24D | −2.04 | −1.20 |
| PPIL6 | −2.04 | −1.27 |
| SRSF4 | −2.03 | −1.18 |
| CDKN2B | −2.02 | −0.96 |
| RXFP2 | −2.00 | −1.08 |
| GJA3 | −1.99 | −1.21 |
| ERMARD | −1.99 | −1.33 |
| AHI1 | −1.96 | −1.31 |
| ANXA10 | −1.94 | −1.11 |
| UGT2A1 | −1.94 | −1.11 |
| TRMT11 | −1.89 | −1.05 |
| UNC93A | −1.89 | −1.06 |
| EPB41L2 | −1.86 | −1.15 |
| SLC6A3 | −1.86 | −1.17 |
| ZNF471 | −1.83 | −1.05 |
| ZNF853 | −1.81 | −1.01 |
| TGIF2LX | −1.80 | −0.96 |
| HDAC1 | −1.78 | −1.32 |
| CDHR2 | −1.78 | −1.16 |
| ZNF777 | −1.78 | −1.28 |
| AMELX | −1.78 | −1.02 |
| ZBTB12 | −1.77 | −1.07 |
| KCTD19 | −1.76 | −1.00 |
| CTTNBP2NL | −1.75 | −1.15 |
| GLOD4 | −1.74 | −0.98 |
| CTBP2 | −1.73 | −1.06 |
| OR51E2 | −1.71 | −1.02 |
| HTR2B | −1.68 | −0.98 |
| PDGFD | −1.67 | −0.98 |
| WDR37 | −1.61 | −0.97 |
| STAG2 | −1.58 | −1.00 |
| GNB1 | −1.56 | −0.95 |
| E2F2 | −1.55 | −1.07 |
| AGPAT3 | −1.54 | −1.04 |
| DHX37 | −4.30 | −1.09 |
| CCNH | −3.38 | −1.09 |
| POLR3A | −3.35 | −1.18 |
| ORC1 | −3.34 | −1.09 |
| MYC | −3.28 | −1.10 |
| PTBP1 | −3.08 | −1.08 |
| HSP90AB1 | −2.79 | −1.20 |
| ALG5 | −2.78 | −0.99 |
| INTS6 | −2.62 | −1.02 |
| ADCY6 | −2.50 | −1.14 |
| RHOA | −2.49 | −0.98 |
| TDRD3 | −2.48 | −1.28 |
| RBM5 | −2.46 | −1.19 |
| ATP1A3 | −2.39 | −1.16 |
| FMNL1 | −2.28 | −1.14 |
| DMRTA1 | −2.28 | −1.31 |
| WEE2 | −2.27 | −1.00 |
| COQ3 | −2.27 | −0.95 |
| STIM1 | −2.26 | −1.51 |
| HOOK3 | −2.25 | −1.27 |
| RRM2 | −2.22 | −1.28 |
| CPEB1 | −2.20 | −1.37 |
| OLFM3 | −2.20 | −1.18 |
| MAPK8 | −2.19 | −1.27 |
| EXO1 | −2.16 | −1.29 |
| NACC1 | −2.14 | −1.27 |
| FOXO3 | −2.12 | −1.19 |
| NT5DC1 | −2.10 | −1.28 |
| TPD52L1 | −2.10 | −1.27 |
| LMOD3 | −2.10 | −1.19 |
| MAPK11 | −2.08 | −1.01 |
| BANK1 | −2.07 | −1.24 |
| EPS8 | −2.05 | −1.22 |
| PDE6B | −2.03 | −1.28 |
| THEG | −2.03 | −1.50 |
| LARP1B | −2.03 | −1.16 |
| HAUS6 | −2.03 | −1.06 |
| GRAMD1B | −2.03 | −1.27 |
| MSMO1 | −2.03 | −1.13 |
| MROH2B | −2.01 | −1.26 |
| AGO2 | −2.00 | −0.99 |
| ATMIN | −1.99 | −0.99 |
| SLC30A5 | −1.98 | −1.02 |
| FAM234B | −1.97 | −1.07 |
| AADAT | −1.94 | −1.13 |
| DCAF12L1 | −1.93 | −1.10 |
| ATG16L1 | −1.90 | −1.21 |
| GLB1 | −1.90 | −1.15 |
| SUMF1 | −1.89 | −1.30 |
| SEC24C | −1.89 | −1.07 |
| FAM120B | −1.88 | −1.06 |
| TRAP1 | −1.88 | −1.12 |
| IL18 | −1.88 | −1.19 |
| CPTP | −1.87 | −1.16 |
| SMPD2 | −1.87 | −1.19 |
| C11orf87 | −1.86 | −1.33 |
| SLC9A1 | −1.84 | −1.05 |
| GPR82 | −1.80 | −1.16 |
| INTU | −1.80 | −1.17 |
| NTNG1 | −1.79 | −1.10 |
| CBR4 | −1.78 | −1.04 |
| SETMAR | −1.77 | −1.19 |
| EDNRB | −1.77 | −1.02 |
| CHAF1B | −1.76 | −0.96 |
| KLHL18 | −1.75 | −1.24 |
| CPSF6 | −1.75 | −0.98 |
| SETDB2 | −1.74 | −1.03 |
| FAM155A | −1.74 | −1.22 |
| UBE2J2 | −1.73 | −1.05 |
| MGAT5 | −1.72 | −1.15 |
| LONP2 | −1.72 | −1.11 |
| C11orf65 | −1.70 | −0.99 |
| SLC22A14 | −1.69 | −1.16 |
| CNR1 | −1.68 | −1.12 |
| CDC34 | −1.67 | −1.20 |
| GLB1L3 | −1.67 | −1.07 |
| POLN | −1.66 | −1.03 |
| FTSJ1 | −1.65 | −1.12 |
| STK25 | −1.61 | −1.01 |
| PPID | −1.59 | −1.11 |
| SIK3 | −1.57 | −0.97 |
| TCP1 | −4.04 | −1.08 |
| SRPRA | −3.01 | −1.13 |
| RAD21 | −2.79 | −1.14 |
| ESRP2 | −2.72 | −1.44 |
| GJB7 | −2.53 | −1.55 |
| GLRX3 | −2.48 | −1.21 |
| EPM2AIP1 | −2.45 | −1.29 |
| DFFB | −2.43 | −1.38 |
| MOS | −2.41 | −1.17 |
| SFN | −2.39 | −1.44 |
| SLC37A2 | −2.39 | −1.03 |
| SMIM15 | −2.32 | −1.16 |
| NHLRC3 | −2.31 | −1.39 |
| LACC1 | −2.30 | −1.02 |
| CDKN1B | −2.26 | −1.27 |
| MIOX | −2.25 | −1.17 |
| PTH1R | −2.22 | −1.22 |
| METTL6 | −2.22 | −1.36 |
| CCR2 | −2.21 | −1.23 |
| FGF22 | −2.21 | −1.29 |
| SMARCAL1 | −2.20 | −1.08 |
| REEP6 | −2.19 | −1.14 |
| NR2C2 | −2.19 | −1.31 |
| FAF1 | −2.18 | −1.08 |
| APP | −2.17 | −1.16 |
| VPS36 | −2.14 | −0.98 |
| CDH5 | −2.12 | −1.29 |
| ASF1A | −2.11 | −1.43 |
| PAK3 | −2.09 | −1.07 |
| RAB39A | −2.09 | −1.21 |
| C11orf53 | −2.08 | −1.18 |
| SLC39A12 | −2.07 | −1.27 |
| ARL8B | −2.06 | −1.14 |
| ELOVL4 | −2.05 | −1.25 |
| ZNF821 | −2.05 | −1.31 |
| ARPP19 | −2.03 | −1.13 |
| ATXN7 | −2.03 | −1.21 |
| ACAA1 | −2.02 | −1.18 |
| AFG1L | −2.01 | −1.16 |
| TENT4B | −2.01 | −1.07 |
| C16orf46 | −2.00 | −1.13 |
| ZDHHC3 | −1.99 | −1.00 |
| KIF2C | −1.98 | −1.37 |
| FMR1 | −1.97 | −1.09 |
| ACTR3B | −1.97 | −1.30 |
| ZRSR2 | −1.96 | −1.10 |
| SERP2 | −1.95 | −1.17 |
| C6orf118 | −1.94 | −1.23 |
| CCR5 | −1.94 | −1.14 |
| PIM3 | −1.91 | −1.29 |
| ATG5 | −1.90 | −0.97 |
| RER1 | −1.89 | −1.06 |
| RPS6KA1 | −1.88 | −1.02 |
| BTD | −1.87 | −1.15 |
| DDX19B | −1.86 | −1.04 |
| VPS11 | −1.84 | −0.99 |
| KCNV1 | −1.82 | −0.98 |
| PUS3 | −1.82 | −1.27 |
| COTL1 | −1.80 | −0.99 |
| AKAP7 | −1.77 | −1.06 |
| CER1 | −1.77 | −1.21 |
| SIDT2 | −1.74 | −0.96 |
| SLC16A14 | −1.71 | −1.15 |
| PLCD1 | −1.71 | −1.07 |
| FOXO1 | −1.71 | −1.07 |
| THRB | −1.68 | −1.09 |
| CALHM4 | −1.68 | −0.98 |
| BIRC3 | −1.67 | −1.04 |
| USP27X | −1.67 | −0.99 |
| JAML | −1.67 | −0.98 |
| CXorf38 | −1.65 | −1.12 |
| SLC22A2 | −1.65 | −1.08 |
| MAB21L1 | −1.62 | −0.95 |
| SGK1 | −1.59 | −1.18 |
| ELAVL2 | −1.59 | −0.96 |
| FBLN1 | −1.58 | −1.25 |
| HSP90AA1 | −1.54 | −1.20 |
| WTAP | −1.45 | −0.95 |
| GAL3ST2 | −1.39 | −1.06 |
| WEE1 | −3.90 | −0.99 |
| CCNA2 | −3.35 | −0.97 |
| MCM5 | −3.18 | −1.15 |
| MAD2L1 | −3.00 | −1.13 |
| UQCRC1 | −2.97 | −1.34 |
| NCLN | −2.63 | −1.20 |
| PPP1R7 | −2.63 | −1.19 |
| CXCR5 | −2.45 | −1.20 |
| TBRG1 | −2.40 | −1.36 |
| GOLGA7 | −2.33 | −1.26 |
| ELMO3 | −2.29 | −1.25 |
| VPS4A | −2.24 | −1.27 |
| RWDD1 | −2.19 | −1.26 |
| TMEM171 | −2.14 | −1.21 |
| TTC21A | −2.09 | −1.25 |
| SHISA5 | −2.08 | −1.25 |
| TBX22 | −2.07 | −1.24 |
| MTRF1 | −2.05 | −1.01 |
| PPP2R2D | −2.04 | −1.09 |
| GZMM | −2.02 | −1.15 |
| GNA15 | −2.00 | −1.23 |
| TSSK2 | −1.98 | −1.35 |
| NCKIPSD | −1.97 | −1.21 |
| LRRC3B | −1.97 | −1.17 |
| VPS26B | −1.96 | −1.04 |
| C11orf1 | −1.96 | −1.14 |
| RCBTB1 | −1.95 | −1.11 |
| FAM3B | −1.93 | −1.10 |
| APLP2 | −1.93 | −1.18 |
| N4BP2L1 | −1.92 | −1.09 |
| ZNF35 | −1.92 | −1.03 |
| SMIM2 | −1.91 | −1.15 |
| COLQ | −1.91 | −1.04 |
| TCAIM | −1.91 | −1.26 |
| SLC6A1 | −1.90 | −1.11 |
| ECT2L | −1.89 | −1.02 |
| DBNDD1 | −1.88 | −1.05 |
| CENPBD1 | −1.87 | −1.07 |
| C6orf58 | −1.85 | −1.37 |
| ZNF470 | −1.85 | −0.99 |
| NEK3 | −1.84 | −1.05 |
| ALB | −1.83 | −1.28 |
| PLN | −1.82 | −1.54 |
| UBP1 | −1.82 | −1.00 |
| SERINC5 | −1.81 | −0.97 |
| SAG | −1.81 | −1.14 |
| PHF10 | −1.80 | −1.12 |
| PLAA | −1.79 | −0.96 |
| OR51I2 | −1.77 | −1.03 |
| TAGAP | −1.77 | −1.03 |
| OXNAD1 | −1.74 | −1.28 |
| DUSP7 | −1.74 | −1.04 |
| SPIRE2 | −1.73 | −1.13 |
| THSD1 | −1.71 | −1.05 |
| CREB3L3 | −1.66 | −0.98 |
| NR1D2 | −1.64 | −0.99 |
| CES2 | −1.56 | −1.01 |
| UTP15 | −1.54 | −1.08 |
| ZNF555 | −1.46 | −1.15 |
| MTSS2 | −1.37 | −1.00 |
| TAMM41 | −4.20 | −1.06 |
| C16orf95 | −2.61 | −1.16 |
| DYNLRB2 | −2.52 | −1.04 |
| ZNF501 | −2.39 | −1.14 |
| BOK | −2.34 | −1.33 |
| CPNE7 | −2.32 | −1.03 |
| FAM107A | −2.24 | −1.17 |
| XCR1 | −2.21 | −1.12 |
| THUMPD3 | −2.17 | −1.34 |
| FDX1 | −2.14 | −1.06 |
| HOMER1 | −2.10 | −1.20 |
| ZNRF1 | −2.10 | −1.26 |
| BNIP3 | −2.09 | −1.26 |
| RNF170 | −2.09 | −1.19 |
| GADD45B | −2.08 | −1.10 |
| DEF8 | −2.06 | −1.08 |
| TPD52L3 | −2.01 | −1.03 |
| UCN2 | −2.01 | −1.17 |
| ATG4B | −2.01 | −1.05 |
| FUOM | −1.99 | −1.16 |
| PRAMEF8 | −1.99 | −1.16 |
| HSF2 | −1.98 | −1.18 |
| AMIGO3 | −1.97 | −1.32 |
| DRC7 | −1.97 | −1.13 |
| SYCE1L | −1.97 | −1.18 |
| SKP1 | −1.97 | −1.20 |
| NOVA2 | −1.97 | −1.06 |
| TBCEL | −1.96 | −1.06 |
| TMEM181 | −1.94 | −0.98 |
| F2RL2 | −1.93 | −1.09 |
| GPR35 | −1.91 | −1.17 |
| ACER2 | −1.86 | −1.09 |
| EIF4E3 | −1.85 | −1.21 |
| SFT2D1 | −1.85 | −1.09 |
| GABARAPL2 | −1.84 | −1.25 |
| SLN | −1.83 | −1.00 |
| FAM3D | −1.83 | −1.00 |
| PRKACA | −1.83 | −1.15 |
| UPK3A | −1.80 | −0.99 |
| CTAG1B | −1.80 | −1.08 |
| POU2AF1 | −1.73 | −1.04 |
| FAM124B | −1.73 | −1.00 |
| HYLS1 | −1.71 | −0.98 |
| ZCWPW2 | −1.67 | −1.03 |
| KIAA0513 | −1.63 | −0.97 |
| CASP8AP2 | −1.62 | −1.09 |
| ACRV1 | −1.59 | −1.04 |
| SMPDL3A | −1.51 | −1.00 |
| TIMM8B | −2.40 | −1.53 |
| E2F4 | −2.40 | −1.57 |
| PPP4R2 | −2.26 | −1.47 |
| TLCD5 | −2.26 | −1.08 |
| RRAGA | −2.17 | −1.21 |
| SCN4B | −2.14 | −1.28 |
| PRAP1 | −2.08 | −1.21 |
| CSRNP1 | −2.06 | −1.19 |
| LTF | −2.05 | −1.42 |
| SNORC | −2.02 | −1.31 |
| KCTD12 | −2.01 | −1.09 |
| PHF11 | −1.98 | −1.09 |
| TMEM170A | −1.98 | −1.32 |
| MYL3 | −1.94 | −1.05 |
| MAPK1 | −1.90 | −1.22 |
| TP53AIP1 | −1.90 | −1.19 |
| SPRN | −1.90 | −1.25 |
| PRR5 | −1.84 | −1.00 |
| FRG2C | −1.83 | −1.00 |
| GTSE1 | −1.83 | −1.22 |
| IFT46 | −1.79 | −1.01 |
| MEMO1 | −1.79 | −0.96 |
| C3orf62 | −1.79 | −0.98 |
| PFKFB4 | −1.78 | −0.95 |
| DLEU7 | −1.76 | −1.17 |
| STK11IP | −1.76 | −0.99 |
| MMP12 | −1.75 | −0.97 |
| TMIE | −1.74 | −1.01 |
| USP2 | −1.72 | −1.00 |
| IGF1 | −1.68 | −1.07 |
| ALG9 | −1.66 | −0.96 |
| EBLN1 | −1.56 | −1.01 |
| UCHL3 | −1.45 | −1.16 |
| RBX1 | −2.36 | −0.98 |
| COMMD6 | −2.34 | −1.47 |
| ESAM | −2.27 | −1.06 |
| C11orf45 | −2.23 | −1.13 |
| MRGPRG | −2.19 | −1.03 |
| PTPRE | −2.10 | −1.07 |
| BARX2 | −2.04 | −1.37 |
| POU4F1 | −1.93 | −1.17 |
| CCNG1 | −1.90 | −1.28 |
| FBXL3 | −1.90 | −1.16 |
| NANS | −1.87 | −1.16 |
| MOB3B | −1.81 | −1.02 |
| MPZL2 | −1.77 | −1.11 |
| FBXO30 | −1.69 | −1.08 |
| CXCR6 | −1.44 | −0.97 |
| PPP3CC | −3.28 | −0.98 |
| PPP2R1B | −4.16 | −1.19 |

TABLE 10
Effect of Biomarker Knockdown in PKMYT1 Knockout PA1 Cells

| Biomarker | LFC | GI |
|---|---|---|
| EGR3 | −2.14 | −2.14 |
| CACNA1E | −2.01 | −2.01 |
| PCDH15 | −1.73 | −1.73 |
| VPS13B | −1.34 | −1.34 |
| CSGALNACT1 | −1.28 | −1.28 |
| UNC13C | −1.09 | −1.09 |
| CHMP7 | −2.70 | −2.14 |
| DYSF | −2.19 | −1.27 |
| CSMD3 | −1.63 | −1.63 |
| ARID2 | −1.54 | −1.50 |
| PRKDC | −3.14 | −1.30 |
| DEFA1 | −2.23 | −2.12 |
| GTSE1 | −2.11 | −2.11 |
| ARID1A | −1.83 | −1.83 |
| SMARCA4 | −2.81 | −1.35 |
| TRMU | −2.30 | −2.16 |
| PTPRD | −2.15 | −2.15 |
| NIPBL | −2.52 | −1.73 |
| ANK1 | −2.51 | −1.73 |
| ASPM | −2.47 | −2.32 |
| EPHA3 | −2.41 | −2.17 |
| TLL1 | −2.36 | −1.46 |
| DMRTA1 | −2.33 | −2.33 |
| CHD1 | −2.33 | −2.03 |
| RYR2 | −2.31 | −1.28 |
| CHD4 | −2.23 | −1.82 |
| NALCN | −2.11 | −1.61 |
| FBN2 | −2.04 | −1.63 |
| NFIB | −2.03 | −2.03 |
| VPS37A | −2.01 | −2.01 |
| NF1 | −2.01 | −1.26 |
| MTNR1A | −1.94 | −1.77 |
| DMXL1 | −1.94 | −1.33 |
| FER | −1.92 | −1.92 |
| DEFB103B | −1.91 | −1.65 |
| PRR5 | −1.91 | −1.91 |
| HERC2 | −1.91 | −1.91 |
| SETBP1 | −1.85 | −1.58 |
| TENM1 | −1.84 | −1.84 |
| NRXN3 | −1.81 | −1.81 |
| PCDH9 | −1.80 | −1.54 |
| KDM6A | −1.79 | −1.79 |
| TRPS1 | −1.78 | −1.68 |
| CDH1 | −1.76 | −1.76 |
| ASH1L | −1.75 | −1.75 |
| MAP2K4 | −1.75 | −1.43 |
| PLAA | −1.73 | −1.65 |
| SMC1B | −1.73 | −1.73 |
| DOCK3 | −1.70 | −1.70 |
| IFT74 | −1.69 | −1.41 |
| MAPK11 | −1.67 | −1.58 |
| EPHA6 | −1.65 | −1.65 |
| FAM86B2 | −1.65 | −1.65 |
| ADAMTS20 | −1.64 | −1.64 |
| CSMD2 | −1.62 | −1.62 |
| PPFIA2 | −1.62 | −1.62 |
| WWC2 | −1.61 | −1.61 |
| SI | −1.61 | −1.61 |
| SCN2A | −1.60 | −1.60 |
| USP34 | −1.56 | −1.56 |
| MDGA2 | −1.56 | −1.45 |
| CDKN2B | −1.54 | −1.51 |
| RBM10 | −1.54 | −1.26 |
| SCN1A | −1.53 | −1.53 |
| TG | −1.51 | −1.51 |
| HCN1 | −1.49 | −1.49 |
| NBEA | −1.49 | −1.49 |
| SPEF2 | −1.41 | −1.06 |
| SCN3A | −1.41 | −1.41 |
| BNC2 | −1.41 | −1.41 |
| XKR5 | −1.40 | −1.35 |
| SNX25 | −1.40 | −1.40 |
| ELAVL2 | −1.39 | −1.39 |
| ACVR2A | −1.38 | −1.32 |
| HECW1 | −1.38 | −1.38 |
| CTNND2 | −1.36 | −1.36 |
| SVIL | −1.35 | −1.26 |
| CTNNA3 | −1.35 | −1.35 |
| FN1 | −1.35 | −1.34 |
| ALB | −1.30 | −1.30 |
| DMXL2 | −1.27 | −1.27 |
| ZFP36L2 | −1.21 | −1.21 |
| CDH10 | −1.21 | −1.21 |
| PPP6R2 | −1.20 | −1.20 |
| SCARA5 | −1.19 | −1.19 |
| LSAMP | −1.19 | −1.19 |
| CHD8 | −1.11 | −1.11 |
| LRRC7 | −1.10 | −1.10 |
| CHRNA2 | −1.09 | −1.09 |
| KLKB1 | −1.06 | −1.06 |
| ZFHX4 | −1.02 | −1.02 |
| OR4F21 | −1.96 | −1.14 |
| FAT1 | −1.81 | −1.81 |
| TBC1D22A | −1.73 | −1.07 |
| SNTG1 | −1.72 | −1.58 |
| RELN | −1.67 | −1.63 |
| NPAP1 | −1.65 | −1.50 |
| BNIP3L | −1.62 | −1.62 |
| GABRB3 | −1.62 | −1.27 |
| CADM2 | −1.58 | −1.58 |
| C9orf72 | −1.47 | −1.47 |
| TCF4 | −1.44 | −1.44 |
| UPK3A | −1.43 | −1.43 |
| MPDZ | −1.43 | −1.43 |
| FSTL5 | −1.37 | −1.37 |
| NFASC | −1.33 | −1.28 |
| ABCA12 | −1.28 | −1.28 |
| CNTN3 | −1.28 | −1.28 |
| LRFN5 | −1.25 | −1.25 |
| LINGO2 | −1.24 | −1.24 |
| PKHD1L1 | −1.08 | −1.08 |

What is claimed:

1. A method of identifying a subject having a disease or disorder for treatment with one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of biomarkers listed in Table 3.

2. A method of determining responsiveness of a subject having a disease or disorder to one or more PKMYT1 therapeutic agents, the method comprising determining the presence of a mutation in, the expression level of, and/or the activity of one or more biomarkers in a diseased tissue sample obtained from the subject, wherein the one or more biomarkers is selected from any one or any combination of biomarkers listed in Table 3.

3. The method of claim 1 or 2, wherein the one or more biomarkers is selected from any one or any combination of biomarkers listed in Table 8-10.

4. The method of any one of claims 1-3, wherein the diseased tissue sample comprises an altered expression level and/or activity of the one or more biomarkers relative to a reference tissue sample.

5. The method of claim 4, wherein the expression level and/or activity of the one or more biomarkers is reduced relative to a reference tissue sample.

6. The method of any one of claims 1-5, wherein the diseased tissue sample comprises a mutation in the one or more biomarkers relative to a reference tissue sample.

7. The method of any one of claims 1-6, wherein the mutation is a loss of function mutation, optionally wherein the loss of function mutation is a deletion of the gene encoding the biomarker.

8. The method of any one of claims 1-7, wherein the mutation is detected by sequencing genomic DNA in the diseased tissue sample, optionally via next generation sequencing.

9. The method of any one of claims 1-8, wherein the subject has a tumor, and wherein the diseased tissue sample comprises a tumor sample, a circulating tumor DNA sample, a tumor biopsy sample, or a fixed tumor sample.

10. The method of claim 9, wherein the tumor comprises a plurality of tumor cells comprising the mutation.

11. The method of any one of claims 1-10, wherein the one or more biomarkers comprises 2, 3, 4, 5, or more biomarkers selected from Table 3 and a PP2 subunit.

12. The method of claim 11, wherein the PP2 subunit is PPP2R1B.

13. The method of claim 11 or 12, wherein the PP2 subunit is PPP2R2A.

14. The method of any one of claims 1-13, further comprising administering one or more PKMYT1 therapeutic agents to the subject.

15. The method of claim 14, wherein the administering results in a reduced expression level and/or activity of PKMYT1 in a tumor of the subject.

16. The method of claim 15, wherein the reduced expression level and/or activity of PKMYT1 induces synthetic lethality in the tumor.

17. The method of claim 16, wherein the synthetic lethality promotes tumor regression.

18. A method of treating a cancer or promoting tumor regression in a subject having a tumor comprising a mutation in, an altered expression level of, and/or an altered activity of one or more biomarkers, wherein the one or more biomarkers is selected from any one or any combination of biomarkers listed in Table 3, the method comprising: administering to the subject a therapeutically effective amount of one or more protein kinase, membrane associated tyrosine/threonine 1 (PKMYT1) therapeutic agents.

19. The method of claim 18, wherein the tumor comprises a loss of function mutation in, a reduced expression level of, and/or a reduced activity of the one or more biomarkers as measured in a tumor sample obtained from the subject relative to a reference tissue sample.

20. A method of identifying a cancer subject to receive one or more PKMYT1 therapeutic agents, comprising
(i) determining the presence of a mutation in, the expression level of, and/or the activity of one or more biomarkers in a tumor sample obtained from the subject, wherein the one or more biomarkers are selected from any one or any combination of biomarkers listed in Table 3; and
(ii) administering one or more PKMYT1 therapeutic agents to the subject based on presence of a mutation in, a reduced expression level of, and/or a reduced activity of the one or more biomarkers relative to a healthy control.

21. The method of any one of claims 18-20, wherein the one or more biomarkers is selected from any one or any combination of biomarkers listed in Table 8 and 9.

22. The method of any one of claims 18-20, wherein the one or more biomarkers is selected from any one or any combination of biomarkers listed in Table 10.

23. The method of any one of claims 19-22, wherein the tumor sample is a circulating tumor DNA sample, a tumor biopsy sample, or a fixed tumor sample.

24. The method of any one of claims 19-23, wherein the tumor sample comprises a mutation in the one or more biomarkers.

25. The method of any one of claims 18-24, wherein the mutation is a loss of function mutation, optionally wherein the loss of function mutation is a deletion of the gene encoding the biomarker.

26. The method of any one of claims 18-25, wherein the mutation is detected by sequencing genomic tumor DNA, optionally via next generation sequencing.

27. The method of any one of claims 19-26, wherein the tumor sample comprises a plurality of tumor cells comprising the mutation.

28. The method of any one of claims 18-27, wherein the one or more biomarkers comprises 2, 3, 4, 5, or more biomarkers selected from Table 3 and a PP2 subunit.

29. The method of claim 28, wherein the PP2 subunit is PPP2R1B.

30. The method of claim 28 or 29, wherein the PP2 subunit is PPP2R2A.

31. The method of any one of claims 18-30, wherein the administering results in a reduced expression level and/or activity of PKMYT1 in a tumor of the subject.

32. The method of claim 31, wherein the reduced expression level and/or activity of PKMYT1 induces synthetic lethality in the tumor.

33. The method of claim 32, wherein the synthetic lethality promotes tumor regression.

34. The method of any one of claims 1-33, wherein the one or more PKMYT1 therapeutic agents is selected from a small molecule, a peptide, a protein, and a nucleic acid.

35. The method of claim 34, wherein the one or more PKMYT1 therapeutic agents comprises an anti-PKMYT1 antibody or fragment thereof.

36. The method of claim 34, wherein the one or more PKMYT1 therapeutic agents comprises an anti-PKMYT1 intrabody or fragment thereof.

37. The method of claim 34, wherein the one or more PKMYT1 therapeutic agents comprises an RNAi molecule or an aptamer.

38. The method of claim 34, wherein the one or more PKMYT1 therapeutic agents comprises a small molecule inhibitor.

39. The method of claim 38, wherein the small molecule inhibitor is selected from 5-((5-methoxy-2-((4-morpholinophenyl)amino)pyrimidin-4-yl)amino)-2-methylphenol, iV-(2-chloro-6-methylphenyl)-2-((6-(4-(2-hydroxyethyl)piperazin-1-yl)-2-methylpyrimidin-4-yl)amino)thiazole-5-carboxamide (dasatinib), 4-((2,4-dichloro-5-methoxyphenyl)amino)-6-methoxy-7-(3-(4-methylpiperazin-1-yl)propoxy)quinoline-3-carbonitrile (bosutinib), A-(5-chlorobenzo[t/][1,3]dioxol-4-yl)-7-(2-(4-methylpiperazin-1-yl)ethoxy)-5-((tetrahydro-2//-pyran-4-yl)oxy)quinazolin-4-amine (saracatinib), (£)-A-(4-((3-chloro-4-fluorophenyl)amino)-3-cyano-7-ethoxyquinolin-6-yl)-4-(dimethylamino)but-2-enamide(pelitinib), A-(3-chlorophenyl)-6,7-dimethoxyquinazolin-4-amine (tyrphostin AG 1478), 6-(2,6-dichlorophenyl)-2-((4-(2-(diethylamino)ethoxy)phenyl)amino)-8-methylpyrido[2,3-<i]pyrimidin-7(8//)-one (PD-0166285), 6-(2,6-dichlorophenyl)-8-methyl-2-((4-morpholinophenyl)amino)pyrido[2,3-cf]pyrimidin-7(8//)-one (PD-173952), 6-(2,6-dichlorophenyl)-8-methyl-2-((3-(methylthio)phenyl)amino)pyrido[2,3-r/Jpyrimidin-7(8//)-one (PD-173955), and 6-(2,6-dichlorophenyl)-2-((4-fluoro-3-methylphenyl)amino)-8-methylpyrido[2,3-«i]pyrimidin-7(8//)-one (PD-180970).

40. The method of claim 34, wherein the one or more PKMYT1 therapeutic agents comprises a gene editing technology for introducing a genetic knockout of the PKMYT1 gene.

41. The method of claim 40, wherein the gene editing technology comprises CRISPR/Cas9.

42. The method of any one of claims 9-41, wherein the cancer is selected from: acute myeloid leukemia (LAML), adrenocortical carcinoma (ACC), bladder urothelial carcinoma (BLCA), brain lower grade glioma (LGG), breast invasive carcinoma (BRCA), cervical squamous cell carcinoma and endocervical adenocarcinoma (CESC), cholangiocarcinoma (CHOL), chronic myelogenous leukemia (LCML), colon adenocarcinoma (COAD), esophageal carcinoma (ESCA), glioblastoma multiforme (GBM), head and neck squamous cell carcinoma (HNSC), kidney chromophobe (KICH), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), lymphoid neoplasm diffuse large B-cell lymphoma (DLBC), mesothelioma (MESO), ovarian serous cystadenocarcinoma (OV), pancreatic adenocarcinoma (PAAD), pheochromocytoma and paraganglioma (PCPG), prostate adenocarcinoma (PRAD), rectum adenocarcinoma (READ), sarcoma (SARC), skin cutaneous melanoma (SKCM), testicular germ cell tumors (TGCT), thymoma (THYM), thyroid carcinoma (THCA), uterine carcinosarcoma (UCS), uterine corpus endometrial carcinoma (UCEC), and uveal melanoma (UVM).

43. Use of one or more PKMYT1 therapeutic agents for treating a cancer or promoting tumor regression in a subject, wherein the subject has been identified based on the presence of a mutation in, an altered expression level and/or altered activity of one or more biomarkers, wherein the one or more biomarkers is selected from any one or any combination of biomarkers listed in Table 3.

44. Use of one or more PKMYT1 therapeutic agents in the manufacture of a medicament for treating a cancer or promoting tumor regression in a subject, wherein the subject has been identified based on the presence of a mutation in, an altered expression level and/or altered activity of one or more biomarkers, wherein the one or more biomarkers is selected from any one or any combination of biomarkers listed in Table 3.

45. A kit comprising a PKMYT1 therapeutic agent, and a package insert comprising instructions for administering the PKMYT1 therapeutic agent to a subject having a cancer comprising a mutation in, an altered expression level and/or altered activity of one or more biomarkers, wherein the one or more biomarkers is selected from any one or any combination of biomarkers listed in Table 3.
